SEQUENCE LISTING (238 sequences)

SEQ ID NO: 1 - protein, 64 aa, Artificial Sequence
  Synthetic Construct
EDCNELPPRRNTEILTGSWSDQTYPEGTQAIYKCRPGYRSLGNVIMVCRKGEWVALNPLRKCQK

SEQ ID NO: 2 - protein, 61 aa, Artificial Sequence
  Synthetic Construct
RPCGHPGDTPFGTFTLTGGNVFEYGVKAVYTCNEGYQLLGEINYRECDTDGWTNDIPICEV

SEQ ID NO: 3 - protein, 64 aa, Artificial Sequence
  Synthetic Construct
VKCLPVTAPENGKIVSSAMEPDREYHFGQAVRFVCNSGYKIEGDEEMHCSDDGFWSKEKPKCVE

SEQ ID NO: 4 - protein, 57 aa, Artificial Sequence
  Synthetic Construct
ISCKSPDVINGSPISQKIIYKENERFQYKCNMGYEYSERGDAVCTESGWRPLPSCEE

SEQ ID NO: 5 - protein, 59 aa, Artificial Sequence
  Synthetic Construct
KSCDNPYIPNGDYSPLRIKHRTGDEITYQCRNGFYPATRGNTAKCTSTGWIPAPRCTLK

SEQ ID NO: 6 - protein, 59 aa, Artificial Sequence
  Synthetic Construct
GKCGPPPPIDNGDITSFPLSVYAPASSVEYQCQNLYQLEGNKRITCRNGQWSEPPKCLH

SEQ ID NO: 7 - protein, 66 aa, Artificial Sequence
  Synthetic Construct
PCVISREIMENYNIALRWTAKQKLYSRTGESVEFVCKRGYRLSSRSHTLRTTCWDGKLEYPTCAKR

SEQ ID NO: 8 - protein, 64 aa, Artificial Sequence
  Synthetic Construct
ISCGSPPPILNGRISYYSTPIAVGTVIRYSCSGTFRLIGEKSLLCITKDKVDGTWDKPAPKCEY

SEQ ID NO: 9 - protein, 67 aa, Artificial Sequence
  Synthetic Construct
FNKYSSCPEPIVPGGYKIRGSTPYRHGDSVTFACKTNFSMNGNKSVWCQANNMWGPTRLPTCVSVFP

SEQ ID NO: 10 - protein, 64 aa, Artificial Sequence
  Synthetic Construct
VFPLECPALPMIHNGHHTSENVGSIAPGLSVTYSCESGYLLVGEKIINCLSSGKWSAVPPTCEE

SEQ ID NO: 11 - protein, 61 aa, Artificial Sequence
  Synthetic Construct
ARCKSLGRFPNGKVKEPPILRVGVTANFFCDEGYRLQGPPSSRCVIAGQGVAWTKMPVCEE

SEQ ID NO: 12 - protein, 11 aa, Artificial Sequence
  Synthetic Construct
DICLPRWGCLW

SEQ ID NO: 13 - protein, 5 aa, Artificial Sequence
  Synthetic Construct
GGGGA

SEQ ID NO: 14 - protein, 15 aa, Artificial Sequence
  Synthetic Construct
GGGGAGGGGAGGGGS

SEQ ID NO: 15 - protein, 8 aa, Artificial Sequence
  Synthetic Construct
GGGGSDAA

SEQ ID NO: 16 - protein, 5 aa, Artificial Sequence
  Synthetic Construct
GGGGS

SEQ ID NO: 17 - protein, 10 aa, Artificial Sequence
  Synthetic Construct
GGGGSGGGGS

SEQ ID NO: 18 - protein, 15 aa, Artificial Sequence
  Synthetic Construct
GGGGSGGGGSGGGGS

SEQ ID NO: 19 - protein, 20 aa, Artificial Sequence
  Synthetic Construct
GGGGSGGGGSGGGGSGGGGS

SEQ ID NO: 20 - protein, 25 aa, Artificial Sequence
  Synthetic Construct
GGGGSGGGGSGGGGSGGGGSGGGGS

SEQ ID NO: 21 - protein, 30 aa, Artificial Sequence
  Synthetic Construct
GGGGSGGGGSGGGGSGGGGSGGGGSGGGGS

SEQ ID NO: 22 - protein, 15 aa, Artificial Sequence
  Synthetic Construct
EAAAKEAAAKEAAAK

SEQ ID NO: 23 - protein, 5 aa, Artificial Sequence
  Synthetic Construct
PAPAP

SEQ ID NO: 24 - protein, 10 aa, Artificial Sequence
  Synthetic Construct
GGGGSPAPAP

SEQ ID NO: 25 - protein, 10 aa, Artificial Sequence
  Synthetic Construct
PAPAPGGGGS

SEQ ID NO: 26 - protein, 12 aa, Artificial Sequence
  Synthetic Construct
GSTSGKSSEGKG

SEQ ID NO: 27 - protein, 10 aa, Artificial Sequence
  Synthetic Construct
GGGDSGGGDS

SEQ ID NO: 28 - protein, 10 aa, Artificial Sequence
  Synthetic Construct
GGGESGGGES

SEQ ID NO: 29 - protein, 10 aa, Artificial Sequence
  Synthetic Construct
GGGDSGGGGS

SEQ ID NO: 30 - protein, 10 aa, Artificial Sequence
  Synthetic Construct
GGGASGGGGS

SEQ ID NO: 31 - protein, 10 aa, Artificial Sequence
  Synthetic Construct
GGGESGGGGS

SEQ ID NO: 32 - protein, 6 aa, Artificial Sequence
  Synthetic Construct
ASTKGP

SEQ ID NO: 33 - protein, 13 aa, Artificial Sequence
  Synthetic Construct
ASTKGPSVFPLAP

SEQ ID NO: 34 - protein, 4 aa, Artificial Sequence
  Synthetic Construct
GGGP

SEQ ID NO: 35 - protein, 8 aa, Artificial Sequence
  Synthetic Construct
GGGGGGGP

SEQ ID NO: 36 - protein, 9 aa, Artificial Sequence
  Synthetic Construct
PAPNLLGGP

SEQ ID NO: 37 - protein, 6 aa, Artificial Sequence
  Synthetic Construct
GGGGGG

SEQ ID NO: 38 - protein, 12 aa, Artificial Sequence
  Synthetic Construct
GGGGGGGGGGGG

SEQ ID NO: 39 - protein, 8 aa, Artificial Sequence
  Synthetic Construct
APELPGGP

SEQ ID NO: 40 - protein, 8 aa, Artificial Sequence
  Synthetic Construct
SEPQPQPG

SEQ ID NO: 41 - protein, 15 aa, Artificial Sequence
  Synthetic Construct
GGGSSGGGSSGGGSS

SEQ ID NO: 42 - protein, 14 aa, Artificial Sequence
  Synthetic Construct
GGGGGGGGGSGGGS

SEQ ID NO: 43 - protein, 15 aa, Artificial Sequence
  Synthetic Construct
GGGGSGGGGGGGGGS

SEQ ID NO: 44 - protein, 15 aa, Artificial Sequence
  Synthetic Construct
GGSSSGGSSSGGSSS

SEQ ID NO: 45 - protein, 15 aa, Artificial Sequence
  Synthetic Construct
GSSSSGSSSSGSSSS

SEQ ID NO: 46 - protein, 15 aa, Artificial Sequence
  Synthetic Construct
GGGGAGGGGSGGGGS

SEQ ID NO: 47 - protein, 14 aa, Artificial Sequence
  Synthetic Construct
GGGGSGGGGAGGGG

SEQ ID NO: 48 - protein, 15 aa, Artificial Sequence
  Synthetic Construct
GGGASGGGGSGGGGS

SEQ ID NO: 49 - protein, 15 aa, Artificial Sequence
  Synthetic Construct
GGGGSGGGASGGGGS

SEQ ID NO: 50 - protein, 15 aa, Artificial Sequence
  Synthetic Construct
GGGGSAGGGSGGGGS

SEQ ID NO: 51 - protein, 15 aa, Artificial Sequence
  Synthetic Construct
GGGGSGGGGSAGGGS

SEQ ID NO: 52 - protein, 15 aa, Artificial Sequence
  Synthetic Construct
GGGGSAGGGSAGGGS

SEQ ID NO: 53 - protein, 15 aa, Artificial Sequence
  Synthetic Construct
GGGGDGGGGSGGGGS

SEQ ID NO: 54 - protein, 15 aa, Artificial Sequence
  Synthetic Construct
GGGGSGGGGDGGGGS

SEQ ID NO: 55 - protein, 15 aa, Artificial Sequence
  Synthetic Construct
GGGGDGGGGDGGGGS

SEQ ID NO: 56 - protein, 15 aa, Artificial Sequence
  Synthetic Construct
GGGGEGGGGSGGGGS

SEQ ID NO: 57 - protein, 15 aa, Artificial Sequence
  Synthetic Construct
GGGGSGGGGEGGGGS

SEQ ID NO: 58 - protein, 15 aa, Artificial Sequence
  Synthetic Construct
GGGGEGGGGEGGGGS

SEQ ID NO: 59 - protein, 18 aa, Artificial Sequence
  Synthetic Construct
KESGSVSSEQLAQFRSLD

SEQ ID NO: 60 - protein, 14 aa, Artificial Sequence
  Synthetic Construct
EGKSSGSGSESKST

SEQ ID NO: 61 - protein, 8 aa, Artificial Sequence
  Synthetic Construct
GGGGGGGG

SEQ ID NO: 62 - protein, 12 aa, Artificial Sequence
  Synthetic Construct
GSAGSAAGSGEF

SEQ ID NO: 63 - protein, 6 aa, Artificial Sequence
  Synthetic Construct
GGGGGG

SEQ ID NO: 64 - protein, 7 aa, Artificial Sequence
  Synthetic Construct
  MISC_FEATURE (2)..(6): The group of amino acids at positions 2-6 (“the group”) in this sequence can be repeated x amount of times, where x is any natural number. The Ala currently at position 7 will always follow the last amino acid of the last repetition of the group.
AEAAAKA

SEQ ID NO: 65 - protein, 20 aa, Artificial Sequence
  Synthetic Construct
LEAGCKNFFPRSFTSCGSLE

SEQ ID NO: 66 - protein, 4 aa, Artificial Sequence
  Synthetic Construct
GSST

SEQ ID NO: 67 - protein, 12 aa, Artificial Sequence
  Synthetic Construct
CRRRRRREAEAC

SEQ ID NO: 68 - protein, 4 aa, Artificial Sequence
  Synthetic Construct
GSGS

SEQ ID NO: 69 - protein, 6 aa, Artificial Sequence
  Synthetic Construct
GSGSGS

SEQ ID NO: 70 - protein, 8 aa, Artificial Sequence
  Synthetic Construct
GSGSGSGS

SEQ ID NO: 71 - protein, 10 aa, Artificial Sequence
  Synthetic Construct
GSGSGSGSGS

SEQ ID NO: 72 - protein, 12 aa, Artificial Sequence
  Synthetic Construct
GSGSGSGSGSGS

SEQ ID NO: 73 - protein, 6 aa, Artificial Sequence
  Synthetic Construct
GGSGGS

SEQ ID NO: 74 - protein, 9 aa, Artificial Sequence
  Synthetic Construct
GGSGGSGGS

SEQ ID NO: 75 - protein, 12 aa, Artificial Sequence
  Synthetic Construct
GGSGGSGGSGGS

SEQ ID NO: 76 - protein, 4 aa, Artificial Sequence
  Synthetic Construct
GGSG

SEQ ID NO: 77 - protein, 8 aa, Artificial Sequence
  Synthetic Construct
GGSGGGSG

SEQ ID NO: 78 - protein, 12 aa, Artificial Sequence
  Synthetic Construct
GGSGGGSGGGSG

SEQ ID NO: 79 - protein, 19 aa, Artificial Sequence
  Synthetic Construct
GGGGAGGGGAGGGAGGGGS

SEQ ID NO: 80 - protein, 10 aa, Artificial Sequence
  Synthetic Construct
GENLYFQSGG

SEQ ID NO: 81 - protein, 8 aa, Artificial Sequence
  Synthetic Construct
SACYCELS

SEQ ID NO: 82 - protein, 5 aa, Artificial Sequence
  Synthetic Construct
RSIAT

SEQ ID NO: 83 - protein, 17 aa, Artificial Sequence
  Synthetic Construct
RPACKIPNDLKQKVMNH

SEQ ID NO: 84 - protein, 36 aa, Artificial Sequence
  Synthetic Construct
GGSAGGSGSGSSGGSSGASGTGTAGGTGSGSGTGSG

SEQ ID NO: 85 - protein, 17 aa, Artificial Sequence
  Synthetic Construct
AAANSSIDLISVPVDSR

SEQ ID NO: 86 - protein, 36 aa, Artificial Sequence
  Synthetic Construct
GGSGGGSEGGGSEGGGSEGGGSEGGGSEGGGSGGGS

SEQ ID NO: 87 - protein, 15 aa, Artificial Sequence
  Synthetic Construct
GGGGAGGGGAGGGGS

SEQ ID NO: 88 - protein, 223 aa, Artificial Sequence
  Synthetic Construct
VECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 20 aa, Artificial Sequence
  Synthetic Construct
GGGGAGGGGAGGGGAGGGGS

SEQ ID NO: 90 - protein, 23 aa, Artificial Sequence
  Synthetic Construct
DAAGGGGSGGGGSGGGGSGGGGS

SEQ ID NO: 91 - protein, 15 aa, Artificial Sequence
  Synthetic Construct
GGGGAGGGGAGGGGA

SEQ ID NO: 92 - protein, 19 aa, Artificial Sequence
  Synthetic Construct
GGGGAGGGGAGGGAGGGGS

SEQ ID NO: 93 - protein, 18 aa, Artificial Sequence
  Synthetic Construct
GGSSRSSSSGGGGAGGGG

SEQ ID NO: 94 - protein, 253 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEE

SEQ ID NO: 95 - protein, 131 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFP

SEQ ID NO: 96 - protein, 253 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEE

SEQ ID NO: 97 - protein, 253 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFAMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEE

SEQ ID NO: 98 - protein, 253 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFAMNGNKAVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEE

SEQ ID NO: 99 - protein, 253 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKAVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEE

SEQ ID NO: 100 - protein, 253 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTQFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEE

SEQ ID NO: 101 - protein, 131 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTQFSMNGQKSVWCQANNMWGPTRLPTCVSVFP

SEQ ID NO: 102 - protein, 131 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFP

SEQ ID NO: 103 - protein, 131 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFAMNGNKSVWCQANNMWGPTRLPTCVSVFP

SEQ ID NO: 104 - protein, 131 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFAMNGNKAVWCQANNMWGPTRLPTCVSVFP

SEQ ID NO: 105 - protein, 131 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKAVWCQANNMWGPTRLPTCVSVFP

SEQ ID NO: 106 - protein, 131 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTQFSMNGNKSVWCQANNMWGPTRLPTCVSVFP

SEQ ID NO: 107 - protein, 253 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTQFSMNGQKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEE

SEQ ID NO: 108 - protein, 305 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 109 - protein, 248 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 110 - protein, 125 aa, Artificial Sequence
  Synthetic Construct
GKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 111 - protein, 228 aa, Artificial Sequence
  Synthetic Construct
ERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 112 - protein, 229 aa, Artificial Sequence
  Synthetic Construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 232 aa, Artificial Sequence
  Synthetic Construct
AEPKSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 809 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 115 - protein, 653 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 116 - protein, 653 aa, Artificial Sequence
  Synthetic Construct
GKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 117 - protein, 479 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 804 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 119 - protein, 794 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 120 - protein, 476 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 121 - protein, 811 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGAGGGGAGGGGSVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 122 - protein, 811 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKAVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGAGGGGAGGGGSVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 123 - protein, 781 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 124 - protein, 796 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 125 - protein, 428 aa, Artificial Sequence
  Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 126 - protein, 681 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 127 - protein, 691 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 128 - protein, 701 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 129 - protein, 711 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 130 - protein, 721 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSGGGGSGGGGSGGGGSEVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSSGGGGSGGGGSGGGGSGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 131 - protein, 681 aa, Artificial Sequence; identical to SEQ ID NO: 126
  Synthetic Construct

SEQ ID NO: 132 - protein, 811 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGAGGGGAGGGGSVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 133 - protein, 123 aa, Artificial Sequence
  Synthetic Construct
EVQLVESGGGLVKPGGSLRLSCAASGRPVSNYAAAWFRQAPGKEREFVSAINWQKTATYADSVKGRFTISRDNAKNSLYLQMNSLRAEDTAVYYCAAVFRVVAPKTQYDYDYWGQGTLVTVSS

SEQ ID NO: 134 - protein, 373 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 135 - protein, 430 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 136 - protein, 194 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKSVWCQANNMWGPTRLPTCVSVFPLPMIHNGHHTSENVGSIAPGLSVTYSCESGYLLVGEKIINCLSSGKWSAVPPTCEEARCKSLG

SEQ ID NO: 137 - protein, 194 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTQFSMNGQKSVWCQANNMWGPTRLPTCVSVFPLPMIHNGHHTSENVGSIAPGLSVTYSCESGYLLVGEKIINCLSSGKWSAVPPTCEEARCKSLG

SEQ ID NO: 138 - protein, 194 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPLPMIHNGHHTSENVGSIAPGLSVTYSCESGYLLVGEKIINCLSSGKWSAVPPTCEEARCKSLG

SEQ ID NO: 139 - protein, 194 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFAMNGNKAVWCQANNMWGPTRLPTCVSVFPLPMIHNGHHTSENVGSIAPGLSVTYSCESGYLLVGEKIINCLSSGKWSAVPPTCEEARCKSLG

SEQ ID NO: 140 - protein, 194 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGNKAVWCQANNMWGPTRLPTCVSVFPLPMIHNGHHTSENVGSIAPGLSVTYSCESGYLLVGEKIINCLSSGKWSAVPPTCEEARCKSLG

SEQ ID NO: 141 - protein, 194 aa, Artificial Sequence; identical to SEQ ID NO: 137
  Synthetic Construct

SEQ ID NO: 142 - protein, 5 aa, Artificial Sequence
  Synthetic Construct
EAAAK

SEQ ID NO: 143 - protein, 7 aa, Artificial Sequence
  Synthetic Construct
AEAAAKA

SEQ ID NO: 144 - protein, 649 aa, Artificial Sequence
  Synthetic Construct
GKCGPPPPIDNGDITSFPLSVYAPASSVEYQCQNLYQLEGNKRITCRNGQWSEPPKCLHSREIMENYNIALRWTAKQKLYSRTGESVEFVCKRGYRLSSRSHTLRTTCWDGKLEYPTCAKRVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 145 - protein, 649 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGKCGPPPPIDNGDITSFPLSVYAPASSVEYQCQNLYQLEGNKRITCRNGQWSEPPKCLHSREIMENYNIALRWTAKQKLYSRTGESVEFVCKRGYRLSSRSHTLRTTCWDGKLEYPTCAKR

SEQ ID NO: 146 - protein, 543 aa, Artificial Sequence
  Synthetic Construct
VECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 147 - protein, 686 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 148 - protein, 629 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 149 - protein, 552 aa, Artificial Sequence
  Synthetic Construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 150 - protein, 537 aa, Artificial Sequence
  Synthetic Construct
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 151 - protein, 547 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGGGGAGGGGAGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 668 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGGGGAGGGGAGGGGSVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 153 - protein, 668 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 154 - protein, 683 aa, Artificial Sequence
  Synthetic Construct
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKGGGGAGGGGAGGGGSVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 155 - protein, 634 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPGGGGSDAAERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 156 - protein, 491 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 157 - protein, 492 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 158 - protein, 492 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSREDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 159 - protein, 487 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGAGGGGSKEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 160 - protein, 487 aa, Artificial Sequence
  Synthetic Construct
CVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGAGGGGSREDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 161 - protein, 490 aa, Artificial Sequence
  Synthetic Construct
VECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 162 - protein, 362 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 163 - protein, 14 aa, Artificial Sequence
  Synthetic Construct
GGGGAGGGAGGGGS

SEQ ID NO: 164 - protein, 7 aa, Artificial Sequence
  Synthetic Construct
GGGGSDA

SEQ ID NO: 165 - DNA, 2427 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctgatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgagccttggaaaaggtggtggcggatctggcggaggtggaagcggaggcggtggaagtggcggtggtggatctgaggattgcaacgagctgcctcctcggagaaacaccgagatcctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctccacggtgcacactgaaa

SEQ ID NO: 166 - DNA, 2085 bp, Artificial Sequence
  Synthetic Construct
gaggattgcaagggccctccacctagagagaacagcgagatcctgtctggctcttggagcgagcagctgtatcctgagggaacccaggccacctacaagtgcagacctggctacagaaccctgggcaccatcgtgaaagtgtgcaagaacggcaaatgggtcgccagcaatcccagccggatctgcagaaagaaaccttgcggacaccccggcgatacccctttcggatcttttagactggccgtgggcagccagtttgagttcggagccaaggtggtgtacacatgcgacgatggctatcagctgctgggcgagatcgactatagagagtgtggcgccgacggctggatcaacgatatccctctgtgcgaggtggtcaagtgcctgcctgtgacagagctggaaaacggcagaattgtgtccggcgctgccgagacagaccaagagtactactttggccaggtcgtcagattcgagtgcaacagcggcttcaagatcgagggccacaaagagatccactgcagcgagaacggcctgtggtccaacgagaagcccagatgcgtggaaatcctgtgcacccctcctagagtggaaaatggcgacggcatcaacgtgaagcccgtgtacaaagagaacgagcgctaccactataagtgcaagcacggctacgtgcccaaagaacggggagatgccgtgtgtacaggctctggatggtccagccagcctttctgcgaagagaagagatgcagccctccttacatcctgaacggcatctacacccctcaccggatcatccacagaagcgacgacgagatcagatacgagtgtaattacggcttctaccccgtgaccggcagcaccgtgtctaagtgtacacctaccggatggatccccgtgcctagatgtacactgaaaggcggcagcagcagaagcagttcttctggcggaggcggagctggtggtggcggagataagaaaatcgtgcccagagactgcggctgcaagccctgtatctgtacagtgcctgagcagagcagcgtgttcatcttcccacctaagcctaaggacgtgctgatgatcagcctgacacctaaagtgacctgcgtggtggtggacatcagcaaggatgaccctgaggtgcagttcagttggttcgtggacgacgtggaagtgcacacagcccagaccaagccaagagaggaacagatcaacagcaccttcagaagcgtgtccgagctgcccattctgcaccaggactggctgaatggcaaagagttcaagtgtagagtgaactccgccgcttttcccgctcctatcgagaaaaccatctccaagaccaagggcagacccaaggctccccaggtctacacaatccctccaccaaaagaacagatggccaaggacaaggtgtccctgacctgcatgatcaccaatttcttcccagaggacatcaccgtggaatggcagtggaatggacagcccgccgagaactacaagaacacccagcctatcatggacaccgacggcagctacttcgtgtacagcaagctgaacgtgcagaagtccaactgggaggccggcaacacctttacctgttctgtgctgcacgagggcctgcacaaccaccacacagagaagtctctgtctcacagccctggcaaaggcggctctagcagatcttcttcatctggtggcggtggtgccggtggcggcggaggaaaatgtggacctcctcctccaatcgacaacggcgacatcacaagcctgagcctgccagtgtatgagcccctgtctagcgtggaataccagtgccagaagtactacctgctgaagggcaaaaagaccatcacctgtcggaacggcaagtggtccgagcctcctacatgtctgcacgcctgcgtgatccccgagaacatcatggaaagccacaacatcatcctgaagtggcggcacaccgagaagatctacagccactctggcgaggacatcgagttcggctgcaaatacggctactacaaggcccgggatagccctccattccggaccaagtgtatcaacggcaccatcaactaccctacctgcgtc

SEQ ID NO: 167 - DNA, 2085 bp, Artificial Sequence
  Synthetic Construct
ggcaagtgtggacctcctcctcctatcgacaacggcgacatcacaagcctgagcctgcctgtgtatgagcccctgagcagcgtggaataccagtgccagaagtactacctgctgaagggcaagaaaaccatcacctgtcggaacggcaagtggtccgagcctcctacatgtctgcacgcctgcgtgatccccgagaacatcatggaaagccacaacatcatcctgaagtggcggcacaccgagaagatctacagccactctggcgaggacatcgagttcggctgcaaatacggctactacaaggcccgggatagccctccattccggaccaagtgtatcaacggcaccatcaactaccctacctgcgtcggcggcagcagcagatctagttcttctggcggaggcggagctggtggcggcggagataagaaaatcgtgcctagagactgcggctgcaagccctgtatctgtacagtgcctgagcagtccagcgtgttcatcttcccacctaagcctaaggacgtgctgatgatcagcctgacacctaaagtgacctgcgtggtggtggacatcagcaaggatgaccctgaggtgcagttcagttggttcgtggacgacgtggaagtgcacacagcccagaccaagcctagagaggaacagatcaacagcaccttcagaagcgtgtccgagctgcccattctgcaccaggactggctgaacggcaaagagttcaagtgcagagtgaacagcgccgcctttcctgctccaatcgaaaagaccatctccaagaccaagggcagacccaaggctccccaggtgtacacaatccctccacctaaagaacagatggccaaggacaaggtgtccctgacctgcatgatcaccaatttcttcccagaggacatcaccgtggaatggcagtggaatggacagcccgccgagaactacaagaacacccagcctatcatggacaccgacggcagctacttcgtgtacagcaagctgaacgtgcagaagtccaactgggaggccggcaacacctttacctgttctgtgctgcacgagggcctgcacaaccaccacacagagaagtctctgtctcacagccctggcaaaggcggcagctctagaagtagttcaagcggaggtggcggagcaggcggtggtggcgaagattgcaaaggaccaccaccaagagagaacagcgagatcctgtctggctcttggagcgagcagctgtatcctgagggaacccaggccacctacaagtgcaggcctggctatagaaccctgggcaccatcgtgaaagtgtgcaagaatggcaaatgggtcgccagcaatcccagccggatctgcagaaagaaaccttgcggacaccccggcgatacccctttcggatcttttagactggccgtgggcagccagtttgagttcggagccaaggtggtgtatacctgcgacgatggctatcagctgctgggcgagatcgactatagagagtgtggcgccgacggctggatcaacgatatccctctgtgcgaggtggtcaagtgcctgccagtgacagagctggaaaacggcagaattgtgtccggcgctgccgagacagaccaagagtactactttggccaggtcgtcagattcgagtgcaacagcggcttcaagatcgagggccacaaagagatccactgcagcgagaacggcctgtggtccaacgagaagcccagatgcgtggaaatcctgtgcacccctcctagagtggaaaatggcgacggcatcaacgtgaagcccgtgtacaaagagaacgagcgctaccactataagtgcaagcacggctacgtgcccaaagaacggggagatgccgtgtgtacaggctctggatggtccagccagcctttctgcgaagagaagagatgcagccctccttacatcctgaacggaatctacacccctcaccggatcatccacagaagcgacgacgagatcagatacgagtgtaattacggcttctaccccgtgaccggcagcaccgtgtctaagtgtacaccaacaggctggatccccgtgcctcggtgcacactgaaa

SEQ ID NO: 168 - DNA, 2451 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcagcagcagatcttctagttctggcggaggcggagctggtggtggcggagttgaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggcaaaggcggctctagcagaagtagttcttctggcggcggtggtgctggcggcggaggcgaagattgcaatgaactgcctcctcggcggaacaccgagatcttgacaggatcttggagcgaccagacataccctgagggcacccaggccatctacaagtgtagacctggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctcctcggtgcacactgaaa

SEQ ID NO: 169 - DNA, 2397 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctgatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgagccttggaaaaggtggtggcggatctggcggaggtggaagcgaagattgcaacgagctgcctcctcggagaaacaccgagatcctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctccacggtgcacactgaaa

SEQ ID NO: 170 - DNA, 2382 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctgatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgagccttggaaaaggcggaggcggaagcgaggattgcaatgagctgcctcctcggagaaacaccgagatcctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctccacggtgcacactgaaa

SEQ ID NO: 171 - DNA, 2370 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgcgaagaggacgccgccgtggaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgctgtggaatgggagagcaacggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctgggcaaagaggactgcaacgagctgcctcctcggagaaataccgagatcctgaccggctcttggagcgaccagacatatccagaaggcacccaggccatctacaagtgccggcctggatacagatccctgggcaatgtgatcatggtctgccggaaaggcgagtgggttgccctgaatcctctgagaaagtgccagaagaggccttgcggacaccccggcgatacaccttttggcacattcaccctgacaggcggcaatgtgttcgagtatggcgtgaaggccgtgtacacctgtaatgagggctaccagctgctgggcgagatcaactacagagagtgtgataccgacggctggaccaacgacatccctatctgcgaggtggtcaagtgcctgccagtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatccctgctccaagatgcacactgaagcaccaccaccatcaccac

SEQ ID NO: 172 - DNA, 2433 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggaggcggagctggtggtggcggtgctggtggcggaggatctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggaaaaggtggtggcggagctggcggaggtggtgcaggcggtggtggatctgaagattgcaacgagctgcctcctcggcggaataccgagattctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccctgccaccagaggcaacaccgccaagtgtacaagcacaggctggatccccgctcctcggtgtacactgaaa

SEQ ID NO: 173 - DNA, 2433 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaaggccgtgtggtgccaggccaacaatatgtggggacctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggaggcggagctggtggtggcggtgctggtggcggaggatctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggaaaaggtggtggcggagctggcggaggtggtgcaggcggtggtggatctgaagattgcaacgagctgcctcctcggcggaataccgagattctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaagccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccctgccaccagaggcaacaccgccaagtgtacaagcacaggctggatccccgctcctcggtgtacactgaaa

SEQ ID NO: 174 - DNA, 915 bp, Artificial Sequence
  Synthetic Construct
gaagattgcaacgagctgcctcctcggcggaataccgagattctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccctgccaccagaggcaacaccgccaagtgtacaagcacaggctggatccccgctcctcggtgtacactgaaa

SEQ ID NO: 175 - DNA, 1674 bp, Artificial Sequence
  Synthetic Construct
atcagctgcggttcccctccaccaatcctgaatggcagaatctcctattactccacaccaatcgccgtcggcactgtgatcagatacagctgttcagggacttttcggctgatcggcgagaaaagcctcctctgcattaccaaggataaggtcgatgggacatgggataaaccagctcctaagtgcgagtacttcaataagtatagttcatgtccagagcccattgttcctggtggctacaagattcgggggagcacaccctatcgccacggtgactcagtgacctttgcttgtaaaaccaacttctcaatgaacggtaataagtcagtgtggtgtcaggccaataatatgtggggtcctacacgactccccacctgtgtgtccgtgttccccttggaatgccccgccctgcccatgatccataatggacaccacaccagcgagaatgtcgggagtatcgcacctggattgagtgtcacctactcatgcgagtctggctacctgcttgtaggtgaaaaaattattaattgcttgtcctccggcaaatggagtgccgttcccccaacttgtgaagaggcccggtgcaaatccctcggccgcttccctaatggtaaagttaaagagcctccaatcctcagagtgggggtgaccgctaacttcttctgtgatgaaggctaccggttgcagggaccacccagtagccggtgtgtcatagctgggcagggagtggcttggacaaagatgcccgtttgtgaggaagaagactgtaatgagctgcccccaagacggaatacagagatcctcacaggctcttggtccgatcaaacttatccagagggtacccaggcaatttacaagtgcagacctggatacaggagcctgggcaatgtgattatggtgtgccgcaagggggagtgggtggcccttaatcctctccggaagtgtcagaaaagaccatgcggacaccctggagatacacctttcggtacctttacccttaccggcggcaatgtcttcgagtatggcgtcaaggccgtgtacacttgtaacgagggataccagctgctgggggaaataaactatcgtgagtgtgacactgacgggtggactaacgacatccccatttgcgaggtggtcaagtgccttcctgtaaccgctcccgaaaatggtaagatcgtatcttccgcaatggagcctgatcgggaataccactttggacaagccgttcggttcgtatgtaattcagggtataaaattgagggcgatgaggagatgcactgcagtgatgacggcttttggtcaaaggaaaagccaaagtgcgtagagatcagttgtaagtctcctgacgttattaacgggagtcccatcagtcagaagatcatttacaaggaaaacgagaggttccagtataaatgcaatatgggatatgagtactccgaaagaggggacgccgtgtgcacagagtccggatggcgacctttgccatcttgtgaagaaaagtcttgtgacaacccctatattcctaacggagattactctcctctgcgcatcaagcaccgaactggggacgagatcacttaccaatgtcgaaacggcttctaccctgctaccagaggtaacactgccaagtgtaccagcaccggttggattcccgcccccagatgcacacttaaa

SEQ ID NO: 176 - DNA, 2499 bp, Artificial Sequence
  Synthetic Construct
gaagattgcaacgagctgcctcctcggagaaacaccgagatcctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctccacggtgcacactgaaagttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgagccttggaaaagaagattgcaacgagctgcctcctcggagaaacaccgagatcctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctccacggtgcacactgaaa

SEQ ID NO: 177 - DNA, 2352 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgcgaagaggacgccgccgtggaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgctgtggaatgggagagcaacggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctgggcaaagaggactgcaacgagctgcctcctcggagaaataccgagatcctgaccggctcttggagcgaccagacatatccagaaggcacccaggccatctacaagtgccggcctggatacagatccctgggcaatgtgatcatggtctgccggaaaggcgagtgggttgccctgaatcctctgagaaagtgccagaagaggccttgcggacaccccggcgatacaccttttggcacattcaccctgacaggcggcaatgtgttcgagtatggcgtgaaggccgtgtacacctgtaatgagggctaccagctgctgggcgagatcaactacagagagtgtgataccgacggctggaccaacgacatccctatctgcgaggtggtcaagtgcctgccagtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatccctgctccacggtgcacactgaaa

SEQ ID NO: 178 - DNA, 2394 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtgtgcgaagaggtggaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggagcaggcggcggaggatctgaagattgcaatgagctgcctcctcggcggaacaccgagattcttaccggatcttggagcgaccagacataccctgagggcacccaggccatctacaagtgtagacctggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctcctagatgcacactgaagtgatga

SEQ ID NO: 179 - DNA, 1284 bp, Artificial Sequence
  Synthetic Construct
gaggtgcagctggttgaatctggcggaggacttgtgaagcctggcggctctctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaagggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttaccgtgtctagcgaggattgcaacgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctcggtgcacactgaaa

SEQ ID NO: 180 - DNA, 2043 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtgtgtgaagaagaggtgcagctggttgagtctggcggcggacttgtgaaacctggcggaagcctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaaaggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttaccgtgtctagcgaggattgcaacgagctgcctcctcggagaaacaccgagatcctgaccggatcttggagcgaccagacataccctgaaggcacccaggccatctacaagtgcagacctggctacagatccctgggcaatgtgatcatggtctgccggaaaggcgagtgggttgccctgaatcctctgagaaagtgccagaagaggccttgcggacaccctggcgataccccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtacaagtgtaacatgggctacgagtacagcgagaggggcgacgccgtgtgtacagaatctggatggcgacctctgcctagctgcgaggaaaagagctgcgacaacccctacattcccaacggcgactacagccctctgcggatcaaacacagaaccggcgacgagatcacctaccagtgcagaaatggcttctaccccgccaccagaggcaataccgccaagtgtacaagcaccggctggatcccagctcctcggtgcacactgaaa

SEQ ID NO: 181 - DNA, 2073 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctgaagtgcagcttgttgagtctggcggcggacttgtgaaacctggcggaagcctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaaaggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttacagtttcttctggcggaggcggcagcgaggattgcaatgaactgcctcctcggcggaacaccgagatcttgacaggatcttggagcgaccagacataccctgagggcacccaggccatctacaagtgcagacctggctacagatccctgggcaatgtgatcatggtctgccggaaaggcgagtgggttgccctgaatcctctgagaaagtgccagaagaggccttgcggacaccctggcgataccccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtacaagtgtaacatgggctacgagtacagcgagaggggcgacgccgtgtgtacagaatctggatggcgacctctgcctagctgcgaggaaaagagctgcgacaacccctacattcccaacggcgactacagccctctgcggatcaaacacagaaccggcgacgagatcacctaccagtgcagaaatggcttctaccccgccaccagaggcaataccgccaagtgtacaagcaccggctggatcccagctcctcggtgcacactgaaa

SEQ ID NO: 182 - DNA, 2103 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctggcggcggaggctctgaagtgcagcttgttgagtctggcggcggacttgtgaaacctggcggaagcctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaaaggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttacagtttcttctggtggcggaggatctggcggaggcggatctgaagattgcaacgagctgcctcctcggcggaataccgagattctgaccggatcttggagcgaccagacataccctgaaggcacccaggccatctacaagtgcagacctggctacagatccctgggcaatgtgatcatggtctgccggaaaggcgagtgggttgccctgaatcctctgagaaagtgccagaagaggccttgcggacaccctggcgataccccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtacaagtgtaacatgggctacgagtacagcgagaggggcgacgccgtgtgtacagaatctggatggcgacctctgcctagctgcgaggaaaagagctgcgacaacccctacattcccaacggcgactacagccctctgcggatcaaacacagaaccggcgacgagatcacctaccagtgcagaaatggcttctaccctgccaccagaggcaacaccgccaagtgtacaagcacaggctggatccccgctcctcggtgcacactgaaa

SEQ ID NO: 183 - DNA, 2133 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctggcggcggaggctctggcggcggaggctctgaagtgcagcttgttgagtctggcggcggacttgtgaaacctggcggaagcctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaaaggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttacagtttcttctggtggcggaggatctggcggaggtggaagcggaggcggtggatctgaagattgcaacgagctgcctcctcggcggaataccgagattctgaccggatcttggagcgaccagacataccctgaaggcacccaggccatctacaagtgcagacctggctacagatccctgggcaatgtgatcatggtctgccggaaaggcgagtgggttgccctgaatcctctgagaaagtgccagaagaggccttgcggacaccctggcgataccccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtacaagtgtaacatgggctacgagtacagcgagaggggcgacgccgtgtgtacagaatctggatggcgacctctgcctagctgcgaggaaaagagctgcgacaacccctacattcccaacggcgactacagccctctgcggatcaaacacagaaccggcgacgagatcacctaccagtgcagaaatggcttctaccctgccaccagaggcaacaccgccaagtgtacaagcacaggctggatccccgctcctcggtgcacactgaaa

SEQ ID NO: 184 - DNA, 2163 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctggcggcggaggctctggcggcggaggctctggcggcggaggctctgaagtgcagcttgttgagtctggcggcggacttgtgaaacctggcggaagcctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaaaggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttacagtttcttctggtggcggaggatctggcggaggtggaagcggaggcggtggtagtggcggtggtggatctgaggattgcaacgagctgcctcctcggagaaacaccgagatcctgaccggatcttggagcgaccagacataccctgaaggcacccaggccatctacaagtgcagacctggctacagatccctgggcaatgtgatcatggtctgccggaaaggcgagtgggttgccctgaatcctctgagaaagtgccagaagaggccttgcggacaccctggcgataccccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtacaagtgtaacatgggctacgagtacagcgagaggggcgacgccgtgtgtacagaatctggatggcgacctctgcctagctgcgaggaaaagagctgcgacaacccctacattcccaacggcgactacagccctctgcggatcaaacacagaaccggcgacgagatcacctaccagtgcagaaatggcttctaccctgccaccagaggcaacaccgccaagtgtacaagcacaggctggatccccgctcctcggtgcacactgaaa

SEQ ID NO: 185 - DNA, 2061 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtgtgtgaagaagaggtgcagctggttgagtctggcggcggacttgtgaaacctggcggaagcctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaaaggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggttaccgtgtctagcgaggattgcaacgagctgcctcctcggagaaacaccgagatcctgaccggatcttggagcgaccagacataccctgaaggcacccaggccatctacaagtgcagacctggctacagatccctgggcaatgtgatcatggtctgccggaaaggcgagtgggttgccctgaatcctctgagaaagtgccagaagaggccttgcggacaccctggcgataccccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtacaagtgtaacatgggctacgagtacagcgagaggggcgacgccgtgtgtacagaatctggatggcgacctctgcctagctgcgaggaaaagagctgcgacaacccctacattcccaacggcgactacagccctctgcggatcaaacacagaaccggcgacgagatcacctaccagtgcagaaatggcttctaccccgccaccagaggcaataccgccaagtgtacaagcaccggctggatcccagctcctagatgcacactgaagcaccaccaccatcaccac

SEQ ID NO: 186 - DNA, 372 bp, Artificial Sequence
  Synthetic Construct
gaggtgcagctggttgaatctggcggaggacttgtgaagcctggcggctctctgagactgtcttgtgctgcttctggcagacccgtgtctaattacgccgctgcctggtttagacaggcccctggcaaagagagagagttcgtcagcgccatcaactggcagaaaaccgccacatacgccgacagcgtgaagggcagattcaccatcagccgggacaacgccaagaacagcctgtacctgcagatgaactccctgagagccgaggacaccgccgtgtattattgtgccgccgtgtttagagtggtggcccctaagacacagtacgactacgattactggggccagggcaccctggtcaccgtgtcatcttaa

SEQ ID NO: 187 - DNA, 1437 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggcaacaagagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaagatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgagccttggaaaa

SEQ ID NO: 188 - DNA, 2439 bp, Artificial Sequence
  Synthetic Construct
atcagctgcggcagccccccccccatcctgaacggccggatcagctactacagcacccccatcgccgtgggcaccgtgatccggtacagctgcagcggcaccttccggctgatcggcgagaagagcctgctgtgcatcaccaaggacaaggtggacggcacctgggacaagcccgcccccaagtgcgagtacttcaacaagtacagcagctgccccgagcccatcgtgcccggcggctacaagatccggggcagcaccccctaccggcacggcgacagcgtgaccttcgcctgcaagaccaacttcagcatgaacggcaacaagagcgtgtggtgccaggccaacaacatgtggggccccacccggctgcccacctgcgtgagcgtgttccccctggagtgccccgccctgcccatgatccacaacggccaccacaccagcgagaacgtgggcagcatcgcccccggcctgagcgtgacctacagctgcgagagcggctacctgctggtgggcgagaagatcatcaactgcctgagcagcggcaagtggagcgccgtgccccccacctgcgaggaggcccggtgcaagagcctgggccggttccccaacggcaaggtgaaggagccccccatcctgcgggtgggcgtgaccgccaacttcttctgcgacgagggctaccggctgcagggcccccccagcagccggtgcgtgatcgccggccagggcgtggcctggaccaagatgcccgtgtgcgaggagggcggcggcggcgccggcggcggcggcgccggcggcggcggcagcgtggagtgccccccctgccccgccccccccgtggccggccccagcgtgttcctgttcccccccaagcccaaggacaccctgatgatcagccggacccccgaggtgacctgcgtggtggtggacgtgagccaggaggaccccgaggtgcagttcaactggtacgtggacggcgtggaggtgcacaacgccaagaccaagccccgggaggagcagttcaacagcacctaccgggtggtgagcgtgctgaccgtgctgcaccaggactggctgaacggcaaggagtacaagtgcaaggtgagcaacaagggcctgcccagcagcatcgagaagaccatcagcaaggccaagggccagccccgggagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagccggctgaccgtggacaagagccggtggcaggagggcaacgtgttcagctgcagcgtgatgcacgaggccctgcacaaccactacacccagaagagcctgagcctgagcctgggcaagggcggcggcggcgccggcggcggcggcgccggcggcggcggcagcgaggactgcaacgagctgcccccccggcggaacaccgagatcctgaccggcagctggagcgaccagacctaccccgagggcacccaggccatctacaagtgccggcccggctaccggagcctgggcaacgtgatcatggtgtgccggaagggcgagtgggtggccctgaaccccctgcggaagtgccagaagcggccctgcggccaccccggcgacacccccttcggcaccttcaccctgaccggcggcaacgtgttcgagtacggcgtgaaggccgtgtacacctgcaacgagggctaccagctgctgggcgagatcaactaccgggagtgcgacaccgacggctggaccaacgacatccccatctgcgaggtggtgaagtgcctgcccgtgaccgcccccgagaacggcaagatcgtgagcagcgccatggagcccgaccgggagtaccacttcggccaggccgtgcggttcgtgtgcaacagcggctacaagatcgagggcgacgaggagatgcactgcagcgacgacggcttctggagcaaggagaagcccaagtgcgtggagatcagctgcaagagccccgacgtgatcaacggcagccccatcagccagaagatcatctacaaggagaacgagcggttccagtacaagtgcaacatgggctacgagtacagcgagcggggcgacgccgtgtgcaccgagagcggctggcggcccctgcccagctgcgaggagaagagctgcgacaacccctacatccccaacggcgactacagccccctgcggatcaagcaccggaccggcgacgagatcacctaccagtgccggaacggcttctaccccgccacccggggcaacaccgccaagtgcaccagcaccggctggatccccgccccccggtgcaccctgaagtgatga

SEQ ID NO: 189 - DNA, 1959 bp, Artificial Sequence
  Synthetic Construct
ggaaaatgtggccctcctcctcctatcgacaacggcgacattaccagctttccactgtctgtgtacgcccctgccagcagcgtggaataccagtgccagaacctgtaccagctggaaggcaacaagcggatcacctgtagaaacggccagtggtccgagcctcctaagtgtctgcacccttgcgtgatcagccgcgagatcatggaaaactacaatatcgccctgcggtggaccgccaagcagaagctgtatagcagaaccggcgagtccgtggaattcgtgtgcaagagaggctaccggctgagcagcagaagccacacactgagaaccacctgttgggacggcaagctggaataccctacctgtgccaagagggtcgagtgccctccttgtccagctcctcctgttgccggacctagcgtgttcctgtttcctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgagcctgggcaaagaggactgtaacgagctgcctcctcggcggaataccgagattctgacaggctcttggagcgaccagacataccctgagggcacccaggccatctacaagtgtagacctggctacagatccctgggcaatgtgatcatggtctgccggaaaggcgagtgggttgccctgaatcctctgcggaagtgtcagaagaggccttgcggacatcctggcgatacccctttcggcacattcaccctgaccggcggcaatgtgtttgagtatggcgtgaaggccgtgtacacatgcaacgagggatatcagctgctgggcgagatcaactacagagagtgtgataccgacggctggaccaacgacatccctatctgcgaggttgtgaagtgcctgcctgtgacagcccctgagaatggcaagatcgtgtccagcgccatggaacccgacagagagtatcactttggccaggccgtcagattcgtgtgtaactccggctacaagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagagctgcgacaacccttacatccccaacggcgattacagcccactgcggatcaaacacagaacaggcgacgagatcacctaccagtgtcggaacggcttttaccccgccacaagaggcaataccgccaagtgtacaagcaccggctggatccctgctcctcggtgcacactgaag

SEQ ID NO: 190 - DNA, 1959 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcaccctgaaggtggaatgccctccttgtcctgctcctccagtggccggaccttccgtgtttctgttcccacctaagcctaaggacacactgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtataagtgcaaggtgtccaacaagggcctgcctagcagcatcgagaaaaccatcagcaaggccaagggccagccaagagagcctcaggtttacaccctgcctccaagccaagaggaaatgaccaagaaccaggtgtccctgacctgcctggtcaagggcttttacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaatgtgttcagctgcagcgtgatgcacgaggccctgcacaaccactacacccagaagtctctgagcctgagcctcggcaagggaaagtgtggacctcctcctcctatcgacaatggcgacatcaccagctttccactgtctgtgtacgcccctgccagcagcgttgagtatcagtgtcagaacctgtaccagctggaaggcaacaagcggatcacctgtagaaacggccagtggtccgagcctcctaagtgtctgcacccttgcgtgatcagccgcgagatcatggaaaactacaatatcgccctgcggtggaccgccaagcagaagctgtattctagaacaggcgagagcgtcgagtttgtgtgcaagagaggctaccggctgagcagcagaagccacacactgagaaccacctgttgggacggcaagctggaataccctacctgcgccaagaga

SEQ ID NO: 191 - DNA, 1629 bp, Artificial Sequence
  Synthetic Construct
gtggaatgccctccatgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgctgtggaatgggagagcaacggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgtctctcggaaaaggcggaggcggagctggtggtggcggagcaggcggcggaggatctgaagattgcaatgagctgcctcctcggcggaacacagagatcttgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagagctgcgacaacccttacatccccaacggcgactacagccctctgcggattaagcacagaaccggcgacgagatcacctaccagtgcagaaacggcttttaccccgccaccagaggcaataccgccaagtgtacaagcaccggctggatccctgctcctagatgcacactgaag

SEQ ID NO: 192 - DNA, 2058 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtttcagtttttccaggcggcggaggctctgatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggtgctggtggcggagctggcggaggtggaagtgaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagagctgcgacaacccttacatccccaacggcgactacagccctctgcggattaagcacagaaccggcgacgagatcacctaccagtgcagaaacggcttttaccctgccaccagaggcaacaccgccaagtgtacaagcacaggctggatccccgctcctcggtgcacactgaaa

SEQ ID NO: 193 - DNA, 1887 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtttcagtttttccaggcggcggaggctctgatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggtgctggtggcggagctggcggaggtggaagtgaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaagagaagtct

SEQ ID NO: 194 - DNA, 1656 bp, Artificial Sequence
  Synthetic Construct
gaaccgaagtcagctgacaagacccacacttgccctccatgccctgcccctgaactgcttggcgggccttccgtgttcctgttccccccgaaacctaaagataccctcatgatctcgcgaaccccggaagtgacttgcgtggtcgtggatgtgtcccacgaggatcctgaagtgaagttcaattggtacgtggatggagtggaagtccataacgctaagacgaagccgagagaggaacagtacaactcgacctaccgcgtggtgtccgtgctcaccgtgctgcaccaagactggctgaacggaaaggaatacaagtgtaaagtgtccaacaaggccttgccagcccctatcgaaaagaccatatcaaaagcaaagggacagcccagagagccccaggtgtacaccctgccaccttcccgggatgagctgaccaagaaccaagtctccctgacctgtctggtcaagggattctacccctccgatatcgcggtcgaatgggagagcaacggacaacccgaaaacaactacaagactacccctcccgtcctcgactccgatggctcgttcttcctgtattcgaagttgactgtggacaagtccagatggcagcagggcaacgtgttcagctgcagcgtgatgcacgaggcgctgcacaatcattacacccaaaagtccctgtccttgagccctggaaaggggggaggaggtgcaggaggaggaggcgcaggaggaggaggttcggaggactgcaacgagcttccaccgcggagaaatactgaaattctgacaggctcatggtctgatcagacttacccggaaggcacccaggccatctacaaatgtcggcccggctacaggtccctcggaaacgtgatcatggtctgcaggaagggggaatgggtcgccctgaacccgctgagaaagtgccagaagcggccatgtggacacccgggagacactcccttcggcacctttaccctgaccggtggaaacgtgttcgaatacggcgtgaaggccgtgtacacttgcaacgaaggatatcagcttctcggcgagatcaactatcgggaatgcgacaccgatggctggaccaacgacatccctatctgcgaagtcgtcaagtgtctccctgtgactgccccggaaaacggaaagatcgtgtcctccgccatggaacctgaccgggaataccactttggccaagccgtgcggttcgtgtgcaacagcggctacaaaattgaaggagatgaagaaatgcattgtagcgatgacggcttctggtccaaggagaagcctaagtgcgtggaaattagctgcaagtcccccgacgtgatcaacggttcccccatctcccaaaagattatctacaaggagaacgagcgcttccagtacaagtgcaacatgggatacgagtacagcgagagaggggacgcggtctgcaccgagtccgggtggaggcctctgccgtcatgcgaagaaaagagctgcgacaacccctacattccgaacggagactacagcccgctcaggatcaagcaccgcaccggggatgaaatcacttaccaatgccgcaacggattctatccagcgactcgcgggaataccgccaaatgcacctcgactggttggattccggccccaaggtgcaccctgaag

SEQ ID NO: 195 - DNA, 1611 bp, Artificial Sequence
  Synthetic Construct
gaaccgaagtcagctgacaagacccacacttgccctccatgccctgcccctgaactgcttggcgggccttccgtgttcctgttccccccgaaacctaaagataccctcatgatctcgcgaaccccggaagtgacttgcgtggtcgtggatgtgtcccacgaggatcctgaagtgaagttcaattggtacgtggatggagtggaagtccataacgctaagacgaagccgagagaggaacagtacaactcgacctaccgcgtggtgtccgtgctcaccgtgctgcaccaagactggctgaacggaaaggaatacaagtgtaaagtgtccaacaaggccttgccagcccctatcgaaaagaccatatcaaaagcaaagggacagcccagagagccccaggtgtacaccctgccaccttcccgggatgagctgaccaagaaccaagtctccctgacctgtctggtcaagggattctacccctccgatatcgcggtcgaatgggagagcaacggacaacccgaaaacaactacaagactacccctcccgtcctcgactccgatggctcgttcttcctgtattcgaagttgactgtggacaagtccagatggcagcagggcaacgtgttcagctgcagcgtgatgcacgaggcgctgcacaatcattacacccaaaagtccctgtccttgagccctggaaaggaggactgcaacgagcttccaccgcggagaaatactgaaattctgacaggctcatggtctgatcagacttacccggaaggcacccaggccatctacaaatgtcggcccggctacaggtccctcggaaacgtgatcatggtctgcaggaagggggaatgggtcgccctgaacccgctgagaaagtgccagaagcggccatgtggacacccgggagacactcccttcggcacctttaccctgaccggtggaaacgtgttcgaatacggcgtgaaggccgtgtacacttgcaacgaaggatatcagcttctcggcgagatcaactatcgggaatgcgacaccgatggctggaccaacgacatccctatctgcgaagtcgtcaagtgtctccctgtgactgccccggaaaacggaaagatcgtgtcctccgccatggaacctgaccgggaataccactttggccaagccgtgcggttcgtgtgcaacagcggctacaaaattgaaggagatgaagaaatgcattgtagcgatgacggcttctggtccaaggagaagcctaagtgcgtggaaattagctgcaagtcccccgacgtgatcaacggttcccccatctcccaaaagattatctacaaggagaacgagcgcttccagtacaagtgcaacatgggatacgagtacagcgagagaggggacgcggtctgcaccgagtccgggtggaggcctctgccgtcatgcgaagaaaagagctgcgacaacccctacattccgaacggagactacagcccgctcaggatcaagcaccgcaccggggatgaaatcacttaccaatgccgcaacggattctatccagcgactcgcgggaataccgccaaatgcacctcgactggttggattccggccccaaggtgcaccctgaag

SEQ ID NO: 196 - DNA, 1641 bp, Artificial Sequence
  Synthetic Construct
gaagattgcaacgagcttccaccgcggagaaatactgaaattctgacaggctcatggtctgatcagacttacccggaaggcacccaggccatctacaaatgtcggcccggctacaggtccctcggaaacgtgatcatggtctgcaggaagggggaatgggtcgccctgaacccgctgagaaagtgccagaagcggccatgtggacacccgggagacactcccttcggcacctttaccctgaccggtggaaacgtgttcgaatacggcgtgaaggccgtgtacacttgcaacgaaggatatcagcttctcggcgagatcaactatcgggaatgcgacaccgatggctggaccaacgacatccctatctgcgaagtcgtcaagtgtctccctgtgactgccccggaaaacggaaagatcgtgtcctccgccatggaacctgaccgggaataccactttggccaagccgtgcggttcgtgtgcaacagcggctacaaaattgaaggagatgaagaaatgcattgtagcgatgacggcttctggtccaaggagaagcctaagtgcgtggaaattagctgcaagtcccccgacgtgatcaacggttcccccatctcccaaaagattatctacaaggagaacgagcgcttccagtacaagtgcaacatgggatacgagtacagcgagagaggggacgcggtctgcaccgagtccgggtggaggcctctgccgtcatgcgaagaaaagagctgcgacaacccctacattccgaacggagactacagcccgctcaggatcaagcaccgcaccggggatgaaatcacttaccaatgccgcaacggattctatccagcgactcgcgggaataccgccaaatgcacctcgactggttggattccggccccaaggtgcaccctgaagggcggtggcggagcgggcggaggaggagctggagggggaggcagcgacaagacccacacttgccctccatgccctgcccctgaactgcttggcgggccttccgtgttcctgttccccccgaaacctaaagataccctcatgatctcgcgaaccccggaagtgacttgcgtggtcgtggatgtgtcccacgaggatcctgaagtgaagttcaattggtacgtggatggagtggaagtccataacgctaagacgaagccgagagaggaacagtacaactcgacctaccgcgtggtgtccgtgctcaccgtgctgcaccaagactggctgaacggaaaggaatacaagtgtaaagtgtccaacaaggccttgccagcccctatcgaaaagaccatatcaaaagcaaagggacagcccagagagccccaggtgtacaccctgccaccttcccgggatgagctgaccaagaaccaagtctccctgacctgtctggtcaagggattctacccctccgatatcgcggtcgaatgggagagcaacggacaacccgaaaacaactacaagactacccctcccgtcctcgactccgatggctcgttcttcctgtattcgaagttgactgtggacaagtccagatggcagcagggcaacgtgttcagctgcagcgtgatgcacgaggcgctgcacaatcattacacccaaaagtccctgtccttgagccctggaaag

SEQ ID NO: 197 - DNA, 2004 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtacacttaaaggcggaggcggagctggtggtggcggagcaggcggcggaggatctgttgaatgtcctccttgtcctgctcctccagtggccggaccttccgtgtttctgttcccacctaagcctaaggacacactgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtataagtgcaaggtgtccaacaagggcctgcctagcagcatcgagaaaaccatcagcaaggccaagggccagccaagagagcctcaggtttacaccctgcctccaagccaagaggaaatgaccaagaaccaggtgtccctgacctgcctggtcaagggcttttacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaatgtgttcagctgcagcgtgatgcacgaggccctgcacaaccactacacccagaagtctctgagcctgagcctcggcaagggaaagtgtggacctcctcctcctatcgacaatggcgacatcaccagctttccactgtctgtgtacgcccctgccagcagcgttgagtatcagtgtcagaacctgtaccagctggaaggcaacaagcggatcacctgtagaaacggccagtggtccgagcctcctaagtgtctgcacccttgcgtgatcagccgcgagatcatggaaaactacaatatcgccctgcggtggaccgccaagcagaagctgtattctagaacaggcgagagcgtcgagtttgtgtgcaagagaggctaccggctgagcagcagaagccacacactgagaaccacctgttgggacggcaagctggaataccctacctgcgccaagaga

SEQ ID NO: 198 - DNA, 2004 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgcaccctgaaggtggaatgccctccttgtcctgctcctccagtggccggaccttccgtgtttctgttcccacctaagcctaaggacacactgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtataagtgcaaggtgtccaacaagggcctgcctagcagcatcgagaaaaccatcagcaaggccaagggccagccaagagagcctcaggtttacaccctgcctccaagccaagaggaaatgaccaagaaccaggtgtccctgacctgcctggtcaagggcttttacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaatgtgttcagctgcagcgtgatgcacgaggccctgcacaaccactacacccagaagtctctgagcctgtctctcggaaaaggcggaggcggagctggtggtggcggagcaggcggcggaggatctggaaaatgtggacctcctcctcctatcgacaatggcgacatcaccagctttccactgtctgtgtacgcccctgccagcagcgttgagtatcagtgtcagaacctgtaccagctggaaggcaacaagcggatcacctgtagaaacggccagtggtccgagcctcctaagtgtctgcacccttgcgtgatcagccgcgagatcatggaaaactacaatatcgccctgcggtggaccgccaagcagaagctgtattctagaacaggcgagagcgtcgagtttgtgtgcaagagaggctaccggctgagcagcagaagccacacactgagaaccacctgttgggacggcaagctggaataccctacctgcgccaagaga

SEQ ID NO: 199 - DNA, 2049 bp, Artificial Sequence
  Synthetic Construct
gaggattgcaatgagctgcctcctcggagaaacaccgagatcctgacaggctcttggagcgaccagacataccctgagggcacccaggccatctacaagtgcagacctggctacagatccctgggcaacgtgatcatggtctgcagaaaaggcgagtgggtcgccctgaatcctctgagaaagtgccagaagaggccttgcggacaccctggcgataccccttttggcacattcacactgaccggcggcaacgtgttcgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcctaagtgcgtggaaatcagctgcaagagccccgacgtgatcaacggcagccctatcagccagaagatcatctacaaagagaacgagcggttccagtacaagtgtaacatgggctacgagtacagcgagaggggcgacgccgtgtgtacagaatctggatggcgacctctgcctagctgcgaggaaaagagctgcgacaacccttacatccccaacggcgactacagccctctgcggattaagcacagaaccggcgacgagatcacctaccagtgcagaaatggcttctaccccgccaccagaggcaataccgccaagtgtacaagcaccggctggatccctgctcctagatgtacacttaaaggcggaggcggagctggtggtggcggagcaggcggcggaggatctgttgaatgtcctccttgtcctgctcctccagtggccggaccttccgtgtttctgttcccacctaagcctaaggacacactgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtataagtgcaaggtgtccaacaagggcctgcctagcagcatcgagaaaaccatcagcaaggccaagggccagccaagagagcctcaggtttacaccctgcctccaagccaagaggaaatgaccaagaaccaggtgtccctgacctgcctggtcaagggcttttacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaatgtgttcagctgcagcgtgatgcacgaggccctgcacaaccactacacccagaagtctctgagcctgtctcttggaaaaggtggcggtggtgctggcggcggtggtgcaggcggtggcggatctggaaaatgtggacctcctcctcctatcgacaatggcgacatcaccagctttccactgtctgtgtacgcccctgccagcagcgttgagtatcagtgtcagaacctgtaccagctggaaggcaacaagcggatcacctgtagaaacggccagtggtccgagcctcctaagtgtctgcacccttgcgtgatcagccgcgagatcatggaaaactacaatatcgccctgcggtggaccgccaagcagaagctgtattctagaacaggcgagagcgtcgagtttgtgtgcaagagaggctaccggctgagcagcagaagccacacactgagaaccacctgttgggacggcaagctggaataccctacctgcgccaagaga

SEQ ID NO: 200 - DNA, 1902 bp, Artificial Sequence
  Synthetic Construct
atttcttgtggctctccacctcctatcctgaacggccggatcagctactacagcacacctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaacatgtggggacctaccagactgcccacctgtgtgtcagtttttccaggcggcggaggatctgatgccgccgagagaaagtgctgcgtggaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggtgctggtggcggagctggcggaggtggaagtgaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgaggaagtgtcagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagtct

SEQ ID NO: 201 - DNA, 1467 bp, Artificial Sequence
  Synthetic Construct
gaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgtctctcggaaaaggcggaggcggagctggtggtggcggagcaggcggcggtgctggcggcggaggatctgaagattgcaatgagctgcctcctcggcggaacacagagatcttgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagtct

SEQ ID NO: 202 - DNA, 1470 bp, Artificial Sequence
  Synthetic Construct
gaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgtctctcggaaaaggcggaggcggagctggtggtggcggagcaggcggcggtgctggcggcggaggatctaaagaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagtct

SEQ ID NO: 203 - DNA, 1470 bp, Artificial Sequence
  Synthetic Construct
gaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgtctctcggaaaaggcggaggcggagctggtggtggcggagcaggcggcggtgctggcggcggaggatctcgggaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagtct

SEQ ID NO: 204 - DNA, 1455 bp, Artificial Sequence
  Synthetic Construct
gaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgtctctcggaaaaggcggaggcggagctggtggtggtgctggcggcggaggatctaaagaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagtct

SEQ ID NO: 205 - DNA, 1455 bp, Artificial Sequence
  Synthetic Construct
gaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgtctctcggaaaaggcggaggcggagctggtggtggtgctggcggcggaggatctcgggaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagtct

SEQ ID NO: 206 - DNA, 1470 bp, Artificial Sequence
  Synthetic Construct
gttgaatgtcctccatgtcctgctcctccagtggccggaccttccgtgtttctgttccctccaaagcctaaggacaccctgatgatcagcagaacccctgaagtgacctgcgtggtggtggacgtgtcccaagaggaccctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatctctaaggccaagggccagcctcgcgaacctcaggtttacaccctgcctccaagccaagaggaaatgaccaagaaccaggtgtccctgacctgcctggtcaagggcttttacccctccgatatcgccgtggaatgggagagcaacggccagcctgagaacaactacaagaccacacctcctgtgctggacagcgacggcagcttttttctgtactcccgcctgaccgtggacaagagcagatggcaagagggcaacgtgttcagctgtagcgtgatgcacgaggccctgcacaaccactacacccagaagtctctgagcctgtctctcggaaaaggcggaggtggtgctggcggaggcggagcaggaggtggtgcaggcggcggaggatctgaagattgcaacgagctgcctcctcggcggaataccgagattctgacaggctcttggagcgaccagacataccctgagggcacccaggccatctacaagtgtagacctggctaccgcagcctgggcaatgtgatcatggtctgcagaaaaggcgagtgggtcgccctgaatcctctgagaaagtgccagaagaggccttgcggacacccaggcgataccccttttggcacattcaccctgaccggcggcaatgtgtttgagtacggcgtgaaggccgtgtacacctgtaatgagggctaccagctgctgggcgagatcaactacagagagtgtgacaccgacggctggaccaacgacatccctatctgcgaggtggtcaagtgcctgcctgtgacagcccctgagaatggcaagatcgtgtccagcgccatggaacccgatagagagtaccacttcggccaggccgtcagattcgtgtgcaacagcggctacaagatcgagggcgacgaggaaatgcactgcagcgacgacggcttctggtccaaagaaaagcccaaatgcgtggaaatcagctgcaagagccccgacgtgatcaacggcagccccatcagccagaagatcatctacaaagagaacgagcggttccagtataagtgcaacatgggctacgagtacagcgagaggggcgacgccgtgtgtacagaatctggatggcggcctctgcctagctgcgaagagaagtcc

SEQ ID NO: 207 - DNA, 1086 bp, Artificial Sequence
  Synthetic Construct
atctcttgtggctctccacctcctatcctgaacggccggatcagctactacagcacccctatcgctgtgggcaccgtgatcagatacagctgcagcggcaccttccggctgatcggagagaagtccctgctgtgcatcaccaaggacaaggtggacggcacctgggacaagcctgctcctaagtgcgagtacttcaacaagtacagcagctgccccgagcctatcgtgcctggcggctataagatcagaggcagcaccccatacagacacggcgacagcgtgacctttgcctgcaagaccaacttcagcatgaacggccagaaaagcgtgtggtgccaggccaacaacatgtggggacctaccagactgcccacctgtgtgtcagtgtttccaggcggcggaggatctgatgccgctgtggaatgtcctccttgtccagctcctccagtggccggaccttccgtgtttctgttccctccaaagcctaaggacaccctgatgatcagcagaacccctgaagtgacctgcgtggtggtggacgtgtcccaagaggatcctgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggctcattcttcctgtacagcagactgaccgtggacaagagcagatggcaagagggcaacgtgttcagctgctccgtgatgcacgaggccctgcacaaccactacacccagaagtctctgagcctgagcctgggcaag

SEQ ID NO: 208 - protein, 175 aa, Artificial Sequence
  Synthetic Construct
VQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 209 - protein, 808 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 210 - protein, 800 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 211 - protein, 678 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 212 - protein, 751 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEGGGGSDAAVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 213 - protein, 713 aa, Artificial Sequence
  Synthetic Construct
CSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPLECPALPMIHNGHHTSENVGSIAPGLSVTYSCESGYLLVGEKIINCLSSGKWSAVPPTCEEARCKSLGRFPNGKVKEPPILRVGVTANFFCDEGYRLQGPPSSRCVIAGQGVAWTKMPVCEEVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 214 - protein, 621 aa, Artificial Sequence
  Synthetic Construct
ISCGSPPPILNGRISYYSTPIAVGTVIRYSCSGTFRLIGEKSLLCITKDKVDGTWDKPAPKCEYFNKYSSCPEPIVPGGYKIRGSTPYRHGDSVTFACKTNFSMNGQKSVWCQANNMWGPTRLPTCVSVFPVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKS

SEQ ID NO: 215 - protein, 683 aa, Artificial Sequence
  Synthetic Construct
GKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKRGGGGAGGGGAGGGGSVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGAGGGGAGGGGSEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLK

SEQ ID NO: 216 - DNA, 2424 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctgatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggagcaggcggcggtgctggtggcggtggatctgaagattgcaacgagctgcctcctcggcggaataccgagattctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccctgccaccagaggcaacaccgccaagtgtacaagcacaggctggatccccgctcctcggtgcacactgaaa

SEQ ID NO: 217 - DNA, 2160 bp, Artificial Sequence
  Synthetic Construct
aagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtgtgcgaagaggtggaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggagcaggcggcggtgctggcggcggaggatctgaagattgcaatgagctgcctcctcggcggaacaccgagattcttaccggatcttggagcgaccagacataccctgagggcacccaggccatctacaagtgtagacctggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaggaaaagagctgcgacaacccctacattcccaacggcgactacagccctctgcggatcaaacacagaaccggcgacgagatcacctaccagtgcagaaacggcttttaccccgccaccagaggcaataccgccaagtgtacaagcaccggctggatcccagctcctagatgcacactgaag

SEQ ID NO: 218 - DNA, 2034 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtgtctgtgttccctgtggaatgccctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggagcaggcggcggtgctggcggcggaggatctgaagattgcaatgagctgcctcctcggcggaacaccgagattcttacaggctcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagagctgcgacaacccttacatccccaacggcgactacagccctctgcggattaagcacagaaccggcgacgagatcacctaccagtgcagaaacggcttttaccccgccaccagaggcaataccgccaagtgtacaagcaccggctggatccctgctccacggtgcacactgaag

SEQ ID NO: 219 - DNA, 2253 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtttgtgaagaaggcggcggaggctctgatgccgctgttgaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaactccacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggagcaggcggcggtgctggtggcggtggatctgaagattgcaacgagctgcctcctcggcggaataccgagattctgaccggatcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaagagaagtct

SEQ ID NO: 220 - DNA, 2229 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtgtctgtgttccctctggaatgccccgctctgcccatgatccacaatggccaccacacaagcgagaacgtgggatctattgcccctggcctgagcgtgacctacagctgtgaatctggctatctgctcgtgggcgagaagatcatcaattgcctgagcagcggcaagtggtccgctgtgcctcctacatgtgaagaggccagatgcaagagcctgggcagattccccaacggcaaagtgaaagagcctccaatcctgagagtgggcgtgaccgccaacttcttctgtgacgagggctatagactgcagggccctcctagctctagatgcgttatcgctggacagggcgtcgcctggacaaagatgcctgtgtgcgaagaggtggaatgtcctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgttctgcaccaggactggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccggctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggagcaggcggcggtgctggcggcggaggatctgaagattgcaatgagctgcctcctcggcggaacaccgagattcttaccggatcttggagcgaccagacataccctgagggcacccaggccatctacaagtgtagacctggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaatgagggctaccagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactccggatacaagatcgagggcgacgaggaaatgcactgcagcgacgacggcttctggtccaaagaaaagcccaaatgcgtggaaatcagctgcaagtcccctgacgtgatcaacggcagccccatcagccagaagattatctacaaagagaacgagcggttccagtataagtgcaacatgggctacgagtacagcgagcggggagatgccgtgtgtacagaatctggatggcggcctctgcctagctgcgaagagaagtct

SEQ ID NO: 221 - DNA, 1863 bp, Artificial Sequence
  Synthetic Construct
atcagctgtggcagccctccacctatcctgaacggcagaatcagctactacagcacccctatcgccgtgggcaccgtgatcagatacagctgctctggcaccttccggctgatcggagagaagtccctgctgtgcatcaccaaggataaggtggacggcacctgggacaagcctgctcctaagtgcgagtacttcaacaagtacagcagctgccccgagcctatcgtgcctggcggctataagatcagaggcagcacaccctacagacacggcgattctgtgaccttcgcctgcaagaccaacttcagcatgaacggccagaaaagcgtgtggtgccaggccaacaatatgtggggccctaccagactgcccacctgtgtgtctgtgttccctgtggaatgccctccttgtccagctcctcctgtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaatggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctaccctagcgacattgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgcagcgtgatgcacgaagccctgcacaaccactacacccagaagtctctgagcctgtctctcggaaaaggcggaggcggagctggtggtggcggagcaggcggcggtgctggcggcggaggatctgaagattgcaatgagctgcctcctcggcggaacaccgagattcttacaggctcttggagcgaccagacataccctgaaggcacccaggccatctacaagtgtagacccggctacagatccctgggcaatgtgatcatggtctgccggaaaggcgagtgggttgccctgaatcctctgagaaagtgccagaagaggccttgcggacaccccggcgatacaccttttggcacattcaccctgaccggcggcaatgtgtttgagtatggcgtgaaggccgtgtacacctgtaacgagggatatcagctgctgggcgagatcaactacagagagtgtgataccgacggctggaccaacgacatccctatctgcgaggtggtcaagtgcctgcctgtgacagcccctgagaatggcaagatcgtgtccagcgccatggaacccgacagagagtatcactttggccaggccgtcagattcgtgtgcaactctggatacaagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaagagaagtct

SEQ ID NO: 222 - DNA, 2049 bp, Artificial Sequence
  Synthetic Construct
ggcaagtgtggacctcctcctcctatcgacaacggcgacatcaccagctttccactgtctgtgtacgcccctgccagcagcgtggaataccagtgccagaacctgtaccagctggaaggcaacaagcggatcacctgtagaaacggccagtggtccgagcctcctaagtgtctgcacccttgcgtgatcagccgcgagatcatggaaaactacaatatcgccctgcggtggaccgccaagcagaagctgtatagcagaacaggcgagtccgtggaatttgtgtgcaagcggggctacagactgagcagcagaagccacacactgcggaccacatgttgggacggcaagctggaataccctacctgtgctaaaagaggcggaggcggagctggtggtggcggagcaggcggcggaggatctgttgaatgtcctccttgtcctgctcctccagtggccggaccttccgtgtttctgttccctccaaagcctaaggacaccctgatgatcagcagaacccctgaagtgacctgcgtggtggtggacgtttcccaagaggatcccgaggtgcagttcaattggtacgtggacggcgtggaagtgcacaacgccaagaccaagcctagagaggaacagttcaacagcacctacagagtggtgtccgtgctgaccgtgctgcaccaggattggctgaacggcaaagagtacaagtgcaaggtgtccaacaagggcctgcctagcagcatcgagaaaaccatcagcaaggccaagggccagccaagagaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggacagcgacggcagcttttttctgtactcccgcctgaccgtggacaagagcagatggcaagagggcaacgtgttcagctgctctgtgatgcacgaggccctgcacaaccactacacccagaagtctctgagcctgtctcttggaaaaggtggcggtggtgctggtggcggaggcgctggcggtggtggatctgaagattgcaatgagctgcctcctcggcggaacacagagatcttgacaggctcttggagcgaccagacataccctgagggcacccaggccatctacaagtgtagacctggctaccgcagcctgggcaatgtgatcatggtctgcagaaaaggcgaatgggtcgccctgaatcctctgcggaagtgtcagaaaagaccttgcggacaccccggcgataccccttttggcacttttacactgaccggcggcaatgtgttcgagtacggcgtgaaggccgtgtacacctgtaatgagggctatcagctgctgggcgagatcaactacagagagtgtgataccgacggctggaccaacgacatccctatctgcgaggttgtgaagtgcctgcctgtgacagcccctgagaatggcaagatcgtgtccagcgccatggaacccgacagagagtatcactttggccaggccgtcagattcgtgtgcaacagcggctataagatcgagggcgacgaggaaatgcactgcagcgacgacggcttctggtccaaagaaaagcccaaatgcgtggaaatcagctgcaagagccccgacgtgatcaacggcagccctatcagccagaagatcatctacaaagagaacgagcggttccagtataagtgcaacatgggctacgagtacagcgagcggggagatgccgtgtgtacagaatctggatggcggcctctgcctagctgcgaggaaaagagctgcgacaacccttacatccccaacggcgattacagcccactgcggattaagcacagaaccggcgacgagatcacctaccagtgtcggaatggcttttaccctgccaccagaggcaataccgccaagtgtacaagcaccggctggatccctgctcctagatgcacactgaag

SEQ ID NO: 223 - protein, 19 aa, Artificial Sequence
  Synthetic Construct
MGWSCIILFLVATATGVHS

SEQ ID NO: 224 - DNA, 45 bp, Artificial Sequence
  Synthetic Construct
ggcggaggcggagctggtggtggcggagcaggcggcggaggatct

SEQ ID NO: 225 - protein, 479 aa, Artificial Sequence; identical to SEQ ID NO: 117
  Synthetic Construct

SEQ ID NO: 226 - protein, 21 aa, Artificial Sequence
  Synthetic Construct
KGGGGAGGGGAGGGAGGGGSK

SEQ ID NO: 227 - protein, 21 aa, Artificial Sequence
  Synthetic Construct
RGGGGAGGGGAGGGAGGGGSR

SEQ ID NO: 228 - protein, 21 aa, Artificial Sequence
  Synthetic Construct
KGGGGAGGGGAGGGAGGGGSR

SEQ ID NO: 229 - protein, 21 aa, Artificial Sequence
  Synthetic Construct
RGGGGAGGGGAGGGAGGGGSK

SEQ ID NO: 230 - protein, 17 aa, Artificial Sequence
  Synthetic Construct
KGGGGAGGGGAGGGGSK

SEQ ID NO: 231 - protein, 17 aa, Artificial Sequence
  Synthetic Construct
KGGGGAGGGGAGGGGSR

SEQ ID NO: 232 - protein, 17 aa, Artificial Sequence
  Synthetic Construct
RGGGGAGGGGAGGGGSK

SEQ ID NO: 233 - protein, 17 aa, Artificial Sequence
  Synthetic Construct
RGGGGAGGGGAGGGGSR

SEQ ID NO: 234 - protein, 7 aa, Artificial Sequence
  Synthetic Construct
ENLYTQS

SEQ ID NO: 235 - protein, 5 aa, Artificial Sequence
  Synthetic Construct
DDDDK

SEQ ID NO: 236 - protein, 4 aa, Artificial Sequence
  Synthetic Construct
LVPR

SEQ ID NO: 237 - protein, 8 aa, Artificial Sequence
  Synthetic Construct
LEVLFQGP

SEQ ID NO: 238 - protein, 5 aa, Artificial Sequence
  Synthetic Construct
IEDGR

(19) United States
(12) Patent Application Publication
(10) Pub. No.: US 2022/0009979 A1
(43) Pub. Date: Jan. 13, 2022

(54) FUSION PROTEINS AND METHODS OF TREATING COMPLEMENT DYSREGULATION USING THE SAME

(71) Applicant: Alexion Pharmaceuticals, Inc., Boston, MA (US)

(72) Inventors: Julian Chandler, New Haven, CT (US); Christian Cobaugh, Newton Highlands, MA (US); Keith Bouchard, New Haven, CT (US); Jeffrey Hunter, Wallingford, CT (US)

(73) Assignee: Alexion Pharmaceuticals, Inc., Boston, MA (US)

(21) Appl. No.: 17/267,308

(22) PCT Filed: Aug. 22, 2019
(86) PCT No.: PCT/US2019/047793

§ 371 Date: Feb. 9, 2021

Related U.S. Application Data
(60) Provisional application No. 62/721,381, filed on Aug. 22, 2018.

(51) Int. Cl. C07K 14/47; C07K 14/705; C07K 16/18; A61P 13/12; A61K 38/17; A61K 39/395; A61P 19/02

(52) U.S. Cl. CPC C07K 14/472; C07K 14/70596; C07K 16/18; A61P 13/12; A61K 38/177; A61K 2039/545; A61K 39/3955; A61P 19/02; C07K 2317/569; C07K 2317/52; C07K 2317/24; A61K 38/1725

(57) ABSTRACT

Described herein are fusion proteins that include two fragments of factor H, a fragment of factor H and an Fc domain, or a fragment of factor H, a fragment of CR2, and an Fc domain. The use of such proteins in methods of treatment for diseases mediated by alternative complement pathway dysmegulation.

### CROSS REFERENCE TO RELATED APPLICATIONS

This application is related to and claims priority benefit of U.S. Application No. 62/721,381, filed Aug. 22, 2018, incorporated fully herein by reference.

### SEQUENCE LISTING

This application contains a Sequence Listing which has been submitted electronically in ASCII format and is hereby incorporated by reference in its entirety. Said ASCII copy, created on Aug. 22 2019, is named 50694-079WO2_Sequence_Listing_08.22.19 and is 472,000 bytes in size.

### BACKGROUND

The complement system plays a central role in the clearance of immune complexes and in immune responses to infectious agents, foreign antigens, virus-infected cells, and tumor cells. Complement activation occurs primarily by three pathways: the classical pathway, the lectin pathway, and the alternative pathway. The alternative pathway of complement activation is in a constant state of low-level activation. Uncontrolled activation or insufficient regulation of the alternative complement pathway can lead to systemic inflammation, cellular injury, and tissue damage. Thus, the alternative complement pathway has been implicated in the pathogenesis of a number of diverse diseases. Inhibition or modulation of alternative complement pathway activity, in the absence of initiation of the lectin and classical pathway, has been recognized as a promising therapeutic strategy. Particularly, the alternative pathway pays a role in amplifying complement activation initiated from all three pathways. The number of treatment options available for these diseases are limited. Thus, developing innovative strategies to treat diseases associated with alternative complement pathway dysregulation is a significant unmet need.

### SUMMARY

Described herein are engineered fusion proteins that include fragments of complement factor H (FH) fused to Fc domains, such as Fc receptor binding domains; fragments of FH and complement receptor 2 (CR2) fused to Fc domains, such as Fc receptor binding domains; and variants thereof. The fusion proteins can be used to treat patients with diseases associated with alternative complement pathway dysregulation.

Provided herein is a fusion protein having the structure, from N-terminus to C-terminus: D1-L1-Fc-L2-D2, wherein D1 includes a fragment of complement factor H (FH) (e.g., a fragment of FH of any one of SEQ ID NOs: 108-110, 134, and 135) and/or a fragment of CR2 (e.g., a fragment of CR2 of any one of SEQ ID NOs: 94-107, and 136-141); L1 is absent or is an amino acid sequence of at least one amino acid; Fc is an Fc domain; L2 is absent or is an amino acid sequence of at least one amino acid; and D2 includes a fragment of FH (e.g., a fragment of FH of any one of SEQ ID NOs: 108-110, 136, and 137) and/or a fragment of CR2 (e.g., a fragment of CR2 of any one of SEQ ID NOs: 94-107), in which at least one of D1 and D2 includes a fragment of FH.

In one embodiment, the fragment of FH of D1 includes one or more FH short consensus repeat (SCR) domains and/or the fragment of FH of D2 includes one or more FH SCR domains. In some embodiments, the one or more SCR domains are selected from the group consisting of SCR 1, 2, 3, 4, 5, 19, and 20. In one embodiment, the FH SCR domains are SCRs 1-4 (e.g., a fragment of FH of SEQ ID NO: 109). In one embodiment, the FH SCR domains are SCRs 1-5 (e.g., a fragment of FH of SEQ ID NO: 108). In one embodiment, the FH SCR domains are SCRs 1-4, 19, and 20 (e.g., a fragment of FH of SEQ ID NO: 134). In one embodiment, the FH SCR domains are SCRs 1-5, 19, and 20 (e.g., a fragment of FH of SEQ ID NO: 135). In one embodiment, the FH SCR domains are SCRs 19 and 20 (e.g., a fragment of FH of SEQ ID NO: 110).

In another embodiment, the fragment of CR2 of D1 includes one or more CR2 SCR domains and/or the fragment of CR2 of D2 includes one or more CR2 SCR domains. In some embodiments, the one or more SCR domains of CR2 are selected from the group consisting of SCR 1, 2, 3, and 4. In one embodiment, the CR2 SCR domains are SCRs 1-2 (e.g., a fragment of CR2 of any one of SEQ ID NOs: 95 and 102-107). In one embodiment, the CR2 SCR domains are SCRs 1-3 (e.g., a fragment of CR2 of any one of SEQ ID NOs: 136-141). In one embodiment, the CR2 SCR domains are SCRs 1-4 (e.g., a fragment of CR2 of any one of SEQ ID NOs: 94 and 96-101).

In other embodiments, D1 or D2 further includes a fragment of FH fused by a linker (L3) to a fragment of FH. In some embodiments, L3 is an amino acid sequence of at least one amino acid. In one embodiment, the fragment of FH includes SCR domains 19 and 20 (e.g., a fragment of FH of SEQ ID NO: 110).

In other embodiments, D1 or D2 further includes a fragment of FH fused by a linker (L3) to a fragment of CR2. In some embodiments, L3 is an amino acid sequence of at least one amino acid.

In one embodiment, the fragment of CR2 includes SCR domains 1-2 (e.g., a fragment of CR2 of any one of SEQ ID NOs: 95 and 102-107).

In some embodiments, L3 is G₄A, (G₄A)₂G₄S, (G₄A)₂G₃AG₄S, G₄AG₃AG₄S, G₄SDA, G₄SDAA, G₄S, (G₄S)₂, (G₄S)₃, (G₄S)₄, (G₄S)₅, (G₄S)₆, EAAAK, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₁₂, APELPGGP, SEPQPQPG, (G₃S₂)₃, GGGGGGGGGSGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, and (G₄E)₂G₄S, (GGGGS)n, wherein n can be any number, KESGSVSSEQLAQFRSLD, EGKSSGSGSESKST, (Gly)₈, GSAGSAAGSGEF, (Gly)₆, A(EAAAK)A, A(EAAAK)nA, wherein n can be any number, (XP)n wherein n can be any number, with X designating any amino acid, LEAGCKNFFPRSFTSCGSLE, GSST, CRRRRRREAEAC, GS, GSGS, GSGSGS, GSGSGSGS, GSGSGSGSGS, GSGSGSGSGSGS, GGS, GGSGGS, GGSGGSGGS, GGSGGSGGSGGS, GGSG, GGSGGGSG, GGSGGGSGGGSG, GGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGGAGGGGAGGGGS, GGGGAGGGGAGGGGAGGGGS, DAAGGGGSGGGGSGGGGSGGGGSGGGGS, GGGGAGGGGAGGGGA, GGGGAGGGGAGGGAGGGGS, GGSSRSSSSGGGGAGGGG, K(G₄A)₂G₃AG₄SK, R(G₄A)₂G₃AG₄SR, K(G₄A)₂G₃AG₄SR, R(G₄A)₂G₃AG₄SK, K(G₄A)₂G₄SK, K(G₄A)₂G₄SR, R(G₄A)₂G₄SK, R(G₄A)₂G₄SR, ENLYTQS, DDDDK, LVPR, LEVLFQGP, or IEDGR.

In some embodiments, L3 is (G₄A)₂G₄S, G₄SDAA, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S₂)₃, GGGGGGGGGSGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, G₄SDA, G₄A, or (G₄A)₃. In some embodiments, L3 is (G₄A)₂G₄S. In some embodiments, L3 is G₄SDAA. In some embodiments, L3 is (G₄S)₄. In some embodiments, L3 is G₄SDA. In some embodiments, L3 is G₄A. In some embodiments, L3 is (G₄A)₃.

In some embodiments, SCR2 of the fragment of CR2 includes an N101Q substitution, an N107Q substitution, and/or a S109A substitution.

In some embodiments, the Fc domain includes a fragment crystallizable (Fc) domain. In some embodiments the Fc domain includes an Fc domain from a human immunoglobulin, or is a chimeric Fc domain. In some embodiments, the human immunoglobulin is IgG1, IgG2, IgG3, or IgG4. In some embodiments the chimeric Fc domain is IgG2/4. The Fc domain can preferably bind an Fc receptor (e.g., FcRn, FcγRI, FcγRII, or FcγRIll).

In some embodiments, the fusion protein forms a dimer.

In some embodiments, L1 and L2 have the same or different amino acid sequences. L1 and L2 can be selected from the group consisting of: G₄A, (G₄A)₂G₄S, (G₄A)₂G₃AG₄S, G₄AG₃AG₄S, G₄SDA, G₄SDAA, G₄S, (G₄S)₂, (G₄S)₃, (G₄S)₄, (G₄S)₅, (G₄S)₈, EAAAK, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₁₂, APELPGGP, SEPQPQPG, (G₃S₂)₃, GGGGGGGGGSGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, and (G₄E)₂G₄S, (GGGGS)n, wherein n can be any number, KESGSVSSEQLAQFRSLD, EGKSSGSGSESKST, (Gly)₈, GSAGSAAGSGEF, (Gly)₆, A(EAAAK)A, A(EAAAK)nA, wherein n can be any number, (XP)n wherein n can be any number, with X designating any amino acid, LEAGCKNFFPRSFTSCGSLE, GSST, CRRRRRREAEAC, GS, GSGS, GSGSGS, GSGSGSGS, GSGSGSGSGS, GSGSGSGSGSGS, GGS, GGSGGS, GGSGGSGGS, GGSGGSGGSGGS, GGSG, GGSGGGSG, GGSGGGSGGGSG, GGGGS, GENLYFQSGG, SACYCELS, RSIAT, RPACKIPNDLKQKVMNH, GGSAGGSGSGSSGGSSGASGTGTAGGTGSGSGTGSG, AAANSSIDLISVPVDSR, GGSGGGSEGGGSEGGGSEGGGSEGGGSEGGGSGGGS, GGGGAGGGGAGGGGS, GGGGAGGGGAGGGGAGGGGS, DAAGGGGSGGGGSGGGGSGGGGSGGGGS, GGGGAGGGGAGGGGA, GGGGAGGGGAGGGAGGGGS, GGSSRSSSSGGGGAGGGG, K(G₄A)₂G₃AG₄SK, R(G₄A)₂G₃AG₄SR, K(G₄A)₂G₃AG₄SR, R(G₄A)₂G₃AG₄SK, K(G₄A)₂G₄SK, K(G₄A)₂G₄SR, R(G₄A)₂G₄SK, R(G₄A)₂G₄SR, ENLYTQS, DDDDK, LVPR, LEVLFQGP, and IEDGR.

In some embodiments, L1 and L2 can be selected from the group consisting of: (G₄A)₂G₃AG₄S, G₄SDAA, (G₄A)₂G₄S, G₄AG₃AG₄S, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S₂)₃, GGGGGGGGGSGGGS, GGGGSGGGGGGGGGS, (GGSSS)₃, (GS₄)₃, G₄A(G₄S)₂, G₄SG₄AG₄S, G₃AS(G₄S)₂, G₄SG₃ASG₄S, G₄SAG₃SG₄S, (G₄S)₂AG₃S, G₄SAG₃SAG₃S, G₄D(G₄S)₂, G₄SG₄DG₄S, (G₄D)₂G₄S, G₄E(G₄S)₂, G₄SG₄EG₄S, (G₄E)₂G₄S, G₄SDA, G₄A, (G₄A)₃, K(G₄A)₂G₃AG₄SK, R(G₄A)₂G₃AG₄SR, K(G₄A)₂G₃AG₄SR, R(G₄A)₂G₃AG₄SK, K(G₄A)₂G₄SK, K(G₄A)₂G₄SR, R(G₄A)₂G₄SK, R(G₄A)₂G₄SR, ENLYTQS, DDDDK, LVPR, LEVLFQGP, and IEDGR. In some embodiments, L1 and L2 are (G₄A)₂G₄S. In some embodiments, L1 and L2 are G₄SDAA. In some embodiments, L1 and L2 are (G₄S)₄. In some embodiments, L1 is (G₄A)₂G₃AG₄S.

In some embodiments, L2 is (G₄A)₂G₃AG₄S. In some embodiments, L1 is G₄SDAA. In some embodiments, L2 is G₄SDAA. In some embodiments, L1 is G₄AG₃AG₄S. In some embodiments, L2 is G₄AG₃AG₄S.

In one embodiment, the fusion protein has the structure, from N-terminus to C-terminus: D1-L1-Fc-L2-D2, wherein D1 is or includes CR2 domains 1-2, wherein CR2 SCR 2 includes an N107Q substitution; L1 is or includes G₄SDAA; Fc is or includes an IgG2-G4 Fc domain (e.g., having the sequence of SEQ ID NO: 88); L2 includes (G₄A)₂G₃AG₄S; and D2 is or includes FH SCRs 1-4. In some embodiments, the fusion protein has the amino acid sequence of SEQ ID NO: 148, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 148.

In one embodiment, the fusion protein has the structure, from N-terminus to C-terminus: D1-L1-Fc-L2-D2, wherein D1 is or includes CR2 domains 1-2, wherein CR2 SCR 2 includes an N107Q substitution; L1 is or includes G₄SDAA; Fc is or includes an IgG2-G4 Fc domain (e.g., having the sequence of SEQ ID NO: 88); L2 is or includes (G₄A)₂G₃AG₄S; and D2 is or includes FH SCRs 1-5.

In some embodiments, the fusion protein has the amino acid sequence of SEQ ID NO: 147, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 147.

In one embodiment, the fusion protein has the structure, from N-terminus to C-terminus: D1-L1-Fc-L2-D2, wherein D1 is or includes CR2 SCR domains 1 and 2, wherein CR2 SCR 2 includes an N107Q substitution; L1 is or includes G₄SDAA; Fc is or includes a FLG2-G₄ Fc domain (e.g., having the sequence of SEQ ID NO: 111); L2 is or includes (G₄A)₂G₃AG₄S; and D2 is or includes FH SCRs 1-4. In some embodiments, the fusion protein has the amino acid sequence of SEQ ID NO: 155, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 155.

In one embodiment, the fusion protein has the structure, from N-terminus to C-terminus: D1-L1-Fc-L2-D2, wherein D1 is or includes FH SCR domains 19 and 20; L1 is absent; Fc is or includes an IgG2-G4 Fc domain (e.g., having the sequence of SEQ ID NO: 88); L2 is absent; and D2 is or includes FH SCRs 1-5. In some embodiments, the fusion protein has the amino acid sequence of SEQ ID NO: 144, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 144.

In one embodiment, the fusion protein has the structure, from N-terminus to C-terminus: D1-L1-Fc-L2-D2, wherein D1 is or includes FH SCR domains 1-5; L1 is absent; Fc is or includes an IgG2-G₄ Fc domain (e.g., having the sequence of SEQ ID NO: 88); L2 is absent; and D2 is or includes FH SCRs 19 and 20. In some embodiments, the fusion protein has the amino acid sequence of SEQ ID NO: 145, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 145.

In one embodiment, the fusion protein has the structure, from N-terminus to C-terminus: D1-L1-Fc-L2-D2, wherein D1 is or includes FH SCR domains 1-5; L1 is or includes (G₄A)₂G₄S; Fc is or includes an IgG2-G4 Fc domain (e.g., having the sequence of SEQ ID NO: 88); L2 is absent; and D2 is or includes FH SCRs 19 and 20. In some embodiments, the fusion protein has the amino acid sequence of SEQ ID NO: 152, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 152.

In one embodiment, the fusion protein has the structure, from N-terminus to C-terminus: D1-L1-Fc-L2-D2, wherein D1 is or includes FH SCR domains 1-5; L1 is absent; Fc is or includes an IgG2-G₄ Fc domain (e.g., having the sequence of SEQ ID NO: 88); L2 is or includes (G₄A)₂G₄S; and D2 is or includes FH SCRs 19 and 20. In some embodiments, the fusion protein has the amino acid sequence of SEQ ID NO: 153, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 153.

In one embodiment, the fusion protein has the structure, from N-terminus to C-terminus: D1-L1-Fc-L2-D2, wherein D1 is or includes FH SCR domains 1-5; L1 is or includes (G₄A)₂G₄S; Fc is or includes an IgG2-G4 Fc domain (e.g., having the sequence of SEQ ID NO: 88); L2 is or includes (G₄A)₂G₄S; and D2 is or includes FH SCRs 19 and 20. In some embodiments, the fusion protein has the amino acid sequence of SEQ ID NO: 154, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 154.

In one embodiment, the fusion protein has the structure, from N-terminus to C-terminus: D1-L1-Fc-L2-D2, wherein D1 includes CR2 SCR domains 1-4; L1 includes (G₄A)₂G₄S; Fc includes an IgG2-G4 Fc domain (e.g., having the sequence of SEQ ID NO: 88); L2 includes (G₄A)₂G₄S; and D2 includes FH SCRs 1-5. In some embodiments, the fusion protein has the amino acid sequence of SEQ ID NO: 132, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 132.

In one embodiment, the fusion protein has the structure, from N-terminus to C-terminus: D1-L1-Fc-L2-D2, wherein D1 includes CR2 SCR domains 1-4, wherein CR2 SCR 2 includes an N107Q substitution; L1 includes (G₄A)₂G₄S; Fc includes an IgG2-G4 Fc domain (e.g., having the sequence of SEQ ID NO: 88); L2 includes (G₄A)₂G₄S; and D2 includes FH SCRs 1-5. In some embodiments, the fusion protein has the amino acid sequence of SEQ ID NO: 121, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 121.

In one embodiment, the fusion protein has the structure, from N-terminus to C-terminus: D1-L1-Fc-L2-D2, wherein D1 includes CR2 SCR domains 1-4, wherein CR2 SCR 2 includes a S109A substitution; L1 includes (G₄A)₂G₄S; Fc includes an IgG2-G4 Fc domain (e.g., having the sequence of SEQ ID NO: 88); L2 includes (G₄A)₂G₄S; and D2 includes FH SCRs 1-5. In some embodiments, the fusion protein has the amino acid sequence of SEQ ID NO: 122, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 122.

In one embodiment, the fusion protein has the structure, from N-terminus to C-terminus: D1-L1-Fc-L2-D2, wherein D1 includes CR2 SCR domains 1-4; L1 includes G₄SDAA; Fc includes an IgG2-G4 Fc domain (e.g., having the sequence of SEQ ID NO: 88); L2 includes (G₄S)₄; and D2 includes FH SCRs 1-5. In some embodiments, the fusion protein has the amino acid sequence of SEQ ID NO: 114, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 114.

In one embodiment, the fusion protein has the structure, from N-terminus to C-terminus: D1-L1-Fc-L2-D2, wherein D1 includes CR2 SCR domains 1-4; L1 includes G₄SDAA; Fc includes an IgG2-G4 Fc domain (e.g., having the sequence of SEQ ID NO: 88); L2 includes (G₄S)₂; and D2 includes FH SCRs 1-5. In some embodiments, the fusion protein has the amino acid sequence of SEQ ID NO: 118, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 118.

In one embodiment, the fusion protein has the structure, from N-terminus to C-terminus: D1-L1-Fc-L2-D2, wherein D1 includes CR2 SCR domains 1-4; L1 includes G₄SDAA; Fc includes an IgG2-G4 Fc domain (e.g., having the sequence of SEQ ID NO: 88); L2 includes G₄S; and D2 includes FH SCRs 1-5. In some embodiments, the fusion protein has the amino acid sequence of SEQ ID NO: 119, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 119.

In one embodiment, the fusion protein has the structure, from N-terminus to C-terminus: D1-L1-Fc-L2-D2, wherein D1 includes CR2 SCR domains 1-4; L1 is absent; Fc includes an IgG2-G4 Fc domain (e.g., having the sequence of SEQ ID NO: 88); L2 is absent; and D2 includes FH SCRs 1-5. In some embodiments, the fusion protein has the amino acid sequence of SEQ ID NO: 116, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 116.

In one embodiment, the fusion protein has the structure, from N-terminus to C-terminus: D1-L1-Fc-L2-D2, wherein D1 includes CR2 SCR domains 1-4; L1 is absent; Fc includes an IgG2-G4 Fc domain (e.g., having the sequence of SEQ ID NO: 88); L2 includes (G₄A)₂G₄S; and D2 includes FH SCRs 1-5. In some embodiments, the fusion protein has the amino acid sequence of SEQ ID NO: 124, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 124.

In some embodiments, the fusion protein has the amino acid sequence of SEQ ID NO: 115, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 115.

In some embodiments, the fusion protein has the amino acid sequence of SEQ ID NO: 117, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 117.

In some embodiments, the fusion protein has the amino acid sequence of SEQ ID NO: 120, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 120.

In some embodiments, the fusion protein has the amino acid sequence of SEQ ID NO: 123, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 123.

In one embodiment, the fusion protein has the structure, from N-terminus to C-terminus: D1-L1-Fc-L2-D2, wherein D1 is or includes CR2 SCR domains 1-4; L1 is or includes G₄SDAA; Fc is or includes an IgG2-G4 Fc domain (e.g., having the sequence of SEQ ID NO: 88); L2 is or includes (G₄A)₂G₃AG₄S; and D2 is or includes FH SCRs 1-5. In some embodiments, the fusion protein has the amino acid sequence of SEQ ID NO: 209, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence with at least 85% (e.g., at least 90%, at least 95%, at least 97%, or at least 99%) sequence identity to SEQ ID NO: 209.

In one embodiment, the fusion protein has the structure, from N-terminus to C-terminus: D1-L1-Fc-L2-D2, wherein D1 is or includes CR2 SCR domains 1-4, wherein CR2 SCR 2 includes an N107Q substitution; L1 is absent; Fc is or includes an IgG2-G4 Fc domain (e.g., having the sequence of SEQ ID NO: 88); L2 is or includes (G₄A)₂G₃AG₄S; and D2 is or includes FH SCRs 1-5. In some embodiments, the fusion protein has the amino acid sequence of SEQ ID NO: 210, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence with at least 85% (e.g., at least 90%, at least 95%, at least 97%, or at least 99%) sequence identity to SEQ ID NO: 210.

In one embodiment, the fusion protein has the structure, from N-terminus to C-terminus: D1-L1-Fc-L2-D2, wherein D1 is or includes CR2 SCR domains 1-2, wherein CR2 SCR 2 includes an N107Q substitution; L1 is absent; Fc is or includes an IgG2-G4 Fc domain (e.g., having the sequence of SEQ ID NO: 88); L2 is or includes (G₄A)₂G₃AG₄S; and D2 is or includes FH SCRs 1-5. In some embodiments, the fusion protein has the amino acid sequence of SEQ ID NO: 211, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence with at least 85% (e.g., at least 90%, at least 95%, at least 97%, or at least 99%) sequence identity to SEQ ID NO: 211.

In one embodiment, the fusion protein has the structure, from N-terminus to C-terminus: D1-L1-Fc-L2-D2, wherein D1 is or includes CR2 SCR domains 1-4, wherein CR2 SCR 2 includes an N107Q substitution; L1 is or includes G₄SDA; Fc is or includes an IgG2-G4 Fc domain (e.g., having the sequence of SEQ ID NO: 88); L2 is or includes (G₄A)₂G₃AG₄S; and D2 is or includes FH SCRs 1-4. In some embodiments, the fusion protein has the amino acid sequence of SEQ ID NO: 212, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence with at least 85% (e.g., at least 90%, at least 95%, at least 97%, or at least 99%) sequence identity to SEQ ID NO: 212.

In one embodiment, the fusion protein has the structure, from N-terminus to C-terminus: D1-L1-Fc-L2-D2, wherein D1 is or includes CR2 SCR domains 1-4, wherein CR2 SCR 2 includes an N107Q substitution; L1 is absent; Fc is or includes an IgG2-G4 Fc domain (e.g., having the sequence of SEQ ID NO: 88); L2 is or includes (G₄A)₂G₃AG₄S; and D2 is or includes FH SCRs 1-4. In some embodiments, the fusion protein has the amino acid sequence of SEQ ID NO: 213, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence with at least 85% (e.g., at least 90%, at least 95%, at least 97%, or at least 99%) sequence identity to SEQ ID NO: 213.

In one embodiment, the fusion protein has the structure, from N-terminus to C-terminus: D1-L1-Fc-L2-D2, wherein D1 is or includes CR2 SCR domains 1-2, wherein CR2 SCR 2 includes an N107Q substitution; L1 is absent; Fc is or includes an IgG2-G4 Fc domain (e.g., having the sequence of SEQ ID NO: 88); L2 is or includes (G₄A)₂G₃AG₄S; and D2 is or includes FH SCRs 1-4. In some embodiments, the fusion protein has the amino acid sequence of SEQ ID NO: 214, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence with at least 85% (e.g., at least 90%, at least 95%, at least 97%, or at least 99%) sequence identity to SEQ ID NO: 214.

In one embodiment, the fusion protein has the structure, from N-terminus to C-terminus: D1-L1-Fc-L2-D2, wherein D1 is or includes FH SCR domains 19-20; L1 is or includes (G₄A)₂G₄S; Fc is or includes an IgG2-G4 Fc domain (e.g., having the sequence of SEQ ID NO: 88); L2 is or includes (G₄A)₂G₄S; and D2 is or includes FH SCRs 1-4. In some embodiments, the fusion protein has the amino acid sequence of SEQ ID NO: 215, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence with at least 85% (e.g., at least 90%, at least 95%, at least 97%, or at least 99%) sequence identity to SEQ ID NO: 215.

Also provided herein is a fusion protein including (a) a moiety including a fragment of complement receptor 2 (CR2); (b) an anti-albumin VHH domain; and (c) a moiety including a fragment of complement factor H (FH). In some embodiments, the fusion protein has the structure, from N-terminus to C-terminus: (a)-(b)-(c). In other embodiments, the fusion protein has the structure (a)-L1-(b)-L2-(c), in which L1 and L2, independently, may be absent or a linker of at least one amino acid.

L1 and L2 can have the sequence selected from those shown above. In some embodiments, one or more, or all, of (a), (b), and/or (c) are fused by a linker.

In one embodiment, fusion protein includes from N-terminus to C-terminus: FH SCR domains 1-5 (e.g., a fragment of FH of SEQ ID NO: 108) fused to an anti-albumin VHH domain, with or without a linker.

In one embodiment, the fusion protein includes from N-terminus to C-terminus: CR2 SCR domains 1-4 (e.g., a fragment of CR2 of any one of SEQ ID NOs: 94 and 96-101) fused to the anti-albumin VHH domain fused to FH SCR domains 1-5 (e.g., a fragment of FH of SEQ ID NO: 108).

In some embodiments, the fusion protein has the amino acid sequence of SEQ ID NO: 125, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 125.

In some embodiments, the fusion protein has the amino acid sequence of SEQ ID NO: 126, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 126.

In some embodiments, the fusion protein has the amino acid sequence of SEQ ID NO: 127, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 127.

In some embodiments, the fusion protein has the amino acid sequence of SEQ ID NO: 128, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 128.

In some embodiments, the fusion protein has the amino acid sequence of SEQ ID NO: 129, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 129.

In some embodiments, the fusion protein has the amino acid sequence of SEQ ID NO: 130, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 130.

In some embodiments, the fusion protein has the amino acid sequence of SEQ ID NO: 131, or a variant thereof having up to 10 (e.g., 10 or fewer, 9 or fewer, 8 or fewer, 7 or fewer, 6 or fewer, 5 or fewer, 4 or fewer, 3 or fewer, 2 or fewer, or 1 or fewer) amino acid substitutions, additions, or deletions. In some embodiments, the fusion protein has an amino acid sequence having at least 85% (e.g., at least 85%, at least 90%, at least 95%, or at least 99%) sequence identity to SEQ ID NO: 131.

In some embodiments, the fusion protein has an increased half-life relative to the fusion protein lacking the Fc domain.

In one embodiment, the fusion protein is formulated in a pharmaceutical composition, with at least one pharmaceutically acceptable carrier. In one embodiment, the at least one pharmaceutically acceptable carrier is saline.

Also provided is a nucleic acid or polynucleotide encoding a fusion protein described herein.

Also provided is a vector including the nucleic acid encoding a fusion protein described herein.

Also provided is a host cell including the nucleic acid and/or vector encoding a fusion protein described herein.

Also provided is a method of treating a disease mediated by alternative complement pathway dysregulation including administering an effective amount of a pharmaceutical composition including a fusion protein described herein to a subject in need thereof.

Also provided is a method of treating a disease mediated by alternative complement pathway dysregulation including administering an effective amount of a polynucleotide encoding a fusion protein described herein to a subject in need thereof.

Also provided is a method of treating a disease mediated by alternative complement pathway dysregulation including administering an effective amount of a host cell including a nucleic acid encoding a fusion protein described herein to a subject in need thereof.

Also provided is a method of producing a fusion protein described herein including the steps of culturing one or more host cells including one or more nucleic acid molecules capable of expressing the fusion protein under conditions suitable for expression of the fusion protein. In some embodiments, the method further includes the step of obtaining the fusion protein from the cell culture or culture medium.

Also provided is a method of treating a disease mediated by alternative complement pathway dysregulation including administering an effective amount of a fusion protein described herein to a subject in need thereof. In some embodiments, the fusion protein is formulated in a pharmaceutical composition, with at least one pharmaceutically acceptable carrier, and is, preferably, rehydrated prior to administration. In some embodiments, the composition is lyophilized. In some embodiments, the at least one pharmaceutically acceptable carrier is saline.

In some embodiments, the fusion protein is formulated for daily, weekly, or monthly administration. In some embodiments, the fusion protein is formulated for intravenous, subcutaneous, intramuscular, oral, nasal, sublingual, intrathecal, or intradermal administration. In some embodiments, the fusion protein is formulated for administration at a dosage of between about 0.1 mg/kg to about 150 mg/kg. In some embodiments, the fusion protein is formulated for administration in combination with an additional therapeutic agent.

In some embodiments, the disease is paroxysmal nocturnal hemoglobinuria (PNH). In some embodiments, the disease is atypical hemolytic uremic syndrome (aHUS). In some embodiments, the disease is IgA nephropathy. In some embodiments, the disease is lupus nephritis. In some embodiments, the disease is C3 glomerulopathy (C3G). In some embodiments, the disease is dermatomyositis. In some embodiments, the disease is systemic sclerosis. In some embodiments, the disease is demyelinating polyneuropathy. In some embodiments, the disease is pemphigus. In some embodiments, the disease is dense deposit disease (DDD). In some embodiments, the disease is age related macular degeneration (AMD). In some embodiments, the disease is thrombic thrombocytopenic purpura (TTP). In some embodiments, the disease is membranous nephropathy.

In some embodiments, the disease is focal segmental glomerular sclerosis (FSGS). In some embodiments, the disease is membranous nephropathy. In some embodiments, the disease is bullous pemphigoid. In some embodiments, the disease is membranous nephropathy. In some embodiments, the disease is epidermolysis bullosa acquisita (EBA). In some embodiments, the disease is ANCA vasculitis. In some embodiments, the disease is membranous nephropathy. In some embodiments, the disease is hypocomplementemic urticarial vasculitis. In some embodiments, the disease is immune complex small vessel vasculitis. In some embodiments, the disease is an autoimmune necrotizing myopathy.

In some embodiments, the disease is rejection of a transplanted organ. In some embodiments, the disease is antiphospholipid (aPL) Ab syndrome. In some embodiments, the disease is glomerulonephritis. In some embodiments, the disease is asthma. In some embodiments, the disease is systemic lupus erythematosus (SLE). In some embodiments, the disease is rheumatoid arthritis (RA). In some embodiments, the disease is multiple sclerosis (MS). In some embodiments, the disease is traumatic brain injury (TBI). In some embodiments, the disease is ischemia reperfusion injury. In some embodiments, the disease is preeclampsia.

In some embodiments, the subject is a mammal. In some embodiments, the mammal is a human.

Also provided is a kit including a fusion protein described herein. In some embodiments, the kit further includes instructions for administering an effective amount of the fusion protein to a subject in need thereof.

Excluded from this disclosure is a construct consisting of CR2 SCR 1-4 directly fused to FH SCR 1-5 (CR2₁₋₄-FH₁₋₅), as described in WO2007/14567.

### BRIEF DESCRIPTION OF THE DRAWINGS

This application file contains at least one drawing executed in color. Copies of this patent or patent application with color drawings will be provided by the Office upon request and payment of the necessary fee.

FIG. 1A is a schematic diagram illustrating exemplary complement factor H (FH) fusion proteins.

FIG. 1B are sequences of CR2 fragments A-F, corresponding to SEQ ID NOs: 99, 97, 98, 96, 100, and 101, respectively, containing various mutations to ablate N-linked glycosylation. Fragments A and C include an S109A mutation. Fragments D and F include an N107Q mutation. Mutated residues are denoted by an asterisk above the residue. Shaded, underlined residues indicate N-glycosylation motifs. Shaded residues with a “+” above the residue denote positively charged residues within the N-glycosylation motifs. Shaded, non-underlined residues indicate positively charged amino acids, none of which were mutated.

FIGS. 2A-2C are a series of SDS-PAGE gels showing the expression of the factor H fusion protein variants from harvested cell culture supernatants. The accompanying tables indicate the predicted molecular weight (MW) in kilodaltons (kDa) of the major band, as well as the yield in μg/mL.

FIGS. 3A-3B are representative SE HPLC chromatograms (280 nm) and SDS-PAGE gels of purified CR2-FH-Fc fusion protein N-linked glycosylation variants.

FIGS. 4A-4D are a series of graphs showing alternative pathway hemolytic activity of fusion proteins containing FH or fusion proteins including CR2 and FH.

FIG. 4E is a schematic diagram illustrating the complement factor H (FH) fusion proteins tested for hemolytic activity (see FIGS. 4C and 40).

FIG. 5A is a schematic diagram illustrating exemplary FH anti-albumin-VHH fusion proteins with glycosylation variants.

FIG. 5B is an SDS-PAGE gel showing the expression of the factor H anti-albumin-VHH fusion protein variants from harvested cell culture supernatants. The accompanying table indicates the predicted molecular weight (MW) in kilodaltons (kDa) of the major band, as well as the yield in μg/mL.

FIG. 5C is an SDS-PAGE gel purifying factor H anti-albumin-VHH fusion proteins from harvested cell culture supernatants fractionated from MEP HYPERCEL™ or CAPTO™ Adhere ImpRes resins.

FIG. 5D is an SDS-PAGE gel determining elution pH profile of the factor H anti-albumin-VHH fusion proteins from harvested cell culture supernatants using MEP HYPERCEL™ or CAPTO™ Adhere ImpRes resin, purified along a pH gradient.

FIG. 5E is a graph showing the yield of the factor H anti-albumin-VHH fusion protein (Compound O) isolated using various small scale purification schemes.

FIG. 5F is a SE HPLC chromatogram showing the purity of the factor H anti-albumin-VHH fusion protein (Compound O) isolated using MEP HYPERCEL™ resin at pH 4.7.

FIG. 56G is a SE HPLC chromatogram showing the purity of the factor H anti-albumin-VHH fusion protein (Compound O) isolated using CAPTO™ Adhere ImpRes resin at pH 4.46.

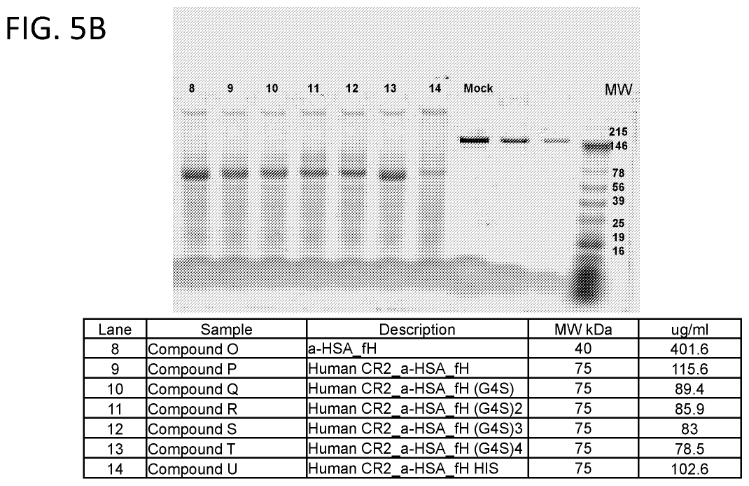

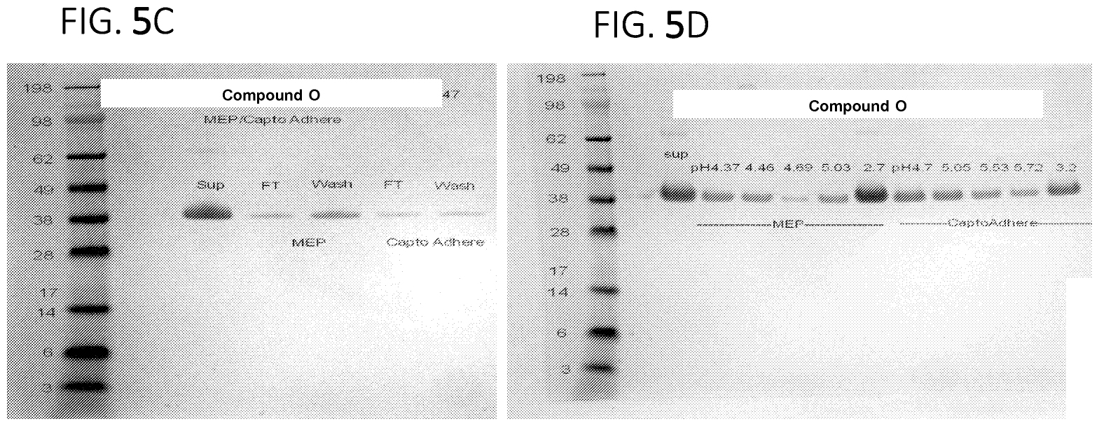

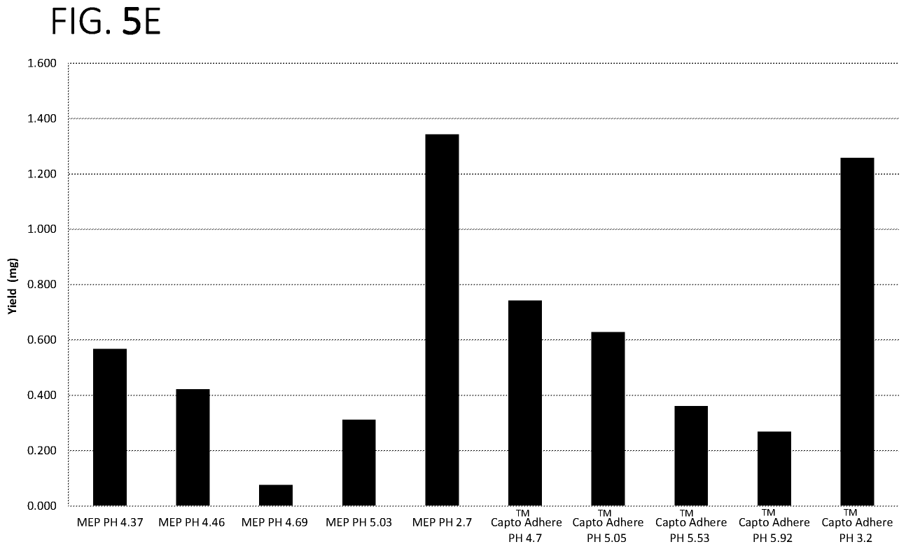

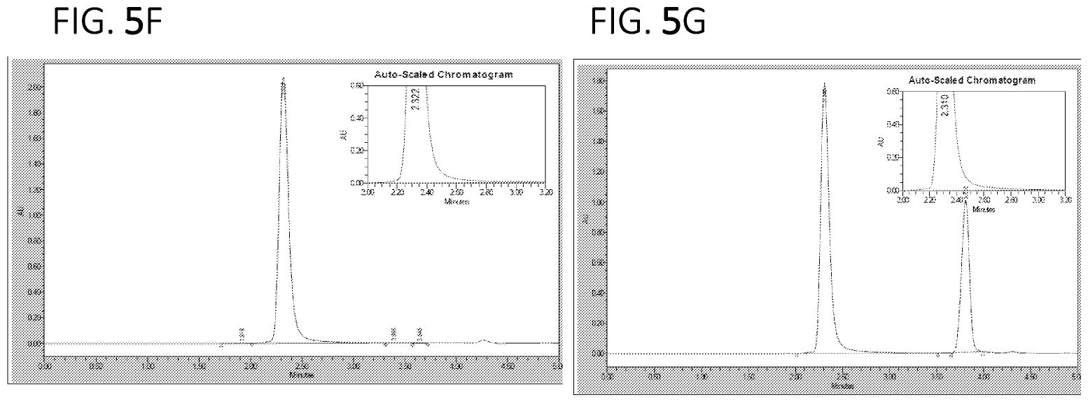

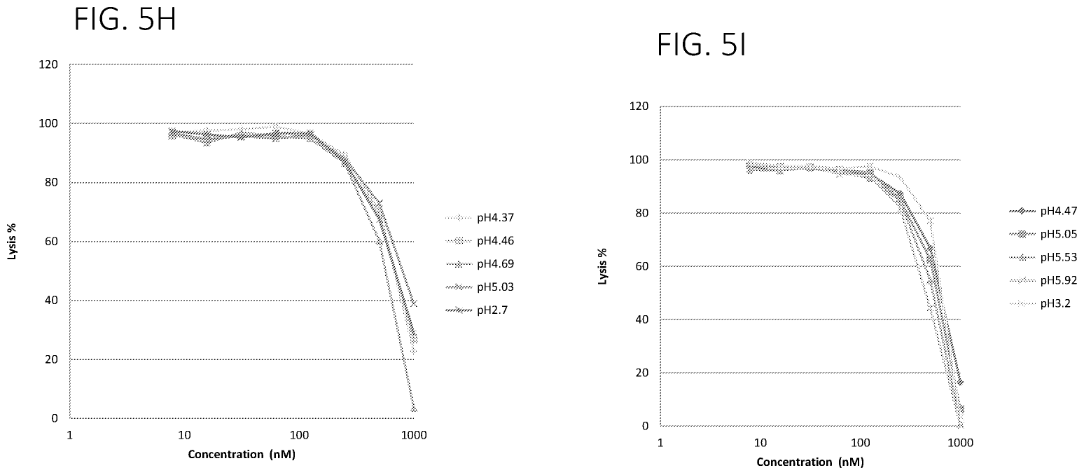

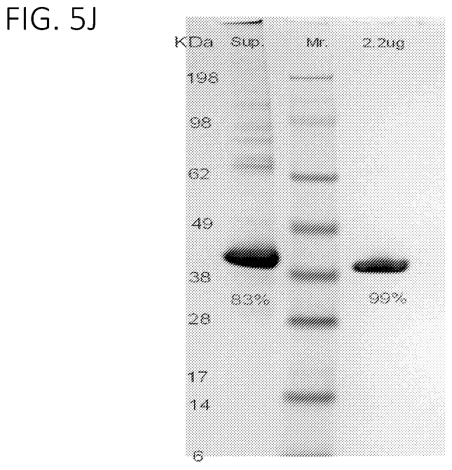

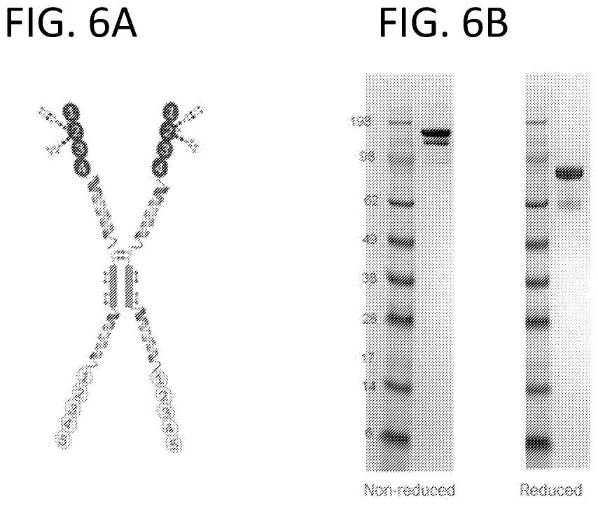

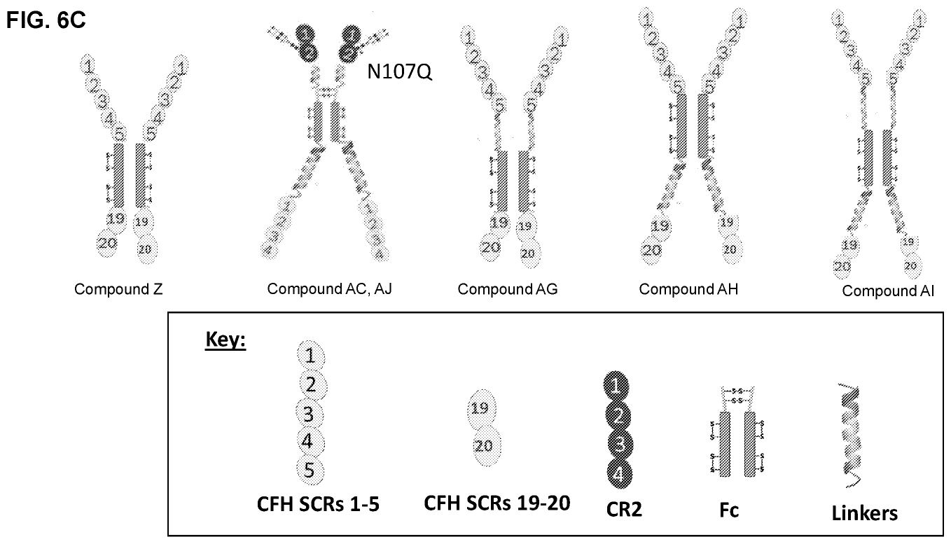

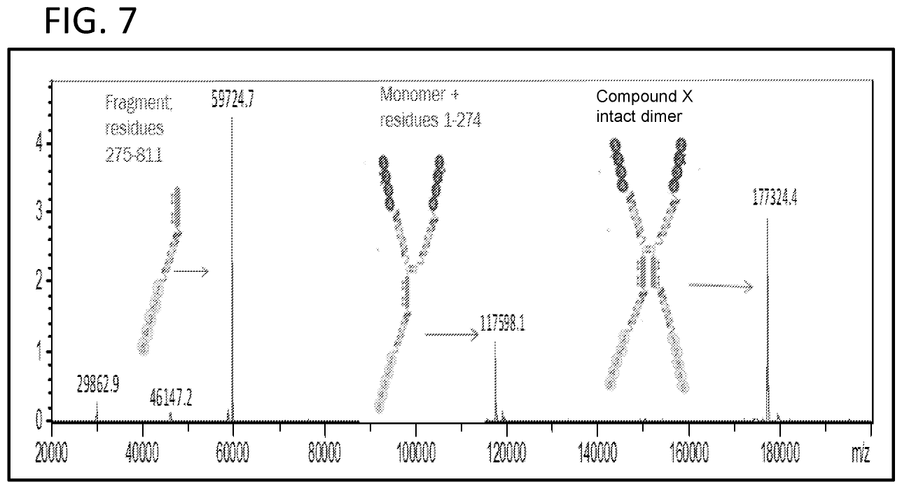

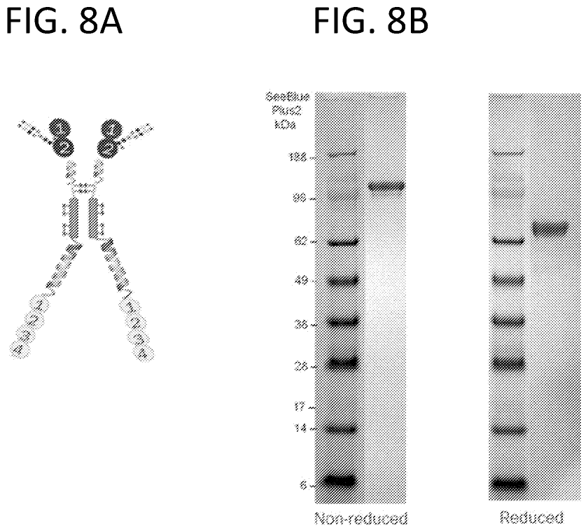

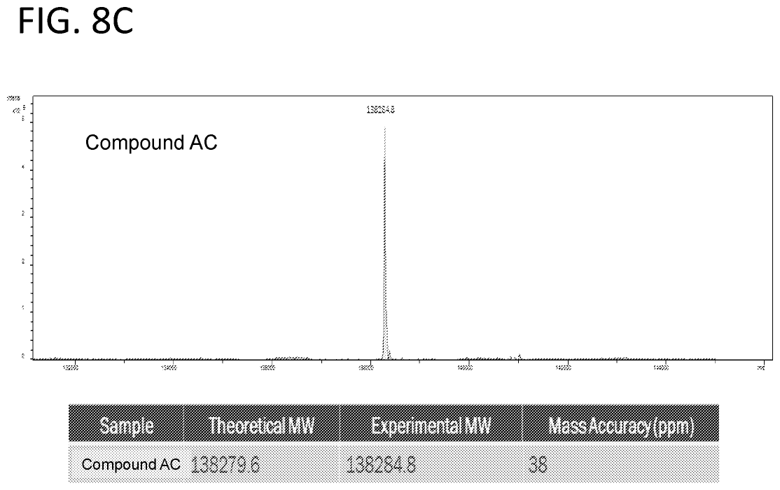

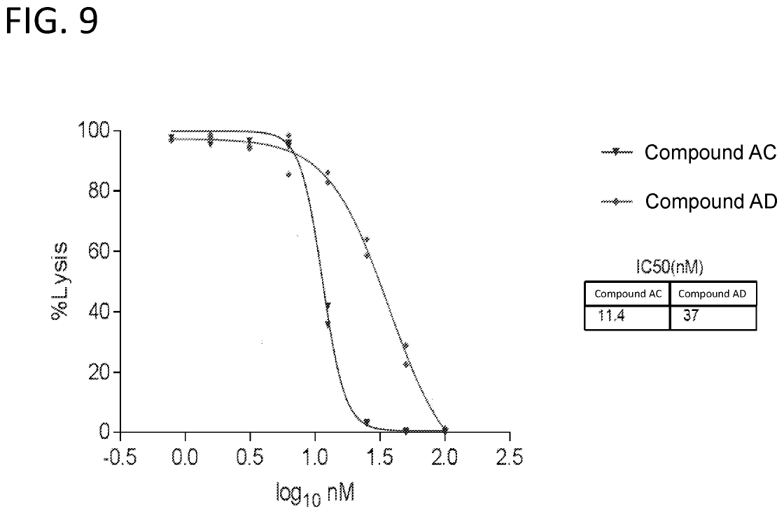

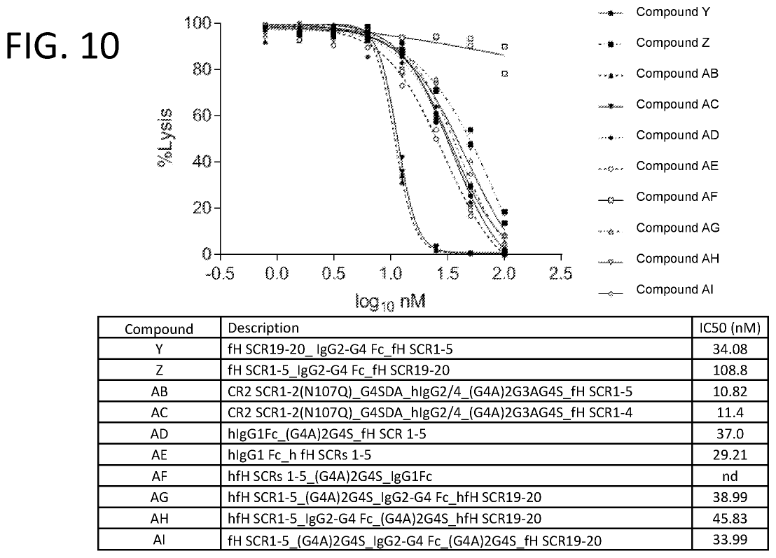

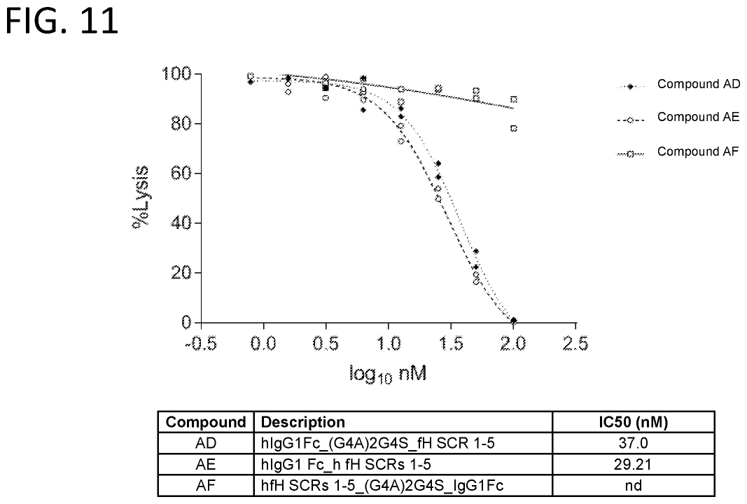

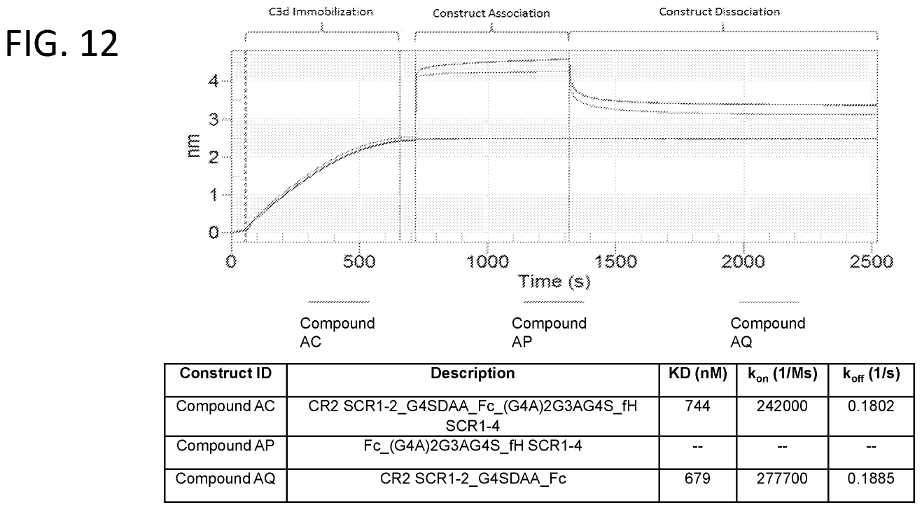

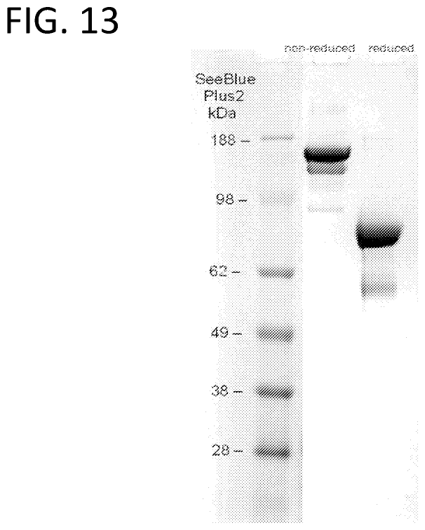

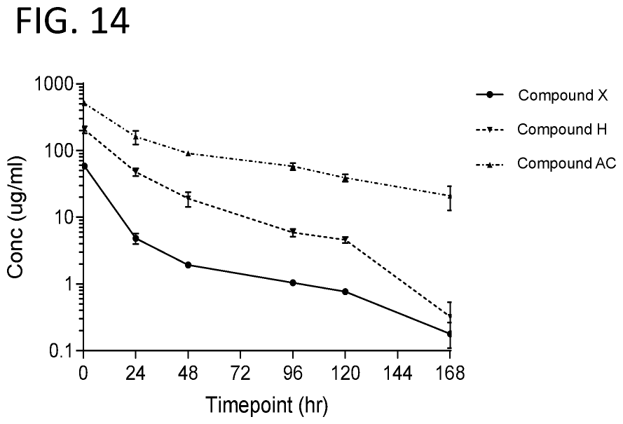

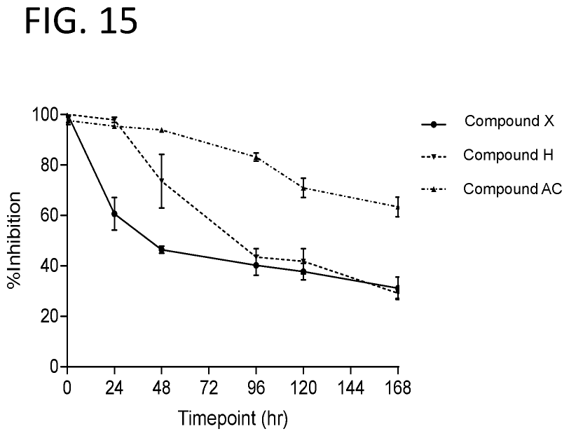

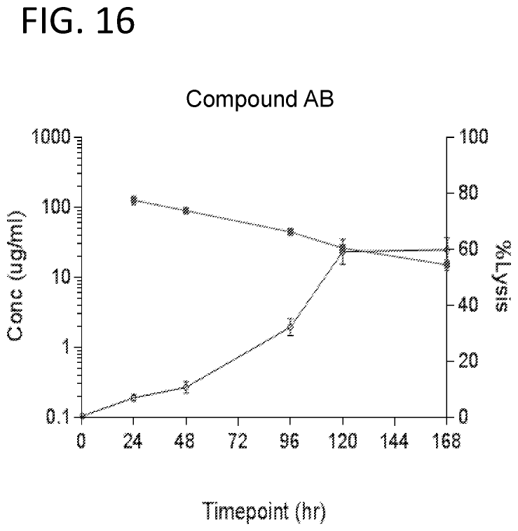

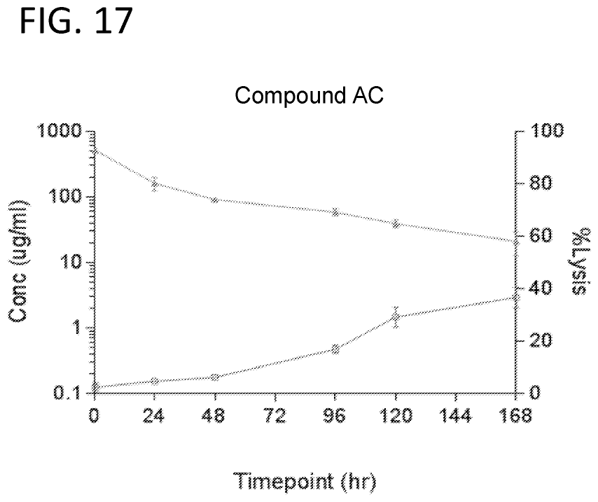

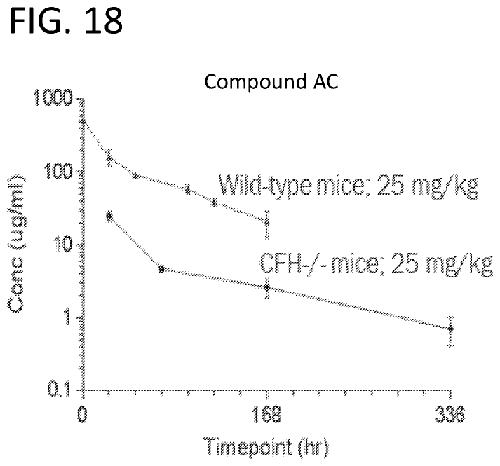

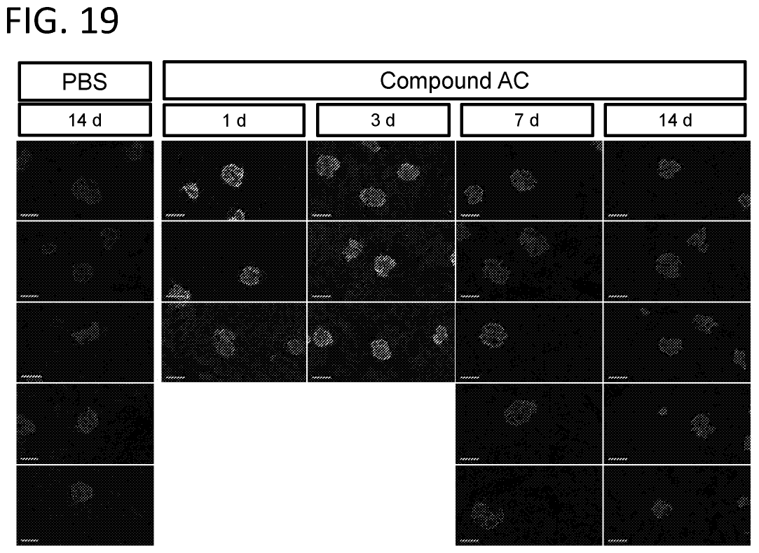

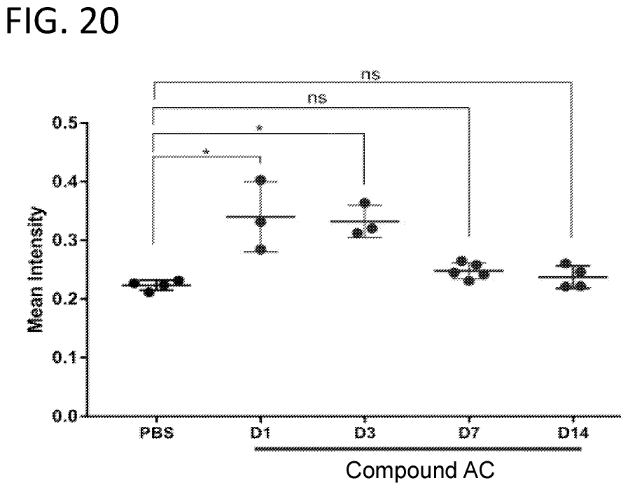

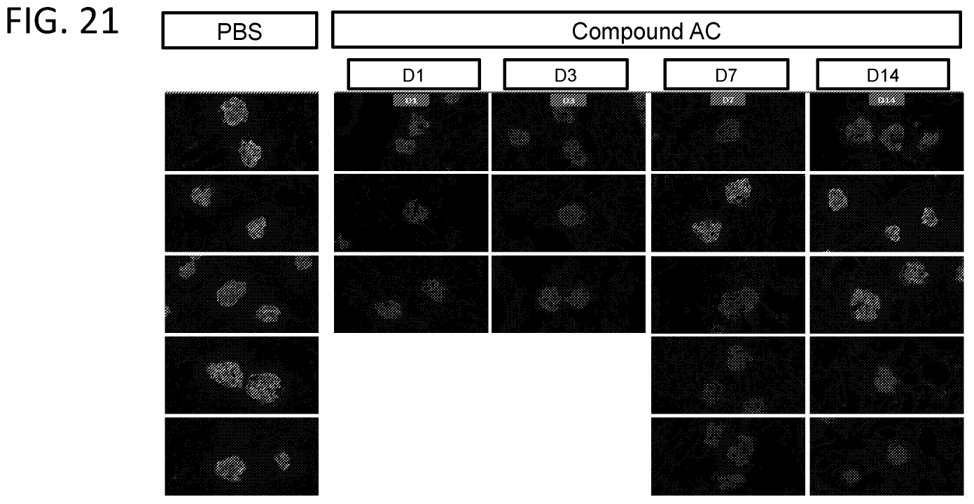

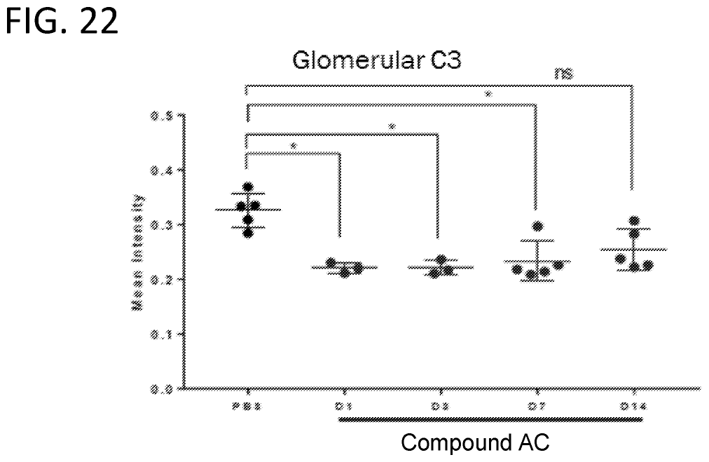

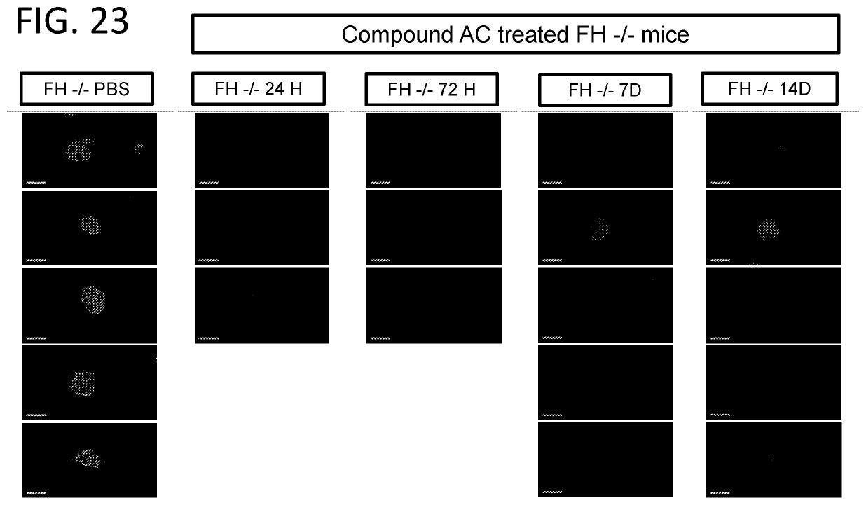

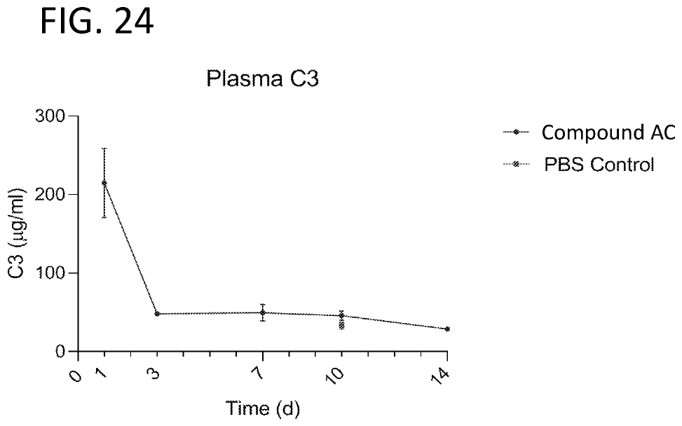

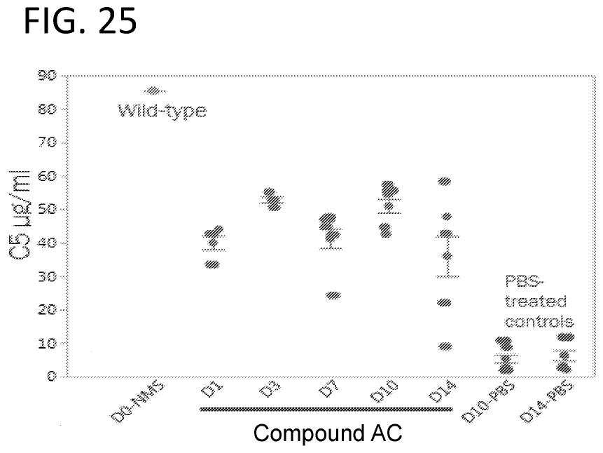

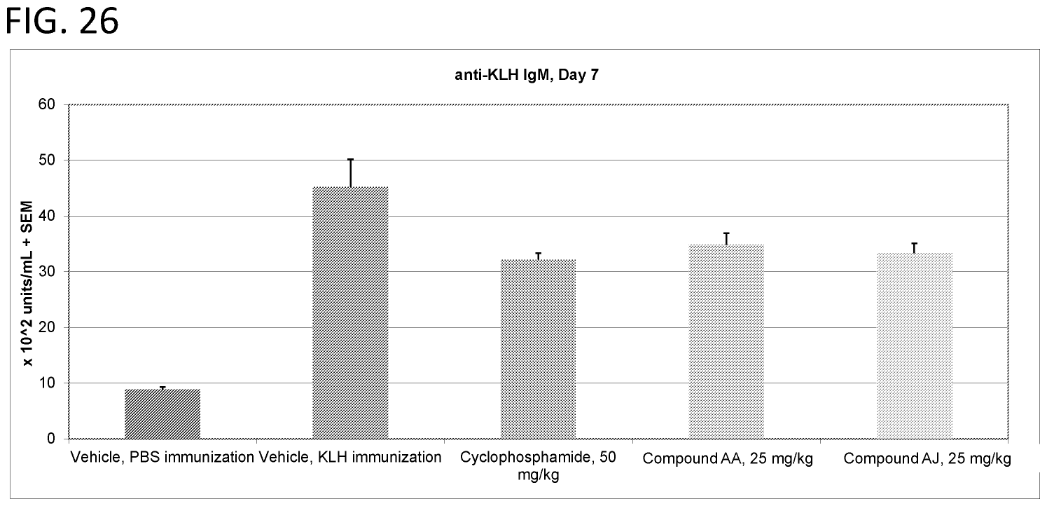

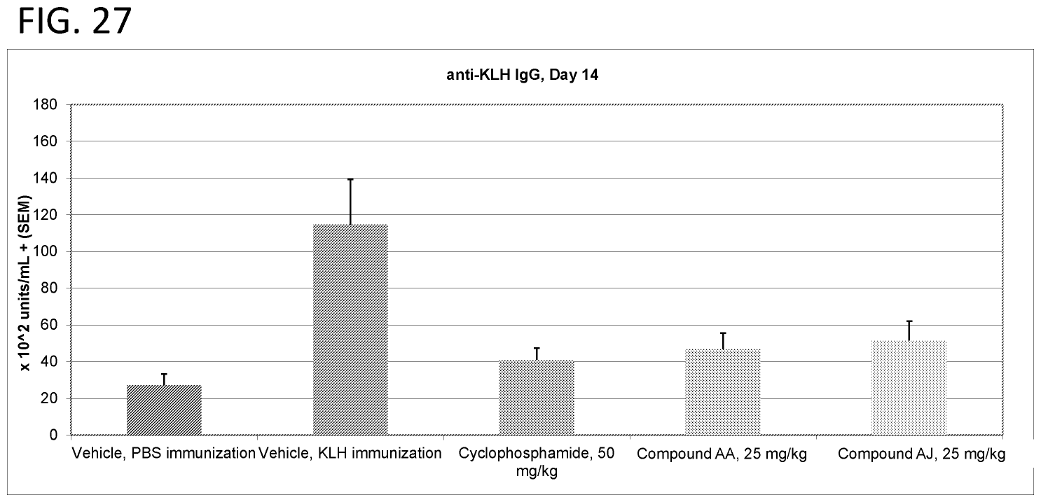

FIG. 5H is a graph showing the alternative pathway hemolytic activity of the factor H anti-albumin-VHH fusion proteins (Compound O) isolated using MEP HYPERCEL™ resin.

FIG. 5I is a graph showing the alternative pathway hemolytic activity of the factor H anti-albumin-VHH fusion proteins (Compound O) isolated using CAPTO™ Adhere ImpRes resin.

FIG. 5J is an SDS-PAGE gel showing the overall purity of the factor H anti-albumin-VHH fusion protein isolated in a large scale purification scheme using a HITRAP CAPTO™ Adhere ImpRes Column.

FIG. 6A is a schematic diagram illustrating Compound X.

FIG. 6B is a pair of SDS-PAGE gels showing the fragmentation of Compound X under reducing or non-reducing conditions.

FIG. 6C is a schematic diagram illustrating exemplary FH fusion proteins evaluated in the structure function analysis studies.

FIG. 7 is a spectra showing the ESI-ToF mass spectrometry of protein A-purified Compound X.

FIG. 8A is a schematic diagram illustrating Compound AC.

FIG. 8B is pair of SDS-PAGE gels showing the fragmentation of Compound AC under reducing or non-reducing conditions.

FIG. 8C is a spectra showing ESI-ToF mass spectrometry of Compound AC.

FIG. 9 is a graph showing inhibition of alternative pathway hemolytic activity of fusion proteins Compound AC and Compound AD.

FIG. 10 is a graph showing inhibition of alternative pathway hemolytic activity of fusion proteins containing FH or fusion proteins including CR2 and FH. Molecular descriptions and IC 50 values are shown in the accompanying table.

FIG. 11 is a graph showing inhibition of alternative pathway hemolytic activity of non-targeted FH-Fc fusion proteins. Molecular descriptions and IC 50 values are shown in the accompanying table.

FIG. 12 is a graph showing association of Compound AC (dark blue trace), Compound AP (red trace), or Compound AQ (light blue trace) with immobilized C3d by Octet BLI detection.

FIG. 13 is an SDS PAGE of Compound H indicating fragmentation under non-reducing or reducing conditions.

FIG. 14 is a graph showing the PK of compounds X, H, and AC in wild-type mice.

FIG. 15 is a graph showing inhibition of mouse alternative pathway hemolysis in mice treated with Compounds X, H, or AC.

FIG. 16 is a graph showing PK and suppression of AP hemolytic activity in wild-type mice following administration of 25 mg/kg Compound A B.

FIG. 17 is a graph showing PK and suppression of AP hemolytic activity in wild-type mice following administration with 25 mg/kg Compound AC.

FIG. 18 is a graph showing the profile of Compound AC when administered as a single 25 mg/kg IV dose to wild-type and FH−/− mice.

FIG. 19 is series of immunohistochemical images showing human factor H (Compound AC) localized to kidney glomeruli of FH−/− mice administered a single 25 mg/kg IV dose of Compound AC. Each frame provides a representative image from an individual animal. The PBS treatment group had individual animals. Three animals were analyzed on day 1 and day 3, and five animals were analyzed on days 7 and 14.

FIG. 20 is a graph showing quantitation of mean fluorescence intensity of glomerular human factor H staining (Compound AC) in FH−/− mice treated with Compound AC. The human factor H-positive pixel count mean signal intensity was calculated as an average from 20 glomeruli for each animal. Statistical significance was determined by one-way ANOVA using the Kruskal-Wallis test for multiple comparisons. An asterisk indicates statistical significance between the treatment group at a given timepoint and the non-treated (PBS) control. NS is not significant.

FIG. 21 is a series of immunohistochemical images of mouse C3 deposited on the glomeruli of FH−/− mice treated with either Compound AC or PBS. Each frame provides a representative image from an individual animal.

FIG. 22 is a graph showing quantitation of mean fluorescence intensity of glomerular C3 staining in FH−/− mice treated with Compound AC. The C3 positive pixel count mean signal intensity was calculated as an average from 20 glomeruli for each animal. Statistical significance was determined by one-way ANOVA using the Kruskal-Wallis test for multiple comparisons. An asterisk indicates statistical significance between the treatment group at a given timepoint and the non-treated (PBS) control. NS is not significant.

FIG. 23 is a series of immunohistochemical images showing deposition of properdin on the glomeruli of FH−/− mice treated with either Compound AC or PBS. Each frame provides a representative image from an individual animal.

FIG. 24 is a graph showing plasma C3 levels of FH−/− mice treated with Compound AC.

FIG. 25 is a graph showing plasma C5 levels in FH−/− and in wild-type control mice treated with Compound AC.

FIG. 26 is a graph showing a reduction in the KLH-specific IgM response in immunized animals administered cyclophosphamide, Compound AA, or Compound AJ.

FIG. 27 is a graph showing a near complete suppression of the KLH-specific IgG response in immunized animals administered cyclophosphamide, Compound AA, or Compound AJ.

### DEFINITIONS

As used herein, the term “fusion protein” refers to a composite polypeptide made up of two (or more) distinct, heterologous polypeptides. The heterologous polypeptides can either be full-length proteins, or fragments of full-length proteins. Fusion proteins herein can be prepared by either synthetic or recombinant techniques known in the art.

As used herein, the term “antibody” refers to an immunoglobulin molecule that specifically or substantially specifically binds to, or is immunologically reactive with, a particular antigen. The antibody can be, for example, a natural or artificial mono- or polyvalent antibody including, but not limited to, a polyclonal, monoclonal, multi-specific, human, humanized, or chimeric antibody. An antibody may be a genetically engineered or otherwise modified form of an antibody, including but not limited to, heteroconjugate antibodies (e.g., bi-, tri-, and tetra-specific antibodies, diabodies, triabodies, and tetrabodies), and antigen binding fragments of antibodies, including, for example, single domain, Fab′, F(ab′)₂, Fab, Fv, rIgG and scFv fragments.

As used herein, the term “single domain antibody” defines molecules where the antigen binding site is present on, and formed by, a single immunoglobulin domain. Single domain antibodies include antibodies whose complementary determining regions (“CDRs”) are part of a single domain polypeptide. Single domain antibodies include an antibody or antigen binding fragment thereof that specifically binds a single antigen. Generally, the antigen binding site of an immunoglobulin single variable domain is formed by no more than three CDRs. The single variable domain may, for example, include a light chain variable domain sequence (a V_{L} sequence) or a suitable fragment thereof; or a heavy chain variable domain sequence (e.g., a V_{H} sequence or V_{HH} sequence), or a suitable fragment thereof; as long as it is capable of forming a single antigen binding unit (i.e., a functional antigen binding unit that essentially is the single variable domain, such that the single antigen binding domain does not need to interact with another variable domain to form a functional antigen binding unit). Such antibodies can be derived, for example, from antibodies raised in Camelidae species, for example, in a camel, dromedary, llama, alpaca, or guanaco. Additional antibodies include, for example, immunoglobulin new antigen receptor (IgNAR) of cartilaginous fishes (e.g., sharks, e.g., nurse sharks). Other species besides Camelidae and cartilaginous fishes may produce antibodies whose CDRs are part of a single polypeptide. Antibodies can be prepared by either synthetic or recombinant techniques known in the art.

As used herein, the term “affinity” refers to the strength of an interaction between binding moiety and its target. For example, an Fc domain, such as an Fc receptor binding domain, interacts through non-covalent forces with an Fc receptor (e.g., FcRn, FcγRI, FcγRII, or FcγRIII). As used herein, the term “high affinity” for an Fc receptor binding domain or fragment thereof (e.g., an Fc domain) refers to an Fc domain having a K_{D} of 10⁻⁸ M or less, 10⁻⁹ M or less, 10⁻¹⁰ M or less, 10⁻¹¹ M or less, 10⁻¹² M or less, or 10⁻¹³ M or less for an Fc receptor. As used herein, the term “low affinity” for an Fc receptor binding domain or fragment thereof (e.g., an Fc domain) refers to an Fc domain having a K_{D} of 10⁻⁷ M or more, 10⁻⁶ M or more, or 10⁻⁵ M or more for an Fc receptor.

The term “Fc domain,” as used herein refers to an antibody (e.g., a monoclonal antibody), or fragment thereof, such as a fragment crystallizable (Fc) region of an antibody. Exemplary Fc domains include an Fc domain comprising the second and third constant domain of a human immunoglobulin (CH2 and CH3), or the hinge, CH2 and CH3. The immunoglobulin may be an IgG (e.g., human IgG1, IgG4, IgG2/4, or IgG4 proline stabilized construct). An Fc domain may also comprise an Fc receptor binding domain.

The term “Fc receptor binding domain,” as used herein refers to a polypeptide or antibody fragment that directly binds to an Fc receptor (e.g., FcRn, FcγRI, FcγRII, or FcγRIII), including to a mammalian Fc receptor (e.g., a human Fc receptor). Antibody fragments capable of binding to an Fc receptor include fragment crystallizable (Fc) domains from an antibody, such as an IgG (e.g., human IgG1, IgG4, IgG2/4, or IgG4 proline stabilized construct).

The term “Fc receptor” as used herein refers to a protein on the surface of immune cells, such as natural killer cells, macrophages, neutrophils, and mast cells. An Fc receptor can bind to an Fc (Fragment, crystallizable) region of an antibody that is attached to infected cells or invading pathogens and this binding can stimulate phagocytic or cytotoxic cells to destroy microbes, or infected cells by antibody-mediated phagocytosis or antibody-dependent cell-mediated cytotoxicity. There are several different types of Fc receptors, which are classified based on the type of antibody that they recognize. Herein, the term “FcRn” refers to the neonatal Fc receptor that binds IgG. FcRn is similar in structure to MHC class I protein, which, in humans, is encoded by the FCGRT gene. An Fc receptor binding domain that binds directly to FcRn includes an antibody Fc domain. Regions capable of binding to a polypeptide such as albumin or IgG, which has human FcRn-binding activity, can indirectly bind to human FcRn via albumin, IgG, or such. Thus, such a human FcRn-binding region may be a region that binds to a polypeptide having human FcRn-binding activity. Other Fc receptors include FcγRI, FcγRII, and FcγRIII.

As used herein, the term “fused” or “joined” refers to the combination or attachment of two or more elements, components, or protein domains, e.g., polypeptides, by means including chemical conjugation, recombinant means, and chemical bonds, e.g., disulfide bonds and amide bonds. For example, two single polypeptides can be joined to form one contiguous protein structure through recombinant expression, chemical conjugation, a chemical bond, a peptide linker, or any other means of covalent linkage.

As used herein, the term “linker” refers to a linkage between two elements, e.g., polypeptides or protein domains. A linker can be a covalent bond. A linker can also be a molecule of any length that can be used to couple, for example, a factor H fragment and/or a CR2 fragment with an Fc domain, such as an Fc receptor binding domain. A linker also refers to a moiety (e.g., a polyethylene glycol (PEG) polymer) or an amino acid sequence (e.g., a 1-200 amino acid, 1-150 amino acid, 1-100, a 5-50, or a 1-10 amino acid sequence, particularly amino acids with smaller side chains and/or flexible amino acid sequences) occurring between two polypeptides or polypeptide domains to provide space and/or flexibility between the two polypeptides or polypeptide domains. An amino acid linker may be part of the primary sequence of a polypeptide (e.g., joined to the linked polypeptides or polypeptide domains via the polypeptide backbone). Non-limiting examples include (G₄A)₂G₄S, G₄SDAA, (G₄S), and (G₄A)₂G₃AG₄S. (SEQ ID NOs: 14-16, and 79).

As used herein, the term “host cell” refers to any kind of cellular system that can be engineered to generate the fusion proteins described herein. Non-limiting examples of host cells include HEK, HEK293, HT-1080, CHO, Pichia pastoris, Saccharomyces cerevisiae, and transformable insect cells such as High Five, Sf9, and Sf21 cells.

As used herein, the term “operatively linked” in the context of a polynucleotide fragment means that the two polynucleotide fragments are joined such that the amino acid sequences encoded by the two polynucleotide fragments remain in-frame.

As used herein, the term “alternative complement pathway” refers to one of three pathways of complement activation (the others being the classical pathway and the lectin pathway).

As used herein, the term “alternative complement pathway dysregulation” refers to any aberration in the ability of the alternative complement pathway to provide host defense against pathogens and clear immune complexes and damaged cells and for immunoregulation. Alternative complement pathway dysregulation can occur in the fluid phase and at the cell surface and can lead to excessive complement activation or insufficient regulation, both causing tissue injury.

As used herein, “Factor H” refers to a protein component of the alternative complement pathway encoded by the complement factor H gene (“FH;” NM000186; GeneID:3075; UniProt ID P08603; Ripoche, J. et al., Biochem. J., 249:593-602,1988). Factor H is translated as a 1,213 amino acid precursor polypeptide that is processed by removal of an 18 amino acid signal peptide, resulting in the mature factor H protein (amino acids 19-1231). Factor H consists of 20 short complement regulator (SCR) domains. Amino acids 1-18 comprise the signal peptide, residues 21-80 comprise SCR1 (SEQ ID NO: 1, residues 85-141 comprise SCR 2 (SEQ ID NO: 2), residues 146-205 comprise SCR3 (SEQ ID NO: 3), residues 201-262 comprise SCR 4 (SEQ ID NO: 4), residues 267-320 comprise SCR 5 (SEQ ID NO: 5), residues 1107-1165 comprise SCR 19 (SEQ ID NO:6), and residues 1167-1230 comprise SCR 20 (SEQ ID NO: 7). Factor H regulates complement activation on self-cells by possessing both cofactor activity for the factor I-mediated C3b cleavage, and decay accelerating activity against the alternative pathway C3 convertase, C3bBb.

As used herein, “Complement receptor 2” or “CR2” refers to human complement receptor 2, also referred to as CD21 (CR2/CD21), is a 145 kD transmembrane protein of the C3 binding protein family comprising 15 or 16 short consensus repeat (SCR) domains, structural units characteristic of such proteins. The SCR domains have a typical framework of highly conserved residues including four cysteines, two prolines, one tryptophan, and several other partially conserved glycines and hydrophobic residues. These SCR domains are separated by short sequences of variable length that serve as spacers. Amino acids 1-20 comprise the leader peptide, amino acids 23-82 comprise SCR1 (SEQ ID NO: 8), amino acids 91-146 comprise SCR2 (SEQ ID NO: 9), amino acids 154-210 comprise SCR3 (SEQ ID NO: 10), and amino acids 215-271 comprise SCR4 (SEQ ID NO: 11). The active site (C3d binding site) is located in SCR1-2 (the first two N-terminal SCR domains). CR2 is expressed on mature B cells and follicular dendritic cells, and plays an important role in humoral immunity. J. Hannan et al., Biochem. Soc. Trans. (2002) 30:983-989; K. A. Young et al., J. Biol. Chem. (2007) 282(50):36614-36625. CR2 protein does not bind intact C3 protein, but binds its breakdown products, including the C3b, iC3b, and C3d cleavage fragments, via a binding site located within the first two amino-terminal SCR domains (“SCRs 1-2”) of the CR2 protein. Consequently, the SCRs 1-2 of CR2 discriminate between cleaved (i.e., activated) forms of C3 generated during complement activation and intact circulating C3. While the affinity of CR2 for C3d is only 620-658 nM (J. Hannan et al., Biochem. Soc. Trans. (2002) 30 983-989; J. M. Guthridge et al., Biochem. (2001) 40:5931-5941), the avidity of CR2 for clustered C3d makes it an effective method of targeting molecules to sites of complement activation.

Cleavage of C3 results initially in the generation and deposition of C3b on the activating cell surface. The C3b fragment is involved in the generation of enzymatic complexes that amplify the complement cascade. On a cell surface, C3b is rapidly converted to inactive iC3b, particularly when deposited on a host surface containing regulators of complement activation (i.e., most host tissue). Even in the absence of membrane-bound complement regulators, substantial levels of iC3b are formed because of the action of serum factor H and serum factor I. iC3b is subsequently digested to the membrane-bound fragments C3dg and then C3d by factor I and other proteases and cofactors, but this process is relatively slow. Thus, the C3 ligands for CR2 are relatively long lived once they are generated and are present in high concentrations at sites of complement activation.

As used herein, a “functional fragment” or a “biologically active fragment” refers to a fragment, or portion, of a protein having some or all of the activities of the full-length protein. For example, a functional or biologically active fragment of factor H, refers to any fragment of a factor H protein having some or all of the activities of factor H, e.g., alternative complement pathway regulatory activity of the full-length factor H protein. Examples include, but are not limited to, factor H fragments, joined from N-terminus to C terminus, containing the following SCRs: [1-4], [1-5], [1-7], [1-20], [19-20], [1-4 and 19-20], and [1-5 and 19-20]. A “functional fragment” or a “biologically active fragment” of CR2 protein is one having some or all of the activities of CR2, e.g., alternative complement pathway regulatory activity of the full-length CR2 protein. Examples include, but are not limited to, CR2 fragments, from N-terminus to C-terminus, containing the following SCRs: [1-2], [1-3], or [1-4].

As used herein, the term “fragment” refers to less than 100 0/0 of the amino acid sequence or a full-length reference protein (e.g., 99%, 90%, 80%, 70%, 60%, 50%, 40%, 30%, 20%, 10%, of the full-length sequence etc.), but including, e.g., 20, 25, 30, 35, 40, 45, 50, 100, 150, 200, 250, 300, 350, or more amino acids. A fragment can be of sufficient length such that a desirable function of the full-length protein is maintained. For example, the regulation of the alternative complement pathway in the fluid phase by fragments of, for example, factor H, is maintained. Such fragments are “biologically active fragments.”

As used herein, the terms “short complement regulator”, or “SCR”, also known as “short consensus repeat”, “sushi domains,” or “complement control protein” or “CCP,” describe domains found in all regulators of complement activation (RCA) gene clusters that contribute to their ability to regulate complement activation in the blood or on the cell surface to which they specifically bind. SCRs typically are composed of about 60 amino acids, with four cysteine residues disulfide bonded in a 1-3, 2-4 arrangement and a hydrophobic core built around an almost invariant tryptophan residue. SCRs are found in proteins including, but not limited to, factor H and CR2.

“Percent (%) sequence identity,” with respect to a reference polynucleotide or polypeptide sequence, is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software, such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by(the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “disease” refers to an interruption, cessation, or disorder of body functions, systems, or organs. Disease(s) or disorders of interest include those that would benefit from treatment with a fusion protein or method described herein. Non-limiting examples of diseases or disorders to be treated herein resulting from the dysregulation of the alternative complement pathway activation include, but are not limited to, kidney disorders, cutaneous disorders, and neurological disorders; for example, paroxysmal nocturnal hemoglobinuria (PNH), atypical hemolytic uremic syndrome (aHUS), IgA nephrology, lupus nephritis, C3 glomerulopathy (C3G), dermatomyositis, systemic sclerosis, demyelinating polyneuropathy, pemphigus, membranous nephropathy, focal segmental glomerular sclerosis (FSGS), bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, dense deposit disease (DDD), age related macular degeneration (AMD), systemic lupus erythematosus (SLE), rheumatoid arthritis (RA), multiple sclerosis (MS), traumatic brain injury (TBI), ischemia reperfusion injury, preeclampsia, or thrombic thrombocytopenic purpura (TTP).

As used herein, the terms “treatment,” “treating,” or “treat” refer to therapeutic treatment, in which the object is to inhibit or lessen an undesired physiological change or disorder or to promote a beneficial phenotype in a patient. For example, “treatment,” “treating” or “treat” refer to clinical intervention in an attempt to alter the natural course of an individual's affliction, disease, or disorder. The terms include, for example, prophylaxis before or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration, or palliation of the disease state, and improved prognosis. In some embodiments, fusion proteins are used to control the cellular and clinical manifestations of kidney disorders, cutaneous disorders, and neurological disorders, such as PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, and TTP.

As used herein, “administering” and “administration” refers refer to any method of providing a pharmaceutical preparation to a subject. Fusion proteins may be administered by any method known to those skilled in the art. Suitable methods for administering the fusion protein may be, for example, orally, by injection (e.g., intravenously, intraperitoneally, intramuscularly, intravitreally, and subcutaneously), drop infusion preparations, inhalation, intranasally, and the like. In particular, administrations is via intravenous and/or subcutaneous infusions. Fusion proteins prepared as described herein may be administered in various forms, depending on the disorder to be treated and the age, condition, and body weight of the subject, as is known in the art. A preparation can be administered prophylactically; that is, administered to decrease the likelihood of developing a disease or condition.

As used herein, the term “effective amount” refers to an amount that is sufficient to achieve the desired result or to have an effect on an undesired condition. For example, an “effective amount” refers to an amount that is sufficient to achieve the desired therapeutic result. The specific therapeutically effective dose for any particular patient will depend upon a variety of factors including the disorder being treated and the severity of the disorder; the specific composition employed; the age, body weight, general health, sex, and diet of the patient; the time of administration; the route of administration; the rate of excretion of the specific compound employed; the duration of the treatment; drugs used in combination or coincidental with the specific compound employed, and like factors known in the art. Dosage can vary, and can be administered in one or more dose administrations daily, weekly, monthly, or yearly, for one or several days.

As used herein, the term “patient in need thereof” or “subject in need thereof,” refers to the identification of a subject based on need for treatment of a disease or disorder. A subject can be identified, for example, as having a need for treatment of a disease or disorder (e.g., PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, and TTP), based upon an earlier diagnosis by a person of skill in the art (e.g., a physician). In particular, a patient is a mammal, particularly a human.

### DETAILED DESCRIPTION

Described herein are alternative complement pathway-specific C3 and C5 convertase inhibitors that regulate alternative complement pathway activity. Diseases mediated by complement dysregulation are often a result of complement overactivity both in the fluid phase and at the cell surface. Described herein are compositions and methods for treating diseases mediated by complement dysregulation. Examples of disorders mediated by alternative complement pathway dysregulation include, for example, kidney disorders, cutaneous disorders, and neurological disorders, such as paroxysmal nocturnal hemoglobinuria (PNH), atypical hemolytic uremic syndrome (aHUS), IgA nephrology, lupus nephritis, C3 glomerulopathy (C3G), dermatomyositis, systemic sclerosis, demyelinating polyneuropathy, pemphigus, membranous nephropathy, focal segmental glomerular sclerosis (FSGS), bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, dense deposit disease (DDD), age related macular degeneration (AMD), systemic lupus erythematosus (SLE), rheumatoid arthritis (RA), multiple sclerosis (MS), traumatic brain injury (TBI), ischemia reperfusion injury, preeclampsia, and thrombic thrombocytopenic purpura (TTP). The compositions and methods described herein feature fusion proteins that include a fragment of complement factor H (FH) fused to an Fc domain (e.g., a monoclonal antibody, or fragment thereof (e.g., an Fc domain)). The fusion proteins may also contain a fragment of CR2. Exemplary fusion proteins for use in the methods of the invention include, but are not limited to, Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222). In some embodiments, the fusion protein is Compound A B, Compound AC, or Compound AJ (e.g., a fusion protein having an amino acid sequence of any one of SEQ ID NO: 147, 148, or 155, or a fusion protein encoded by the nucleic acid sequence of any one of SEQ ID NO: 192, 193, or 200).

The fusion protein or fusion proteins according to the disclosure herein regulate(s) alternative complement pathway activity, by attenuating C3 and C5 convertase activity. Moreover, the Fc domain increases the serum half-life of the fusion protein, may stabilize the fusion protein overall, and aids in manufacturing, i.e., via protein A affinity chromatography. The overall design targets the alternative complement pathway and leaves activation (protection) via classical and lectin pathways intact.

### Fusion Proteins

As described herein, fusion proteins that include a fragment of factor H and an Fc domain (e.g., an IgG or a functional fragment thereof, e.g., an Fc domain, such as an Fc domain that binds an Fc receptor) can be used as therapeutic agents to treat diseases mediated by alternative complement pathway dysregulation. In humans, several regulatory proteins are encoded by a cluster of genes located on the long arm of chromosome 1. This region is called the regulator of complement activation (RCA) gene cluster. Although the proteins within the RCA family vary in size, they share significant primary amino acid structure similarities. The best studied members of the RCA family are factor H, FHL-1, CR1, DAF, MCP, and C4b-binding protein (C4BP). The members are organized in tandem structural units termed short consensus repeats (SCRs), which are present in multiple copies in the protein. Each SCR consists of 60-70 highly conserved amino acids, including 4 cysteines.

In some embodiments, the portion of the fusion protein suitable for inhibiting activity of the alternative complement pathway is fused with a larger polypeptide, e.g., human albumin, an antibody, an antibody fragment, or Fc, for increased duration of effect.

In certain embodiments, the portion of the fusion protein suitable for inhibiting activity of the alternative complement pathway includes a fragment of factor H. The fragment of factor H may include at least the first four N-terminal SCR domains of factor H (e.g., SCRs 1, 2, 3, and 4). In certain embodiments, the fragment of factor H includes at least the first five N-terminal SCR domains of factor H (e.g., SCRs 1, 2, 3, 4, and 5) (also known as the cofactor and decay accelerating domains). In certain embodiments, the fragment of factor H may also include at least the first four or five N-terminal SCRs and the last two N-terminal SCR domains of factor H (e.g., SCRs 1, 2, 3, 4, 19, and 20 or SCRs 1, 2, 3, 4, 5, 19, and 20).

The fusion protein may include, in addition to a fragment of factor H, a fragment of complement receptor 2 (CR2). The fragment of factor H in the fusion protein may include at least the first four or five N-terminal SCR domains of factor H and the fragment of CR2 in the fusion protein may include at least the first two N-terminal SCR domains of CR2 (e.g., SCRs 1 and 2). In other embodiments, the fragment of CR2 may include at least the first three or four N-terminal SCR domains of CR2 (e.g., SCRs 1, 2 and 3 or SCRs 1, 2, 3, and 4).

In certain embodiments, the fragment of factor H includes at least the first five N-terminal SCR domains of factor H (e.g., SCRs 1, 2, 3, 4, and 5), and the fragment of CR2 includes at least the first two N-terminal SCR domains of CR2 (e.g., SCRs 1 and 2). In certain embodiments, the fragment of factor H includes at least the first five N-terminal SCR domains of factor H (e.g., SCRs 1, 2, 3, 4, and 5), and the fragment of CR2 includes at least the first three N-terminal SCR domains of CR2 (e.g., SCRs 1, 2 and 3). In certain embodiments, the fragment of factor H includes at least the first five N-terminal SCR domains of factor H (e.g., FH SCRs 1, 2, 3, 4, and 5), and the fragment of CR2 includes at least the first four N-terminal SCR domains of CR2 (e.g., CR2 SCRs 1, 2, 3, and 4).

In certain embodiments, the fragment of factor H includes at least the first four and the last two N-terminal SCR domains of factor H (e.g., SCRs 1, 2, 3, 4, 19, and 20), and the fragment of CR2 includes at least the first two N-terminal SCR domains of CR2 (e.g., SCRs 1 and 2). In certain embodiments, the fragment of factor H includes at least the first four and the last two N-terminal SCR domains of factor H (e.g., SCRs 1, 2, 3, 4, 19, and 20), and the fragment of CR2 includes at least the first three N-terminal SCR domains of CR2 (e.g., SCRs 1, 2 and 3). In certain embodiments, the fragment of factor H includes at least the first four and the last two N-terminal SCR domains of factor H (e.g., SCRs 1, 2, 3, 4, 19, and 20), and the fragment of CR2 includes at least the first four N-terminal SCR domains of CR2 (e.g., SCRs 1, 2, 3, and 4).

In certain embodiments, the fragment of factor H includes at least the first five and last two N-terminal SCR domains of factor H (e.g., SCRs 1, 2, 3, 4, 5, 19, and 20), and the fragment of CR2 includes at least the first two N-terminal SCR domains of CR2 (e.g., SCRs 1 and 2). In certain embodiments, the fragment of factor H includes at least the first five and last two N-terminal SCR domains of factor H (e.g., SCRs 1, 2, 3, 4, 5, 19, and 20), and the fragment of CR2 includes at least the first three N-terminal SCR domains of CR2 (e.g., SCRs 1, 2 and 3). In certain embodiments, the fragment of factor H includes at least the first five and last two N-terminal SCR domains of factor H (e.g., SCRs 1, 2, 3, 4, 5, 19, and 20), and the fragment of CR2 includes at least the first four N-terminal SCR domains of CR2 (e.g., SCRs 1, 2, 3, and 4).

In some embodiments, the fragment of factor H portion of the fusion protein is a functional fragment of wild-type factor H. In some embodiments, the factor H, or fragment thereof portion of the fusion protein is derived from a substituted (e.g., conservatively substituted) factor H or an engineered factor H (e.g., a factor H engineered to increase stability, activity, and/or other desirable properties of the protein, as determined by a predictive model or assay known to one of skill in the art, such as described herein).

In some embodiments, the fragment of CR2 portion of the fusion protein is a functional fragment of wild-type CR2. In some embodiments, the CR2 or fragment thereof portion of the fusion protein composition is derived from a substituted (e.g., conservatively substituted) CR2 or an engineered CR2 (e.g., aCR2 engineered to increase stability, activity, and/or other desirable properties of the protein, as determined by a predictive model or assay known to one of skill in the art, such as an assay described herein).

Amino acid substitutions can be introduced into the fusion proteins described herein to improve functionality. For example, amino acid substitutions can be introduced into the fragment of factor H or CR2, wherein an amino acid substitution increases binding affinity of fragment of factor H or CR2 for its ligand(s). Similarly, amino acid substitutions can be introduced into the fragment of factor H, CR2, or the Fc, or fragment thereof, to increase functionality and/or to improve the pharmacokinetics of the fusion protein. In some embodiments, the N107 residue of CR2 SCR 2 is changed to GIn (N107Q). In some embodiments, the S109 residue of CR2 SCR 2 is changed to Ala (S109A). In some embodiments, the N107 residue of CR2 SCR 2 is changed to GIn (N107Q) and the S109 residue of CR2 SCR 2 is changed to Ala (S109A). In some embodiments, the S103 residue of CR2 SCR 2 is changed to Ala (S103A). In some embodiments, the N101 residue of CR2 SCR 2 is changed to GIn (N1010). In some embodiments, the first or the second, or both, N-linked glycosylation consensus sequences may be mutated to eliminate the consensus sequence so that it is no longer glycosylated.

In certain embodiments, the fusion proteins described herein can be fused with another compound, such as a compound to increase the half-life of the polypeptide and/or to reduce potential immunogenicity of the fusion protein (for example, polyethylene glycol (PEG)). PEG can be used to improve water solubility, reduce the rate of kidney clearance, and reduce immunogenicity of the fusion protein (see, e.g., U.S. Pat. No. 6,214,966, the disclosure of which is incorporated herein by reference). The fusion proteins described herein can be PEGylated by any means known to one skilled in the art.

The fragment of factor H and/or CR2 may be prepared by a number of synthetic methods of peptide synthesis by fragment condensation of one or more amino acid residues, according to conventional peptide synthesis methods known in the art (Amblard, M. et al., Mol. Biotechnol., 33′239-54, 2006).

Alternatively, a fragment of factor H and/or CR2 may be produced by expression in a suitable prokaryotic or eukaryotic system. In some embodiments, a DNA construct may be inserted into a plasmid vector adapted for expression in a suitable host cell (such as E. coli) or a yeast cell (such as S. cerevisiae or P. pastoris), or into a baculovirus vector for expression in an insect cell, or a viral vector for expression in a mammalian cell. Examples of suitable mammalian cells for recombinant expression include, e.g., a human embryonic kidney cell (HEK) (e.g., HEK 293), a Chinese Hamster Ovary (CHO) cell, L cell, C127 cell, 3T3 cell, BHK cell, or COS-7 cell. Suitable expression vectors include the regulatory elements necessary and sufficient for expression of the DNA in the host cell. In some embodiments, a leader or secretory sequence or a sequence that is employed for purification of the fusion protein, can be included in the fusion protein. The fragment of factor H and/or CR2 produced by gene expression in a recombinant prokaryotic or eukaryotic system may be purified according to methods known in the art (See, e.g., Structural Genomics Consortium, Nat. Methods, 5:135-46, 2008).

In some embodiments, the fusion protein has the structure, from N-terminus to C-terminus, of Formula I:

D1-L1-Fc-L2-D2 Formula I

wherein

D1 is a fragment of FH (e.g., a fragment of FH of any one of SEQ ID NOs: 108-110, 134, and 135) and/or a fragment of CR2 (e.g., a fragment of CR2 of any one of SEQ ID NOs: 94-107 and 136-141);

L1 is absent (e.g., is a covalent bond between D1 and Fc), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between D1 and Fc;

Fc is an Fc domain, such as an Fc receptor binding domain (e.g., the Fc domain has the sequence of any one of SEQ ID NOs: 88 and 111-113, and, preferably, the sequence of SEQ ID NO: 88);

L2 is absent (e.g., is a covalent bond between Fc and D2), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between Fc and D2; and

D2 is a fragment of FH (e.g., a fragment of FH of any one of SEQ ID NOs: 108-110, 134, and 135) and/or a fragment of CR2 (e.g., a fragment of CR2 of any one of SEQ ID NOs: 94-107 and 136-141).

In an embodiment, D1 and D2 do not both comprise a fragment of CR2.

In some embodiments the fragment of FH of D1 includes one or more FH SCR domains, preferably wherein the one or more SCR domains are selected from the group consisting of SCR 1, 2, 3, 4, 5, 19, and 20, and/or the fragment of FH of D2 includes one or more FH SCR domains, preferably wherein the one or more SCR domains are selected from the group consisting of SCR 1, 2, 3, 4, 5, 19, and 20. In some embodiments, the FH SCR domains are selected from the group consisting of SCR [1-4] (e.g., a fragment of FH of SEQ ID NO: 109); [1-5] (e.g., a fragment of FH of SEQ ID NO: 108); [1-4, 19, and 20] (e.g., a fragment of FH of SEQ ID NO: 134); [1-5, 19, and 20](e.g., a fragment of FH of SEQ ID NO: 135); and [19 and 20] (e.g., a fragment of FH of SEQ ID NO: 110).

In some embodiments, the fragment of CR2 of D1 includes one or more CR2 SCR domains, preferably wherein the one or more SCR domains are selected from the group consisting of SCR 1, 2, 3, and 4, and/or the fragment of CR2 of D2 includes one or more CR2 SCR domains, preferably wherein the one or more SCR domains are selected from the group consisting of SCR 1, 2, 3, and 4.

In some embodiments, the CR2 SCR domains are selected from the group consisting of: SCR [1-2](e.g., a fragment of CR2 of any one of SEQ ID NOs: 95 and 102-107), [1-3] (e.g., a fragment of CR2 of any one of SEQ ID NOs: 136-141), and [1-4] (e.g., a fragment of CR2 of any one of SEQ ID NOs: 94 and 96-101).

In some embodiments, D1 or D2 is a fragment of FH fused by L3 to a fragment of FH, wherein L3 is an amino acid sequence of at least one amino acid. In some embodiments, the fragment of FH includes SCR domains 19 and 20 (e.g., a fragment of FH of SEQ ID NO: 110).

In some embodiments, D1 or D2 is a fragment of FH fused by L3 to a fragment of CR2, wherein L3 is an amino acid sequence of at least one amino acid (e.g., the linker of any one of SEQ ID NOs 13-87, 142, 143, 163, 169, and 226-238, and preferably, of any one of SEQ ID NOs: 14, 15, 16, 79, 163, and 226-238). In some embodiments, the fragment of FH comprises SCR domains 19 and 20, and the fragment of CR2 comprises SCR domains 1-2 (e.g., a fragment of CR2 of any one of SEQ ID NOs: 95 and 102-107).

L1, L2, and L3 may be linkers of the same type and/or sequence or of a different type and/or sequence.

In some embodiments, the fusion protein has the structure, from N-terminus to C-terminus, of Formula II:

D1-L1-Fc-L2-D2 Formula II

wherein D1 is a fragment of FH (e.g., a fragment of FH of any one of SEQ ID NOs: 108-110, 134, and 135);

L1 is absent (e.g., is a covalent bond between D1 and Fc), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between D1 and Fc;

Fc is an Fc domain, such as an Fc receptor binding domain (e.g., the Fc domain has the sequence of any one of SEQ ID NOs: 88 and 111-113, and, preferably, the sequence of SEQ ID NO: 88);

L2 is absent (e.g., is a covalent bond between Fc and D2), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between Fc and D2; and

D2 is a fragment of FH (e.g., a fragment of FH of any one of SEQ ID NOs: 108-110, 134, and 135).

In some embodiments, the fusion protein has the structure, from N-terminus to C-terminus, of Formula III:

D1-L1-Fc-L2-D2 Formula III

wherein D1 is a fragment of FH (e.g., a fragment of FH of any one of SEQ ID NOs: 108-110, 134, and 135);

L1 is absent (e.g., is a covalent bond between D1 and Fc), or is a linker of an amino acid sequence of at least 1 amino acid (e.g., the linker of any one of SEQ ID NOs 13-87, 142, 143, 163, and 169, and preferably, of any one of SEQ ID NOs:14, 15, 16, 79, and 163) between D1 and Fc;

Fc is an Fc domain, such as an Fc receptor binding domain (e.g., the Fc domain has the sequence of any one of SEQ ID NOs: 88 and 111-113, and, preferably, the sequence of SEQ ID NO: 88);

L2 is absent (e.g., is a covalent bond between Fc and D2), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between Fc and D2; and

D2 is a fragment of CR2 (e.g., a fragment of CR2 of any one of SEQ ID NOs: 94-107 and 136-141).

In some embodiments, the fusion protein has the structure, from N-terminus to C-terminus, of Formula IV:

D1-L1-Fc-L2-D2 Formula IV

wherein D1 is a fragment of CR2 (e.g., a fragment of CR2 of any one of SEQ ID NOs: 94-107 and 136-141);

L1 is absent (e.g., is a covalent bond between D1 and Fc), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between D1 and Fc;

Fc is an Fc domain, such as an Fc receptor binding domain (e.g., the Fc domain has the sequence of any one of SEQ ID NOs: 88 and 111-113, and, preferably, the sequence of SEQ ID NO: 88);

L2 is absent (e.g., is a covalent bond between Fc and D2), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between Fc and D2; and

D2 is a fragment of FH (e.g., a fragment of FH of any one of SEQ ID NOs: 108-110, 134, and 135).

In some embodiments, the fusion protein has the structure, from N-terminus to C-terminus, of Formula V:

D1-L1-Fc-L2-D2 Formula V

wherein D1 is a fragment of FH (e.g., a fragment of FH of any one of SEQ ID NOs: 108-110, 134, and 135);

L1 is absent (e.g., is a covalent bond between D1 and Fc), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between D1 and Fc;

Fc is an Fc domain, such as an Fc receptor binding domain (e.g., the Fc domain has the sequence of any one of SEQ ID NOs: 88 and 111-113, and, preferably, the sequence of SEQ ID NO: 88);

L2 is absent (e.g., is a covalent bond between Fc and D2), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between Fc and D2; and

D2 is a polypeptide having the structure, from N-terminus to C-terminus, CR2-L3-FH, wherein CR2 is a fragment of CR2 comprising CR2 SCR domains 1-2 (e.g., a fragment of CR2 of any one of SEQ ID NOs: 95 and 102-107), L3 is an amino acid sequence of at least one amino acid (e.g., the linker of any one of SEQ ID NOs 13-87, 142, 143, 163, 169, and 226-238, and preferably, of any one of SEQ ID NOs: 14, 15, 16, 79, 163, and 226-238), and FH is a fragment of FH comprising FH SCR domains 19-20 (e.g., a fragment of FH of SEQ ID NO: 110).

In some embodiments, the fusion protein has the structure, from N-terminus to C-terminus, of Formula VI:

D1-L1-Fc-L2-D2 Formula VI

wherein D1 is a polypeptide having the structure, from N-terminus to C-terminus, CR2-L3-FH, wherein CR2 is a fragment of CR2 comprising CR2 SCR domains 1-2 (e.g., a fragment of CR2 of any one of SEQ ID NOs: 95 and 102-107), L3 is an amino acid sequence of at least one amino acid, and FH is a fragment of FH comprising FH SCR domains 19-20 (e.g., a fragment of FH of SEQ ID NO: 110);

L1 is absent (e.g., is a covalent bond between D1 and Fc), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between D1 and Fc;

Fc is an Fc domain, such as an Fc receptor binding domain (e.g., the Fc domain has the sequence of any one of SEQ ID NOs: 88 and 111-113, and, preferably, the sequence of SEQ ID NO: 88);

L2 is absent (e.g., is a covalent bond between Fc and D2), or is a linker of an amino acid sequence of at least 1 amino acid (e.g., the linker of any one of SEQ ID NOs 13-87, 142, 143, 163, 169, and 226-238, and preferably, of any one of SEQ ID NOs: 14, 15, 16, 79, 163, and 226-238) between Fc and D2; and

D2 is a fragment of FH (e.g., a fragment of FH of any one of SEQ ID NOs: 108-110, 134, and 135).

In some embodiments, a fragment of FH is fused to an Fc which is fused to a fragment of FH. In some embodiments, a fragment of FH is fused to an Fc which is fused to a fragment of CR2. In some embodiments, a fragment of FH is fused to a fragment of FH, which is fused to an Fc, which is fused to a fragment of FH. In some embodiments, a fragment of CR2 is fused to a fragment of FH, which is fused to an Fc, which is fused to a fragment of FH. In some embodiments, a fragment of FH is fused to an Fc, which is fused to a fragment of FH, fused to a fragment of FH. In some embodiments, a fragment of FH is fused to an Fc, which is fused to a fragment of CR2, fused to a fragment of FH.

Exemplary fusion proteins for use in the methods as described herein are found in Tables 1-4, below.

### Immunoglobulin Proteins and Fc Domains

Factor H fusion proteins, as described herein, include either a fragment of factor H fused to an Fc domain or a fragment of factor H and a fragment of CR2 fused to an Fc domain. In some embodiments, the Fc domain is an antibody, or a functional fragment thereof, such as an Fc receptor binding domain. The Fc domain may be from an IgA, IgD, IgE, IgG, or IgM antibody, or a fragment thereof.

The fusion proteins described herein may utilize a wide variety of antibodies or antibody fragments containing an Fc domain. In some instances, the Fc domain includes a complete monoclonal antibody (e.g., an IgG). In some embodiments, the Fc domain includes only the fragment crystallizable (Fc) domain of an antibody. In some embodiments, the full length antibody (e.g., an IgG molecule) may comprise a constant region, or a portion thereof, from any type of antibody isotype, including, for example, IgG (including IgG1, IgG2, IgG3, and IgG4), or a hybrid constant region, or a portion thereof (e.g., a chimera), such as a G₂/G₄ hybrid constant region (see e.g., Burton D R and Woof J M, Adv. Immun. 51:1-18 (1992); Canfield S M and Morrison S L, J. Exp. Med. 173: 1483-1491 (1991); Mueller J P, et al., Mol. Immunol. 34(6): 441-452 (1997)). Exemplary Fc domains include an Fc region comprising the second and third constant domain of a human immunoglobulin (CH2 and CH3), or the hinge, CH2, and CH3. An Fc domain may or may not include a hinge region (e.g., residues ERKCC of the human IgG2 upper hinge region). For example, the Fc domain may be an IgG 2/4 Fc domain having the sequence VECPPCPAPPVAGPSVFLFPPKPKDTLMISRTPEVTCVVV DVSQEDPE VQFNWYVDGVEVHNAKTKPR EEQFNSTYRVVSVLTVLHQDWLNGKEYKCKVSNKGLPSSIEKTISKAKGQPREPQVYTLPPSQEEMT KNQVSLTCLVKGFYPSDIAVEWESNGQPENNYKTTPPVLDSDGSFFLYSRLTVDKSRWQEGNVFSC SVMHEALHNHYTQKSLSLSLGK (SEQ ID NO: 88) or ERKCCVECPPCPAPPVAGPSVFLFPPKPKDTLMISRTPEVTCVVVDVSQEDPEVQFNWYVDGVEVH NAKTKPREEQFNSTYRVVSVLTVLHQDWLNGKEYKCKVSNKGLPSSIEKTISKAKGQPREPQVYTLP PSQEEMTKNQVSLTCLVKGFYPSDIAVEWESNGQPENNYKTTPPVLDSDGSFFLYSRLTVDKSRWQ EGNVFSCSVMHEALHNHYTQKSLSLSLGK (SEQ ID NO: 111). Additional exemplary Fc domains include a proline-stabilized hinge, CH2, and CH3 of IgG4 having the sequence ESKYGPPCPPCPAPEFLGGPSVFLFPPKPKDTLMISRTPEVTCVVVDVSQEDPEVQFNWYVDGVEV HNAKTKPREEQFNSTYRVVSVLTVLHQDWLNGKEYKCKVSNKGLPSSIEKTISKAKGQPREPQVYTL PPSQEEMTKNQVSLTCLVKGFYPSDIAVEWESNGQPENNYKTTPPVLDSDGSFFLYSRLTVDKSRW QEGNVFSCSVMHEALHNHYTQKSLSLSLGK (SEQ ID NO: 112). The Fc domain may be that from an IgG (e.g., human IgG1, e.g., of the hinge, CH2, and CH3 regions of IgG1 having the sequence of AEPKSDKTHTCPPCPAPELLGGPSVFLFPPKPKDTLMISRTPEVTCVVVDVSHEDPEVKFNWYVDGV EVHNAKTKPREEQYNSTYRVVSVLTVLHQDWLNGKEYKCKVSNKALPAPIEKTISKAKGQPREPQVY TLPPSRDELTKNQVSLTCLVKGFYPSDIAVEWESNGQPENNYKTTPPVLDSDGSFFLYSKLTVDKSR WQQGNVFSCSVMHEALHNHYTQKSLSLSPGK (SEQ ID NO: 113)).

In some embodiments, the factor H fusion protein including an Fc domain has an increased half-life relative to a fusion protein lacking the Fc domain.

### Serum Protein-Binding Peptides

The fusion protein may also have a serum-binding peptide, which can improve the pharmacokinetics of the fusion protein. The serum-binding peptide may replace the Fc domain of the fusion protein or the serum protein-binding peptide may be added as an additional domain to the fusion protein.

As one example, the serum-binding peptide may be an albumin-binding peptide. For example, the albumin-binding peptide may have the sequence DICLPRWGCLW (SEQ ID NO: 12). Different variants of albumin-binding peptides can be constructed and attached to the fusion protein.

In some embodiments, the fusion protein includes (a) a moiety including a fragment of complement receptor 2 (CR2) (e.g., a fragment of CR2 of any one of SEQ ID NOs: 94-107 and 136-141); (b) a moiety including a fragment of complement factor H (FH) (e.g., a fragment of FH of any one of SEQ ID NOs: 108-110, 134, and 135); and (c) an anti-albumin V_{HH} domain, wherein optionally (a), (b), and/or (c) may be fused by a linker (e.g., the linker of any one of SEQ ID NOs 13-87, 142, 143, 163, 169, and 226-238, and preferably, of any one of SEQ ID NOs: 14, 15, 16, 79, 163, and 226-238). Fusion proteins can also include albumin binding peptides that can be attached to the N- or C-terminus of the fusion protein. Within a fusion protein described herein, a serum-binding peptide (e.g., an albumin binding peptide) may be attached to the N-terminus or to the C-terminus of: (a) an Fc domain, such as an Fc receptor binding domain; (b) a fragment of factor H; or (c) a fragment of CR2.

In some embodiments, the fusion protein includes (a) a moiety including a fragment of FH (e.g., a fragment of FH of any one of SEQ ID NOs: 108-110, 134, and 135), and (b) an anti-albumin V_{HH} domain, wherein optionally (a) and (b) may be fused by a linker (e.g., the linker of any one of SEQ ID NOs 13-87, 142, 143, 163, 169, and 226-238, and preferably, of any one of SEQ ID NOs: 14, 15, 16, 79, 163, and 226-238).

Albumin binding peptides and human serum albumin can be fused genetically to a regulator of the alternative complement pathway or through chemical means, e.g., chemical conjugation. If desired, a linker can be inserted between the fragment of factor H, Fc domain, such as an Fc receptor binding domain, and the albumin binding peptide. If desired, a linker can be inserted between the fragment of CR2, Fc domain, such as an Fc receptor binding domain, and the albumin binding peptide. Without being bound to a particular theory, it is expected that inclusion of an albumin binding peptide or human serum albumin in a fusion protein may lead to prolonged retention of the therapeutic protein in vivo and ex vivo.

### Linkers for the Fusion Proteins

The L1, L2, and L3 domains of the fusion proteins described herein are linkers. A linker is used to create a linkage or connection between, for example, polypeptides, or protein domains. For example, a fragment of factor H may be linked directly to an Fc domain (e.g., an IgG, or a functional fragment thereof, e.g., an Fc domain) by one or more suitable linkers. A linker can be a simple covalent bond, e.g., a peptide bond, a synthetic polymer, e.g., a PEG polymer, or any kind of bond created from a chemical reaction, e.g., chemical conjugation. The peptide linker can be, for example, a linker of one or more amino acid residues inserted or included at the transition between the two domains (e.g., a fragment of the FH domain and an Fc receptor binding domain). The identity and sequence of amino acid residues in the linker may vary depending on the desired secondary structure. For example, glycine, serine, and alanine are useful for linkers given their flexibility. Any amino acid residue can be considered as a linker in combination with one or more other amino acid residues, which may be the same as or different from the first amino acid residue, to construct larger peptide linkers as necessary depending on the desired length and/or properties.

A variety of linkers can be used to fuse two or more protein domains together (e.g., a fragment of factor H and an Fc domain). Linkers may be flexible, rigid, or cleavable. Linkers may be structured or unstructured. The residues for the linker may be selected from naturally occurring amino acids, non-naturally occurring amino acids, and modified amino acids. The linker may include at least 1 or more, 2 or more, 5 or more, 10 or more, 15 or more, or 20 or more amino acid residues. Peptide linkers can include, but are not limited to, glycine linkers, glycine-rich linkers, serine-glycine linkers, and the like. A glycine-rich linker includes at least about 50% glycine.

In some embodiments, the linker(s) used confer one or more other favorable properties or functionality to the polypeptide(s) described herein, and/or provide one or more sites for the formation of derivatives and/or for the attachment of functional groups. For example, linkers containing one or more charged amino acid residues can provide improved hydrophilic properties, whereas linkers that form or contain small epitopes or tags can be used for the purposes of detection, identification, and/or purification. A skilled artisan will be able to determine the optimal linkers for use in a specific polypeptide.

When two or more linkers are used for a polypeptide, the linkers may be the same or different.

Linkers can contain motifs, e.g., multiple or repeating motifs. In one embodiment, the linker has the amino acid sequence GS, or repeats thereof (Huston, J. et al., Methods Enzymol., 203:46-88, 1991). In another embodiment, the linker includes the amino acid sequence EK, or repeats thereof (Whitlow, M. et al., Protein Eng., 6:989-95, 1993). In another embodiment, the linker includes the amino acid sequence GGS, or repeats thereof.

In another embodiment, the linker includes the amino acid sequence GGGGS (SEQ ID NO: 13), or repeats thereof. In certain embodiments, the linker contains more than one repeat of GGS or GGGGS (U.S. Pat. No. 6,541,219, the entire contents of which are herein incorporated by reference). In one embodiment, the peptide linker may be rich in small or polar amino acids, such as G and S, but can contain additional amino acids, such as T and A, to maintain flexibility, as well as polar amino acids, such as K and E, to improve solubility.

Exemplary linkers include, but are not limited to: G₄A (SEQ ID NO: 13), (G₄A)₂G₄S (SEQ ID NO: 14), (G₄A)₂G₃AG₄S (SEQ ID NO: 79), G₄AG₃AG₄S (SEQ ID NO: 163), G₄SDA (SEQ ID NO: 164), G₄SDAA (SEQ ID NO: 15), G₄S (SEQ ID NO: 16), (G₄S)₂ (SEQ ID NO: 17), (G₄S)₃ (SEQ ID NO: 18), (G₄S)₄ (SEQ ID NO: 19), (G₄S)₅ (SEQ ID NO: 20), (G₄S)₆ (SEQ ID NO: 21), EAAAK (SEQ ID NO: 142), (EAAAK)₃ (SEQ ID NO: 22), PAPAP (SEQ ID NO: 23), G₄SPAPAP (SEQ ID NO: 24), PAPAPG₄S (SEQ ID NO: 25), GSTSGKSSEGKG (SEQ ID NO: 26), (GGGDS)₂ (SEQ ID NO: 27), (GGGES)₂ (SEQ ID NO: 28), GGGDSGGGGS (SEQ ID NO: 29), GGGASGGGGS (SEQ ID NO: 30), GGGESGGGGS (SEQ ID NO: 31), ASTKGP (SEQ ID NO: 32), ASTKGPSVFPLAP (SEQ ID NO: 33), G₃P (SEQ ID NO: 34), G₇P (SEQ ID NO: 35), PAPNLLGGP (SEQ ID NO: 36), Go (SEQ ID NO: 37), G₁₂ (SEQ ID NO: 38), APELPGGP (SEQ ID NO: 39), SEPQPQPG (SEQ ID NO: 40), (G₃S₂)₃ (SEQ ID NO: 41), GGGGGGGGGSGGGS (SEQ ID NO: 42), GGGGSGGGGGGGGGS (SEQ ID NO: 43), (GGSSS)₃ (SEQ ID NO: 44), (GS₄)₃ (SEQ ID NO: 45), G₄A(G₄S)₂ (SEQ ID NO: 46), G₄SG₄AG₄S (SEQ ID NO: 47), G₃AS(G₄S)₂ (SEQ ID NO: 48), G₄SG₃ASG₄S (SEQ ID NO: 49), G₄SAG₃SG₄S (SEQ ID NO: 50), (G₄S)₂AG₃S (SEQ ID NO: 51), G₄SAG₃SAG₃S (SEQ ID NO: 52), G₄D(G₄S)₂ (SEQ ID NO: 53), G₄SG₄DG₄S (SEQ ID NO: 54), (G₄D)₂G₄S (SEQ ID NO: 55), G₄E(G₄S)₂ (SEQ ID NO: 56), G₄SG₄EG₄S (SEQ ID NO: 57), and (G₄E)₂G₄S (SEQ ID NO: 58), (GGGGS)n, wherein n can be any number, KESGSVSSEQLAQFRSLD (SEQ ID NO: 59), and EGKSSGSGSESKST (SEQ ID NO: 60), (Gly)₈ (SEQ ID NO: 61), GSAGSAAGSGEF(SEQ ID NO: 62), and (Gly)₈ (SEQ ID NO: 63). Exemplary rigid linkers include but are not limited to A(EAAAK)A (SEQ ID NO: 143), A(EAAAK)nA (SEQ ID NO: 64), wherein n can be any number, or (XP)n wherein n can be any number, with X designating any amino acid. Exemplary in vivo cleavable linkers include, for example, LEAGCKNFFPRSFTSCGSLE (SEQ ID NO: 65), GSST (SEQ ID NO: 66), and CRRRRRREAEAC (SEQ ID NO: 67). In some embodiments, a linker can contain 2 to 12 amino acids including motifs of GS, e.g., GS, GSGS (SEQ ID NO: 68), GSGSGS (SEQ ID NO: 69), GSGSGSGS (SEQ ID NO: 70), GSGSGSGSGS (SEQ ID NO: 71), or GSGSGSGSGSGS (SEQ ID NO: 72). In certain other embodiments, a linker can contain 3 to 12 amino acids including motifs of GGS, e.g., GGS, GGSGGS (SEQ ID NO: 73), GGSGGSGGS (SEQ ID NO: 74), and GGSGGSGGSGGS (SEQ ID NO: 75). In yet other embodiments, a linker can contain 4 to 12 amino acids including motifs of GGSG, e.g., GGSG (SEQ ID NO: 76), GGSGGGSG (SEQ ID NO: 77), or GGSGGGSGGGSG (SEQ ID NO: 78). In other embodiments, a linker can contain motifs of GGGGS (SEQ ID NO: 13). In other embodiments, a linker can also contain amino acids other than glycine and serine, e.g., GENLYFQSGG (SEQ ID NO: 80), SACYCELS (SEQ ID NO: 81), RSIAT (SEQ ID NO: 82), RPACKIPNDLKQKVMNH (SEQ ID NO: 83), GGSAGGSGSGSSGGSSGASGTGTAGGTGSGSGTGSG (SEQ ID NO: 84), AAANSSIDLISVPVDSR (SEQ ID NO: 85), GGSGGGSEGGGSEGGGSEGGGSEGGGSEGGGSGGGS (SEQ ID NO: 86), GGGGAGGGGAGGGGS (SEQ ID NO: 87), GGGGAGGGGAGGGGAGGGGS (SEQ ID NO: 89), DAAGGGGSGGGGSGGGGSGGGGSGGGGS (SEQ ID NO: 90), GGGGAGGGGAGGGGA (SEQ ID NO: 91), GGGGAGGGGAGGGAGGGGS (SEQ ID NO: 92), or GGSSRSSSSGGGGAGGGG (SEQ ID NO: 93).

In one embodiment, the linker is a cleavable linker, such as an enzymatically cleavable linker. Inclusion of a cleavable linker can aid in detection of the fusion protein. An enzymatically cleavable linker can be cleavable, for example, by trypsin, Human Rhinovirus 3C Protease (3C), enterokinase (Ekt), Factor Xa (FXa), Tobacco Etch Virus protease (TEV), or thrombin (Thr). Cleavage sequences for each of these enzymes are well known in the art. For example, trypsin cleaves peptides on the C-terminal side of lysine and arginine amino acid residues. If a proline residue is on the carboxyl side of the cleavage site, the cleavage will not occur. If an acidic residue is on either side of the cleavage site, the rate of hydrolysis has been shown to be slower. The following linkers are examples of linkers that can be excised using trypsin: K(G₄A)₂G₃AG₄SK (SEQ ID NO:226), R(G₄A)₂G₃AG₄SR (SEQ ID NO:227), K(G₄A)₂G₃AG₄SR (SEQ ID NO:228), R(G₄A)₂G₃AG₄SK (SEQ ID NO:229), K(G₄A)₂G₄SK (SEQ ID NO230), K(G₄A)₂G₄SR (SEQ ID NO:231), R(G₄A)₂G₄SK (SEQ ID NO:232), and R(G₄A)₂G₄SR (SEQ ID NO:233).

A particular example of a protease cleavage site that can be included in an enzymatically cleavable linker is a tobacco etch virus (TEV) protease cleavage site, e.g., ENLYTQS (SEQ ID NO: 234), where the protease cleaves between the glutamine and the serine. Another example of a protease cleavage site that can be included in an enzymatically cleavable linker is an enterokinase cleavage site, e.g., DDDDK (SEQ ID NO: 235), where cleavage occurs after the lysine residue. Another example of a protease cleavage site that can be included in an enzymatically cleavable linker is a thrombin cleavage site, e.g., LVPR (SEQ ID NO: 236). For Human Rhinovirus 3C Protease, the cleavage site is LEVLFQGP (SEQ ID NO: 237) where cleavage occurs between the glutamine and glycine residues. The preferred cleavage site for Factor Xa protease is IEDGR (SEQ ID NO: 238), where cleavage occurs between the glutamic acid and aspartic acid residues.

The inclusion of the cleavable linker is useful in that it has a sequence of amino acids that is unique from other peptides in the human proteome that are generated with the above mentioned enzymes. As such this excised linker may serve as a unique identifying peptide of the fusion protein when administered as a pharmaceutical preparation to humans. In this way the cleavable linker may be detected and quantitated by mass spectrometry and be used to monitor the pharmacokinetics of the fusion protein.

In another embodiment, the linker is a polymeric or oligomeric glycine linker, and can include a lysine at the N-terminus, the C-terminus, or both the N- and the C-termini.

With reference to formulas I-VI above, the C-terminus of D1 may be linked to the N-terminus of Fc. In a certain embodiment, the C-terminus of Fc may be linked to the N-terminus of D2. In a certain embodiment, the C-terminus of FH may be linked to the N-terminus of FH. In a certain embodiment, the C-terminus of FH may be linked to the N-terminus of CR2. In a certain embodiment, the C-terminus of CR2 may be linked to the N-terminus of FH. In a certain embodiment, the C-terminus of FH may be linked to the N-terminus of Fc. In a certain embodiment, the C-terminus of CR2 may be linked to the N-terminus of Fc. In a certain embodiment, the C-terminus of Fc may be linked to the N-terminus of FH. In a certain embodiment, the C-terminus of Fc may be linked to the N-terminus of CR2.

TABLE 1
Exemplary Fusion Proteins having the sequence, from N-terminus to C-terminus, of D1-L1-FC-L2-D2

| | | | | | | Amino |
|---|---|---|---|---|---|---|
| | | | | | | Acid/Nucleic |
| Compound | | | | | | Acid |
| Name | D1 (SCRs) | L1 | Fc | L2 | D2 (SCRs) | Sequence |
| Compound | CR2 1-4 | G₄SDAA | IgG2-G4-Fc | (G₄S)₄ | FH 1-5 | (SEQ ID NOs: |
| A | (SEQ ID | (SEQ ID | (SEQ ID | (SEQ ID | (SEQ ID | 114 and 165) |
| | NO: 94) | NO: 15) | NO: 88) | NO: 19) | NO: 108) | |
| Compound | Mouse FH | — | Mouse IgG1 | — | Mouse FH | (SEQ ID NOs: |
| B | 1-5 | | (SEQ ID | | 19-20 | 115 and 166) |
| | (SEQ ID | | NO: 113) | | (SEQ ID | |
| | NO: 108) | | | | NO: 110) | |
| Compound | Mouse FH | — | Mouse IgG1 | — | Mouse FH | (SEQ ID NOs: |
| C | 19-20 | | (SEQ ID | | 1-5 | 116 and 167) |
| | (SEQ ID | | NO: 88) | | (SEQ ID | |
| | NO: 110) | | | | NO: 108) | |
| Compound | CR2 1-4 | — | IgG2-G4-Fc | GGSSRSSSSGGGGAGGGG | FH 1-5 | (SEQ ID NOs: |
| D | (SEQ ID | | (SEQ ID | SEQ ID | (SEQ ID | 117 and 168) |
| | NO: 94) | | NO: 88) | NO: 93 | NO: 108) | |
| Compound | CR2 1-4 | G₄SDAA | IgG2-G4-Fc | (G₄S)₂ | FH 1-5 | (SEQ ID NOs: |
| E | (SEQ ID | (SEQ ID | (SEQ ID | (SEQ ID | (SEQ ID | 118 and 169) |
| | NO: 94) | NO: 15) | NO: 88) | NO: 17) | NO: 108) | |
| Compound | CR2 1-4 | G₄SDAA | IgG2-G4-Fc | G₄S | FH 1-5 | Compound F |
| F | (SEQ ID | (SEQ ID | (SEQ ID | (SEQ ID | (SEQ ID | (SEQ ID NOs: |
| | NO: 94) | NO: 15) | NO: 88) | NO: 16) | NO: 108) | 119 and 170) |
| Compound | CR2 1-4 | -DAA linker | IgG2-G4-Fc | — | FH 1-5 | (SEQ ID NOs: |
| G | (SEQ ID | | (SEQ ID | | (SEQ ID | 120 and 171) |
| | NO: 94) | | NO: 88) | | NO: 108) | |
| Compound | CR2 1-4 | (G₄A)₂G₄S | IgG2-G4-Fc | (G₄A)₂G₄S | FH 1-5 | (SEQ ID NOs: |
| H | (N107Q) | (SEQ ID | (SEQ ID | (SEQ ID | (SEQ ID | 121 and 172) |
| | (SEQ ID | NO: 14) | NO: 88) | NO: 14) | NO: 108) | |
| | NO: 96) | | | | | |
| Compound | CR2 1-4 | (G₄A)₂G₄S | IgG2-G4-Fc | (G₄A)₂G₄S | FH 1-5 | Compound I |
| I | (S109A) | (SEQ ID | (SEQ ID | (SEQ ID | (SEQ ID | (SEQ ID NOs: |
| | (SEQ ID | NO: 14) | NO: 88) | NO: 14) | NO: 108) | 122 and 173) |
| | NO: 99) | | | | | |
| Compound | CR2 1-4 | DAA linker- | IgG2-G4-Fc | — | FH 1-5 | (SEQ ID NOs: |
| M | (SEQ ID | | (SEQ ID | | (SEQ ID | 123 and 177) |
| | NO: 94) | | NO: 88) | | NO: 108) | |
| Compound | CR2 1-4 | — | IgG2-G4-Fc | (G₄A)₂G₄S | FH 1-5 | (SEQ ID NOs: |
| N | (SEQ ID | | (SEQ ID | (SEQ ID | (SEQ ID | 124 and 178) |
| | NO: 94) | | NO: 88) | NO: 14) | NO: 108) | |
| Compound | — | — | α-HSA-VHH | — | FH 1-5 | (SEQ ID NOs: |
| O | | | (SEQ ID | | (SEQ ID | 125 and 179) |
| | | | NO: 133) | | NO: 108) | |
| Compound | CR2 1-4 | — | α-HSA-VHH | — | FH 1-5 | (SEQ ID NOs: |
| P | (SEQ ID | | (SEQ ID | | (SEQ ID | 126 and 180) |
| | NO: 94) | | NO: 133) | | NO: 108) | |
| Compound | CR2 1-4 | (G₄S) | α-HSA-VHH | (G₄S) | FH 1-5 | (SEQ ID NOs: |
| Q | (SEQ ID | (SEQ ID | (SEQ ID | (SEQ ID | (SEQ ID | 127 and 181) |
| | NO: 94) | NO: 16) | NO: 133) | NO: 16) | NO: 108) | |
| Compound | CR2 1-4 | (G₄S)₂ | α-HSA-VHH | (G₄S)₂ | FH 1-5 | (SEQ ID NOs: |
| R | (SEQ ID | (SEQ ID | (SEQ ID | (SEQ ID | (SEQ ID | 128 and 183) |
| | NO: 94) | NO: 17) | NO: 133) | NO: 17) | NO: 108) | |
| Compound | CR2 1-4 | (G₄S)₃ | α-HSA-VHH | (G₄S)₃ | FH 1-5 | (SEQ ID NOs: |
| S | (SEQ ID | (SEQ ID | (SEQ ID | (SEQ ID | (SEQ ID | 129 and 183) |
| | NO: 94) | NO: 18) | NO: 133) | NO: 18) | NO: 108) | |
| Compound | CR2 1-4 | (G₄S)₄ | α-HSA-VHH | (G₄S)₄ | FH 1-5 | (SEQ ID NOs: |
| T | (SEQ ID | (SEQ ID | (SEQ ID | (SEQ ID | (SEQ ID | 130 and 184) |
| | NO: 94) | NO: 19) | NO: 133) | NO: 19) | NO: 108) | |
| Compound | CR2 1-4 | — | α-HSA-VHH | — | FH 1-5 | (SEQ ID NOs: |
| U | (SEQ ID | | (SEQ ID | | (SEQ ID | 131 and 185) |
| | NO: 94) | | NO: 133) | | NO: 108) | |
| Compound | CR2 1-4 | (G₄A)₂G₄S | IgG2-G4-Fc | (G₄A)₂G₄S | FH 1-5 | (SEQ ID NOs: |
| X | (SEQ ID | (SEQ ID | (SEQ ID | (SEQ ID | (SEQ ID | 132 and 188) |
| | NO: 94) | NO: 14) | NO: 88) | NO: 14) | NO: 108) | |
| Compound | FH 19-20 | — | IgG2-G4-Fc | — | FH 1-5 | (SEQ ID NOs: |
| Y | (SEQ ID | | (SEQ ID | | (SEQ ID | 144 and 189) |
| | NO: 110) | | NO: 88) | | NO: 108) | |
| Compound | FH 1-5 | — | IgG2-G4-Fc | — | FH 19-20 | (SEQ ID NOs: |
| Z | (SEQ ID | | (SEQ ID | | (SEQ ID | 145 and 190) |
| | NO: 108) | | NO: 88) | | NO: 110) | |
| Compound | CR2 1-2 | G₄SDAA | IgG2-G4-Fc | (G₄A)₂G₃AG₄S | FH 1-5 | (SEQ ID NOs: |
| AB | (N107Q) | (SEQ ID | (SEQ ID | (SEQ ID | (SEQ ID | 147 and 192) |
| | (SEQ ID | NO: 15) | NO: 88) | NO: 79) | NO: 108) | |
| | NO: 102) | | | | | |
| Compound | CR2 1-2 | G₄SDAA | IgG2-G4-Fc | (G₄A)₂G₃AG₄S | FH 1-4 | (SEQ ID NOs: |
| AC | (N107Q) | (SEQ ID | (SEQ ID | (SEQ ID | (SEQ ID | 148 and 193) |
| | (SEQ ID | NO: 15) | NO: 88) | NO: 79) | NO: 109) | |
| | NO: 102) | | | | | |
| Compound | FH 1-5 | (G₄A)₂G₄S | IgG2-G4-Fc | — | FH 19-20 | (SEQ ID NOs: |
| AG | (SEQ ID | (SEQ ID | (SEQ ID | | (SEQ ID | 152 and 197) |
| | NO: 108) | NO: 14) | NO: 88) | | NO: 110) | |
| Compound | FH 1-5 | — | IgG2-G4-Fc | (G₄A)₂G₄S | FH 19-20 | (SEQ ID NOs: |
| AH | (SEQ ID | | (SEQ ID | (SEQ ID | (SEQ ID | 153 and 198) |
| | NO: 108) | | NO: 88) | NO: 14) | NO: 110) | |
| Compound | FH 1-5 | (G₄A)₂G₄S | IgG2-G4-Fc | (G₄A)₂G₄S | FH 19-20 | (SEQ ID NOs: |
| Al | (SEQ ID | (SEQ ID | (SEQ ID | (SEQ ID | (SEQ ID | 154 and 199) |
| | NO: 108) | NO: 14) | NO: 88) | NO: 14) | NO: 110) | |
| Compound | CR2 1 -2 | G₄SDAA | FLG2-G4-FC | (G₄A)₂G₃AG₄S | FH 1-4 | (SEQ ID NOs: |
| AJ | (N107Q) | (SEQ ID | (SEQ ID | (SEQ ID | (SEQ ID | 155 and 200) |
| | (SEQ ID | NO: 15) | NO: 111) | NO: 79) | NO: 109) | |
| | NO: 102) | | | | | |
| Compound | CR2 1-4 | G₄SDAA | IgG2-G4-Fc | (G₄A)₂G3AG₄S | FH 1-5 | (SEQ ID NOs: |
| AR | (N107Q) | (SEQ ID | (SEQ ID | (SEQ ID | (SEQ ID | 209 and 216) |
| | (SEQ ID | NO: 15) | NO: 88) | NO: 79) | NO: 108) | |
| | NO: 96) | | | | | |
| Compound | CR2 1-4 | — | IgG2-G4-Fc | (G₄A)₂G3AG₄S | FH 1-5 | (SEQ ID NOs: |
| AS | (N107Q) | | (SEQ ID | (SEQ ID | (SEQ ID | 210 and 217) |
| | (SEQ ID | | NO: 88) | NO: 79) | NO: 108) | |
| | NO: 96) | | | | | |
| Compound | CR2 1-2 | — | IgG2-G4-Fc | (G₄A)₂G3AG₄S | FH 1-5 | (SEQ ID NOs: |
| AT | (N107Q) | | (SEQ ID | (SEQ ID | (SEQ ID | 211 and 218) |
| | (SEQ ID | | NO: 88) | NO: 79) | NO: 108) | |
| | NO: 102) | | | | | |
| Compound | CR2 1-4 | G₄SDAA | IgG2-G4-Fc | (G₄A)₂G3AG₄S | FH 1-4 | (SEQ ID NOs: |
| AU | (N107Q) | (SEQ ID | (SEQ ID | (SEQ ID | (SEQ ID | 212 and 219) |
| | (SEQ ID | NO: 15) | NO: 88) | NO: 79) | NO: 109) | |
| | NO: 96) | | | | | |
| Compound | CR2 1-4 | — | IgG2-G4-Fc | (G₄A)₂G3AG₄S | FH 1-4 | (SEQ ID NOs: |
| AV | (N107Q) | | (SEQ ID | (SEQ ID | (SEQ ID | 213 and 220) |
| | (SEQ ID | | NO: 88) | NO: 79) | NO: 109) | |
| | NO: 96) | | | | | |
| Compound | CR2 1 -2 | — | IgG2-G4-Fc | (G₄A)₂G3AG₄S | FH 1-4 | (SEQ ID NOs: |
| AW | (N107Q) | | (SEQ ID | (SEQ ID | (SEQ ID | 214 and 221) |
| | (SEQ ID | | NO: 88) | NO: 79) | NO: 109) | |
| | NO: 102) | | | | | |
| Compound | FH 19-20 | (G₄A)₂G₄S | IgG2-G4-Fc | (G₄A)₂G₄S | FH 1-4 | (SEQ ID NOs: |
| AX | (SEQ ID | (SEQ ID | (SEQ ID | (SEQ ID | (SEQ ID | 215 and 222) |
| | NO: 110) | NO: 14) | NO: 88) | NO: 14) | NO: 109) | |

“—” indicates the absence of a feature.

TABLE 2
Exemplary Fusion Proteins having the sequence, from
N-terminus to C-terminus, of D1-L1-FC-L2-D2

| D1 (SCRs) | L1 | Fc | L2 | D2 (SCRs) |
|---|---|---|---|---|
| FH 1-4 | + | + | + | FH 1-4 |
| FH 1-4 | + | + | + | FH 1-5 |
| FH 1-4 | + | + | + | FH 1-4, 19, 20 |
| FH 1-4 | + | + | + | FH 1-5, 19, 20 |
| FH 1-4 | + | + | + | FH 19, 20 |
| FH 1-4 | + | + | + | CR2 1-2 |
| FH 1-4 | + | + | + | CR2 1-3 |
| FH 1-4 | + | + | + | CR2 1-4 |
| FH 1-4 | + | + | + | CR2 1-2 (L3) FH 19-20 |
| FH 1-4 | + | + | + | FH 19-20 (L3) FH 19-20 |
| FH 1-5 | + | + | + | FH 1-4 |
| FH 1-5 | + | + | + | FH 1-5 |
| FH 1-5 | + | + | + | FH 1-4, 19, 20 |
| FH 1-5 | + | + | + | FH 1-5, 19, 20 |
| FH 1-5 | + | + | + | FH 19, 20 |
| FH 1-5 | + | + | + | CR2 1-2 |
| FH 1-5 | + | + | + | CR2 1-3 |
| FH 1-5 | + | + | + | CR2 1-4 |
| FH 1-5 | + | + | + | CR2 1-2 (L3) FH 19-20 |
| FH 1-5 | + | + | + | FH 19-20 (L3) FH 19-20 |
| FH 1-4, 19, 20 | + | + | + | FH 1-4 |
| FH 1-4, 19, 20 | + | + | + | FH 1-5 |
| FH 1-4, 19, 20 | + | + | + | FH 1-4, 19, 20 |
| FH 1-4, 19, 20 | + | + | + | FH 1-5, 19, 20 |
| FH 1-4, 19, 20 | + | + | + | FH 19, 20 |
| FH 1-4, 19, 20 | + | + | + | CR2 1-2 |
| FH 1-4, 19, 20 | + | + | + | CR2 1-3 |
| FH 1-4, 19, 20 | + | + | + | CR2 1-4 |
| FH 1-5, 19, 20 | + | + | + | FH 1-4 |
| FH 1-5, 19, 20 | + | + | + | FH 1-5 |
| FH 1-5, 19, 20 | + | + | + | FH 1-4, 19, 20 |
| FH 1-5, 19, 20 | + | + | + | FH 1-5, 19, 20 |
| FH 1-5, 19, 20 | + | + | + | FH 19, 20 |
| FH 1-5, 19, 20 | + | + | + | CR2 1-2 |
| FH 1-5, 19, 20 | + | + | + | CR2 1-3 |
| FH 1-5, 19, 20 | + | + | + | CR2 1-4 |
| FH 19-20 | + | + | + | FH 1-4 |
| FH 19-20 | + | + | + | FH 1-5 |
| FH 19-20 | + | + | + | FH 1-4, 19, 20 |
| FH 19-20 | + | + | + | FH 1-5, 19, 20 |
| CR2 1-2 | + | + | + | FH 1-4 |
| CR2 1-2 | + | + | + | FH 1-5 |
| CR2 1-2 | + | + | + | FH 1-4, 19, 20 |
| CR2 1-2 | + | + | + | FH 1-5, 19, 20 |
| CR2 1-3 | + | + | + | FH 1-4 |
| CR2 1-3 | + | + | + | FH 1-5 |
| CR2 1-3 | + | + | + | FH 1-4, 19, 20 |
| CR2 1-3 | + | + | + | FH 1-5, 19, 20 |
| CR2 1-4 | + | + | + | FH 1-4 |
| CR2 1-4 | + | + | + | FH 1-5 |
| CR2 1-4 | + | + | + | FH 1-4, 19, 20 |
| CR2 1-4 | + | + | + | FH 1-5, 19, 20 |
| CR2 1-2 (L3) FH 19-20 | + | + | + | FH 1-4 |
| CR2 1-2 (L3) FH 19-20 | + | + | + | FH 1-5 |
| FH 19-20 (L3) FH 19-20 | + | + | + | FH 1-4 |
| FH 19-20 (L3) FH 19-20 | + | + | + | FH 1-5 |
| FH 1-4 | + | + | − | FH 1-4 |
| FH 1-4 | + | + | − | FH 1-5 |
| FH 1-4 | + | + | − | FH 1-4, 19, 20 |
| FH 1-4 | + | + | − | FH 1-5, 19, 20 |
| FH 1-4 | + | + | − | FH 19, 20 |
| FH 1-4 | + | + | − | CR2 1-2 |
| FH 1-4 | + | + | − | CR2 1-3 |
| FH 1-4 | + | + | − | CR2 1-4 |
| FH 1-4 | + | + | − | CR2 1-2 (L3) FH 19-20 |
| FH 1-4 | + | + | − | FH 19-20 (L3) FH 19-20 |
| FH 1-5 | + | + | − | FH 1-4 |
| FH 1-5 | + | + | − | FH 1-5 |
| FH 1-5 | + | + | − | FH 1-4, 19, 20 |
| FH 1-5 | + | + | − | FH 1-5, 19, 20 |
| FH 1-5 | + | + | − | FH 19, 20 |
| FH 1-5 | + | + | − | CR2 1-2 |
| FH 1-5 | + | + | − | CR2 1-3 |
| FH 1-5 | + | + | − | CR2 1-4 |
| FH 1-5 | + | + | − | CR2 1-2 (L3) FH 19-20 |
| FH 1-5 | + | + | − | FH 19-20 (L3) FH 19-20 |
| FH 1-4, 19, 20 | + | + | − | FH 1-4 |
| FH 1-4, 19, 20 | + | + | − | FH 1-5 |
| FH 1-4, 19, 20 | + | + | − | FH 1-4, 19, 20 |
| FH 1-4, 19, 20 | + | + | − | FH 1-5, 19, 20 |
| FH 1-4, 19, 20 | + | + | − | FH 19, 20 |
| FH 1-4, 19, 20 | + | + | − | CR2 1-2 |
| FH 1-4, 19, 20 | + | + | − | CR2 1-3 |
| FH 1-4, 19, 20 | + | + | − | CR2 1-4 |
| FH 1-5, 19, 20 | + | + | − | FH 1-4 |
| FH 1-5, 19, 20 | + | + | − | FH 1-5 |
| FH 1-5, 19, 20 | + | + | − | FH 1-4, 19, 20 |
| FH 1-5, 19, 20 | + | + | − | FH 1-5, 19, 20 |
| FH 1-5, 19, 20 | + | + | − | FH 19, 20 |
| FH 1-5, 19, 20 | + | + | − | CR2 1-2 |
| FH 1-5, 19, 20 | + | + | − | CR2 1-3 |
| FH 1-5, 19, 20 | + | + | − | CR2 1-4 |
| FH 19-20 | + | + | − | FH 1-4 |
| FH 19-20 | + | + | − | FH 1-5 |
| FH 19-20 | + | + | − | FH 1-4, 19, 20 |
| FH 19-20 | + | + | − | FH 1-5, 19, 20 |
| CR2 1-2 | + | + | − | FH 1-4 |
| CR2 1-2 | + | + | − | FH 1-5 |
| CR2 1-2 | + | + | − | FH 1-4, 19, 20 |
| CR2 1-2 | + | + | − | FH 1-5, 19, 20 |
| CR2 1-3 | + | + | − | FH 1-4 |
| CR2 1-3 | + | + | − | FH 1-5 |
| CR2 1-3 | + | + | − | FH 1-4, 19, 20 |
| CR2 1-3 | + | + | − | FH 1-5, 19, 20 |
| CR2 1-4 | + | + | − | FH 1-4 |
| CR2 1-4 | + | + | − | FH 1-5 |
| CR2 1-4 | + | + | − | FH 1-4, 19, 20 |
| CR2 1-4 | + | + | − | FH 1-5, 19, 20 |
| CR2 1-2 (L3) FH 19-20 | + | + | − | FH 1-4 |
| CR2 1-2 (L3) FH 19-20 | + | + | − | FH 1-5 |
| FH 19-20 (L3) FH 19-20 | + | + | − | FH 1-4 |
| FH 19-20 (L3) FH 19-20 | + | + | − | FH 1-5 |
| FH 1-4 | − | + | + | FH 1-4 |
| FH 1-4 | − | + | + | FH 1-5 |
| FH 1-4 | − | + | + | FH 1-4, 19, 20 |
| FH 1-4 | − | + | + | FH 1-5, 19, 20 |
| FH 1-4 | − | + | + | FH 19, 20 |
| FH 1-4 | − | + | + | CR2 1-2 |
| FH 1-4 | − | + | + | CR2 1-3 |
| FH 1-4 | − | + | + | CR2 1-4 |
| FH 1-4 | − | + | + | CR2 1-2 (L3) FH 19-20 |
| FH 1-4 | − | + | + | FH 19-20 (L3) FH 19-20 |
| FH 1-5 | − | + | + | FH 1-4 |
| FH 1-5 | − | + | + | FH 1-5 |
| FH 1-5 | − | + | + | FH 1-4, 19, 20 |
| FH 1-5 | − | + | + | FH 1-5, 19, 20 |
| FH 1-5 | − | + | + | FH 19, 20 |
| FH 1-5 | − | + | + | CR2 1-2 |
| FH 1-5 | − | + | + | CR2 1-3 |
| FH 1-5 | − | + | + | CR2 1-4 |
| FH 1-5 | − | + | + | CR2 1-2 (L3) FH 19-20 |
| FH 1-5 | − | + | + | FH 19-20 (L3) FH 19-20 |
| FH 1-4, 19, 20 | − | + | + | FH 1-4 |
| FH 1-4, 19, 20 | − | + | + | FH 1-5 |
| FH 1-4, 19, 20 | − | + | + | FH 1-4, 19, 20 |
| FH 1-4, 19, 20 | − | + | + | FH 1-5, 19, 20 |
| FH 1-4, 19, 20 | − | + | + | FH 19, 20 |
| FH 1-4, 19, 20 | − | + | + | CR2 1-2 |
| FH 1-4, 19, 20 | − | + | + | CR2 1-3 |
| FH 1-4, 19, 20 | − | + | + | CR2 1-4 |
| FH 1-5, 19, 20 | − | + | + | FH 1-4 |
| FH 1-5, 19, 20 | − | + | + | FH 1-5 |
| FH 1-5, 19, 20 | − | + | + | FH 1-4, 19, 20 |
| FH 1-5, 19, 20 | − | + | + | FH 1-5, 19, 20 |
| FH 1-5, 19, 20 | − | + | + | FH 19, 20 |
| FH 1-5, 19, 20 | − | + | + | CR2 1-2 |
| FH 1-5, 19, 20 | − | + | + | CR2 1-3 |
| FH 1-5, 19, 20 | − | + | + | CR2 1-4 |
| FH 19-20 | − | + | + | FH 1-4 |
| FH 19-20 | − | + | + | FH 1-5 |
| FH 19-20 | − | + | + | FH 1-4, 19, 20 |
| FH 19-20 | − | + | + | FH 1-5, 19, 20 |
| CR2 1-2 | − | + | + | FH 1-4 |
| CR2 1-2 | − | + | + | FH 1-5 |
| CR2 1-2 | − | + | + | FH 1-4, 19, 20 |
| CR2 1-2 | − | + | + | FH 1-5, 19, 20 |
| CR2 1-3 | − | + | + | FH 1-4 |
| CR2 1-3 | − | + | + | FH 1-5 |
| CR2 1-3 | − | + | + | FH 1-4, 19, 20 |
| CR2 1-3 | − | + | + | FH 1-5, 19, 20 |
| CR2 1-4 | − | + | + | FH 1-4 |
| CR2 1-4 | − | + | + | FH 1-5 |
| CR2 1-4 | − | + | + | FH 1-4, 19, 20 |
| CR2 1-4 | − | + | + | FH 1-5, 19, 20 |
| CR2 1-2 (L3) FH 19-20 | − | + | + | FH 1-4 |
| CR2 1-2 (L3) FH 19-20 | − | + | + | FH 1-5 |
| FH 19-20 (L3) FH 19-20 | − | + | + | FH 1-4 |
| FH 19-20 (L3) FH 19-20 | − | + | + | FH 1-5 |
| FH 1-4 | − | + | − | FH 1-4 |
| FH 1-4 | − | + | − | FH 1-5 |
| FH 1-4 | − | + | − | FH 1-4, 19, 20 |
| FH 1-4 | − | + | − | FH 1-5, 19, 20 |
| FH 1-4 | − | + | − | FH 19, 20 |
| FH 1-4 | − | + | − | CR2 1-2 |
| FH 1-4 | − | + | − | CR2 1-3 |
| FH 1-4 | − | + | − | CR2 1-4 |
| FH 1-4 | − | + | − | CR2 1-2 (L3) FH 19-20 |
| FH 1-4 | − | + | − | FH 19-20 (L3) FH 19-20 |
| FH 1-5 | − | + | − | FH 1-4 |
| FH 1-5 | − | + | − | FH 1-5 |
| FH 1-5 | − | + | − | FH 1-4, 19, 20 |
| FH 1-5 | − | + | − | FH 1-5, 19, 20 |
| FH 1-5 | − | + | − | FH 19, 20 |
| FH 1-5 | − | + | − | CR2 1-2 |
| FH 1-5 | − | + | − | CR2 1-3 |
| FH 1-5 | − | + | − | CR2 1-4 |
| FH 1-5 | − | + | − | CR2 1-2 (L3) FH 19-20 |
| FH 1-5 | − | + | − | FH 19-20 (L3) FH 19-20 |
| FH 1-4, 19, 20 | − | + | − | FH 1-4 |
| FH 1-4, 19, 20 | − | + | − | FH 1-5 |
| FH 1-4, 19, 20 | − | + | − | FH 1-4, 19, 20 |
| FH 1-4, 19, 20 | − | + | − | FH 1-5, 19, 20 |
| FH 1-4, 19, 20 | − | + | − | FH 19, 20 |
| FH 1-4, 19, 20 | − | + | − | CR2 1-2 |
| FH 1-4, 19, 20 | − | + | − | CR2 1-3 |
| FH 1-4, 19, 20 | − | + | − | CR2 1-4 |
| FH 1-5, 19, 20 | − | + | − | FH 1-4 |
| FH 1-5, 19, 20 | − | + | − | FH 1-5 |
| FH 1-5, 19, 20 | − | + | − | FH 1-4, 19, 20 |
| FH 1-5, 19, 20 | − | + | − | FH 1-5, 19, 20 |
| FH 1-5, 19, 20 | − | + | − | FH 19, 20 |
| FH 1-5, 19, 20 | − | + | − | CR2 1-2 |
| FH 1-5, 19, 20 | − | + | − | CR2 1-3 |
| FH 1-5, 19, 20 | − | + | − | CR2 1-4 |
| FH 19-20 | − | + | − | FH 1-4 |
| FH 19-20 | − | + | − | FH 1-5 |
| FH 19-20 | − | + | − | FH 1-4, 19, 20 |
| FH 19-20 | − | + | − | FH 1-5, 19, 20 |
| CR2 1-2 | − | + | − | FH 1-4 |
| CR2 1-2 | − | + | − | FH 1-5 |
| CR2 1-2 | − | + | − | FH 1-4, 19, 20 |
| CR2 1-2 | − | + | − | FH 1-5, 19, 20 |
| CR2 1-3 | − | + | − | FH 1-4 |
| CR2 1-3 | − | + | − | FH 1-5 |
| CR2 1-3 | − | + | − | FH 1-4, 19, 20 |
| CR2 1-3 | − | + | − | FH 1-5, 19, 20 |
| CR2 1-4 | − | + | − | FH 1-4 |
| CR2 1-4 | − | + | − | FH 1-5 |
| CR2 1-4 | − | + | − | FH 1-4, 19, 20 |
| CR2 1-4 | − | + | − | FH 1-5, 19, 20 |
| CR2 1-2 (L3) FH 19-20 | − | + | − | FH 1-4 |
| CR2 1-2 (L3) FH 19-20 | − | + | − | FH 1-5 |
| FH 19-20 (L3) FH 19-20 | − | + | − | FH 1-4 |
| FH 19-20 (L3) FH 19-20 | − | + | − | FH 1-5 |

“+” indicates the inclusion of a feature, “−” while indicates the absence of a feature.

TABLE 3
Exemplary Fusion Proteins having the sequence, from
N-terminus to C-terminus, of D1-L1-VHH-L2-D2

| D1 (SCRs) | L1 | VHH | L2 | D2 (SCRs) |
|---|---|---|---|---|
| FH 1-4 | + | + | + | FH 1-4 |
| FH 1-4 | + | + | + | FH 1-5 |
| FH 1-4 | + | + | + | FH 1-4, 19, 20 |
| FH 1-4 | + | + | + | FH 1-5, 19, 20 |
| FH 1-4 | + | + | + | FH 19, 20 |
| FH 1-4 | + | + | + | CR2 1-2 |
| FH 1-4 | + | + | + | CR2 1-3 |
| FH 1-4 | + | + | + | CR2 1-4 |
| FH 1-4 | + | + | + | CR2 1-2 (L3) FH 19-20 |
| FH 1-4 | + | + | + | FH 19-20 (L3) FH 19-20 |
| FH 1-5 | + | + | + | FH 1-4 |
| FH 1-5 | + | + | + | FH 1-5 |
| FH 1-5 | + | + | + | FH 1-4, 19, 20 |
| FH 1-5 | + | + | + | FH 1-5, 19, 20 |
| FH 1-5 | + | + | + | FH 19, 20 |
| FH 1-5 | + | + | + | CR2 1-2 |
| FH 1-5 | + | + | + | CR2 1-3 |
| FH 1-5 | + | + | + | CR2 1-4 |
| FH 1-5 | + | + | + | CR2 1-2 (L3) FH 19-20 |
| FH 1-5 | + | + | + | FH 19-20 (L3) FH 19-20 |
| FH 1-4, 19, 20 | + | + | + | FH 1-4 |
| FH 1-4, 19, 20 | + | + | + | FH 1-5 |
| FH 1-4, 19, 20 | + | + | + | FH 1-4, 19, 20 |
| FH 1-4, 19, 20 | + | + | + | FH 1-5, 19, 20 |
| FH 1-4, 19, 20 | + | + | + | FH 19, 20 |
| FH 1-4, 19, 20 | + | + | + | CR2 1-2 |
| FH 1-4, 19, 20 | + | + | + | CR2 1-3 |
| FH 1-4, 19, 20 | + | + | + | CR2 1-4 |
| FH 1-5, 19, 20 | + | + | + | FH 1-4 |
| FH 1-5, 19, 20 | + | + | + | FH 1-5 |
| FH 1-5, 19, 20 | + | + | + | FH 1-4, 19, 20 |
| FH 1-5, 19, 20 | + | + | + | FH 1-5, 19, 20 |
| FH 1-5, 19, 20 | + | + | + | FH 19, 20 |
| FH 1-5, 19, 20 | + | + | + | CR2 1-2 |
| FH 1-5, 19, 20 | + | + | + | CR2 1-3 |
| FH 1-5, 19, 20 | + | + | + | CR2 1-4 |
| FH 19-20 | + | + | + | FH 1-4 |
| FH 19-20 | + | + | + | FH 1-5 |
| FH 19-20 | + | + | + | FH 1-4, 19, 20 |
| FH 19-20 | + | + | + | FH 1-5, 19, 20 |
| CR2 1-2 | + | + | + | FH 1-4 |
| CR2 1-2 | + | + | + | FH 1-5 |
| CR2 1-2 | + | + | + | FH 1-4, 19, 20 |
| CR2 1-2 | + | + | + | FH 1-5, 19, 20 |
| CR2 1-3 | + | + | + | FH 1-4 |
| CR2 1-3 | + | + | + | FH 1-5 |
| CR2 1-3 | + | + | + | FH 1-4, 19, 20 |
| CR2 1-3 | + | + | + | FH 1-5, 19, 20 |
| CR2 1-4 | + | + | + | FH 1-4 |
| CR2 1-4 | + | + | + | FH 1-5 |
| CR2 1-4 | + | + | + | FH 1-4, 19, 20 |
| CR2 1-4 | + | + | + | FH 1-5, 19, 20 |
| CR2 1-2 (L3) FH 19-20 | + | + | + | FH 1-4 |
| CR2 1-2 (L3) FH 19-20 | + | + | + | FH 1-5 |
| FH 19-20 (L3) FH 19-20 | + | + | + | FH 1-4 |
| FH 19-20 (L3) FH 19-20 | + | + | + | FH 1-5 |
| FH 1-4 | + | + | − | FH 1-4 |
| FH 1-4 | + | + | − | FH 1-5 |
| FH 1-4 | + | + | − | FH 1-4, 19, 20 |
| FH 1-4 | + | + | − | FH 1-5, 19, 20 |
| FH 1-4 | + | + | − | FH 19, 20 |
| FH 1-4 | + | + | − | CR2 1-2 |
| FH 1-4 | + | + | − | CR2 1-3 |
| FH 1-4 | + | + | − | CR2 1-4 |
| FH 1-4 | + | + | − | CR2 1-2 (L3) FH 19-20 |
| FH 1-4 | + | + | − | FH 19-20 (L3) FH 19-20 |
| FH 1-5 | + | + | − | FH 1-4 |
| FH 1-5 | + | + | − | FH 1-5 |
| FH 1-5 | + | + | − | FH 1-4, 19, 20 |
| FH 1-5 | + | + | − | FH 1-5, 19, 20 |
| FH 1-5 | + | + | − | FH 19, 20 |
| FH 1-5 | + | + | − | CR2 1-2 |
| FH 1-5 | + | + | − | CR2 1-3 |
| FH 1-5 | + | + | − | CR2 1-4 |
| FH 1-5 | + | + | − | CR2 1-2 (L3) FH 19-20 |
| FH 1-5 | + | + | − | FH 19-20 (L3) FH 19-20 |
| FH 1-4, 19, 20 | + | + | − | FH 1-4 |
| FH 1-4, 19, 20 | + | + | − | FH 1-5 |
| FH 1-4, 19, 20 | + | + | − | FH 1-4, 19, 20 |
| FH 1-4, 19, 20 | + | + | − | FH 1-5, 19, 20 |
| FH 1-4, 19, 20 | + | + | − | FH 19, 20 |
| FH 1-4, 19, 20 | + | + | − | CR2 1-2 |
| FH 1-4, 19, 20 | + | + | − | CR2 1-3 |
| FH 1-4, 19, 20 | + | + | − | CR2 1-4 |
| FH 1-5, 19, 20 | + | + | − | FH 1-4 |
| FH 1-5, 19, 20 | + | + | − | FH 1-5 |
| FH 1-5, 19, 20 | + | + | − | FH 1-4, 19, 20 |
| FH 1-5, 19, 20 | + | + | − | FH 1-5, 19, 20 |
| FH 1-5, 19, 20 | + | + | − | FH 19, 20 |
| FH 1-5, 19, 20 | + | + | − | CR2 1-2 |
| FH 1-5, 19, 20 | + | + | − | CR2 1-3 |
| FH 1-5, 19, 20 | + | + | − | CR2 1-4 |
| FH 19-20 | + | + | − | FH 1-4 |
| FH 19-20 | + | + | − | FH 1-5 |
| FH 19-20 | + | + | − | FH 1-4, 19, 20 |
| FH 19-20 | + | + | − | FH 1-5, 19, 20 |
| CR2 1-2 | + | + | − | FH 1-4 |
| CR2 1-2 | + | + | − | FH 1-5 |
| CR2 1-2 | + | + | − | FH 1-4, 19, 20 |
| CR2 1-2 | + | + | − | FH 1-5, 19, 20 |
| CR2 1-3 | + | + | − | FH 1-4 |
| CR2 1-3 | + | + | − | FH 1-5 |
| CR2 1-3 | + | + | − | FH 1-4, 19, 20 |
| CR2 1-3 | + | + | − | FH 1-5, 19, 20 |
| CR2 1-4 | + | + | − | FH 1-4 |
| CR2 1-4 | + | + | − | FH 1-5 |
| CR2 1-4 | + | + | − | FH 1-4, 19, 20 |
| CR2 1-4 | + | + | − | FH 1-5, 19, 20 |
| CR2 1-2 (L3) FH 19-20 | + | + | − | FH 1-4 |
| CR2 1-2 (L3) FH 19-20 | + | + | − | FH 1-5 |
| FH 19-20 (L3) FH 19-20 | + | + | − | FH 1-4 |
| FH 19-20 (L3) FH 19-20 | + | + | − | FH 1-5 |
| FH 1-4 | − | + | + | FH 1-4 |
| FH 1-4 | − | + | + | FH 1-5 |
| FH 1-4 | − | + | + | FH 1-4, 19, 20 |
| FH 1-4 | − | + | + | FH 1-5, 19, 20 |
| FH 1-4 | − | + | + | FH 19, 20 |
| FH 1-4 | − | + | + | CR2 1-2 |
| FH 1-4 | − | + | + | CR2 1-3 |
| FH 1-4 | − | + | + | CR2 1-4 |
| FH 1-4 | − | + | + | CR2 1-2 (L3) FH 19-20 |
| FH 1-4 | − | + | + | FH 19-20 (L3) FH 19-20 |
| FH 1-5 | − | + | + | FH 1-4 |
| FH 1-5 | − | + | + | FH 1-5 |
| FH 1-5 | − | + | + | FH 1-4, 19, 20 |
| FH 1-5 | − | + | + | FH 1-5, 19, 20 |
| FH 1-5 | − | + | + | FH 19, 20 |
| FH 1-5 | − | + | + | CR2 1-2 |
| FH 1-5 | − | + | + | CR2 1-3 |
| FH 1-5 | − | + | + | CR2 1-4 |
| FH 1-5 | − | + | + | CR2 1-2 (L3) FH 19-20 |
| FH 1-5 | − | + | + | FH 19-20 (L3) FH 19-20 |
| FH 1-4, 19, 20 | − | + | + | FH 1-4 |
| FH 1-4, 19, 20 | − | + | + | FH 1-5 |
| FH 1-4, 19, 20 | − | + | + | FH 1-4, 19, 20 |
| FH 1-4, 19, 20 | − | + | + | FH 1-5, 19, 20 |
| FH 1-4, 19, 20 | − | + | + | FH 19, 20 |
| FH 1-4, 19, 20 | − | + | + | CR2 1-2 |
| FH 1-4, 19, 20 | − | + | + | CR2 1-3 |
| FH 1-4, 19, 20 | − | + | + | CR2 1-4 |
| FH 1-5, 19, 20 | − | + | + | FH 1-4 |
| FH 1-5, 19, 20 | − | + | + | FH 1-5 |
| FH 1-5, 19, 20 | − | + | + | FH 1-4, 19, 20 |
| FH 1-5, 19, 20 | − | + | + | FH 1-5, 19, 20 |
| FH 1-5, 19, 20 | − | + | + | FH 19, 20 |
| FH 1-5, 19, 20 | − | + | + | CR2 1-2 |
| FH 1-5, 19, 20 | − | + | + | CR2 1-3 |
| FH 1-5, 19, 20 | − | + | + | CR2 1-4 |
| FH 19-20 | − | + | + | FH 1-4 |
| FH 19-20 | − | + | + | FH 1-5 |
| FH 19-20 | − | + | + | FH 1-4, 19, 20 |
| FH 19-20 | − | + | + | FH 1-5, 19, 20 |
| CR2 1-2 | − | + | + | FH 1-4 |
| CR2 1-2 | − | + | + | FH 1-5 |
| CR2 1-2 | − | + | + | FH 1-4, 19, 20 |
| CR2 1-2 | − | + | + | FH 1-5, 19, 20 |
| CR2 1-3 | − | + | + | FH 1-4 |
| CR2 1-3 | − | + | + | FH 1-5 |
| CR2 1-3 | − | + | + | FH 1-4, 19, 20 |
| CR2 1-3 | − | + | + | FH 1-5, 19, 20 |
| CR2 1-4 | − | + | + | FH 1-4 |
| CR2 1-4 | − | + | + | FH 1-5 |
| CR2 1-4 | − | + | + | FH 1-4, 19, 20 |
| CR2 1-4 | − | + | + | FH 1-5, 19, 20 |
| CR2 1-2 (L3) FH 19-20 | − | + | + | FH 1-4 |
| CR2 1-2 (L3) FH 19-20 | − | + | + | FH 1-5 |
| FH 19-20 (L3) FH 19-20 | − | + | + | FH 1-4 |
| FH 19-20 (L3) FH 19-20 | − | + | + | FH 1-5 |
| FH 1-4 | − | + | − | FH 1-4 |
| FH 1-4 | − | + | − | FH 1-5 |
| FH 1-4 | − | + | − | FH 1-4, 19, 20 |
| FH 1-4 | − | + | − | FH 1-5, 19, 20 |
| FH 1-4 | − | + | − | FH 19, 20 |
| FH 1-4 | − | + | − | CR2 1-2 |
| FH 1-4 | − | + | − | CR2 1-3 |
| FH 1-4 | − | + | − | CR2 1-4 |
| FH 1-4 | − | + | − | CR2 1-2 (L3) FH 19-20 |
| FH 1-4 | − | + | − | FH 19-20 (L3) FH 19-20 |
| FH 1-5 | − | + | − | FH 1-4 |
| FH 1-5 | − | + | − | FH 1-5 |
| FH 1-5 | − | + | − | FH 1-4, 19, 20 |
| FH 1-5 | − | + | − | FH 1-5, 19, 20 |
| FH 1-5 | − | + | − | FH 19, 20 |
| FH 1-5 | − | + | − | CR2 1-2 |
| FH 1-5 | − | + | − | CR2 1-3 |
| FH 1-5 | − | + | − | CR2 1-4 |
| FH 1-5 | − | + | − | CR2 1-2 (L3) FH 19-20 |
| FH 1-5 | − | + | − | FH 19-20 (L3) FH 19-20 |
| FH 1-4, 19, 20 | − | + | − | FH 1-4 |
| FH 1-4, 19, 20 | − | + | − | FH 1-5 |
| FH 1-4, 19, 20 | − | + | − | FH 1-4, 19, 20 |
| FH 1-4, 19, 20 | − | + | − | FH 1-5, 19, 20 |
| FH 1-4, 19, 20 | − | + | − | FH 19, 20 |
| FH 1-4, 19, 20 | − | + | − | CR2 1-2 |
| FH 1-4, 19, 20 | − | + | − | CR2 1-3 |
| FH 1-4, 19, 20 | − | + | − | CR2 1-4 |
| FH 1-5, 19, 20 | − | + | − | FH 1-4 |
| FH 1-5, 19, 20 | − | + | − | FH 1-5 |
| FH 1-5, 19, 20 | − | + | − | FH 1-4, 19, 20 |
| FH 1-5, 19, 20 | − | + | − | FH 1-5, 19, 20 |
| FH 1-5, 19, 20 | − | + | − | FH 19, 20 |
| FH 1-5, 19, 20 | − | + | − | CR2 1-2 |
| FH 1-5, 19, 20 | − | + | − | CR2 1-3 |
| FH 1-5, 19, 20 | − | + | − | CR2 1-4 |
| FH 19-20 | − | + | − | FH 1-4 |
| FH 19-20 | − | + | − | FH 1-5 |
| FH 19-20 | − | + | − | FH 1-4, 19, 20 |
| FH 19-20 | − | + | − | FH 1-5, 19, 20 |
| CR2 1-2 | − | + | − | FH 1-4 |
| CR2 1-2 | − | + | − | FH 1-5 |
| CR2 1-2 | − | + | − | FH 1-4, 19, 20 |
| CR2 1-2 | − | + | − | FH 1-5, 19, 20 |
| CR2 1-3 | − | + | − | FH 1-4 |
| CR2 1-3 | − | + | − | FH 1-5 |
| CR2 1-3 | − | + | − | FH 1-4, 19, 20 |
| CR2 1-3 | − | + | − | FH 1-5, 19, 20 |
| CR2 1-4 | − | + | − | FH 1-4 |
| CR2 1-4 | − | + | − | FH 1-5 |
| CR2 1-4 | − | + | − | FH 1-4, 19, 20 |
| CR2 1-4 | − | + | − | FH 1-5, 19, 20 |
| CR2 1-2 (L3) FH 19-20 | − | + | − | FH 1-4 |
| CR2 1-2 (L3) FH 19-20 | − | + | − | FH 1-5 |
| FH 19-20 (L3) FH 19-20 | − | + | − | FH 1-4 |
| FH 19-20 (L3) FH 19-20 | − | + | − | FH 1-5 |

“+” indicates the inclusion of a feature, “−” while indicates the absence of a feature.

TABLE 4
Exemplary Fusion Proteins having the sequence, from
N-terminus to C-terminus, of D1-L1-VHH-L2-D2

| D1 (SCRs) | L1 | VHH | L2 | D2 (SCRs) |
|---|---|---|---|---|
| FH 1-4 | + | + | + | FH 1-4 |
| FH 1-4 | + | + | + | FH 1-5 |
| FH 1-4 | + | + | + | FH 1-4, 19, 20 |
| FH 1-4 | + | + | + | FH 1-5, 19, 20 |
| FH 1-4 | + | + | + | FH 19, 20 |
| FH 1-4 | + | + | + | − |
| FH 1-4 | + | + | + | − |
| FH 1-4 | + | + | + | − |
| FH 1-4 | + | + | + | − |
| FH 1-4 | + | + | + | FH 19-20 (L3) FH 19-20 |
| FH 1-5 | + | + | + | FH 1-4 |
| FH 1-5 | + | + | + | FH 1-5 |
| FH 1-5 | + | + | + | FH 1-4, 19, 20 |
| FH 1-5 | + | + | + | FH 1-5, 19, 20 |
| FH 1-5 | + | + | + | FH 19, 20 |
| FH 1-5 | + | + | + | − |
| FH 1-5 | + | + | + | − |
| FH 1-5 | + | + | + | − |
| FH 1-5 | + | + | + | − |
| FH 1-5 | + | + | + | FH 19-20 (L3) FH 19-20 |
| FH 1-4, 19, 20 | + | + | + | FH 1-4 |
| FH 1-4, 19, 20 | + | + | + | FH 1-5 |
| FH 1-4, 19, 20 | + | + | + | FH 1-4, 19, 20 |
| FH 1-4, 19, 20 | + | + | + | FH 1-5, 19, 20 |
| FH 1-4, 19, 20 | + | + | + | FH 19, 20 |
| FH 1-4, 19, 20 | + | + | + | − |
| FH 1-4, 19, 20 | + | + | + | − |
| FH 1-4, 19, 20 | + | + | + | − |
| FH 1-5, 19, 20 | + | + | + | FH 1-4 |
| FH 1-5, 19, 20 | + | + | + | FH 1-5 |
| FH 1-5, 19, 20 | + | + | + | FH 1-4, 19, 20 |
| FH 1-5, 19, 20 | + | + | + | FH 1-5, 19, 20 |
| FH 1-5, 19, 20 | + | + | + | FH 19, 20 |
| FH 1-5, 19, 20 | + | + | + | − |
| FH 1-5, 19, 20 | + | + | + | − |
| FH 1-5, 19, 20 | + | + | + | − |
| FH 19-20 | + | + | + | FH 1-4 |
| FH 19-20 | + | + | + | FH 1-5 |
| FH 19-20 | + | + | + | FH 1-4, 19, 20 |
| FH 19-20 | + | + | + | FH 1-5, 19, 20 |
| − | + | + | + | FH 1-4 |
| − | + | + | + | FH 1-5 |
| − | + | + | + | FH 1-4, 19, 20 |
| − | + | + | + | FH 1-5, 19, 20 |
| − | + | + | + | FH 1-4 |
| − | + | + | + | FH 1-5 |
| − | + | + | + | FH 1-4, 19, 20 |
| − | + | + | + | FH 1-5, 19, 20 |
| − | + | + | + | FH 1-4 |
| − | + | + | + | FH 1-5 |
| − | + | + | + | FH 1-4, 19, 20 |
| − | + | + | + | FH 1-5, 19, 20 |
| − | + | + | + | FH 1-4 |
| − | + | + | + | FH 1-5 |
| FH 19-20 (L3) FH 19-20 | + | + | + | FH 1-4 |
| FH 19-20 (L3) FH 19-20 | + | + | + | FH 1-5 |
| FH 1-4 | + | + | − | FH 1-4 |
| FH 1-4 | + | + | − | FH 1-5 |
| FH 1-4 | + | + | − | FH 1-4, 19, 20 |
| FH 1-4 | + | + | − | FH 1-5, 19, 20 |
| FH 1-4 | + | + | − | FH 19, 20 |
| FH 1-4 | + | + | − | − |
| FH 1-4 | + | + | − | − |
| FH 1-4 | + | + | − | − |
| FH 1-4 | + | + | − | − |
| FH 1-4 | + | + | − | FH 19-20 (L3) FH 19-20 |
| FH 1-5 | + | + | − | FH 1-4 |
| FH 1-5 | + | + | − | FH 1-5 |
| FH 1-5 | + | + | − | FH 1-4, 19, 20 |
| FH 1-5 | + | + | − | FH 1-5, 19, 20 |
| FH 1-5 | + | + | − | FH 19, 20 |
| FH 1-5 | + | + | − | − |
| FH 1-5 | + | + | − | − |
| FH 1-5 | + | + | − | − |
| FH 1-5 | + | + | − | − |
| FH 1-5 | + | + | − | FH 19-20 (L3) FH 19-20 |
| FH 1-4, 19, 20 | + | + | − | FH 1-4 |
| FH 1-4, 19, 20 | + | + | − | FH 1-5 |
| FH 1-4, 19, 20 | + | + | − | FH 1-4, 19, 20 |
| FH 1-4, 19, 20 | + | + | − | FH 1-5, 19, 20 |
| FH 1-4, 19, 20 | + | + | − | FH 19, 20 |
| FH 1-4, 19, 20 | + | + | − | − |
| FH 1-4, 19, 20 | + | + | − | − |
| FH 1-4, 19, 20 | + | + | − | − |
| FH 1-5, 19, 20 | + | + | − | FH 1-4 |
| FH 1-5, 19, 20 | + | + | − | FH 1-5 |
| FH 1-5, 19, 20 | + | + | − | FH 1-4, 19, 20 |
| FH 1-5, 19, 20 | + | + | − | FH 1-5, 19, 20 |
| FH 1-5, 19, 20 | + | + | − | FH 19, 20 |
| FH 1-5, 19, 20 | + | + | − | − |
| FH 1-5, 19, 20 | + | + | − | − |
| FH 1-5, 19, 20 | + | + | − | − |
| FH 19-20 | + | + | − | FH 1-4 |
| FH 19-20 | + | + | − | FH 1-5 |
| FH 19-20 | + | + | − | FH 1-4, 19, 20 |
| FH 19-20 | + | + | − | FH 1-5, 19, 20 |
| − | + | + | − | FH 1-4 |
| − | + | + | − | FH 1-5 |
| − | + | + | − | FH 1-4, 19, 20 |
| − | + | + | − | FH 1-5, 19, 20 |
| − | + | + | − | FH 1-4 |
| − | + | + | − | FH 1-5 |
| − | + | + | − | FH 1-4, 19, 20 |
| − | + | + | − | FH 1-5, 19, 20 |
| − | + | + | − | FH 1-4 |
| − | + | + | − | FH 1-5 |
| − | + | + | − | FH 1-4, 19, 20 |
| − | + | + | − | FH 1-5, 19, 20 |
| − | + | + | − | FH 1-4 |
| − | + | + | − | FH 1-5 |
| FH 19-20 (L3) FH 19-20 | + | + | − | FH 1-4 |
| FH 19-20 (L3) FH 19-20 | + | + | − | FH 1-5 |
| FH 1-4 | − | + | + | FH 1-4 |
| FH 1-4 | − | + | + | FH 1-5 |
| FH 1-4 | − | + | + | FH 1-4, 19, 20 |
| FH 1-4 | − | + | + | FH 1-5, 19, 20 |
| FH 1-4 | − | + | + | FH 19, 20 |
| FH 1-4 | − | + | + | − |
| FH 1-4 | − | + | + | − |
| FH 1-4 | − | + | + | − |
| FH 1-4 | − | + | + | − |
| FH 1-4 | − | + | + | FH 19-20 (L3) FH 19-20 |
| FH 1-5 | − | + | + | FH 1-4 |
| FH 1-5 | − | + | + | FH 1-5 |
| FH 1-5 | − | + | + | FH 1-4, 19, 20 |
| FH 1-5 | − | + | + | FH 1-5, 19, 20 |
| FH 1-5 | − | + | + | FH 19, 20 |
| FH 1-5 | − | + | + | − |
| FH 1-5 | − | + | + | − |
| FH 1-5 | − | + | + | − |
| FH 1-5 | − | + | + | − |
| FH 1-5 | − | + | + | FH 19-20 (L3) FH 19-20 |
| FH 1-4, 19, 20 | − | + | + | FH 1-4 |
| FH 1-4, 19, 20 | − | + | + | FH 1-5 |
| FH 1-4, 19, 20 | − | + | + | FH 1-4, 19, 20 |
| FH 1-4, 19, 20 | − | + | + | FH 1-5, 19, 20 |
| FH 1-4, 19, 20 | − | + | + | FH 19, 20 |
| FH 1-4, 19, 20 | − | + | + | − |
| FH 1-4, 19, 20 | − | + | + | − |
| FH 1-4, 19, 20 | − | + | + | − |
| FH 1-5, 19, 20 | − | + | + | FH 1-4 |
| FH 1-5, 19, 20 | − | + | + | FH 1-5 |
| FH 1-5, 19, 20 | − | + | + | FH 1-4, 19, 20 |
| FH 1-5, 19, 20 | − | + | + | FH 1-5, 19, 20 |
| FH 1-5, 19, 20 | − | + | + | FH 19, 20 |
| FH 1-5, 19, 20 | − | + | + | − |
| FH 1-5, 19, 20 | − | + | + | − |
| FH 1-5, 19, 20 | − | + | + | − |
| FH 19-20 | − | + | + | FH 1-4 |
| FH 19-20 | − | + | + | FH 1-5 |
| FH 19-20 | − | + | + | FH 1-4, 19, 20 |
| FH 19-20 | − | + | + | FH 1-5, 19, 20 |
| − | − | + | + | FH 1-4 |
| − | − | + | + | FH 1-5 |
| − | − | + | + | FH 1-4, 19, 20 |
| − | − | + | + | FH 1-5, 19, 20 |
| − | − | + | + | FH 1-4 |
| − | − | + | + | FH 1-5 |
| − | − | + | + | FH 1-4, 19, 20 |
| − | − | + | + | FH 1-5, 19, 20 |
| − | − | + | + | FH 1-4 |
| − | − | + | + | FH 1-5 |
| − | − | + | + | FH 1-4, 19, 20 |
| − | − | + | + | FH 1-5, 19, 20 |
| − | − | + | + | FH 1-4 |
| − | − | + | + | FH 1-5 |
| FH 19-20 (L3) FH 19-20 | − | + | + | FH 1-4 |
| FH 19-20 (L3) FH 19-20 | − | + | + | FH 1-5 |
| FH 1-4 | − | + | − | FH 1-4 |
| FH 1-4 | − | + | − | FH 1-5 |
| FH 1-4 | − | + | − | FH 1-4, 19, 20 |
| FH 1-4 | − | + | − | FH 1-5, 19, 20 |
| FH 1-4 | − | + | − | FH 19, 20 |
| FH 1-4 | − | + | − | − |
| FH 1-4 | − | + | − | − |
| FH 1-4 | − | + | − | − |
| FH 1-4 | − | + | − | − |
| FH 1-4 | − | + | − | FH 19-20 (L3) FH 19-20 |
| FH 1-5 | − | + | − | FH 1-4 |
| FH 1-5 | − | + | − | FH 1-5 |
| FH 1-5 | − | + | − | FH 1-4, 19, 20 |
| FH 1-5 | − | + | − | FH 1-5, 19, 20 |
| FH 1-5 | − | + | − | FH 19, 20 |
| FH 1-5 | − | + | − | − |
| FH 1-5 | − | + | − | − |
| FH 1-5 | − | + | − | − |
| FH 1-5 | − | + | − | − |
| FH 1-5 | − | + | − | FH 19-20 (L3) FH 19-20 |
| FH 1-4, 19, 20 | − | + | − | FH 1-4 |
| FH 1-4, 19, 20 | − | + | − | FH 1-5 |
| FH 1-4, 19, 20 | − | + | − | FH 1-4, 19, 20 |
| FH 1-4, 19, 20 | − | + | − | FH 1-5, 19, 20 |
| FH 1-4, 19, 20 | − | + | − | FH 19, 20 |
| FH 1-4, 19, 20 | − | + | − | − |
| FH 1-4, 19, 20 | − | + | − | − |
| FH 1-4, 19, 20 | − | + | − | − |
| FH 1-5, 19, 20 | − | + | − | FH 1-4 |
| FH 1-5, 19, 20 | − | + | − | FH 1-5 |
| FH 1-5, 19, 20 | − | + | − | FH 1-4, 19, 20 |
| FH 1-5, 19, 20 | − | + | − | FH 1-5, 19, 20 |
| FH 1-5, 19, 20 | − | + | − | FH 19, 20 |
| FH 1-5, 19, 20 | − | + | − | − |
| FH 1-5, 19, 20 | − | + | − | − |
| FH 1-5, 19, 20 | − | + | − | − |
| FH 19-20 | − | + | − | FH 1-4 |
| FH 19-20 | − | + | − | FH 1-5 |
| FH 19-20 | − | + | − | FH 1-4, 19, 20 |
| FH 19-20 | − | + | − | FH 1-5, 19, 20 |
| − | − | + | − | FH 1-4 |
| − | − | + | − | FH 1-5 |
| − | − | + | − | FH 1-4, 19, 20 |
| − | − | + | − | FH 1-5, 19, 20 |
| − | − | + | − | FH 1-4 |
| − | − | + | − | FH 1-5 |
| − | − | + | − | FH 1-4, 19, 20 |
| − | − | + | − | FH 1-5, 19, 20 |
| − | − | + | − | FH 1-4 |
| − | − | + | − | FH 1-5 |
| − | − | + | − | FH 1-4, 19, 20 |
| − | − | + | − | FH 1-5, 19, 20 |
| − | − | + | − | FH 1-4 |
| − | − | + | − | FH 1-5 |
| FH 19-20 (L3) FH 19-20 | − | + | − | FH 1-4 |
| FH 19-20 (L3) FH 19-20 | − | + | − | FH 1-5 |

“+” indicates the inclusion of a feature, “−” while indicates the absence of a feature.

### Production of Fusion Proteins

Described herein are methods for producing a fusion protein described herein using nucleic acid molecules encoding the fusion proteins, such as the fusion proteins shown in Tables 1-4. The nucleic acid molecule can be operably linked to a suitable control sequence to form an expression unit encoding the protein. An exemplary signal peptide (leader sequence) is that of mouse Ig heavy chain V region 102 (SEQ ID NO: 223; UniProt Accession Number P01750). The expression unit is used to transform a suitable host cell, and the transformed host cell is cultured under conditions that allow the production of the recombinant protein. Optionally, the recombinant protein is isolated from the medium or from the cells; recovery and purification of the protein may not be necessary in some instances where some impurities may be tolerated. Additional residues may be included at the N- or C-terminus of the protein-coding sequence to facilitate purification (e.g., a histidine tag).

The fusion proteins of the present disclosure may include naturally-occurring or a non-naturally-occurring components; preferably at least one component is non-naturally occurring, e.g., with respect to its structure (e.g., sequence) and/or its association (e.g., how it is linked to other components). As used herein, the term “non-naturally occurring” refers to any molecule, e.g., fusion protein, produced with the aid of human manipulation, including, without limitation, molecules produced by genetic engineering using random mutagenesis or rational design and molecules produced by chemical synthesis. Non-limiting examples of non-naturally occurring molecules include, e.g., conservatively substituted variants, non-conservatively substituted variants, and active hybrids (e.g., chimeras) or fragments. Non-natural molecules further include natural molecules that have been modified, e.g., post-translationally, e.g., via addition of chemical moieties, tags, ligands. Preferably, non-natural molecules include the fusion proteins of the present disclosure.

The fusion protein can be expressed from a single polynucleotide that encodes the entire fusion protein or as multiple (e.g., two or more) polynucleotides that may be expressed by suitable expression systems or may be co-expressed. Polypeptides encoded by polynucleotides that are co-expressed may associate through, e.g., disulfide bonds or other means to form a functional fusion protein. For example, the light chain portion of monoclonal antibody may be encoded by a separate polynucleotide from the heavy chain portion of a monoclonal antibody. When co-expressed in a host cell, the heavy chain polypeptides will associate with the light chain polypeptides to form the monoclonal antibody.

It is envisioned that any and all polynucleotide molecules that can encode the fusion proteins disclosed in the present specification can be useful, including, without limitation naturally-occurring and non-naturally-occurring DNA molecules and naturally-occurring and non-naturally-occurring RNA molecules. Non-limiting examples of naturally-occurring and non-naturally-occurring DNA molecules include single-stranded DNA molecules, double-stranded DNA molecules, genomic DNA molecules, cDNA molecules, vector constructs, such as, e.g., plasmid constructs, phagemid constructs, bacteriophage constructs, retroviral constructs and artificial chromosome constructs. Non-limiting examples of naturally-occurring and non-naturally-occurring RNA molecules include single-stranded RNA, double stranded RNA and mRNA. The present disclosure also provides synthetic nucleic acids, e.g., non-natural nucleic acids, comprising nucleotide sequence encoding one or more of the aforementioned fusion proteins. Included herein are nucleic acids encoding the fusion proteins, including the complementary strand thereto, or the RNA equivalent thereof, or a complementary RNA equivalent thereof.

Typically, a nucleic acid encoding the desired fusion protein is generated using molecular cloning methods, and is generally placed within a vector, such as a plasmid constructs, phagemid constructs, bacteriophage constructs, retroviral constructs and artificial chromosome constructs. Non-limiting examples of naturally-occurring and non-naturally-occurring RNA molecules include single-stranded RNA, double stranded RNA and mRNA. The vector is used to transform the nucleic acid into a host cell appropriate for the expression of the fusion polypeptide. Representative methods are disclosed, for example, in Maniatis et al. (Cold Springs Harbor Laboratory, 1989). Many cell types can be used as appropriate host cells, although mammalian cells are preferable because they are able to confer appropriate post-translational modifications. Host cells can include, e.g., a Human Embryonic Kidney (HEK) (e.g., HEK 293) cell, Chinese Hamster Ovary (CHO) cell, L cell, C127 cell, 3T3 cell, BHK cell, COS-7 cell, or any other suitable host cell known in the art.

In addition, prokaryotic cells including, without limitation, strains of aerobic, microaerophilic, capnophilic, facultative, anaerobic, gram-negative and gram-positive bacterial cells such as those derived from, e.g., Escherichia coli, Bacillus subdlis, Bacillus licheniformis, Bacteroides fragilis, Clostridia perfringens, Clostridia difficile, Caulobacter crescentus, Lactococcus lacts, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca Sexta; and mammalian cells and cell-lines derived from mammalian cells, such as, e.g., those derived from mouse, rat, hamster, porcine, bovine, equine, primate and human may be used. Cell lines may be obtained from the American Type Culture Collection (2004); European Collection of Cell Cultures (2204); and the German Collection of Microorganisms and Cell Cultures (2004).

Included herein are codon-optimized sequences of the aforementioned nucleic acid sequences and vectors. Codon optimization for expression in a host cell, e.g., bacteria such as E. coli or insect Hi5 cells, may be performed using Codon Optimization Tool (CODONOPT), available freely from Integrated DNA Technologies, Inc., Coralville, Iowa, USA. In one embodiment, a nucleic acid or polynucleotide encoding the fusion protein is provided. In one embodiment, a vector including a nucleic acid or polynucleotide encoding the fusion protein is provided. In one embodiment, a host cell including one or more polynucleotides encoding the fusion protein is provided. In certain embodiments a host cell including one or more fusion expression vectors is provided. The fusion proteins can be produced by expression of a nucleotide sequence in any suitable expression system known in the art. Any expression system may be used, including yeast, bacterial, animal, plant, eukaryotic, and prokaryotic systems. In some embodiments, yeast systems that have been modified to reduce native yeast glycosylation, hyper-glycosylation or proteolytic activity may be used. Furthermore, any in vivo expression systems designed for high level expression of recombinant proteins within organisms known in the art can be used for producing the fusion proteins specified herein. In some embodiments, the factor H fusion protein, as described herein, is produced by culturing one or more host cells including one or more nucleic acid molecules capable of expressing the fusion protein under conditions suitable for expression of the fusion protein. In some embodiments, the factor H fusion protein is obtained from the cell culture or culture medium.

The fusion protein can also be produced using chemical methods to synthesize the desired amino acid sequence, in whole or in part. For example, polypeptides can be synthesized by solid phase techniques, cleaved from the resin, and purified by preparative high performance liquid chromatography (e.g., Creighton (1983) Proteins: Structures And Molecular Principles, WH Freeman and Co, New York N.Y.). The composition of the synthetic polypeptides can be confirmed by amino acid analysis or sequencing. Additionally, the amino acid sequence of a fusion protein or any part thereof, can be altered during direct synthesis and/or combined using chemical methods with a sequence from other subunits, or any part thereof, to produce a variant polypeptide.

### Isolation/Purification of Fusion Proteins

Secreted, biologically active fusion proteins described herein, such as those described in Tables 1-4, may be purified by techniques such as high performance liquid chromatography, ion exchange chromatography, gel electrophoresis, affinity chromatography, e.g., protein A affinity chromatography, size exclusion chromatography, and the like. The conditions used to purify a particular protein depend, in part, on factors such as net charge, hydrophobicity, hydrophilicity etc., as would be apparent to a skilled artisan.

### Assays for Fusion Protein Activity

### Hemolytic Assay

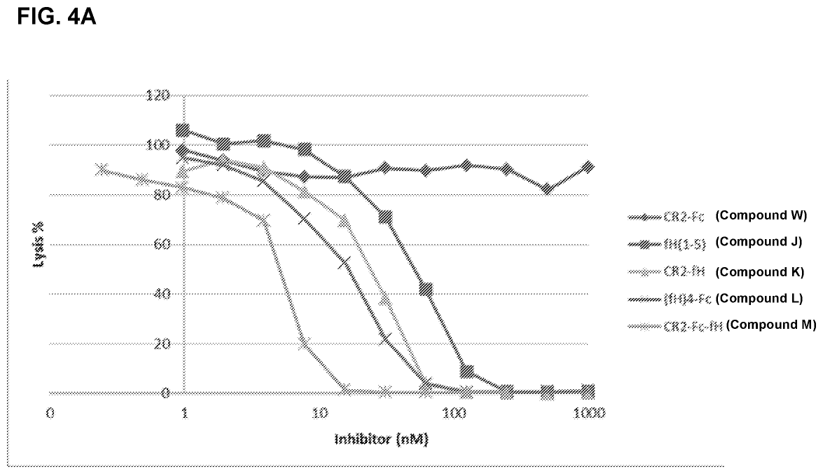

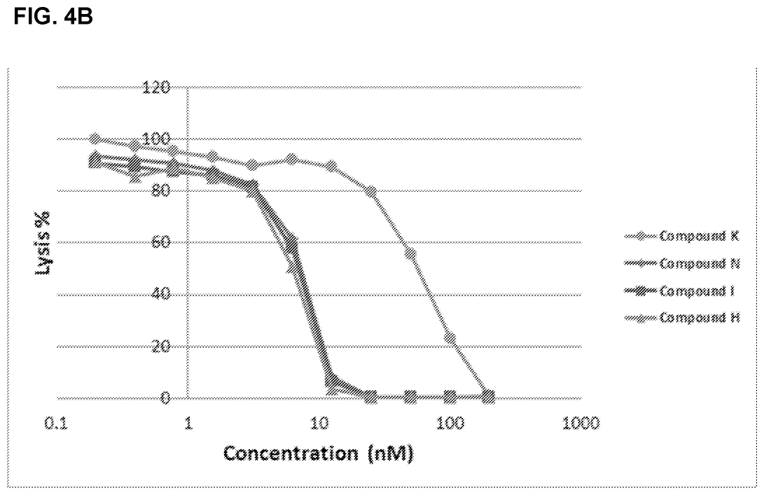

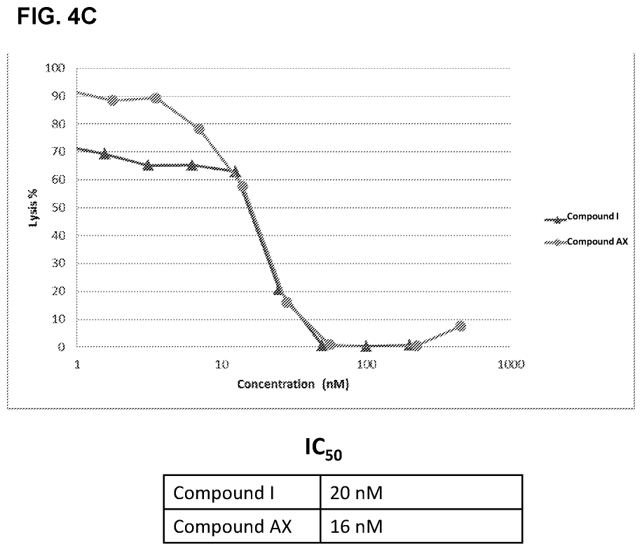

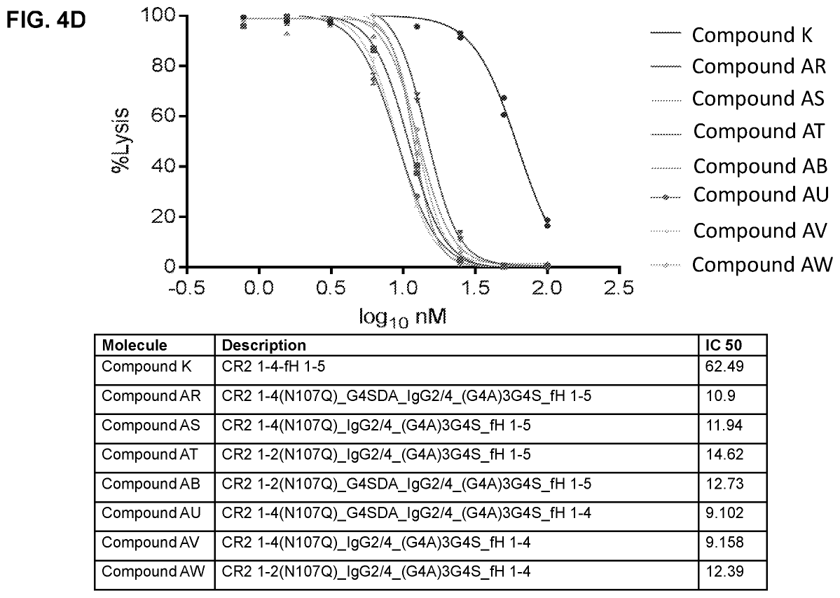

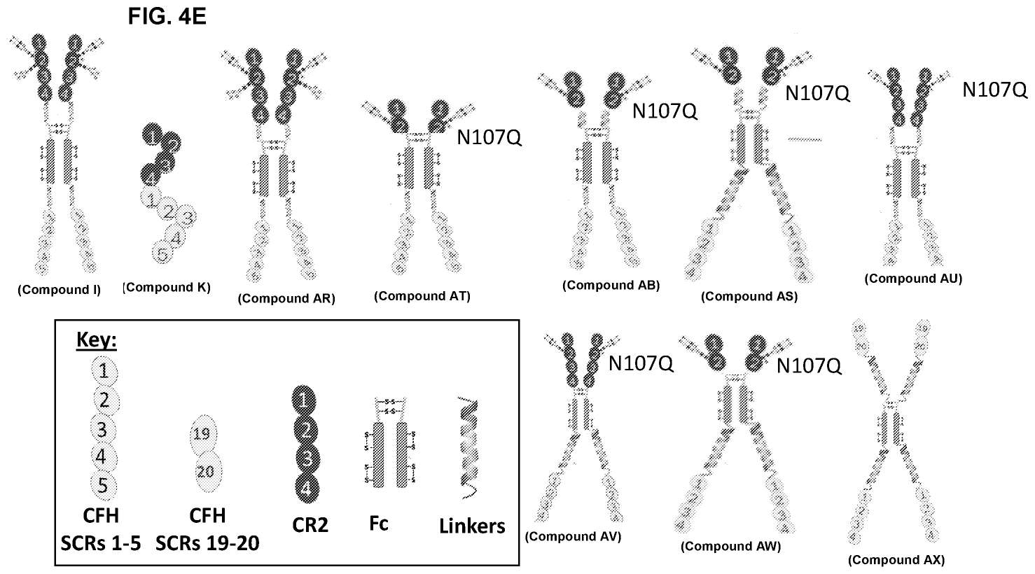

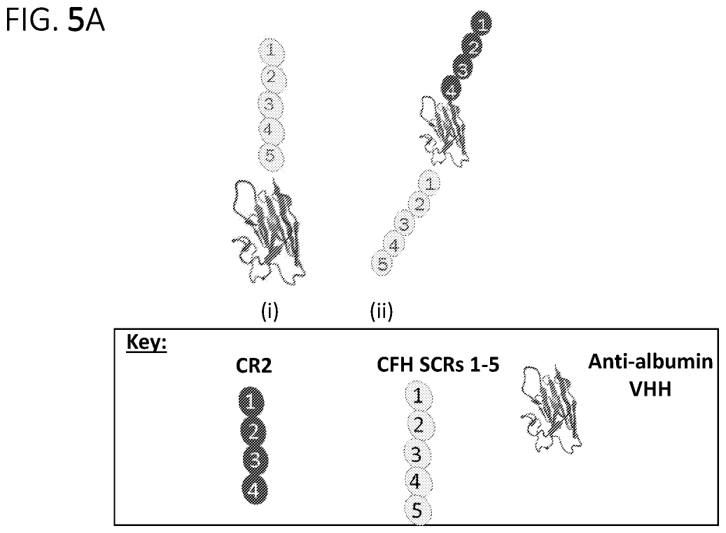

The fusion proteins described herein were assessed for activity using a complement pathway hemolysis assay, which measures complement-mediated lysis of rabbit erythrocytes secondary to activation of the alternative pathway on a cell surface. Rabbit erythrocytes generally activate complement-mediated lysis in mouse or human serum. As serum C3 is activated, C3 convertases, C3 activation fragments, and C5 convertases are deposited on rabbit RBCs. Serum alternative complement pathway activity in the presence of a fusion protein comprising a fragment of factor H and an Fc domain (e.g., an IgG, or a functional fragment thereof, e.g., an Fc receptor binding domain) or a fragment of factor H, a fragment of CR2, and an Fc (e.g., an IgG, or a functional fragment thereof, e.g., an Fc receptor binding domain; see, e.g., the fusion proteins of Tables 1-4), for example, were evaluated in a concentration-dependent manner in human or mouse serum supplemented with Mg++ and EGTA as Ca sequestrant, thus favoring the alternative pathway of complement activation. Incubation of rabbit erythrocytes in normal mouse or human serum causes cell lysis, while addition of nanomolar quantities of a fusion protein comprising a fragment of factor H and an Fc domain, or a fragment of factor H, a fragment of CR2, and an Fc domain, for example, is decreased the degree of lysis (see FIGS. 4A-4D, FIG. 6B, and FIGS. 9-11). Fusion proteins of the disclosure may exhibit a half maximal inhibitory concentration (ICo) of between about 9 nM to about 65 nM (e.g., between about 9 nM to about 50 nM, between about 9 nM to about 40 nM, between about 9 nM to about 30 nM, between about 9 nM to about 20 nM, between about 30 nM to about 60 nM, between about 40 nM to about 60 nM, or between about 50 nM to about 60 nM. For example, Compound A B may have an IC₅₀ of between about 9 nM to about 11 nM (e.g., 10.82 nM), Compound AC may have an IC₅₀ of between about 10 nM to about 12 nM (e.g., 11.4 nM).

### Complement Activity Assay

The fusion proteins described herein (e.g., the fusion proteins of Tables 1-4) can be evaluated for alternative complement pathway activity can be evaluated in the fluid phase using an alternative complement pathway assay kit, for example, Complement system Alternative Pathway WIESLAB®, Lund, Sweden. This method combines principles of the hemolytic assay for complement activation with the use of labeled antibodies specific for a neoantigen produced as a result of complement activation. The amount of neoantigen generated is proportional to the functional activity of the alternative pathway. In the Complement system Alternative Pathway kit, wells of the plate are coated with specific activators of the alternative pathway. Serum is diluted in diluent containing specific blockers to ensure that only the alternative pathway is activated. Anti-properdin V_{HH} for example, can be spiked into the patient's blood in a concentration-dependent manner. During the incubation of the diluted patient serum in the wells, complement is activated by the specific coating. The wells are then washed and C5b-9 is detected with a specific alkaline phosphatase-labelled antibody to the neoantigen as a result of complement activation. The amount of complement activation correlates with the color intensity and is measured in terms of absorbance (optical density (OD)) at 405 nm. The addition of nanomolar quantities of a factor H fusion protein according to the disclosure, for example, decreases the degree of activity. Additional exemplary assays for determining complement pathway activity include those described in Hebell et al., (Science (1991) 254(5028):102-105).

### Pharmaceutical Compositions, Dosage, and Administration

The fusion proteins described herein (see, e.g., Tables 1-4, in particular those described in Table 1) can be incorporated into pharmaceutical compositions suitable for administration to a subject. Pharmaceutical compositions including factor H fusion proteins described herein can be formulated for administration at individual doses ranging, e.g., from 0.01 mg/kg to 500 mg/kg. The pharmaceutical composition may contain, e.g., from 0.1 μg/0.5 mL to 1 g/5 mL of the fusion protein.

Compositions including factor H fusion proteins can also be formulated for either a single or multiple dosage regimens. Doses can be formulated for administration, e.g., hourly, bihourly, daily, bidaily, twice a week, three times a week, four times a week, five times a week, six times a week, weekly, biweekly, monthly, bimonthly, or yearly. Alternatively, doses can be formulated for administration, e.g., twice, three times, four times, five times, six times, seven times, eight times, nine times, ten times, eleven times, or twelve times per day.

The pharmaceutical compositions including factor H fusion proteins can be formulated according to standard methods. Pharmaceutical formulation is a well-established art, and is further described in, e.g., Gennaro (2000) Remington: The Science and Practice of Pharmacy, 20th Edition, Lippincott, Williams & Wilkins (ISBN: 0683306472); Ansel et al. (1999) Pharmaceutical Dosage Forms and Drug Delivery Systems, 7th Edition, Lippincott Williams & Wilkins Publishers (ISBN: 0683305727); and Kibbe (2000) Handbook of Pharmaceutical Excipients, American Pharmaceutical Association, 3rd Edition (ISBN: 091733096X).

The pharmaceutical composition can include the fusion protein and at least one pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The term “pharmaceutically acceptable carrier” excludes tissue culture medium including bovine or horse serum. Pharmaceutically acceptable carriers or adjuvants, by themselves, do not induce the production of antibodies harmful to the individual receiving the composition nor do they elicit protection. Therefore, pharmaceutically acceptable carriers are inherently non-toxic and nontherapeutic, and are known to the person skilled in the art. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable substances include minor amounts of auxiliary substances such as wetting or emulsifying agents, preservatives, or buffers, which enhance the shelf life or effectiveness of the antibody.

The compositions described herein may be prepared in a variety of forms. These include, for example, liquid, semi-solid, and solid dosage forms, such as liquid solutions (e.g., injectable and infusible solutions), dispersions or suspensions, tablets, pills, powders, liposomes and suppositories. Such formulations can be prepared by methods known in the art such as, e.g., the methods described in Epstein et al. (1985) Proc Nad Acad Sci USA 82:3688; Hwang et al. (1980) Proc Nad Acad Sci USA 77:4030; and U.S. Pat. Nos. 4,485,045 and 4,544,545. Liposomes with enhanced circulation time are disclosed in, e.g., U.S. Pat. No. 5,013,556.

Pharmaceutical compositions including factor H fusion proteins can also be formulated with a carrier that will protect the composition (e.g., a factor H fusion protein) against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) Sustained and Controlled Release Drug Delivery Systems, Marcel Dekker, Inc., New York.

The final form depends on the intended mode of administration and therapeutic application. Typical compositions are in the form of injectable or infusible solutions, such as compositions similar to those used for passive immunization of humans with other antibodies. The composition(s) can delivered by, for example, parenteral injection (e.g., intravenous, subcutaneous, intraperitoneal, intramuscular).

The pharmaceutical compositions can be provided in a sterile form and stable under the conditions of manufacture and storage. The composition can be formulated as a solution, microemulsion, dispersion, liposome, or other ordered structure suitable to high drug concentration. Sterile injectable solutions can be prepared by incorporating the fusion protein in the required amount in an appropriate solvent with one or a combination of ingredients enumerated above, as required, followed by filter sterilization. Generally, dispersions are prepared by incorporating the fusion protein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin. The preferred form depends, in part, on the intended mode of administration and therapeutic application. For example, compositions intended for systemic or local delivery can be in the form of injectable or infusible solutions. The composition can be formulated, for example, as a buffered solution at a suitable concentration and suitable for storage at 2-8° C. (e.g., 4° C.). A composition can also be formulated for storage at a temperature below 0° C. (e.g., −20° C. or −80° C.). A composition can further be formulated for storage for up to 2 years (e.g., one month, two months, three months, four months, five months, six months, seven months, eight months, nine months, 10 months, 11 months, 1 year, 11% years, or 2 years) at 2-8° C. (e.g., 4° C.). Thus, the compositions described herein can be stable in storage for at least 1 year at 2-8° C. (e.g., 4° C.).

The fusion proteins described herein can be administered by a variety of methods known in the art, although for many therapeutic applications, the preferred route/mode of administration is intravenous injection or infusion. The fusion proteins can also be administered by intramuscular or subcutaneous injection. As will be appreciated by the skilled artisan, the route and/or mode of administration will vary depending upon the desired results.

In certain embodiments, the fusion protein may be prepared with a carrier that will protect the antibody against rapid release, such as a controlled release formulation, including implants, transdermal patches, and microencapsulated delivery systems.

Biodegradable, biocompatible polymers can be used, such as ethylene vinyl acetate, polyanhydrides, polyglycolic acid, collagen, polyorthoesters, and polylactic acid. Prolonged absorption of injectable compositions can be attained by including in the composition an agent that delays absorption, for example, monostearate salts and gelatin. Many methods for the preparation of such formulations are known to those skilled in the art (e.g., Sustained and Controlled Release Drug Delivery Systems, J. R. Robinson, ed., Marcel Dekker, Inc., New York, 1978). Additional methods applicable to the controlled or extended release of fusion proteins disclosed herein are described, for example, in WO 2016081884, the entire contents of which are incorporated herein by reference.

The pharmaceutical composition(s) may have a pH of about 5.6-10.0, about 6.0-8.8, or about 6.5-8.0. For example, the pH may be about 6.2, 6.5, 6.75, 7.0, or 7.5. The pharmaceutical compositions may be formulated for oral, sublingual, intranasal, intraocular, rectal, transdermal, mucosal, topical, intravitreal, or parenteral administration. Parenteral administration may include intradermal, subcutaneous (s.c, s.q., sub-Q, Hypo), intramuscular (i.m.), intravenous (i.v.), intraperitoneal (i.p.), intra-arterial, intramedulary, intracardiac, intravitreal (eye), intra-articular (joint), intrasynovial (joint fluid area), intracranial, intraspinal, and intrathecal (spinal fluids) injection or infusion. Any device suitable for parenteral injection or infusion of drug formulations may be used for such administration. For example, the pharmaceutical composition may be contained in a sterile pre-filled syringe.

Additional active compounds can also be incorporated into the composition. In certain embodiments, a fusion protein is co-formulated with and/or co-administered with one or more additional therapeutic agents. When compositions are to be used in combination with a second active agent, the compositions can be co-formulated with the second agent, or the compositions can be formulated separately from the second agent formulation. For example, the respective pharmaceutical compositions can be mixed, e.g., just prior to administration, and administered together or can be administered separately, e.g., at the same or different times. In some embodiments, a fusion protein can be co-formulated and/or co-administered with one or more additional antibodies that bind other targets (e.g., antibodies that bind regulators of the alternative complement pathway). Such combination therapies may utilize lower dosages of the administered therapeutic agents, thus avoiding possible toxicities or complications associated with the various monotherapies. Additionally, the compositions described herein can be co-formulated or co-administered with other therapeutic agents to ameliorate side effects of administering the compositions described herein (e.g., therapeutic agents that minimize risk of infection in an immunocompromised environment, for example, anti-bacterial agents, anti-fungal agents and anti-viral agents).

Preparations of compositions containing factor H fusion proteins can be provided to a subject in combination with pharmaceutically acceptable sterile aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions, or suspensions, including saline and buffered medical parenteral vehicles including sodium chloride solution, Ringer's dextrose solution, dextrose plus sodium chloride solution, Ringer's solution containing lactose, or fixed oils.

Intravenous vehicles can include fluid and nutrient replenishers, electrolyte replenishers, such as those based upon Ringer's dextrose, and the like. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, can be present in such vehicles. A thorough discussion of pharmaceutically acceptable carriers is available in Remington's Pharmaceutical Sciences (Mack Pub. Co., N.J. 1991).

The pharmaceutical compositions can include a “therapeutically effective amount” or a “prophylactically effective amount” of a fusion protein. A “therapeutically effective amount” refers to an amount effective, at dosages, and for periods of time necessary, to achieve the desired therapeutic result. A therapeutically effective amount of the antibody can vary according to factors such as the disease state, age, sex, and weight of the individual, and the ability of the fusion protein to elicit a desired response in the individual. A “prophylactically effective amount” refers to an amount effective, at dosages, and for periods of time necessary, to achieve the desired prophylactic result. In some embodiments, a prophylactic dose is used in subjects prior to or at an earlier stage of disease where the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time, or the dose may be proportionally reduced or increased as indicated by the exigencies of the therapeutic situation. It is advantageous to formulate parenteral compositions in dosage unit form for ease of administration and uniformity of dosage. Dosage unit form as used herein refers to physically discrete units suited as unitary dosages for the mammalian subjects to be treated: each unit containing a predetermined quantity of active compound calculated to produce the desired therapeutic effect in association with the required pharmaceutical carrier. It is to be noted that dosage values can vary with the type and severity of the condition to be alleviated. It is to be further understood that for any particular subject, specific dosage regimens should be adjusted over time according to the individual need and the professional judgment of the administering clinician.

The efficacy of treatment with a fusion protein as described herein can be assessed based on an improvement in one or more symptoms or indicators of the disease state or disorder being treated. An improvement of at least 10% (increase or decrease, depending upon the indicator being measured) in one or more clinical indicators is considered “effective treatment,” although greater improvements are preferred, such as 20%, 30%, 40%, 50%, 75%, 90%, or even 100%, or, depending upon the indicator being measured, more than 100% (e.g., two-fold, three-fold, ten-fold, etc., up to and including attainment of a disease-free state.

### Methods of Treatment Using the Fusion Proteins

The complement factor H fusion proteins described herein (see e.g., Tables 1-4) can be used to treat diseases mediated by alternative complement pathway dysregulation by inhibiting the alternative complement pathway activation in a mammal (e.g., a human). The fusion protein(s) described herein can be used to treat a variety of alternative complement pathway-associated disorders. Such disorders include, without limitation, paroxysmal nocturnal hemoglobinuria (PNH), atypical hemolytic uremic syndrome (aHUS), IgA nephrology, lupus nephritis, C3 glomerulopathy (C3G), dermatomyositis, systemic sclerosis, demyelinating polyneuropathy, pemphigus, membranous nephropathy, focal segmental glomerular sclerosis (FSGS), bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, dense deposit disease (DDD), age related macular degeneration (AMD), systemic lupus erythematosus (SLE), rheumatoid arthritis (RA), multiple sclerosis (MS), traumatic brain injury (TBI), ischemia reperfusion injury, preeclampsia, or thrombic thrombocytopenic purpura (TTP).

A therapeutically effective amount of a complement factor H fusion protein, as disclosed herein (e.g., a fusion protein having the sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), is administered to a mammalian subject in need of such treatment. The preferred subject is a human patient. The amount administered should be sufficient to inhibit complement activation and/or restore normal alternative complement pathway regulation. The determination of a therapeutically effective dose is within the capability of practitioners in this art; however, as an example, in embodiments of the method described herein utilizing systemic administration of a fusion protein for the treatment diseases mediated by alternative complement pathway dysregulation, an effective human dose will be in the range of 0.01 mg/kg-150 mg/kg ((e.g., from 0.05 mg/kg to 500 mg/kg, from 0.1 mg/kg to 20 mg/kg, from 5 mg/kg to 500 mg/kg, from 0.1 mg/kg to 100 mg/kg, from 10 mg/kg to 100 mg/kg, from 0.1 mg/kg to 50 mg/kg, from 0.5 mg/kg to 25 mg/kg, from 1.0 mg/kg to 10 mg/kg, from 1.5 mg/kg to 5 mg/kg, or from 2.0 mg/kg to 3.0 mg/kg) or from 1 μg/kg to 1,000 μg/kg (e.g., from 5 μg/kg to 1,000 μg/kg, from 1 μg/kg to 750 μg/kg, from 5 μg/kg to 750 μg/kg, from 10 μg/kg to 750 μg/kg, from 1 μg/kg to 500 μg/kg, from 5 μg/kg to 500 μg/kg, from 10 μg/kg to 500 μg/kg, from 1 μg/kg to 100 μg/kg, from 5 μg/kg to 100 μg/kg, from 10 μg/kg to 100 μg/kg, from 1 μg/kg to 50 μg/kg, from 5 μg/kg to 50 μg/kg, or from 10 μg/kg to 50 μg/kg). The route of administration may affect the recommended dose. Repeated systemic doses are contemplated to maintain an effective level, e.g., to attenuate or inhibit complement activation in a patient's system, depending on the mode of administration adopted.

The methods proteins described herein are particularly useful for treating renal lesions characterized histologically by predominant C3 accumulation the glomerular basement membrane in the absence of significant deposition of immunoglobulin (Nester, C. & Smith, R., Curr. Opin. Nephrol. Hypertens., 22:231-7, 2013) from aberrant regulation of the alternative pathway of complement, also known as C3 glomerulopathy (C3G).

The methods described herein are particularly useful for treating dense deposit disease (DDD), DDD is a rare kidney disease leading to persisting proteinuria, hematuria, and nephritic syndrome. Factor H deficiency and dysfunction in DDD has been reported in several cases. For example, mutations in factor H have been found in human patients with DDD. Symptoms of DDD include, e.g., one or both of hematuria and proteinuria; acute nephritic syndrome; drusen development and/or visual impairment; acquired partial lipodystrophy and complications thereof; and the presence of serum C3 nephritic factor (C3NeF), an autoantibody directed against C3bBb, the C3 convertase of the alternative complement pathway (Appel, G. et al., J. Am. Soc. Nephrol., 16:1392-404, 2005). Targeting factor H to complement activation sites has therapeutic effects on an individual having DDD. In some embodiments, administering an effective dose to the individual a composition including a fusion molecule described herein is effective in treating DDD. The route of administration may affect the recommended dose. Repeated systemic doses are contemplated to maintain an effective level, e.g., to attenuate or inhibit complement activation in a patient's system, depending on the mode of administration adopted.

The compositions and methods described herein are particularly useful for treatment of renal inflammation caused by systemic lupus erythematosus (SLE), such as lupus nephritis. Lupus glomerulonephritis, includes diverse and complex morphological lesions, depending on the proportion of glomeruli affected by active or chronic lesions, the degree of interstitial inflammation or fibrosis, as well as vascular lesions (Weening, J. et al., J. Am. Soc. Nephrol., 15:241-50, 2004). Lupus nephritis is a serious complication that occurs in a subpopulation of patients with SLE. SLE is the prototypic autoimmune disease resulting in multi-organ involvement. This anti-self response is characterized by autoantibodies directed against a variety of nuclear and cytoplasmic cellular components. These autoantibodies bind to their respective antigens, forming immune complexes that circulate and eventually deposit in tissues. This immune complex deposition causes chronic inflammation and tissue damage. Complement pathways (including the alternative complement pathway) are implicated in the pathology of SLE, and thus fusion proteins provided herein are thus useful for treating lupus nephritis.

The methods described herein are particularly useful for treatment treating macular degeneration, such as AMD. AMD refers to age-related deterioration or breakdown of the eye's macula, resulting in the loss of integrity of the histoarchitecture of the cells and/or extracellular matrix of the normal macula and/or the loss of function of the cells of the macula. It is clinically characterized by progressive loss of central vision that occurs as a result of damage to the photoreceptor cells in an area of the retina called the macula. AMD encompasses all stages of AMD, including Category 2 (early stage), Category 3 (intermediate), and Category 4 (advanced) AMD. Also encompassed are the two clinical states for which AMD has been broadly classified: a wet form and a dry form, with the dry form making up to 80-90% of total cases. The proteins of the alternative complement pathway are central to the development of age-related macular degeneration (Zipfel, P. et at, Adv. Exp. Med. Biol., 703:9-24, 2010). Analysis of ocular deposits in AMD patients has shown a large number of inflammatory proteins including amyloid proteins, coagulation factors, and proteins of the complement pathway. A genetic variation in the complement factor H substantially raises the risk of AMD, suggesting that uncontrolled complement activation underlies the pathogenesis of AMD (Edwards, A. et al., Science, 308:421-4, 2005; Haines, J. et al., Science, 308:419-21, 2005; Klein, R. et al., Science, 308:385-9, 2005; Hageman, G. et al., Proc. Natl. Acad. Sci. USA, 102:7227-32, 2005). In some embodiments, methods of treating AMD, include, but are not limited to, formation of ocular drusen, inflammation in the eye or eye tissue, loss of photoreceptor cells, loss of vision (including for example visual acuity and visual field), neovascularization (such as choroidal neovascularization or CNV), and retinal detachment. Other related aspects, such as photoreceptor degeneration, RPE degeneration, retinal degeneration, chorioretinal degeneration, cone degeneration, retinal dysfunction, retinal damage in response to light exposure (such as constant light exposure), damage of the Bruch's membrane, loss of RPE function, loss of integrity of the histoarchitecture of the cells and/or extracellular matrix of the normal macular, loss of function of the cells in the macula, photoreceptor dystrophy, mucopolysaccharidoses, rod-cone dystrophies, cone-rod dystrophies, anterior and posterior uvitis, and diabetic neuropathy, are also included.

The compositions and methods described herein are particularly useful for treatment of PNH. PNH is a consequence of clonal expansion of one or more hematopoietic stem cells with mutant PIG-A. The extent to which the PIG-A mutant clone expands varies widely among patients. Another feature of PNH is its phenotypic mosaicism based on the PIG-A genotype that determines the degree of GPI-AP deficiency. For example, PNH III cells are completely deficient in GPI-APs, PNH II cells are partially (−90%) deficient, and PNH I cells, which are progeny of residual normal stem cells, express GPI-AP at normal density. Classic PNH is characterized by a large population of GPI-AP deficient PMNs, cellular marrow with erythroid hyperplasia and normal or near-normal morphology and frequent or persistent florid macroscopic hemoglobinuria. PNH in the setting of another bone marrow failure is characterized by a relatively small percentage (<30%) of GPI-AP deficient PMNs, evidence of a concomitant bone marrow failure syndrome and intermittent or absent mild to moderate macroscopic hemoglobinuria. Subclinical or latent PNH is characterized by a small (<1%) population of GPI-AP deficient PMNs, evidence of a concomitant bone marrow failure syndrome and no clinical or biochemical evidence of intravascular hemolysis. Complement pathways (including the alternative complement pathway) are implicated in the pathology of PNH, and thus fusion proteins provided herein are thus useful for treating PNH.

The compositions and methods described herein are particularly useful for treatment of aHUS, an extremely rare disease characterized by low levels of circulating red blood cells due to their destruction (hemolytic anemia), low platelet count (thrombocytopenia) due to their consumption and inability of the kidneys to process waste products from the blood and excrete them into the urine (acute kidney failure), a condition known as uremia. Complement pathways (including the alternative complement pathway) are implicated in the pathology of aHUS, and thus fusion proteins provided herein are thus useful for treating aHUS.

The compositions and methods described herein are particularly useful for treatment of dermatomyositis, a group of acquired muscle diseases called inflammatory myopathies which are characterized by chronic muscle inflammation accompanied by muscle weakness. The cardinal symptom is a skin rash that precedes or accompanies progressive muscle weakness. Dermatomyositis may occur at any age, but is most common in adults in their late 40s to early 60s, or children between 5 and 15 years of age. Complement pathways (including the alternative complement pathway) are implicated in the pathology of dermatomyositis, and thus fusion proteins provided herein are thus useful for treating dermatomyositis.

The compositions and methods described herein are particularly useful for treatment of systemic scleroderma. Also called diffuse scleroderma or systemic sclerosis, it is a chronic disease characterized by diffuse fibrosis and vascular abnormalities in the skin, joints, and internal organs (especially the esophagus, lower GI tract, lungs, heart, and kidneys). Common symptoms include Raynaud phenomenon, polyarthralgia, dysphagia, heartburn, and swelling and eventually skin tightening and contractures of the fingers. Complement pathways (including the alternative complement pathway) are implicated in the pathology of systemic scleroderma, and thus fusion proteins provided herein are thus useful for treating systemic scleroderma.

The compositions and methods described herein are particularly useful for treatment of demyelinating polyneuropathy, a neurological disorder characterized by progressive weakness and impaired sensory function in the legs and arms. The disorder, which is sometimes called chronic relapsing polyneuropathy, is caused by damage to the myelin sheath of the peripheral nerves. Complement pathways (including the alternative complement pathway) are implicated in the pathology of demyelinating polyneuropathy, and thus fusion proteins provided herein are thus useful for treating demyelinating polyneuropathy

The compositions and methods described herein are particularly useful for treatment of pemphigus, a group of rare autoimmune skin disorders that cause blisters and sores on the skin or mucous membranes, such as in the mouth or on the genitals. Complement pathways (including the alternative complement pathway) are implicated in the pathology of pemphigus, and thus fusion proteins provided herein are thus useful for treating pemphigus.

The methods described herein are particularly useful for treatment of thrombotic thrombocytopenic purpura (TTP). TTP features numerous microscopic clots, or thromboses, in small blood vessels throughout the body. Red blood cells are subjected to shear stress that damages their membranes, leading to intravascular hemolysis. The resulting reduced blood flow and endothelial injury results in organ damage, including brain, heart, and kidneys. TTP is clinically characterized by thrombocytopenia, microangiopathic hemolytic anemia, neurological changes, renal failure, and fever. TTP is caused by autoimmune or hereditary dysfunctions that activate the coagulation cascade or the complement system (George, J., N. Engl. J. Med., 354:1927-35, 2006). TTP may arise from genetic or acquired inhibition of the enzyme ADAMTS13, a metalloprotease responsible for cleaving large multimers of von Willebrand factor (vWF) into smaller units, ADAMTS13 inhibition or deficiency ultimately results in increased coagulation (Tsai, H., J. Am. Soc. Nephrol., 14:1072-81, 2003). Patients suffering from TTP typically present in the emergency room with one or more of the following; purpura, renal failure, low platelets, anemia, and/or thrombosis, including stroke. Thrombocytopenia can be diagnosed by a medical professional as one or more of: (i) a platelet count that is less than 150,000/mm³ (e.g., less than 60,000/mm³); (ii) a reduction in platelet survival time, reflecting enhanced platelet disruption in the circulation; and (iii) giant platelets observed in a peripheral smear, which is consistent with secondary activation of thrombocytopoiesis. Because TTP is a disorder that arises from dysregulation of alternative complement pathway activation, treatment with fusion proteins described herein to inhibit the alternative complement pathway activation may aid in stabilizing and/or correcting the disease.

The compositions and methods described herein are particularly useful for treatment of Membranous nephropathy (MN), a glomerular disease and the most common cause of idiopathic nephrotic syndrome in nondiabetic white adults. If untreated, about one-third of MN patients progress to end stage renal disease over 10 years. The incidence of ESRD due to MN in the United States is about 1.9/million per year. Most cases of PMN (70%) have circulating pathogenic IgG4 autoantibodies to the podocyte membrane antigen PLA2R. Complement components including C3, C4d, and C5b-9 are also commonly present, but not Clq, indicating that the lectin and potentially the alternative pathways of complement activation are involved. Over time, IgG4 and C5b-9 deposition leads to podocyte injury, urine protein excretion and nephrotic syndrome (William G. Couser Primary Membranous Nephropathy Clin J Am Soc Nephrol 12: 983-997, 2017). Mice lacking factor B, an essential component of the alternative pathway of complement activation, did not exhibit C3 and C5b-9 deposition and did not develop albuminurea in a mouse model of MN (Wentian et al., Front Immunol. 9:1433, 2018). Therefore, complement inhibitors that reduce the amount of C3 and C5 convertases deposited in glomerular lesions may be effective treatments for this disease.

The compositions and methods described herein are particularly useful for treatment of focal segmental glomerulosclerosis (FSGS). FSGS is characterized by obliteration of glomerular capillary tufts with increased matrix deposition and scarring (D'Agati V D, Fogo A B, Bruijn J A, Jennette J C Pathologic classification of focal segmental glomerulosclerosis: a working proposal. Am J Kidney Dis. 2004 February; 43(2):368-82.). The incidence of FSGS has increased over the past decades and it is one of the leading causes of nephrotic syndrome in adults (Korbet S M Treatment of primary FSGS in adults. J Am Soc Nephrol. 2012 November; 23(11):1769-76). Spontaneous remission is rare (<5%) and presence of persistent nephrotic syndrome indicates a poor prognosis with 50% of patients progressing to end-stage renal disease (ESRD) 6-8 years after initial diagnosis (Korbet S M Clinical picture and outcome of primary focal segmental glomerulosclerosis Nephrol Dial Transplant. 1999; 14 Suppl 3:68-73). Primary FSGS is responsible for 3.3% of all the cases of end-stage renal disease (ESRD) resulting from primary kidney disease in the United States. The complement system has been shown to be activated in patients with primary FSGS and elevated levels of plasma Ba, indicative of activation of the alternative pathway, correlates with disease severity. Patients with low serum C3 had a significantly higher percentage of interstitial injury. Furthermore, renal survival was found to be significantly higher in patients with normal serum C3 as compared to those with low serum C3. Low serum C3 is indicative of complement activation. Therefore, activation of the complement system may play a crucial role in the pathogenesis and outcome of FSGS (Jian Liu, Jingyuan Xie, Xiaoyan Zhang, Jun Tong, Xu Hao, Hong Ren, Weiming, Wang, & Nan Chen. Serum C3 and Renal Outcome in Patients with Primary Focal Segmental Glomerulosclerosis. Scientific Reports, 2017, 7: 4095). In humans, tubulointerstitial deposition of the complement membrane attack complex (C5b-9) is correlated with interstitial myofibroblast accumulation and proteinurea. In the experimental focal segmental glomerulosclerosis, the intratubular formation of C5b-9 was found to promote peritubular myofibroblast accumulation. Myofibroblasts may act as sentinel inflammatory cells and deposit extracellular matrix. These cells may also constrict kidney tubules leading to atubular glomeruli. By this mechanism, complement activation may contribute to tubulointerstitial injury and fibrosis in FSGS. (Rangan G K, Pippin J W, Couser W G. C5b-9 regulates peritubular myofibroblast accumulation in experimental focal segmental glomerulosclerosis. Kidney Int. 2004; 66:1838-1848). Factor B and factor D-deficient mice have lower proteinuria than WT controls in the adriamycin-induced FSGS model, suggesting that activation of AP has a pathogenic role (Lenderink A M, Liegel K, Ljubanović D, Coleman K E, Gilkeson G S, Holers V M, Thurman J M. The alternative pathway of complement is activated in the glomeruli and tubulointerstitium of mice with adriamycin nephropathy. Am J Physiol Renal Physiol. 2007 August; 293(2):F555-64) (Turnberg D, Lewis M, Moss J, Xu Y, Botto M, Cook H T. Complement activation contributes to both glomerular and tubulointerstitial damage in adriamycin nephropathy in mice. J Immunol. 2006 Sep. 15; 177(6):4094-102. Furthermore, complement factor H deficient mice display higher C3b glomerular deposition and more severe kidney damage than wild-type controls. (Morigi M, Locatelli M, Rota C, Buelli S, Corna D, Rizzo P, Abbate M, Conti D, Perico L, Longaretti L, Benigni A, Zoja C, Remuzzi G A previously unrecognized role of C3a in proteinuric progressive nephropathy. Sci Rep. 2016 Jun. 27; 6( )28445). Therefore, an inhibitor of the alternative pathway of complement activation may have clinical utility in FSGS.

In some embodiments, the method involves treating a subject having systemic lupus erythromatosus by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having lupus nephritis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having membranous nephropathy by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having FSGS by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having bullous pemphigoid by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having epidermolysis bullosa acquisita by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having ANCA vasculitis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having hypocomplementemic urticarial vasculitis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having immune complex small vessel vasculitis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having rheumatoid arthritis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having aPL by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having glomerulonephritis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having PNH syndrome by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having C3G by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having dermatomyositis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having autoimmune necrotizing myopathies by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having systemic sclerosis by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having demyelinating polyneuropathy by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having pemphigus by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having inflammation by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having organ transplantation by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having intestinal and renal I/R injury by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having asthma by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having spontaneous fetal loss by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having DDD by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having IgA nephropathy by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having HUS by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having aHUS by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having macular degeneration by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to anyone of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

In some embodiments, the method involves treating a subject having TTP by administering to the subject a therapeutically effective amount of fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21). In some embodiments, the method involves administering to the subject a therapeutically effective amount of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200.

The disclosure further relates to a composition comprising the fusion proteins, as provided above, for use in treatment of a disease selected from the group consisting of PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, and TTP; preferably, SLE, lupus nephritis, membranous nephropathy, IgA nephropathy, FSGS, pemphigus, bullous pemphigoid, epidermolysis bullosa acquisita, systemic sclerosis, ANCA vasculitis, hypocomplementemic urticarial vasculitis, immune complex small vessel vasculitis, PNH, AHUS, dermatomyositis, and autoimmune necrotizing myopathies.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of SLE. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of SLE.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of lupus nephritis. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of lupus nephritis.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of membranous nephropathy. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of membranous nephropathy.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of IgA nephropathy. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of IgA nephropathy.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of FSGS. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of FSGS.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of Pemphigus. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of Pemphigus.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of bullous pemphigoid. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of bullous pemphigoid.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of epidermolysis bullosa acquisita. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of epidermolysis bullosa acquisita.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of systemic sclerosis. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of systemic sclerosis.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of ANCA vasculitis. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of ANCA vasculitis.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of hypocomplementemic urticarial vasculitis. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of hypocomplementemic urticarial vasculitis.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of immune complex small vessel vasculitis. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of immune complex small vessel vasculitis.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of PNH. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of PNH.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of AHUS. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of AHUS.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of dermatomyositis. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of dermatomyositis.

The disclosure further relates to a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for use in treatment of autoimmune necrotizing myopathies. In some embodiments, the disclosure relates to a composition comprising Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200 for use in treatment of autoimmune necrotizing myopathies.

In some embodiments, the disclosure relates to a pharmaceutical composition for treating PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, or TTP, or preferably, SLE, lupus nephritis, membranous nephropathy, IgA nephropathy, FSGS, pemphigus, bullous pemphigoid, epidermolysis bullosa acquisita, systemic sclerosis, ANCA vasculitis, hypocomplementemic urticarial vasculitis, immune complex small vessel vasculitis, PNH, AHUS, dermatomyositis, and autoimmune necrotizing myopathies, as an active ingredient.

In some embodiments, the disclosure relates to a pharmaceutical composition for treating SLE, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating SLE, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating lupus nephritis, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound AB, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating lupus nephritis, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating membranous nephropathy, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating membranous nephropathy, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating IgA nephropathy, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound AB, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating IgA nephropathy, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating FSGS, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating FSGS, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating Pemphigus, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound AB, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating Pemphigus, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating bullous pemphigoid, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound AB, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating bullous pemphigoid, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating epidermolysis bullosa acquisita, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating epidermolysis bullosa acquisita, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating systemic sclerosis, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound AB, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating systemic sclerosis, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating ANCA vasculitis, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound AB, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating ANCA vasculitis, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating hypocomplementemic urticarial vasculitis, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient.

In some embodiments, the disclosure relates to a pharmaceutical composition for treating hypocomplementemic urticarial vasculitis, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating immune complex small vessel vasculitis, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient.

In some embodiments, the disclosure relates to a pharmaceutical composition for treating immune complex small vessel vasculitis, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating PNH, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating PNH, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating AHUS, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating AHUS, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating dermatomyositis, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound AB, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating dermatomyositis, containing a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to a pharmaceutical composition for treating autoimmune necrotizing myopathies, containing a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21) as an active ingredient. In some embodiments, the disclosure relates to a pharmaceutical composition for treating autoimmune necrotizing myopathies, containing a fusion protein selected from the group consisting of Compound AB (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200).

In some embodiments, the disclosure relates to use of a composition comprising a fusion protein, as provided above, for the manufacture of a medicament for treating a disease selected from the group consisting of PNH, aHUS, IgA nephrology, lupus nephritis, C3G, dermatomyositis, systemic sclerosis, demyelinating polyneuropathy, pemphigus, membranous nephropathy, FSGS, bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, DDD, AMD, SLE, RA, MS, TBI, ischemia reperfusion injury, preeclampsia, and TTP; preferably, SLE, lupus nephritis, membranous nephropathy, IgA nephropathy, FSGS, pemphigus, bullous pemphigoid, epidermolysis bullosa acquisita, systemic sclerosis, ANCA vasculitis, hypocomplementemic urticarial vasculitis, immune complex small vessel vasculitis, PNH, AHUS, dermatomyositis, and autoimmune necrotizing myopathies.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for SLE. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for SLE.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for lupus nephritis. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for lupus nephritis.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for membranous nephropathy. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for membranous nephropathy.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for IgA nephropathy. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for IgA nephropathy.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for FSGS. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for FSGS.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for Pemphigus. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for Pemphigus.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for bullous pemphigoid. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for bullous pemphigoid.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for epidermolysis bullosa acquisita. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for epidermolysis bullosa acquisita.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for systemic sclerosis. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for systemic sclerosis.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for ANCA vasculitis. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for ANCA vasculitis.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for hypocomplementemic urticarial vasculitis. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for hypocomplementemic urticarial vasculitis.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for immune complex small vessel vasculitis. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for immune complex small vessel vasculitis.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for PNH. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for PNH.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for AHUS. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for AHUS.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for dermatomyositis. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for dermatomyositis.

In some embodiments, the disclosure relates to use of a composition comprising a fusion protein selected from the group consisting of Compound A, Compound B, Compound C, Compound D, Compound E, Compound F, Compound G, Compound H, Compound I, Compound M, Compound N, Compound O, Compound P, Compound Q, Compound R, Compound S, Compound T, Compound U, Compound X, Compound Y, Compound Z, Compound A B, Compound AC, Compound AG, Compound AH, Compound AI, Compound AJ, Compound AR, Compound AS, Compound AT, Compound AU, Compound AV, Compound AW, and Compound AX, (e.g., a fusion protein having the amino acid sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222), or a variant thereof (e.g., a fusion protein having at least 85% sequence identity to any one of SEQ ID NOs: 114-132, 144, 145, 147, 148, 152-155, and 209-21), for the manufacture of a medicament for autoimmune necrotizing myopathies. In some embodiments, the disclosure relates to use of a composition comprising a fusion protein selected from the group consisting of Compound A B (SEQ ID NO: 147), Compound AC (SEQ ID NO: 148), or Compound AJ (SEQ ID NO: 155), or a variant thereof (e.g., a fusion protein having at least 85% sequence identity to any one of SEQ ID NOs: 147, 148, or 155), or a fusion protein encoded by any one of SEQ ID NOs: 194, 195, or 200)), for the manufacture of a medicament for autoimmune necrotizing myopathies.

### EXAMPLES

The following examples are put forth so as to provide those of ordinary skill in the art with a disclosure and description of how the methods and compounds claimed herein are performed, made. They are intended to be purely exemplary and are not intended to limit the scope of the disclosure.

### Example 1. In Silico Design and Construction of the Factor H Fc Fusion Proteins

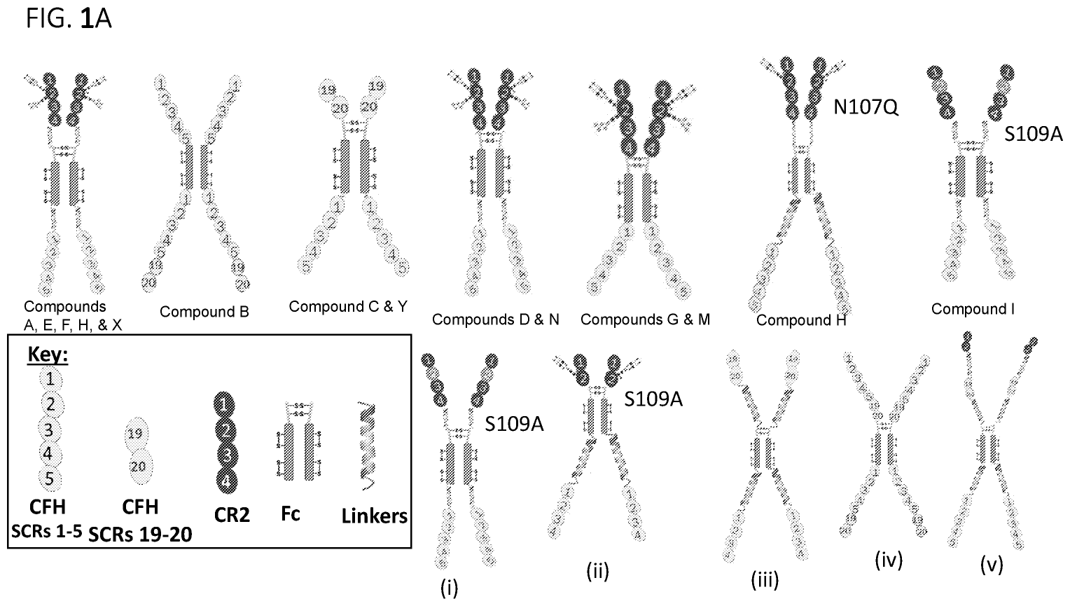

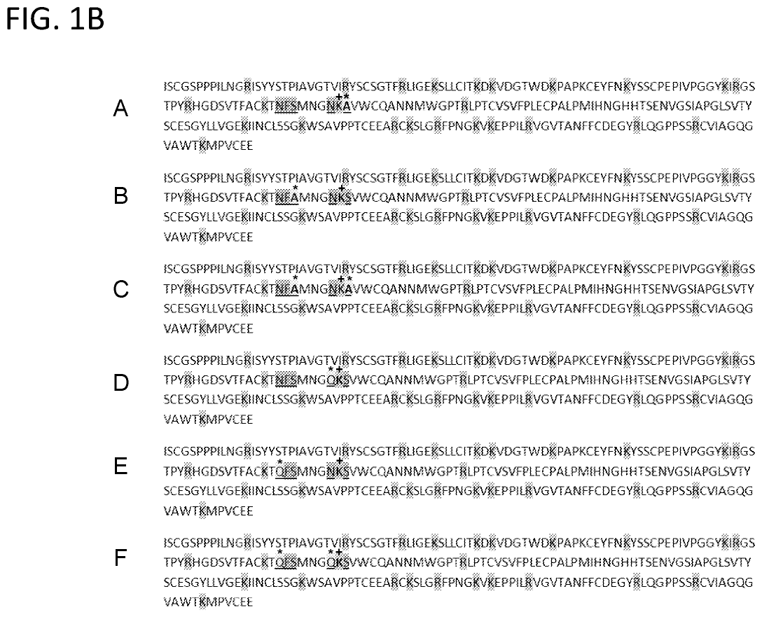

Constructs including various combinations of SCR domains of FH, SCR domains of CR2, Fc domains, such as Fc receptor binding domains, were designed in silico. Exemplary constructs are illustrated in FIG. 1A.

CR2 SCR domains 1-4 inhibit auto-antibodies, bind to C3b/C3d, and are useful for increasing the B cell activation threshold. FH SCR domains 1-5 bind to C3b and can inhibit the alternative complement pathway (AP). FH SCR domains 19-20 can interact with the negatively-charged extracellular matrix components on host cell surfaces, and can bind to C3b. The Fc domain allows for prolonged stability and pharmacokinetics properties.

In one example, the amino acid sequence of human complement receptor 2 (CR2) (Genbank accession number NP_001006659.1) encompassing short consensus repeats (SCRs) 1-4 was added to the N-terminus of the human IgG2/IgG4 hybrid heavy chain constant region at position 4 of the hinge region. The amino acid sequence of human complement factor H (Genbank accession number NP_000177.2) SCRs 1-5 was added to the C-terminus of the hybrid human IgG2/IgG4 heavy chain constant region.

Some variants were constructed with peptide linkers having the sequence (G₄S)₄, (G₄A)₂G₄S, G₄SDA, or G₄SDAA inserted between the CR2 region and the Fc region. Additional variants had (G₄S)₄, (G₄A)₃G₄S, or (G₄A)₂G₄S linker sequences inserted between the IgG region and the human complement factor H region. Some variants had linkers in both positions.

Certain variants were designed with one of the N-linked glycosylation sites of CR2 eliminated by introducing either an N107Q or S109A mutation (amino acid residue numbering according to mature CR2, excluding the 20 amino acid signal peptide) (FIG. 1B). This glycosylation site is known to be variably occupied by heterogeneous high mannose glycans in a fusion protein comprising the first four SCR domains of factor H and the first 4 domains of CR2 in the absence of an Fc domain (CR2₁₋₄FH₁₋₅).

The amino acid sequences of the constructs shown in FIG. 1A were provided to GeneArt (ThermoFisher) for codon optimization and gene synthesis. Nucleotide sequences encoding the polypeptides of the compounds shown in Table 1 were cloned into an expression vector for production in mammalian cells. Plasmid DNA was then transiently transfected into human HEK293 cells. After 4-5 days, supernatants were harvested. The concentration of fusion proteins were determined by SDS-PAGE and densitometry. Fusion proteins were purified by Protein A chromatography. The concentrations of purified fusion proteins were determined by UV spectroscopy absorbance at 280 nm corrected for molar extinction coefficient. Purity was assessed by SDS-PAGE and size-exclusion HPLC.

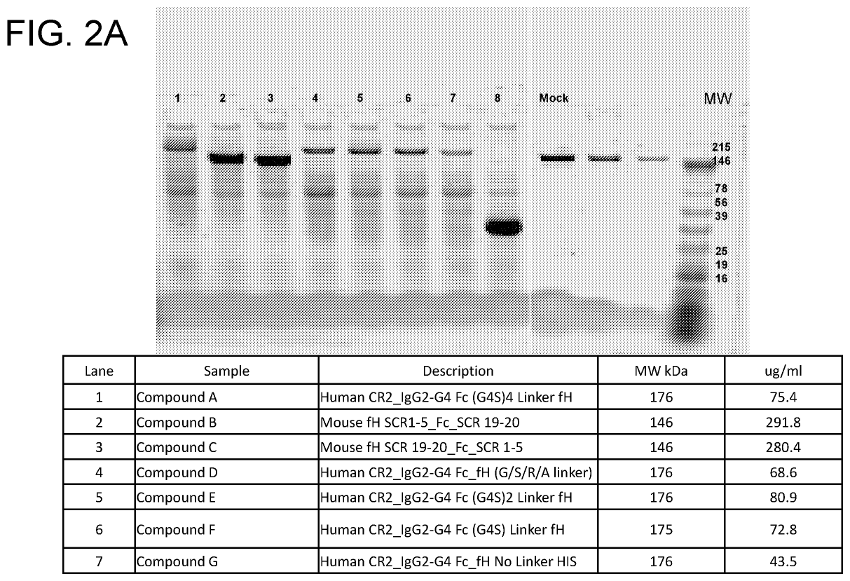

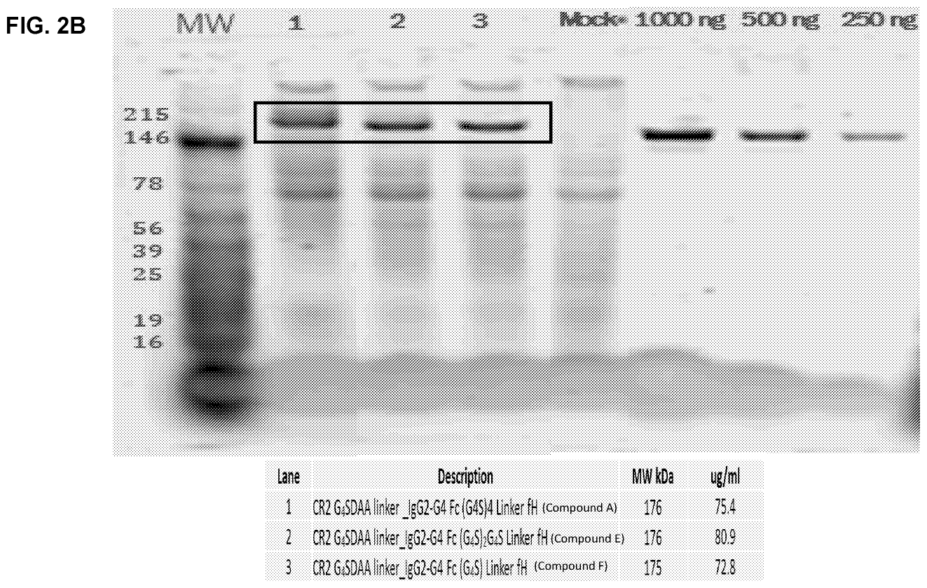

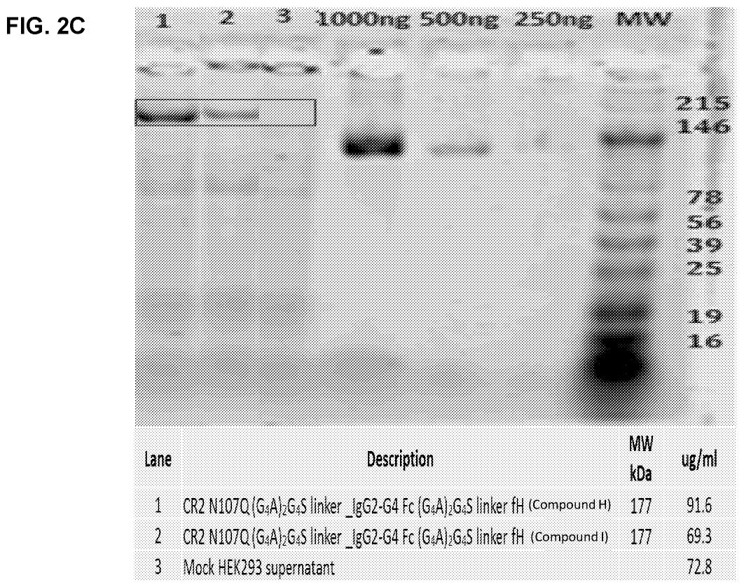

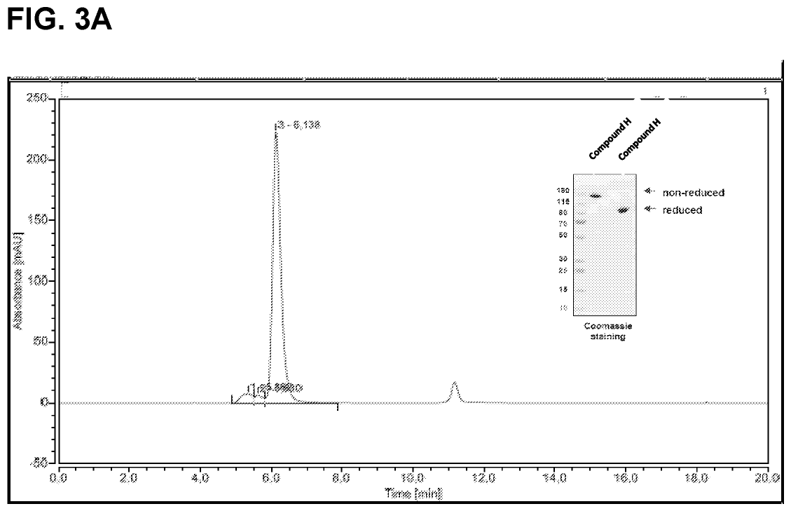

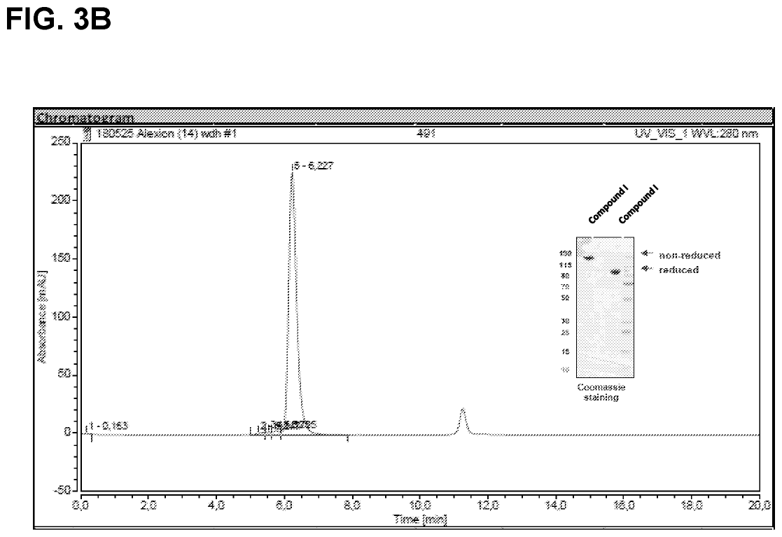

CR2-FH-Fc fusion proteins expressed well in transiently transfected HEK293 cells. Exemplary SDS PAGE gels of harvested cell culture supernatants are shown in FIGS. 2A-2C. These fusion proteins were readily purified by Protein A chromatography to high levels of purity (See FIGS. 3A-3B). In addition, the N-linked glycosylation site at position 107 of CR2 SCR2 can be removed without compromising expression levels, however the N107Q variant appeared to be more prone to aggregation than the S109A variant (FIG. 2C).

### Example 2. Functional Evaluation of Factor H Fusion Proteins

Fusion proteins were tested for their ability to inhibit the alternative pathway using the AP-specific hemolytic assay. Briefly, rabbit red blood cells were washed and added to 10% human serum containing Mg² and EGTA. Serial dilutions of inhibitors were added and the cells were incubated for 30 min at 37′C. Cells were removed by centrifugation and the amount of cell lysis was determined by measuring the absorbance of the supernatant at 415 nm.

Factor H fusion proteins including an Fc domain and a fragment of CR2 were at least 4 times more potent than CR2₁₋₄FH₁₋₅ in the AP hemolytic assay (FIGS. 4A and 4B). CR2 increased the potency when incorporated into a fusion protein containing factor H SCRs 1-4 or 1-5. CR2 alone had no effect on AP hemolysis (FIG. 4A). Fusion proteins containing FH SCRs 19-20 in addition to FH SCRs 1-4 appeared to be equipotent to fusion proteins containing factor H and CR2 (FIG. 4C). CR2 SCRs 3-4 and FH SCR 5 can be excluded from the fusion proteins without a loss of potency (FIG. 40).

### Example 3. In Silicao Design, Production, and Functional Evaluation of Factor H Anti-Abumin-VHH Fusion Proteins

A variety of constructs including the first 5 N-terminal SCR domains of FH and/or the first four N-terminal SCR domains of CR2, and anti-human serum albumin (α-HSA) V_{HH} were designed in silico, and is illustrated in FIG. 5A. FH SCR domains 1-5 bind to C3b and can inhibit the alternative complement pathway (AP). CR2 SCR domains 1-4 inhibit auto-antibodies, bind to C3b/C3d, and are useful for increasing the B cell activation threshold. The α-HSA-V_{HH} allows for prolonged stability and pharmacokinetics properties. Expression was accomplished similarly to Example 1.

The FH₁₋₅-α-HSA-V_{HH} and CR2₁₋₄-α-HSA-VHH-FH₁₋₅ fusion proteins were purified from cell supernatant using MEP HYPERCELm or CAPTO™ Adhere ImpRes resin at a variety of pH conditions. The yield and purity from these purification conditions are shown in FIGS. 5B-5G.

Fusion proteins were tested for inhibition of the alternative pathway using the AP-specific hemolytic assay. Briefly, rabbit red blood cells were washed and added to 10% human serum containing Mg²⁺ and EGTA. Serial dilutions of inhibitors were added and the cells were incubated for 30 min at 37′C. Cells were removed by centrifugation and the amount of cell lysis was determined by measuring the absorbance of the supernatant at 415 nm.

All fractions purified using MEP HYPERCEL™ or CAPTO™ Adhere ImpRes resin at a variety of pH conditions retained similar inhibition activity (FIGS. 5H and 5I).

HiTrap CAPTO™ Adhere ImpRes was used for a large scale purification. The final product eluted at pH 4.5 and was isolated to 99% purity (FIG. 5J).

### Example 4. Optimization and Structure-Function Analysis of Factor H Fc Fusion Proteins

Compound X (SEQ ID NO: 132) was designed (FIG. 6A), expressed transiently in CHO cells, and purified by protein A chromatography, as described above. As indicated by the multiple bands in the reduced and non-reduced SDS-PAGE analysis (FIG. 6B), the fusion protein was determined to be susceptible to fragmentation.

Compound X was then enzymatically de-glycosylated by PNGase F treatment and analyzed by electrospray ionization time-of-flight (ESI-ToF) mass spectrometry. Following deconvolution of the mass spectra, three major species were observed with m/z values corresponding to masses of 177,324.4 Da, 117,598.1 Da, and 59,724.7 Da, corresponding to the intact dimer, a larger fragment formed by a single cleavage occurring in the hinge region of the Fc domain, and a smaller fragment consisting of the Fc, linker and FH domain, respectively. The masses of the fragments indicated that the cleavage had occurred at the junction between the lower hinge and CH2 domain of the Fc region (FIG. 7).

Compound X was then modified in the following manner: (1) shorten the CR2 SCRs to delete SCRs 3-4; (2) change the linker from (G₄A)₂(G₄S) to GGGGSDAA; (3) modify the FH to exclude SCR5 (i.e., use SCR1-4 vs. SCR1-5); and (4) other modifications such as C-terminal modification of SCR4 to add Serine (S); and (5) further optional modification to substitute N107Q (FIG. 8A). The resultant fusion protein (Compound AC), was assessed by SDS PAGE. Human CR2 contains two consensus N-linked glycosylation sites at positions 101 and 107. Analysis of Compound K, which consists of CR2 SCRs 1-4 directly fused to FH SCRs 1-5, indicated that the N101 glycosylation site is populated by complex type N-linked oligosaccharides while the N107 site is partially occupied with high mannose type glycans. Glycan analysis of Compound X indicated that the N107 glycosylation site was also occupied predominantly with high mannose glycans. Monoclonal antibodies that have high mannose glycans on the Fc region exhibit faster clearance rates than those that have Fc regions with complex glycans. Therefore, the N107 glycosylation site of the CR2 domain of certain compounds was eliminated by introducing a N107Q mutation. CR2 produced in E. coli cells, which do not add N-linked glycans to proteins, was shown to bind similarly to its ligands as CR2 produced in mammalian cells. Therefore, the N107Q substitution was not expected to negatively impact the binding properties of the CR2 domain.

As shown in FIG. 8B, these modifications improved the resistance to cleavage of this compound. Compound AC was further assessed by ESI ToF mass spectrometry. As indicated by the de-convoluted mass spectra, no fragmented species were detected (FIG. 5C).

The contribution of the targeting domain (CR2) to in vitro potency was then investigated by comparing Compound AC to Compound AD, a variant that does not contain a CR2 targeting domain. Compound AD contains the hinge, CH2, and CH3 regions of a human IgG1 Fc region fused via a flexible linker to FH SCRs 1-5 at the C-terminus. Both compounds were tested for inhibition of the human complement alternative pathway in a rabbit red blood cell hemolysis assay. Briefly, rabbit red blood cells were incubated with titrations of both inhibitors for 30 minutes in 10% complement preserved human serum supplemented with 10 mM EGTA and 2 mM MgCl² in gelatin veronal buffer (GVB). These conditions allow for the activation of the complement alternative pathway but not the complement classical pathway. Red blood cell lysis was monitored by measuring the release of hemoglobin at 415 nM. In this experiment, Compound AC was found to have an IC50 of 11.4 nM, while Compound AD was found to have an IC50 of 37 nM. FIG. 9 provides the dose response curves for the inhibition of human alternative pathway-mediated hemolysis for these compounds. The inclusion of the CR2 targeting domain was found to improve the in vitro potency by 3.2 fold.

SCRs 19 and 20 of complement factor H function to localize the molecule to cellular surfaces and extracellular matrix. Factor H SCRs 19-20 were therefore included in certain compounds as targeting domains in place of CR2. Additionally, the position of the targeting domains and factor H domains at the N- or C-terminus was investigated by generating variants containing these domains at either termini of a human Fc region. As a control, compounds with no targeting domain were included and the complement regulatory domains of FH were fused to either the N- or C-terminus of a human Fc region. These compounds were tested for inhibition of the human complement alternative pathway in a rabbit red blood cell hemolysis assay. Here, rabbit red blood cells were incubated with titrations of both inhibitors for 30 minutes in 10% complement preserved human serum supplemented with 10 mM EGTA and 2 mM MgCl², buffer conditions in which the alternative pathway but not the classical pathway of complement may be activated. Red blood cell lysis was monitored by measuring the release of hemoglobin at 415 nM. FIG. 10 provides the titration inhibitory curves and IC50 values for these molecules.

The in vitro potency of factor H-Fc fusions without targeting domains was determined by testing serial dilutions of these compounds in the human alternative pathway complement hemolytic assay. FIG. 11 provides the dose-response curves for compounds Compound AD, Compound AE, and Compound AF. As shown in the dose response curve, non-targeted compounds in which the FH domain is attached to the C-terminus of the Fc region are active in this assay (Compound AD and Compound AE) while Compound AF having the FH domain attached to the N-terminus of the Fc region was not active at the concentrations tested.

### Example 5. Factor H Fusion Protein C3d Interaction Study

Purified C3d (Quidel, San Diego, Calif.) was biotinylated via sulfo-NHS-LC linkage (ThermoFisher, Waltham, Mass.) and immobilized to streptavidin-coated biosensors at 1 ug/ml on an Octet Red bio-layer interferometry detector (ForteBio, San Jose, Calif.) for 600s. Biosensors were then rinsed in buffer for 60s, followed by incubation in Compound AC, Compound AP, or Compound AQ at 2 uM for 600s. This association measurement phase was followed by a dissociation phase measurement in buffer alone for 1200s. Data and binding kinetics measurements are shown in FIG. 12. Both Compound AC and Compound AQ, which contain the CR2 SCR1-2 domain and the FH domain, bind to C3d, while Compound AP, which has the FH domain but lacks the CR2 domain, does not associate with C3d.

### Example 6. In Vivo Pharmacodynamics and Pharmacokinetics Evaluation of Factor H Fusion Proteins

A single dose of a factor H fusion protein (e.g., a CR2-FH-Fc fusion protein, a FH₁₉₋₂₀-Fc-FH₁₋₅ fusion protein; a fusion protein having the sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222) can be administered to a mouse model of complement activity (e.g., C47BL/6J male mice) to test the pharmacokinetic properties of the fusion protein. Plasma samples can be collected at various time points following administration.

Pharmacokinetic properties of the factor H fusion proteins can be assessed by testing the plasma samples using an enzyme-linked immunosorbent assay (ELISA). Alternative pathway (AP) hemolytic activity can be monitored in the collected plasma samples using methods known in the art.

The effects of the fusion protein in the mouse model can be compared to effects with an isotype-matched control antibody, and can be measured as a function of dose and exposure. Sustained inhibition of plasma complement alternative pathway hemolytic activity is indicative of fusion protein efficacy and sustained bioavailability.

In one example, the pharmacokinetics (PK) and pharmacodynamics (PD) of compounds described herein were evaluated in single dose studies in wild-type C57 black 6 (C57BL/6) mice. In this experiment, compounds in which the potential for fragmentation was retained or limited and the second N-linked glycosylation site was retained or eliminated were evaluated. Compound X was selected because it was found to be susceptible to fragmentation and it has both N-linked glycosylation sites present in the CR2 domain. Compound H was selected because it has the N107Q mutation which eliminated the second N-linked glycosylation site of CR2. However, Compound H contains a longer (G₄A)₂G₄S linker between the CR2 domain and the Fc region and thus is susceptible to fragmentation. FIG. 13 provides the SDS-PAGE analysis of Compound H expressed in CHO cells and purified by protein A chromatography. Fragmentation is evident by the presence of multiple bands on the reduced and non-reduced SDS-PAGE.

Compound AC was also evaluated for PK and PD effects in wild-type mice as it contains the shorter linker between the CR2 domain and the Fc and thus has minimal fragmentation. Compound AC also has the N107Q mutation that eliminates the second N-linked glycosylation site of CR2.

Male C57Bl/6 mice were administered single 25 mg/kg IV doses of either Compound X, Compound H, or Compound AC. Blood samples were taken at 30 minutes, 1 day, 2 days, 4 days, 5 days, and 7 days after dosing. The serum concentrations of the compounds were determined using an immuno-assay in which the compounds were captured using either an anti-human CR2 monoclonal antibody (clone 1148) or an anti-human IgG polyclonal antibody (Jackson ImmunoResearch, catalog number 109-065-088). The compounds were detected using an anti-human factor H antibody (Quidel, catalog number A254). Similar results were obtained when either the anti-CR2 or the anti-human IgG antibody was used to capture the compounds. FIG. 14 provides the PK data. Compound X, being susceptible to fragmentation and having the second-N-linked glycosylation site present in CR2, had the poorest PK. Compound H, which was susceptible to fragmentation but does not contain the second N-linked glycosylation site had better PK, and compound AC, having no fragmentation and the second N-linked glycosylation site of CR2 eliminated had the most favorable PK.

In vivo PD was evaluated using the mouse alternative pathway hemolytic assay. Briefly, serum from treated animals was added to washed rabbit red blood cells that were re-suspended in GVB buffer containing 1.2 mM MgCl2+ and 6.2 mM EGTA. These buffer conditions prevent the activation of the classical pathway but allow for the activation of the alternative pathway of complement. FIG. 15 provides the percent inhibition of mouse alternative pathway mediated lysis of rabbit red blood cells over time in animals treated with Compound X, Compound H, or Compound AC. Inhibition of alternative pathway hemolysis correlated with the PK data and Compound AC provided the most complete inhibition of alternative pathway hemolysis.

The effect of removing SCR5 from the FH domain was further investigated in wild-type mice. Here, C57BL/6 mice were administered a single 25 mg/kg IV dose of Compound A B. Compound A B is identical to Compound AC except for the inclusion of SCR5 in the FH domain. FIG. 16 provides the PK and PD data for Compound A B and FIG. 17 provides the PK and PD data of Compound AC. Note that the PD data are expressed as percent lysis or the remaining hemolytic activity present in the serum of treated animals. A single dose of Compound AC was found to suppress alternative pathway hemolysis more effectively than Compound A B.

### Example 7. Efficacy and Pharmacodynamcs of Factor H Fusion Proteins in a Mouse Model of C3 Glomerulopathy

A single dose of a factor H fusion protein (e.g., a CR2-FH-Fc fusion protein, a FH₁₉₋₂₀-Fc-FH₁₋₅ fusion protein; a fusion protein having the sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222) can be administered to factor H deficient mice, and plasma samples can be collected at various time points following administration.

Pharmacokinetic and pharmacodynamic properties of the factor H fusion proteins can be assessed by testing the plasma samples using an ELISA. C3 and factor B levels can be assessed by ELISA and/or western blot. Glomeruli C3 deposition can be examined by immunohistochemistry (IHC).

Normalization and/or restoration of plasma levels of complement components, such as C3 and factor B, to levels observed in factor H sufficient littermates, elimination of glomerular C3 deposits, and/or sustained prevention of glomerular C3 deposition can be indicative of fusion protein efficacy and prolonged bioavailability.

In one example, in vivo mechanistic studies were performed by administering Compound AC to factor H deficient C57BL/6 mice. Both alleles encoding complement factor H are inactivated in this strain using CRISPR technology. These mice exhibit uncontrolled AP activation of complement resulting in depletion of plasma C3 and C5 and deposition of C3 fragments and properdin along the glomerular basement membrane in kidneys. Factor H deficient mice have been shown to develop membranoproliferative glomerulonephritis and are predisposed to developing renal injury caused by immune complexes. In this experiment, a single 25 mg/kg IV dose of Compound AC was administered to FH−/− mice on day 0. Serum was sampled on days 1, 3, 7, 10, and 14 for PK and to measure levels of complement C3 and C5. PK was determined by an immunoassay in which Compound AC was captured using a polyclonal anti-human IgG antibody and detected with an anti-human FH antibody. Plasma levels of complement C3 were determined by an immunoassay using the Gyros xPlore system (Gyros Protein Technologies, Uppsala, Sweden). Mouse C3 was captured using a biotinylated rat monoclonal anti-C3 antibody, clone 11H9 (Novus Biologicals catalog number NB200-5408) and detected with Alexa Fluor 647 labeled goat anti-mouse C3 polyclonal antibody (MP Biomedicals catalog number 55463). Mouse C3 (Complement Technologies catalog number M113) was used as a standard. Plasma C5 levels were determined by ELISA using anti-mouse C5 monoclonal antibody BB5.1 (Alexion Pharmaceuticals, Inc,) and detected with Alexa Fluor-647 labeled anti-mouse C5 monoclonal antibody ATM587 (Alexion Pharmaceuticals, Inc,). Recombinant mouse C5 was used as a standard.

Groups of animals were euthanized on days 1, 3, 7 and 14. Kidneys removed and sectioned for immunohistochemistry. Compound AC was detected in the kidneys of treated animals using a goat polyclonal anti-human factor H monoclonal antibody (Quidel catalog number A312), which was detected with an Alexa Fluor-488 labeled rabbit anti-goat IgG polyclonal antibody (Life Technologies A11080). Glomerular deposition of mouse properdin was detected by staining kidney sections with Alexa Fluor-647 labeled anti-mouse properdin monoclonal antibody 14E1. Glomerular deposition of complement component C3 was determined using a FITC-conjugated goat anti-mouse C3 polyclonal antibody (MP Biomedical catalog number 55500).

The PK profile of Compound AC was different when administered to FH−/− mice as compared to wild-type mice. In FH−/− mice, plasma levels of Compound AC decreased more rapidly, presumably due to the localization of Compound AC to tissues such as the kidney glomeruli where C3 deposition had occurred. FIG. 18 provides the PK profile form wild-type and FH−/− mice administered a single 25 mg/kg IV dose of Compound AC.

Compound AC was found to localize to the kidneys of FH−/− mice. Fluorescence detection of Compound AC was statistically significant at the day 1 and day 3 time-point. FIG. 19 provides the IHC of human factor H (Compound AC) on the glomerular basement membrane of FH−/− mice administered a single 25 mg/kg IV dose. FIG. 20 provides the mean fluorescence intensity and statistical analysis for the localization of Compound AC.

Complement C3 forms deposits along the glomerular basement membrane in the kidneys of FH−/− mice. A single 25 mg/kg dose of Compound AC dramatically reduced C3 deposition by day 1 post dosing and remained significantly reduced for 7 days (FIGS. 21 and 22).

Similar to complement C3, properdin is also deposited along the glomerular basement membrane of FH−/− mice. Animals treated with Compound AC showed dramatically reduced properdin deposition from day 1 post dosing through the end of the experiment at day 14 (FIG. 23).

Administration of a single dose of Compound AC to FH−/− mice resulted in a partial restoration of plasma C3 levels at one day post-dose. The average C3 plasma concentration is approximately 420 μg/mL (data not shown). At day 1 after dosing, plasma C3 levels had increased to an average of 215 μg/mL. However, plasma C3 levels had returned to baseline by day 3 after dosing (FIG. 24).

Interestingly, plasma C5 levels were significantly elevated to near wild-type levels for 14 days post administration of Compound AC to FH−/− mice. C5 is predominantly cleaved by surface phase C5 convertases. When administered to FH−/− mice, Compound AC effectively disrupted the properdin-containing C3/C5 convertases that had formed at the glomeruli resulting in the prolonged stabilization of plasma C5 levels. FIG. 25 provides the plasma C5 levels of FH−/− mice treated with Compound AC. Plasma C5 levels of normal mouse serum (NMS) at day zero and PBS-treated control FH−/− mice at day 10 and day 14 are also shown. C5 levels were significantly elevated from day 1 to day 14 when compared to the day 10 PBS control group using Dunnett's test for multiple comparisons.

### Example 8. Efficacy of Factor H Fusion Proteins in a Mouse Model of Lupus Nephritis

A weekly dose of either a factor H fusion protein (e.g., a CR2-FH-Fc fusion protein, a FH₁₉₋₂₀-Fc-FH₁₋₅fusion protein; a fusion protein having the sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222) or a placebo can be administered to a mouse model of inflammatory glomerular nephritis (e.g., MRL/MpJ-Fas^{lpr} mice) to test the efficacy of the fusion protein. Plasma and urine samples can be collected at various time points following administration.

C3 and factor B levels can be assessed by ELISA and/or western blot. Glomeruli C3, IgG, and C1q deposition can be examined by immunohistochemistry (IHC). Levels of anti-dsDNA autoantibodies and/or immune complexes can be assessed by ELISA. Proteinuria and biological urea nitrogen (BUN) levels can be assessed according to routine methods known in the art.

The reduction and/or prevention of glomerular C3 deposition, normalization of plasma C3 and factor B levels, reduction and/or prevention of glomerular IgG and C1q deposition, reduction in circulating anti-dsDNA autoantibodies and/or immune complexes, and/or restoration of kidney function as indicated by amelioration of proteinuria and normalization of BUN can be indicative of fusion protein efficacy in this model.

### Example 9. Efficacy of Factor H Fusion Proteins in a Collagen-Induced Arthritis Mouse Model

C57BL/6J and DBA la1/mice can be immunized with bovine collagen type II with Freund's incomplete/M. tuberculosis adjuvant to trigger collagen-induced arthritis. A booster injection can be administered after three weeks.

Clinical disease activity can be determined by gross examination of the mice; the extent of inflammation, joint ankylosis, and loss of function can be used to generate a clinical disease activity score 35 days post collagen immunization booster.

A factor H fusion proteins (e.g., a CR2-FH-Fc fusion protein, a FH₁₉₋₂₀-Fc-FH₁₋₅ fusion protein; a fusion protein having the sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222) can be administered prophylactically, or immediately following, the second administration of bovine collagen II, with weekly administrations thereafter.

The efficacy of the factor H fusion protein therapy can be assessed by monitoring changes in clinical disease activity, examination of complement activation, and monitoring of anti-collagen antibody titers. Clinical disease activity (e.g., inflammation, joint ankylosis, and loss of function) can be assessed by gross examination. Complement activation and/or complement-mediated inflammation in the joints can be assessed by quantifying C3 deposition in knee joint, ankle, and paw by IHC, and histopathological changes including inflammation, pannus, and cartilage and bone damage. The levels of anti-collagen antibodies can be quantified by ELISA performed on plasma samples. A reduction in clinical disease activity, as determined by gross examination, prevention of complement activation and/or inflammation in the joints (e.g., prevention of C3 deposition in the knee joint, ankle, and/or paw), prevention of histological changes (e.g., inflammation, pannus, and/or cartilage and bone damage), and/or a reduction in the formation of anti-collagen antibodies in plasma can be indicative of therapeutic efficacy of the fusion protein in this model.

### Example 10. Suppression of B-Cell Activation and Antibody Formation in the Mouse KLH Immunization Model

Complement receptor 2 (CD21) is expressed on mature B-lymphocytes, T cells and follicular dendritic cells. The binding of CR2 on mature B-cells to C3d-opsonized antigens stabilizes a signaling complex composed of CR2, CD81, Leu-13 and CD19. This complex amplifies the signal transmitted by the B-cell receptor upon binding to its specific antigen. In this way, the binding of CR2 to C3d-opsonized antigens reduces the threshold of antigen required for B-cell activation and antibody formation, expressed on B-cells may facilitate the internalization of C3d-obsonized antigens, which may then be presented by B-cells on HLA/MHC class II molecules. A fusion protein consisting of SCRs 1-2 of CR2 fused to the N-terminus of the heavy chain of an antibody has been previously shown to suppress the antibody response in mice immunized with keyhole limpet hemocyanin (KLH).

Factor H deficient mice have enhanced B-cell receptor activation, germinal center hyperactivity and increased double-stranded autoantibodies, caused by increased exposure of splenic B-cells to activated C3 fragments. Therefore, administration of factor H may reduce B-cell activation and autoantibody formation by inhibiting alternative pathway C3 convertases. Additionally, the pathology of certain diseases such as membranous nephropathy, IgA nephropathy, lupus, epidermolysis bullosa acquisita, dermatomyositis, and others involve the formation of autoantibodies that bind to self-structures, form immune complexes and activate complement. The alternative pathway can further contribute to tissue damage by amplifying complement activation. Therefore, a therapeutic that can reduce alternative complement pathway activation and limit the complement-mediated stimulation of autoreactive B-cells may be effective in these diseases.

Compounds were evaluated for suppression of B-cell activation and antibody formation in the mouse KLH immunization model. Briefly, female C57BL/6 mice in groups of five were immunized with 0.5 mg KLH in 0.2 mL PBS by intraperitoneal injection (I.P.). On the day of immunization, mice were administered a single, 25 mg/kg I.P. dose of compounds AA and AJ. As a positive control for inhibition of B-cell activation, one group of immunized mice received a 50 mg/kg dose of cyclophosphamide on the day of immunization and a second dose seven days later. Cyclophosphamide has been shown to reduce autoantibody formation in patients with lupus nephritis. One group of animals was immunized with KLH alone. As a negative control, one group of animals was sham-immunized with PBS. Serum samples were collected before immunization, 1 hour after immunization/dosing, on day 7 and on day 14. KLH specific IgM (early antibody response) and IgG (later response following class switching and affinity maturation) levels were determined by ELISA using KLH as the capture reagent. KLH immune serum from non-treated KLH immunized mice was used as a positive control in the ELISA. The statistical significance of antibody titers in treatment groups compared to the non-treated KLH immunized controls was determined using the Student's T-test. FIG. 26 provides the anti-KLH IgM data and FIG. 27 provides the anti-KLH IgG data. Statistically significant reductions in anti-KLH IgM titers compared to non-treated, immunized controls were observed for Compounds AA and AJ and cyclophosphamide. The degree of suppression of the specific IgM response for these compounds was similar to that observed in the cyclophosphamide treated, immunized controls.

### Example 11. Treatment of Diseases Associated with Alternative Complement Pathway Dysregulation

A subject diagnosed as having a disease associated with alternative complement pathway dysregulation (e.g., kidney disorders, cutaneous disorders, and neurological disorders, such as PNH, aHUS, IgA nephropathy, lupus nephritis, C3G, dermatomyositis, systemic sclerosis, demyelinating polyneuropathy, pemphigus, membranous nephropathy, focal segmental glomerular sclerosis (FSGS), bullous pemphigoid, epidermolysis bullosa acquisita, ANCA vasculitis, hypocomplementemic urticarial vasculitis, immune complex small vessel vasculitis, autoimmune necrotizing myopathies, DDD, AMD, or TTP) can be treated with a fusion protein containing a fragment of factor H and an Fc domain, or a fragment of factor H, a fragment of CR2, and an Fc domain (e.g., a fusion protein having the sequence of any one of SEQ ID NOs: 114-132, 144, 145, 147, 148, 152-155, and 209-215; or a fusion protein encoded by the nucleic acid sequence of any one of SEQ ID NOs: 165-173, 177-185, 188-190, 192, 193, 197-200, and 216-222). The fusion protein can be administered at an effective dose to treat the subject diagnosed with disease associated with alternative complement pathway dysregulation (e.g., kidney disorders, cutaneous disorders, and neurological disorders, such as PNH, aHUS, IgA nephropathy, lupus nephritis, C3G, dermatomyositis, systemic sclerosis, demyelinating polyneuropathy, pemphigus, membranous nephropathy, FSGS, bullous pemphigoid, epidermolysis bullosa acquisita, ANCA vasculitis, hypocomplementemic urticarial vasculitis, immune complex small vessel vasculitis, DDD, AMD, or TTP). When effectively treated, the subject shows normal levels of biomarkers of dense deposit disease (e.g., urinary protein, serum creatinine, plasma C5b-9 for dense deposit disease, or e.g., urinary protein, 51Cr-EDTA renal clearance, plasma C5b-9 for C3 glomerulonephritis) following treatment.

The subject can be diagnosed prior to treatment by a variety of diagnostic methods known in the art. For example, a subject can be diagnosed as having dense deposit disease from electron microscopy analysis of biopsied tissue. A subject may exhibit plasma complement C3 lower than the normal range found in a healthy individual. The subject may exhibit nephrotic-range proteinuria, presented as elevated urinary protein excretion during a 24 hour time period. The subject may show elevated C3 nephritic factor, an autoantibody that stabilizes the alternative pathway C3 convertase activity. Genetic screening of the subject may reveal a tyrosine-402-histidine (Y402H) of factor H, or other mutation in a regulator of the alternative complement pathway that is associated with dense-deposit disease. A low level of plasma C5, combined with a high level of the terminal complement complex sC5b-9 and C5b-9 glomerular deposits can indicate abnormally high levels of alternative complement pathway activation.

In another example a subject may be diagnosed with C3 glomerulonephritis by a renal biopsy. The renal biopsy of a subject may demonstrate expansion of the mesangial matrix and increased glomerular cellularity, segmental capillary wall thickening and focal tubular atrophy. Electron microscopy may show sub-endothelial and mesangial electron dense deposits with infrequent sub-epithelial deposits. The biopsy may show positive staining for complement C3. The subject may exhibit proteinuria and renal impairment. The subject may have a family history of renal disease

### OTHER EMBODIMENTS

All publications, patents, and patent applications mentioned in this specification are herein incorporated by reference to the same extent as if each independent publication or patent application was specifically and individually indicated to be incorporated by reference. While particular embodiments are herein described one of skill in the art will appreciate that further modifications and embodiments are encompassed including variations, uses or adaptations generally following the principles described herein and including such departures from the present disclosure that come within known or customary practice within the art and may be applied to the essential features hereinbefore set forth, and follows in the scope of the claims.

SEQUENCE APPENDIX
Compound A:
Amino Acid (SEQ ID NO: 114):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGSDAAVECPPCPAPPVAGPSVFLF
PPKPKDTLMISRTPEVTCVVVDVSQEDPEVQFNWY
VDGVEVHNAKTKPREEQFNSTYRVVSVLTVLHQDW
LNGKEYKCKVSNKGLPSSIEKTISKAKGQPREPQV
YTLPPSQEEMTKNQVSLTCLVKGFYPSDIAVEWES
NGQPENNYKTTPPVLDSDGSFFLYSRLTVDKSRWQ
EGNVFSCSVMHEALHNHYTQKSLSLSLGKGGGGSG
GGGSGGGGSGGGGSEDCNELPPRRNTEILTGSWSD
QTYPEGTQAIYKCRPGYRSLGNVIMVCRKGEWVAL
NPLRKCQKRPCGHPGDTPFGTFTLTGGNVFEYGVK
AVYTCNEGYQLLGEINYRECDTDGWTNDIPICEVV
KCLPVTAPENGKIVSSAMEPDREYHFGQAVRFVCN
SGYKIEGDEEMHCSDDGFWSKEKPKCVEISCKSPD
VINGSPISQKIIYKENERFQYKCNMGYEYSERGDA
VCTESGWRPLPSCEEKSCDNPYIPNGDYSPLRIKH
RTGDEITYQCRNGFYPATRGNTAKCTSTGWIPAPR
CTLK
Nucleic Acid: (SEQ ID NO: 165):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCGGAGG
CTCTGATGCCGCTGTTGAATGTCCTCCTTGTCCAG
CTCCTCCTGTGGCCGGACCTTCCGTGTTTCTGTTC
CCTCCAAAGCCTAAGGACACCCTGATGATCAGCAG
AACCCCTGAAGTGACCTGCGTGGTGGTGGACGTTT
CCCAAGAGGATCCCGAGGTGCAGTTCAATTGGTAC
GTGGACGGCGTGGAAGTGCACAACGCCAAGACCAA
GCCTAGAGAGGAACAGTTCAACTCCACCTACAGAG
TGGTGTCCGTGCTGACCGTTCTGCACCAGGACTGG
CTGAATGGCAAAGAGTACAAGTGCAAGGTGTCCAA
CAAGGGCCTGCCTAGCAGCATCGAGAAAACCATCA
GCAAGGCCAAGGGCCAGCCAAGAGAACCCCAGGTT
TACACCCTGCCTCCAAGCCAAGAGGAAATGACCAA
GAACCAGGTGTCCCTGACCTGCCTGGTCAAGGGCT
TCTACCCTAGCGACATTGCCGTGGAATGGGAGAGC
AATGGCCAGCCTGAGAACAACTACAAGACCACACC
TCCTGTGCTGGACAGCGACGGCAGCTTTTTTCTGT
ACTCCCGGCTGACCGTGGACAAGAGCAGATGGCAA
GAGGGCAACGTGTTCAGCTGCAGCGTGATGCACGA
AGCCCTGCACAACCACTACACCCAGAAGTCTCTGA
GCCTGAGCCTTGGAAAAGGTGGTGGCGGATCTGGC
GGAGGTGGAAGCGGAGGCGGTGGAAGTGGCGGTGG
TGGATCTGAGGATTGCAACGAGCTGCCTCCTCGGA
GAAACACCGAGATCCTGACCGGATCTTGGAGCGAC
CAGACATACCCTGAAGGCACCCAGGCCATCTACAA
GTGTAGACCCGGCTACAGATCCCTGGGCAATGTGA
TCATGGTCTGCCGGAAAGGCGAGTGGGTTGCCCTG
AATCCTCTGAGAAAGTGCCAGAAGAGGCCTTGCGG
ACACCCCGGCGATACACCTTTTGGCACATTCACCC
TGACCGGCGGCAATGTGTTTGAGTATGGCGTGAAG
GCCGTGTACACCTGTAATGAGGGCTACCAGCTGCT
GGGCGAGATCAACTACAGAGAGTGTGATACCGACG
GCTGGACCAACGACATCCCTATCTGCGAGGTGGTC
AAGTGCCTGCCTGTGACAGCCCCTGAGAATGGCAA
GATCGTGTCCAGCGCCATGGAACCCGACAGAGAGT
ATCACTTTGGCCAGGCCGTCAGATTCGTGTGCAAC
TCTGGATACAAGATCGAGGGCGACGAGGAAATGCA
CTGCAGCGACGACGGCTTCTGGTCCAAAGAAAAGC
CCAAATGCGTGGAAATCAGCTGCAAGTCCCCTGAC
GTGATCAACGGCAGCCCCATCAGCCAGAAGATTAT
CTACAAAGAGAACGAGCGGTTCCAGTATAAGTGCA
ACATGGGCTACGAGTACAGCGAGCGGGGAGATGCC
GTGTGTACAGAATCTGGATGGCGGCCTCTGCCTAG
CTGCGAGGAAAAGAGCTGCGACAACCCCTACATTC
CCAACGGCGACTACAGCCCTCTGCGGATCAAACAC
AGAACCGGCGACGAGATCACCTACCAGTGCAGAAA
CGGCTTTTACCCCGCCACCAGAGGCAATACCGCCA
AGTGTACAAGCACCGGCTGGATCCCAGCTCCACGG
TGCACACTGAAA
Compound B:
Amino Acid (SEQ ID NO: 115):
EDCNELPPRRNTEILTGSWSDQTYPEGTQAIYKCR
PGYRSLGNVIMVCRKGEWVALNPLRKCQKRPCGHP
GDTPFGTFTLTGGNVFEYGVKAVYTCNEGYQLLGE
INYRECDTDGWTNDIPICEVVKCLPVTAPENGKIV
SSAMEPDREYHFGQAVRFVCNSGYKIEGDEEMHCS
DDGFWSKEKPKCVEISCKSPDVINGSPISQKIIYK
ENERFQYKCNMGYEYSERGDAVCTESGWRPLPSCE
EKSCDNPYIPNGDYSPLRIKHRTGDEITYQCRNGF
YPATRGNTAKCTSTGWIPAPRCTLKVECPPCPAPP
VAGPSVFLFPPKPKDTLMISRTPEVTCVVVDVSQE
DPEVQFNWYVDGVEVHNAKTKPREEQFNSTYRVVS
VLTVLHQDWLNGKEYKCKVSNKGLPSSIEKTISKA
KGQPREPQVYTLPPSQEEMTKNQVSLTCLVKGFYP
SDIAVEWESNGQPENNYKTTPPVLDSDGSFFLYSR
LTVDKSRWQEGNVFSCSVMHEALHNHYTQKSLSLS
LGKGKCGPPPPIDNGDITSFPLSVYAPASSVEYQC
QNLYQLEGNKRITCRNGQWSEPPKCLHPCVISREI
MENYNIALRWTAKQKLYSRTGESVEFVCKRGYRLS
SRSHTLRTTCWDGKLEYPTCAKR
Nucleic Acid: (SEQ ID NO: 166):
GAGGATTGCAAGGGCCCTCCACCTAGAGAGAACAG
CGAGATCCTGTCTGGCTCTTGGAGCGAGCAGCTGT
ATCCTGAGGGAACCCAGGCCACCTACAAGTGCAGA
CCTGGCTACAGAACCCTGGGCACCATCGTGAAAGT
GTGCAAGAACGGCAAATGGGTCGCCAGCAATCCCA
GCCGGATCTGCAGAAAGAAACCTTGCGGACACCCC
GGCGATACCCCTTTCGGATCTTTTAGACTGGCCGT
GGGCAGCCAGTTTGAGTTCGGAGCCAAGGTGGTGT
ACACATGCGACGATGGCTATCAGCTGCTGGGCGAG
ATCGACTATAGAGAGTGTGGCGCCGACGGCTGGAT
CAACGATATCCCTCTGTGCGAGGTGGTCAAGTGCC
TGCCTGTGACAGAGCTGGAAAACGGCAGAATTGTG
TCCGGCGCTGCCGAGACAGACCAAGAGTACTACTT
TGGCCAGGTCGTCAGATTCGAGTGCAACAGCGGCT
TCAAGATCGAGGGCCACAAAGAGATCCACTGCAGC
GAGAACGGCCTGTGGTCCAACGAGAAGCCCAGATG
CGTGGAAATCCTGTGCACCCCTCCTAGAGTGGAAA
ATGGCGACGGCATCAACGTGAAGCCCGTGTACAAA
GAGAACGAGCGCTACCACTATAAGTGCAAGCACGG
CTACGTGCCCAAAGAACGGGGAGATGCCGTGTGTA
CAGGCTCTGGATGGTCCAGCCAGCCTTTCTGCGAA
GAGAAGAGATGCAGCCCTCCTTACATCCTGAACGG
CATCTACACCCCTCACCGGATCATCCACAGAAGCG
ACGACGAGATCAGATACGAGTGTAATTACGGCTTC
TACCCCGTGACCGGCAGCACCGTGTCTAAGTGTAC
ACCTACCGGATGGATCCCCGTGCCTAGATGTACAC
TGAAAGGCGGCAGCAGCAGAAGCAGTTCTTCTGGC
GGAGGCGGAGCTGGTGGTGGCGGAGATAAGAAAAT
CGTGCCCAGAGACTGCGGCTGCAAGCCCTGTATCT
GTACAGTGCCTGAGCAGAGCAGCGTGTTCATCTTC
CCACCTAAGCCTAAGGACGTGCTGATGATCAGCCT
GACACCTAAAGTGACCTGCGTGGTGGTGGACATCA
GCAAGGATGACCCTGAGGTGCAGTTCAGTTGGTTC
GTGGACGACGTGGAAGTGCACACAGCCCAGACCAA
GCCAAGAGAGGAACAGATCAACAGCACCTTCAGAA
GCGTGTCCGAGCTGCCCATTCTGCACCAGGACTGG
CTGAATGGCAAAGAGTTCAAGTGTAGAGTGAACTC
CGCCGCTTTTCCCGCTCCTATCGAGAAAACCATCT
CCAAGACCAAGGGCAGACCCAAGGCTCCCCAGGTC
TACACAATCCCTCCACCAAAAGAACAGATGGCCAA
GGACAAGGTGTCCCTGACCTGCATGATCACCAATT
TCTTCCCAGAGGACATCACCGTGGAATGGCAGTGG
AATGGACAGCCCGCCGAGAACTACAAGAACACCCA
GCCTATCATGGACACCGACGGCAGCTACTTCGTGT
ACAGCAAGCTGAACGTGCAGAAGTCCAACTGGGAG
GCCGGCAACACCTTTACCTGTTCTGTGCTGCACGA
GGGCCTGCACAACCACCACACAGAGAAGTCTCTGT
CTCACAGCCCTGGCAAAGGCGGCTCTAGCAGATCT
TCTTCATCTGGTGGCGGTGGTGCCGGTGGCGGCGG
AGGAAAATGTGGACCTCCTCCTCCAATCGACAACG
GCGACATCACAAGCCTGAGCCTGCCAGTGTATGAG
CCCCTGTCTAGCGTGGAATACCAGTGCCAGAAGTA
CTACCTGCTGAAGGGCAAAAAGACCATCACCTGTC
GGAACGGCAAGTGGTCCGAGCCTCCTACATGTCTG
CACGCCTGCGTGATCCCCGAGAACATCATGGAAAG
CCACAACATCATCCTGAAGTGGCGGCACACCGAGA
AGATCTACAGCCACTCTGGCGAGGACATCGAGTTC
GGCTGCAAATACGGCTACTACAAGGCCCGGGATAG
CCCTCCATTCCGGACCAAGTGTATCAACGGCACCA
TCAACTACCCTACCTGCGTC
Compound C:
Amino Acid (SEQ ID NO: 116):
GKCGPPPPIDNGDITSFPLSVYAPASSVEYQCQNL
YQLEGNKRITCRNGQWSEPPKCLHPCVISREIMEN
YNIALRWTAKQKLYSRTGESVEFVCKRGYRLSSRS
HTLRTTCWDGKLEYPTCAKRVECPPCPAPPVAGPS
VFLFPPKPKDTLMISRTPEVTCVVVDVSQEDPEVQ
FNWYVDGVEVHNAKTKPREEQFNSTYRVVSVLTVL
HQDWLNGKEYKCKVSNKGLPSSIEKTISKAKGQPR
EPQVYTLPPSQEEMTKNQVSLTCLVKGFYPSDIAV
EWESNGQPENNYKTTPPVLDSDGSFFLYSRLTVDK
SRWQEGNVFSCSVMHEALHNHYTQKSLSLSLGKED
CNELPPRRNTEILTGSWSDQTYPEGTQAIYKCRPG
YRSLGNVIMVCRKGEWVALNPLRKCQKRPCGHPGD
TPFGTFTLTGGNVFEYGVKAVYTCNEGYQLLGEIN
YRECDTDGWTNDIPICEVVKCLPVTAPENGKIVSS
AMEPDREYHFGQAVRFVCNSGYKIEGDEEMHCSDD
GFWSKEKPKCVEISCKSPDVINGSPISQKIIYKEN
ERFQYKCNMGYEYSERGDAVCTESGWRPLPSCEEK
SCDNPYIPNGDYSPLRIKHRTGDEITYQCRNGFYP
ATRGNTAKCTSTGWIPAPRCTLK
Nucleic Acid: (SEQ ID NO: 167):
GGCAAGTGTGGACCTCCTCCTCCTATCGACAACGG
CGACATCACAAGCCTGAGCCTGCCTGTGTATGAGC
CCCTGAGCAGCGTGGAATACCAGTGCCAGAAGTAC
TACCTGCTGAAGGGCAAGAAAACCATCACCTGTCG
GAACGGCAAGTGGTCCGAGCCTCCTACATGTCTGC
ACGCCTGCGTGATCCCCGAGAACATCATGGAAAGC
CACAACATCATCCTGAAGTGGCGGCACACCGAGAA
GATCTACAGCCACTCTGGCGAGGACATCGAGTTCG
GCTGCAAATACGGCTACTACAAGGCCCGGGATAGC
CCTCCATTCCGGACCAAGTGTATCAACGGCACCAT
CAACTACCCTACCTGCGTCGGCGGCAGCAGCAGAT
CTAGTTCTTCTGGCGGAGGCGGAGCTGGTGGCGGC
GGAGATAAGAAAATCGTGCCTAGAGACTGCGGCTG
CAAGCCCTGTATCTGTACAGTGCCTGAGCAGTCCA
GCGTGTTCATCTTCCCACCTAAGCCTAAGGACGTG
CTGATGATCAGCCTGACACCTAAAGTGACCTGCGT
GGTGGTGGACATCAGCAAGGATGACCCTGAGGTGC
AGTTCAGTTGGTTCGTGGACGACGTGGAAGTGCAC
ACAGCCCAGACCAAGCCTAGAGAGGAACAGATCAA
CAGCACCTTCAGAAGCGTGTCCGAGCTGCCCATTC
TGCACCAGGACTGGCTGAACGGCAAAGAGTTCAAG
TGCAGAGTGAACAGCGCCGCCTTTCCTGCTCCAAT
CGAAAAGACCATCTCCAAGACCAAGGGCAGACCCA
AGGCTCCCCAGGTGTACACAATCCCTCCACCTAAA
GAACAGATGGCCAAGGACAAGGTGTCCCTGACCTG
CATGATCACCAATTTCTTCCCAGAGGACATCACCG
TGGAATGGCAGTGGAATGGACAGCCCGCCGAGAAC
TACAAGAACACCCAGCCTATCATGGACACCGACGG
CAGCTACTTCGTGTACAGCAAGCTGAACGTGCAGA
AGTCCAACTGGGAGGCCGGCAACACCTTTACCTGT
TCTGTGCTGCACGAGGGCCTGCACAACCACCACAC
AGAGAAGTCTCTGTCTCACAGCCCTGGCAAAGGCG
GCAGCTCTAGAAGTAGTTCAAGCGGAGGTGGCGGA
GCAGGCGGTGGTGGCGAAGATTGCAAAGGACCACC
ACCAAGAGAGAACAGCGAGATCCTGTCTGGCTCTT
GGAGCGAGCAGCTGTATCCTGAGGGAACCCAGGCC
ACCTACAAGTGCAGGCCTGGCTATAGAACCCTGGG
CACCATCGTGAAAGTGTGCAAGAATGGCAAATGGG
TCGCCAGCAATCCCAGCCGGATCTGCAGAAAGAAA
CCTTGCGGACACCCCGGCGATACCCCTTTCGGATC
TTTTAGACTGGCCGTGGGCAGCCAGTTTGAGTTCG
GAGCCAAGGTGGTGTATACCTGCGACGATGGCTAT
CAGCTGCTGGGCGAGATCGACTATAGAGAGTGTGG
CGCCGACGGCTGGATCAACGATATCCCTCTGTGCG
AGGTGGTCAAGTGCCTGCCAGTGACAGAGCTGGAA
AACGGCAGAATTGTGTCCGGCGCTGCCGAGACAGA
CCAAGAGTACTACTTTGGCCAGGTCGTCAGATTCG
AGTGCAACAGCGGCTTCAAGATCGAGGGCCACAAA
GAGATCCACTGCAGCGAGAACGGCCTGTGGTCCAA
CGAGAAGCCCAGATGCGTGGAAATCCTGTGCACCC
CTCCTAGAGTGGAAAATGGCGACGGCATCAACGTG
AAGCCCGTGTACAAAGAGAACGAGCGCTACCACTA
TAAGTGCAAGCACGGCTACGTGCCCAAAGAACGGG
GAGATGCCGTGTGTACAGGCTCTGGATGGTCCAGC
CAGCCTTTCTGCGAAGAGAAGAGATGCAGCCCTCC
TTACATCCTGAACGGAATCTACACCCCTCACCGGA
TCATCCACAGAAGCGACGACGAGATCAGATACGAG
TGTAATTACGGCTTCTACCCCGTGACCGGCAGCAC
CGTGTCTAAGTGTACACCAACAGGCTGGATCCCCG
TGCCTCGGTGCACACTGAAA
Compound D:
Amino Acid (SEQ ID NO: 117):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGSSRSSSSGGGGAGGGGVECPPCPAP
PVAGPSVFLFPPKPKDTLMISRTPEVTCVVVDVSQ
EDPEVQFNWYVDGVEVHNAKTKPREEQFNSTYRVV
SVLTVLHQDWLNGKEYKCKVSNKGLPSSIEKTISK
AKGQPREPQVYTLPPSQEEMTKNQVSLTCLVKGFY
PSDIAVEWESNGQPENNYKTTPPVLDSDGSFFLYS
RLTVDKSRWQEGNVFSCSVMHEALHNHYTQKSLSL
SLGKGGSSRSSSSGGGGAGGGGEDCNELPPRRNTE
ILTGSWSDQTYPEGTQAIYKCRPGYRSLGNVIMVC
RKGEWVALNPLRKCQKRPCGHPGDTPFGTFTLTGG
NVFEYGVKAVYTCNEGYQLLGEINYRECDTDGWTN
DIPICEVVKCLPVTAPENGKIVSSAMEPDREYHFG
QAVRFVCNSGYKIEGDEEMHCSDDGFWSKEKPKCV
EISCKSPDVINGSPISQKIIYKENERFQYKCNMGY
EYSERGDAVCTESGWRPLPSCEEKSCDNPYIPNGD
YSPLRIKHRTGDEITYQCRNGFYPATRGNTAKCTS
TGWIPAPRCTLK
Nucleic Acid: (SEQ ID NO: 168):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCAGCAG
CAGATCTTCTAGTTCTGGCGGAGGCGGAGCTGGTG
GTGGCGGAGTTGAATGTCCTCCTTGTCCTGCTCCT
CCAGTGGCCGGACCTTCCGTGTTTCTGTTCCCTCC
AAAGCCTAAGGACACCCTGATGATCAGCAGAACCC
CTGAAGTGACCTGCGTGGTGGTGGACGTTTCCCAA
GAGGATCCCGAGGTGCAGTTCAATTGGTACGTGGA
CGGCGTGGAAGTGCACAACGCCAAGACCAAGCCTA
GAGAGGAACAGTTCAACAGCACCTACAGAGTGGTG
TCCGTGCTGACCGTTCTGCACCAGGACTGGCTGAA
TGGCAAAGAGTACAAGTGCAAGGTGTCCAACAAGG
GCCTGCCTAGCAGCATCGAGAAAACCATCAGCAAG
GCCAAGGGCCAGCCAAGAGAACCCCAGGTTTACAC
CCTGCCTCCAAGCCAAGAGGAAATGACCAAGAACC
AGGTGTCCCTGACCTGCCTGGTCAAGGGCTTCTAC
CCTAGCGACATTGCCGTGGAATGGGAGAGCAATGG
CCAGCCTGAGAACAACTACAAGACCACACCTCCTG
TGCTGGACAGCGACGGCAGCTTTTTTCTGTACTCC
CGGCTGACCGTGGACAAGAGCAGATGGCAAGAGGG
CAACGTGTTCAGCTGCAGCGTGATGCACGAAGCCC
TGCACAACCACTACACCCAGAAGTCTCTGAGCCTG
TCTCTCGGCAAAGGCGGCTCTAGCAGAAGTAGTTC
TTCTGGCGGCGGTGGTGCTGGCGGCGGAGGCGAAG
ATTGCAATGAACTGCCTCCTCGGCGGAACACCGAG
ATCTTGACAGGATCTTGGAGCGACCAGACATACCC
TGAGGGCACCCAGGCCATCTACAAGTGTAGACCTG
GCTACAGATCCCTGGGCAATGTGATCATGGTCTGC
CGGAAAGGCGAGTGGGTTGCCCTGAATCCTCTGAG
AAAGTGCCAGAAGAGGCCTTGCGGACACCCCGGCG
ATACACCTTTTGGCACATTCACCCTGACCGGCGGC
AATGTGTTTGAGTATGGCGTGAAGGCCGTGTACAC
CTGTAATGAGGGCTACCAGCTGCTGGGCGAGATCA
ACTACAGAGAGTGTGATACCGACGGCTGGACCAAC
GACATCCCTATCTGCGAGGTGGTCAAGTGCCTGCC
TGTGACAGCCCCTGAGAATGGCAAGATCGTGTCCA
GCGCCATGGAACCCGACAGAGAGTATCACTTTGGC
CAGGCCGTCAGATTCGTGTGCAACTCCGGATACAA
GATCGAGGGCGACGAGGAAATGCACTGCAGCGACG
ACGGCTTCTGGTCCAAAGAAAAGCCCAAATGCGTG
GAAATCAGCTGCAAGTCCCCTGACGTGATCAACGG
CAGCCCCATCAGCCAGAAGATTATCTACAAAGAGA
ACGAGCGGTTCCAGTATAAGTGCAACATGGGCTAC
GAGTACAGCGAGCGGGGAGATGCCGTGTGTACAGA
ATCTGGATGGCGGCCTCTGCCTAGCTGCGAGGAAA
AGAGCTGCGACAACCCCTACATTCCCAACGGCGAC
TACAGCCCTCTGCGGATCAAACACAGAACCGGCGA
CGAGATCACCTACCAGTGCAGAAACGGCTTTTACC
CCGCCACCAGAGGCAATACCGCCAAGTGTACAAGC
ACCGGCTGGATCCCAGCTCCTCGGTGCACACTGAA
A
Compound E:
Amino Acid (SEQ ID NO: 118):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGSDAAVECPPCPAPPVAGPSVFLF
PPKPKDTLMISRTPEVTCVVVDVSQEDPEVQFNWY
VDGVEVHNAKTKPREEQFNSTYRVVSVLTVLHQDW
LNGKEYKCKVSNKGLPSSIEKTISKAKGQPREPQV
YTLPPSQEEMTKNQVSLTCLVKGFYPSDIAVEWES
NGQPENNYKTTPPVLDSDGSFFLYSRLTVDKSRWQ
EGNVFSCSVMHEALHNHYTQKSLSLSLGKGGGGAG
GGGAGGGGSEDCNELPPRRNTEILTGSWSDQTYPE
GTQAIYKCRPGYRSLGNVIMVCRKGEWVALNPLRK
CQKRPCGHPGDTPFGTFTLTGGNVFEYGVKAVYTC
NEGYQLLGEINYRECDTDGWTNDIPICEVVKCLPV
TAPENGKIVSSAMEPDREYHFGQAVRFVCNSGYKI
EGDEEMHCSDDGFWSKEKPKCVEISCKSPDVINGS
PISQKIIYKENERFQYKCNMGYEYSERGDAVCTES
GWRPLPSCEEKSCDNPYIPNGDYSPLRIKHRTGDE
ITYQCRNGFYPATRGNTAKCTSTGWIPAPRCTLK
Nucleic Acid: (SEQ ID NO: 169):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCGGAGG
CTCTGATGCCGCTGTTGAATGTCCTCCTTGTCCAG
CTCCTCCTGTGGCCGGACCTTCCGTGTTTCTGTTC
CCTCCAAAGCCTAAGGACACCCTGATGATCAGCAG
AACCCCTGAAGTGACCTGCGTGGTGGTGGACGTTT
CCCAAGAGGATCCCGAGGTGCAGTTCAATTGGTAC
GTGGACGGCGTGGAAGTGCACAACGCCAAGACCAA
GCCTAGAGAGGAACAGTTCAACTCCACCTACAGAG
TGGTGTCCGTGCTGACCGTTCTGCACCAGGACTGG
CTGAATGGCAAAGAGTACAAGTGCAAGGTGTCCAA
CAAGGGCCTGCCTAGCAGCATCGAGAAAACCATCA
GCAAGGCCAAGGGCCAGCCAAGAGAACCCCAGGTT
TACACCCTGCCTCCAAGCCAAGAGGAAATGACCAA
GAACCAGGTGTCCCTGACCTGCCTGGTCAAGGGCT
TCTACCCTAGCGACATTGCCGTGGAATGGGAGAGC
AATGGCCAGCCTGAGAACAACTACAAGACCACACC
TCCTGTGCTGGACAGCGACGGCAGCTTTTTTCTGT
ACTCCCGGCTGACCGTGGACAAGAGCAGATGGCAA
GAGGGCAACGTGTTCAGCTGCAGCGTGATGCACGA
AGCCCTGCACAACCACTACACCCAGAAGTCTCTGA
GCCTGAGCCTTGGAAAAGGTGGTGGCGGATCTGGC
GGAGGTGGAAGCGAAGATTGCAACGAGCTGCCTCC
TCGGAGAAACACCGAGATCCTGACCGGATCTTGGA
GCGACCAGACATACCCTGAAGGCACCCAGGCCATC
TACAAGTGTAGACCCGGCTACAGATCCCTGGGCAA
TGTGATCATGGTCTGCCGGAAAGGCGAGTGGGTTG
CCCTGAATCCTCTGAGAAAGTGCCAGAAGAGGCCT
TGCGGACACCCCGGCGATACACCTTTTGGCACATT
CACCCTGACCGGCGGCAATGTGTTTGAGTATGGCG
TGAAGGCCGTGTACACCTGTAATGAGGGCTACCAG
CTGCTGGGCGAGATCAACTACAGAGAGTGTGATAC
CGACGGCTGGACCAACGACATCCCTATCTGCGAGG
TGGTCAAGTGCCTGCCTGTGACAGCCCCTGAGAAT
GGCAAGATCGTGTCCAGCGCCATGGAACCCGACAG
AGAGTATCACTTTGGCCAGGCCGTCAGATTCGTGT
GCAACTCTGGATACAAGATCGAGGGCGACGAGGAA
ATGCACTGCAGCGACGACGGCTTCTGGTCCAAAGA
AAAGCCCAAATGCGTGGAAATCAGCTGCAAGTCCC
CTGACGTGATCAACGGCAGCCCCATCAGCCAGAAG
ATTATCTACAAAGAGAACGAGCGGTTCCAGTATAA
GTGCAACATGGGCTACGAGTACAGCGAGCGGGGAG
ATGCCGTGTGTACAGAATCTGGATGGCGGCCTCTG
CCTAGCTGCGAGGAAAAGAGCTGCGACAACCCCTA
CATTCCCAACGGCGACTACAGCCCTCTGCGGATCA
AACACAGAACCGGCGACGAGATCACCTACCAGTGC
AGAAACGGCTTTTACCCCGCCACCAGAGGCAATAC
CGCCAAGTGTACAAGCACCGGCTGGATCCCAGCTC
CACGGTGCACACTGAAA
Compound O:
Amino Acid (SEQ ID NO: 125):
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSEDCNELPPRRNTEILTG
SWSDQTYPEGTQAIYKCRPGYRSLGNVIMVCRKGE
WVALNPLRKCQKRPCGHPGDTPFGTFTLTGGNVFE
YGVKAVYTCNEGYQLLGEINYRECDTDGWTNDIPI
CEVVKCLPVTAPENGKIVSSAMEPDREYHFGQAVR
FVCNSGYKIEGDEEMHCSDDGFWSKEKPKCVEISC
KSPDVINGSPISQKIIYKENERFQYKCNMGYEYSE
RGDAVCTESGWRPLPSCEEKSCDNPYIPNGDYSPL
RIKHRTGDEITYQCRNGFYPATRGNTAKCTSTGWI
PAPRCTLK
Nucleic Acid: (SEQ ID NO: 179):
GAGGTGCAGCTGGTTGAATCTGGCGGAGGACTTGT
GAAGCCTGGCGGCTCTCTGAGACTGTCTTGTGCTG
CTTCTGGCAGACCCGTGTCTAATTACGCCGCTGCC
TGGTTTAGACAGGCCCCTGGCAAAGAGAGAGAGTT
CGTCAGCGCCATCAACTGGCAGAAAACCGCCACAT
ACGCCGACAGCGTGAAGGGCAGATTCACCATCAGC
CGGGACAACGCCAAGAACAGCCTGTACCTGCAGAT
GAACTCCCTGAGAGCCGAGGACACCGCCGTGTATT
ATTGTGCCGCCGTGTTTAGAGTGGTGGCCCCTAAG
ACACAGTACGACTACGATTACTGGGGCCAGGGCAC
CCTGGTTACCGTGTCTAGCGAGGATTGCAACGAGC
TGCCTCCTCGGAGAAACACCGAGATCCTGACAGGC
TCTTGGAGCGACCAGACATACCCTGAGGGCACCCA
GGCCATCTACAAGTGCAGACCTGGCTACAGATCCC
TGGGCAACGTGATCATGGTCTGCAGAAAAGGCGAG
TGGGTCGCCCTGAATCCTCTGAGAAAGTGCCAGAA
GAGGCCTTGCGGACACCCTGGCGATACCCCTTTTG
GCACATTCACACTGACCGGCGGCAACGTGTTCGAG
TATGGCGTGAAGGCCGTGTACACCTGTAACGAGGG
ATATCAGCTGCTGGGCGAGATCAACTACAGAGAGT
GTGATACCGACGGCTGGACCAACGACATCCCTATC
TGCGAGGTGGTCAAGTGCCTGCCTGTGACAGCCCC
TGAGAATGGCAAGATCGTGTCCAGCGCCATGGAAC
CCGACAGAGAGTATCACTTTGGCCAGGCCGTCAGA
TTCGTGTGCAACAGCGGCTATAAGATCGAGGGCGA
CGAGGAAATGCACTGCAGCGACGACGGCTTCTGGT
CCAAAGAAAAGCCTAAGTGCGTGGAAATCAGCTGC
AAGAGCCCCGACGTGATCAACGGCAGCCCTATCAG
CCAGAAGATCATCTACAAAGAGAACGAGCGGTTCC
AGTACAAGTGTAACATGGGCTACGAGTACAGCGAG
AGGGGCGACGCCGTGTGTACAGAATCTGGATGGCG
ACCTCTGCCTAGCTGCGAGGAAAAGAGCTGCGACA
ACCCTTACATCCCCAACGGCGACTACAGCCCTCTG
CGGATTAAGCACAGAACCGGCGACGAGATCACCTA
CCAGTGCAGAAATGGCTTCTACCCCGCCACCAGAG
GCAATACCGCCAAGTGTACAAGCACCGGCTGGATC
CCTGCTCCTCGGTGCACACTGAAA
Compound F:
Amino Acid (SEQ ID NO: 119):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGSDAAVECPPCPAPPVAGPSVFLF
PPKPKDTLMISRTPEVTCVVVDVSQEDPEVQFNWY
VDGVEVHNAKTKPREEQFNSTYRVVSVLTVLHQDW
LNGKEYKCKVSNKGLPSSIEKTISKAKGQPREPQV
YTLPPSQEEMTKNQVSLTCLVKGFYPSDIAVEWES
NGQPENNYKTTPPVLDSDGSFFLYSRLTVDKSRWQ
EGNVFSCSVMHEALHNHYTQKSLSLSLGKGGGGSE
DCNELPPRRNTEILTGSWSDQTYPEGTQAIYKCRP
GYRSLGNVIMVCRKGEWVALNPLRKCQKRPCGHPG
DTPFGTFTLTGGNVFEYGVKAVYTCNEGYQLLGEI
NYRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KSCDNPYIPNGDYSPLRIKHRTGDEITYQCRNGFY
PATRGNTAKCTSTGWIPAPRCTLK
Nucleic Acid: (SEQ ID NO: 170):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCGGAGG
CTCTGATGCCGCTGTTGAATGTCCTCCTTGTCCAG
CTCCTCCTGTGGCCGGACCTTCCGTGTTTCTGTTC
CCTCCAAAGCCTAAGGACACCCTGATGATCAGCAG
AACCCCTGAAGTGACCTGCGTGGTGGTGGACGTTT
CCCAAGAGGATCCCGAGGTGCAGTTCAATTGGTAC
GTGGACGGCGTGGAAGTGCACAACGCCAAGACCAA
GCCTAGAGAGGAACAGTTCAACTCCACCTACAGAG
TGGTGTCCGTGCTGACCGTTCTGCACCAGGACTGG
CTGAATGGCAAAGAGTACAAGTGCAAGGTGTCCAA
CAAGGGCCTGCCTAGCAGCATCGAGAAAACCATCA
GCAAGGCCAAGGGCCAGCCAAGAGAACCCCAGGTT
TACACCCTGCCTCCAAGCCAAGAGGAAATGACCAA
GAACCAGGTGTCCCTGACCTGCCTGGTCAAGGGCT
TCTACCCTAGCGACATTGCCGTGGAATGGGAGAGC
AATGGCCAGCCTGAGAACAACTACAAGACCACACC
TCCTGTGCTGGACAGCGACGGCAGCTTTTTTCTGT
ACTCCCGGCTGACCGTGGACAAGAGCAGATGGCAA
GAGGGCAACGTGTTCAGCTGCAGCGTGATGCACGA
AGCCCTGCACAACCACTACACCCAGAAGTCTCTGA
GCCTGAGCCTTGGAAAAGGCGGAGGCGGAAGCGAG
GATTGCAATGAGCTGCCTCCTCGGAGAAACACCGA
GATCCTGACCGGATCTTGGAGCGACCAGACATACC
CTGAAGGCACCCAGGCCATCTACAAGTGTAGACCC
GGCTACAGATCCCTGGGCAATGTGATCATGGTCTG
CCGGAAAGGCGAGTGGGTTGCCCTGAATCCTCTGA
GAAAGTGCCAGAAGAGGCCTTGCGGACACCCCGGC
GATACACCTTTTGGCACATTCACCCTGACCGGCGG
CAATGTGTTTGAGTATGGCGTGAAGGCCGTGTACA
CCTGTAATGAGGGCTACCAGCTGCTGGGCGAGATC
AACTACAGAGAGTGTGATACCGACGGCTGGACCAA
CGACATCCCTATCTGCGAGGTGGTCAAGTGCCTGC
CTGTGACAGCCCCTGAGAATGGCAAGATCGTGTCC
AGCGCCATGGAACCCGACAGAGAGTATCACTTTGG
CCAGGCCGTCAGATTCGTGTGCAACTCTGGATACA
AGATCGAGGGCGACGAGGAAATGCACTGCAGCGAC
GACGGCTTCTGGTCCAAAGAAAAGCCCAAATGCGT
GGAAATCAGCTGCAAGTCCCCTGACGTGATCAACG
GCAGCCCCATCAGCCAGAAGATTATCTACAAAGAG
AACGAGCGGTTCCAGTATAAGTGCAACATGGGCTA
CGAGTACAGCGAGCGGGGAGATGCCGTGTGTACAG
AATCTGGATGGCGGCCTCTGCCTAGCTGCGAGGAA
AAGAGCTGCGACAACCCCTACATTCCCAACGGCGA
CTACAGCCCTCTGCGGATCAAACACAGAACCGGCG
ACGAGATCACCTACCAGTGCAGAAACGGCTTTTAC
CCCGCCACCAGAGGCAATACCGCCAAGTGTACAAG
CACCGGCTGGATCCCAGCTCCACGGTGCACACTGA
AA
Compound G:
Amino Acid (SEQ ID NO: 120):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEDAAVECPPCPAPPVAGPSVFLFPPKPK
DTLMISRTPEVTCVVVDVSQEDPEVQFNWYVDGVE
VHNAKTKPREEQFNSTYRVVSVLTVLHQDWLNGKE
YKCKVSNKGLPSSIEKTISKAKGQPREPQVYTLPP
SQEEMTKNQVSLTCLVKGFYPSDIAVEWESNGQPE
NNYKTTPPVLDSDGSFFLYSRLTVDKSRWQEGNVF
SCSVMHEALHNHYTQKSLSLSLGKEDCNELPPRRN
TEILTGSWSDQTYPEGTQAIYKCRPGYRSLGNVIM
VCRKGEWVALNPLRKCQKRPCGHPGDTPFGTFTLT
GGNVFEYGVKAVYTCNEGYQLLGEINYRECDTDGW
TNDIPICEVVKCLPVTAPENGKIVSSAMEPDREYH
FGQAVRFVCNSGYKIEGDEEMHCSDDGFWSKEKPK
CVEISCKSPDVINGSPISQKIIYKENERFQYKCNM
GYEYSERGDAVCTESGWRPLPSCEEKSCDNPYIPN
GDYSPLRIKHRTGDEITYQCRNGFYPATRGNTAKC
TSTGWIPAPRCTLKHHHHHH
Nucleic Acid: (SEQ ID NO: 171):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGCGAAGAGGACGCCGCCGT
GGAATGTCCTCCTTGTCCTGCTCCTCCAGTGGCCG
GACCTTCCGTGTTTCTGTTCCCTCCAAAGCCTAAG
GACACCCTGATGATCAGCAGAACCCCTGAAGTGAC
CTGCGTGGTGGTGGACGTTTCCCAAGAGGATCCCG
AGGTGCAGTTCAATTGGTACGTGGACGGCGTGGAA
GTGCACAACGCCAAGACCAAGCCTAGAGAGGAACA
GTTCAACAGCACCTACAGAGTGGTGTCCGTGCTGA
CCGTTCTGCACCAGGACTGGCTGAATGGCAAAGAG
TACAAGTGCAAGGTGTCCAACAAGGGCCTGCCTAG
CAGCATCGAGAAAACCATCAGCAAGGCCAAGGGCC
AGCCAAGAGAACCCCAGGTTTACACCCTGCCTCCA
AGCCAAGAGGAAATGACCAAGAACCAGGTGTCCCT
GACCTGCCTGGTCAAGGGCTTCTACCCTAGCGACA
TTGCTGTGGAATGGGAGAGCAACGGCCAGCCTGAG
AACAACTACAAGACCACACCTCCTGTGCTGGACAG
CGACGGCAGCTTTTTTCTGTACTCCCGGCTGACCG
TGGACAAGAGCAGATGGCAAGAGGGCAACGTGTTC
AGCTGCAGCGTGATGCACGAAGCCCTGCACAACCA
CTACACCCAGAAGTCTCTGAGCCTGTCTCTGGGCA
AAGAGGACTGCAACGAGCTGCCTCCTCGGAGAAAT
ACCGAGATCCTGACCGGCTCTTGGAGCGACCAGAC
ATATCCAGAAGGCACCCAGGCCATCTACAAGTGCC
GGCCTGGATACAGATCCCTGGGCAATGTGATCATG
GTCTGCCGGAAAGGCGAGTGGGTTGCCCTGAATCC
TCTGAGAAAGTGCCAGAAGAGGCCTTGCGGACACC
CCGGCGATACACCTTTTGGCACATTCACCCTGACA
GGCGGCAATGTGTTCGAGTATGGCGTGAAGGCCGT
GTACACCTGTAATGAGGGCTACCAGCTGCTGGGCG
AGATCAACTACAGAGAGTGTGATACCGACGGCTGG
ACCAACGACATCCCTATCTGCGAGGTGGTCAAGTG
CCTGCCAGTGACAGCCCCTGAGAATGGCAAGATCG
TGTCCAGCGCCATGGAACCCGACAGAGAGTATCAC
TTTGGCCAGGCCGTCAGATTCGTGTGCAACTCCGG
ATACAAGATCGAGGGCGACGAGGAAATGCACTGCA
GCGACGACGGCTTCTGGTCCAAAGAAAAGCCCAAA
TGCGTGGAAATCAGCTGCAAGTCCCCTGACGTGAT
CAACGGCAGCCCCATCAGCCAGAAGATTATCTACA
AAGAGAACGAGCGGTTCCAGTATAAGTGCAACATG
GGCTACGAGTACAGCGAGCGGGGAGATGCCGTGTG
TACAGAATCTGGATGGCGGCCTCTGCCTAGCTGCG
AGGAAAAGAGCTGCGACAACCCCTACATTCCCAAC
GGCGACTACAGCCCTCTGCGGATCAAACACAGAAC
CGGCGACGAGATCACCTACCAGTGCAGAAACGGCT
TTTACCCCGCCACCAGAGGCAATACCGCCAAGTGT
ACAAGCACCGGCTGGATCCCTGCTCCAAGATGCAC
ACTGAAGCACCACCACCATCACCAC
Compound H:
Amino Acid (SEQ ID NO: 121):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GQKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGAGGGGAGGGGSVECPPCPAPPVA
GPSVFLFPPKPKDTLMISRTPEVTCVVVDVSQEDP
EVQFNWYVDGVEVHNAKTKPREEQFNSTYRVVSVL
TVLHQDWLNGKEYKCKVSNKGLPSSIEKTISKAKG
QPREPQVYTLPPSQEEMTKNQVSLTCLVKGFYPSD
IAVEWESNGQPENNYKTTPPVLDSDGSFFLYSRLT
VDKSRWQEGNVFSCSVMHEALHNHYTQKSLSLSLG
KGGGGAGGGGAGGGGSEDCNELPPRRNTEILTGSW
SDQTYPEGTQAIYKCRPGYRSLGNVIMVCRKGEWV
ALNPLRKCQKRPCGHPGDTPFGTFTLTGGNVFEYG
VKAVYTCNEGYQLLGEINYRECDTDGWTNDIPICE
VVKCLPVTAPENGKIVSSAMEPDREYHFGQAVRFV
CNSGYKIEGDEEMHCSDDGFWSKEKPKCVEISCKS
PDVINGSPISQKIIYKENERFQYKCNMGYEYSERG
DAVCTESGWRPLPSCEEKSCDNPYIPNGDYSPLRI
KHRTGDEITYQCRNGFYPATRGNTAKCTSTGWIPA
PRCTLK
Nucleic Acid: (SEQ ID NO: 172):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCCAGAAAAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGAGGCGG
AGCTGGTGGTGGCGGTGCTGGTGGCGGAGGATCTG
TTGAATGTCCTCCTTGTCCAGCTCCTCCTGTGGCC
GGACCTTCCGTGTTTCTGTTCCCTCCAAAGCCTAA
GGACACCCTGATGATCAGCAGAACCCCTGAAGTGA
CCTGCGTGGTGGTGGACGTTTCCCAAGAGGATCCC
GAGGTGCAGTTCAATTGGTACGTGGACGGCGTGGA
AGTGCACAACGCCAAGACCAAGCCTAGAGAGGAAC
AGTTCAACAGCACCTACAGAGTGGTGTCCGTGCTG
ACCGTTCTGCACCAGGACTGGCTGAATGGCAAAGA
GTACAAGTGCAAGGTGTCCAACAAGGGCCTGCCTA
GCAGCATCGAGAAAACCATCAGCAAGGCCAAGGGC
CAGCCAAGAGAACCCCAGGTTTACACCCTGCCTCC
AAGCCAAGAGGAAATGACCAAGAACCAGGTGTCCC
TGACCTGCCTGGTCAAGGGCTTCTACCCTAGCGAC
ATTGCCGTGGAATGGGAGAGCAATGGCCAGCCTGA
GAACAACTACAAGACCACACCTCCTGTGCTGGACA
GCGACGGCAGCTTTTTTCTGTACTCCCGGCTGACC
GTGGACAAGAGCAGATGGCAAGAGGGCAACGTGTT
CAGCTGCAGCGTGATGCACGAAGCCCTGCACAACC
ACTACACCCAGAAGTCTCTGAGCCTGTCTCTCGGA
AAAGGTGGTGGCGGAGCTGGCGGAGGTGGTGCAGG
CGGTGGTGGATCTGAAGATTGCAACGAGCTGCCTC
CTCGGCGGAATACCGAGATTCTGACCGGATCTTGG
AGCGACCAGACATACCCTGAAGGCACCCAGGCCAT
CTACAAGTGTAGACCCGGCTACAGATCCCTGGGCA
ATGTGATCATGGTCTGCCGGAAAGGCGAGTGGGTT
GCCCTGAATCCTCTGAGAAAGTGCCAGAAGAGGCC
TTGCGGACACCCCGGCGATACACCTTTTGGCACAT
TCACCCTGACCGGCGGCAATGTGTTTGAGTATGGC
GTGAAGGCCGTGTACACCTGTAATGAGGGCTACCA
GCTGCTGGGCGAGATCAACTACAGAGAGTGTGATA
CCGACGGCTGGACCAACGACATCCCTATCTGCGAG
GTGGTCAAGTGCCTGCCTGTGACAGCCCCTGAGAA
TGGCAAGATCGTGTCCAGCGCCATGGAACCCGACA
GAGAGTATCACTTTGGCCAGGCCGTCAGATTCGTG
TGCAACTCTGGATACAAGATCGAGGGCGACGAGGA
AATGCACTGCAGCGACGACGGCTTCTGGTCCAAAG
AAAAGCCCAAATGCGTGGAAATCAGCTGCAAGTCC
CCTGACGTGATCAACGGCAGCCCCATCAGCCAGAA
GATTATCTACAAAGAGAACGAGCGGTTCCAGTATA
AGTGCAACATGGGCTACGAGTACAGCGAGCGGGGA
GATGCCGTGTGTACAGAATCTGGATGGCGGCCTCT
GCCTAGCTGCGAGGAAAAGAGCTGCGACAACCCCT
ACATTCCCAACGGCGACTACAGCCCTCTGCGGATC
AAACACAGAACCGGCGACGAGATCACCTACCAGTG
CAGAAACGGCTTTTACCCTGCCACCAGAGGCAACA
CCGCCAAGTGTACAAGCACAGGCTGGATCCCCGCT
CCTCGGTGTACACTGAAA
Compound I:
Amino Acid (SEQ ID NO: 122):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKAVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGAGGGGAGGGGSVECPPCPAPPVA
GPSVFLFPPKPKDTLMISRTPEVTCVVVDVSQEDP
EVQFNWYVDGVEVHNAKTKPREEQFNSTYRVVSVL
TVLHQDWLNGKEYKCKVSNKGLPSSIEKTISKAKG
QPREPQVYTLPPSQEEMTKNQVSLTCLVKGFYPSD
IAVEWESNGQPENNYKTTPPVLDSDGSFFLYSRLT
VDKSRWQEGNVFSCSVMHEALHNHYTQKSLSLSLG
KGGGGAGGGGAGGGGSEDCNELPPRRNTEILTGSW
SDQTYPEGTQAIYKCRPGYRSLGNVIMVCRKGEWV
ALNPLRKCQKRPCGHPGDTPFGTFTLTGGNVFEYG
VKAVYTCNEGYQLLGEINYRECDTDGWTNDIPICE
VVKCLPVTAPENGKIVSSAMEPDREYHFGQAVRFV
CNSGYKIEGDEEMHCSDDGFWSKEKPKCVEISCKS
PDVINGSPISQKIIYKENERFQYKCNMGYEYSERG
DAVCTESGWRPLPSCEEKSCDNPYIPNGDYSPLRI
KHRTGDEITYQCRNGFYPATRGNTAKCTSTGWIPA
PRCTLK
Nucleic Acid: (SEQ ID NO: 173):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGGCCGTGTGGTGCCAGGCCAACAATAT
GTGGGGACCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGAGGCGG
AGCTGGTGGTGGCGGTGCTGGTGGCGGAGGATCTG
TTGAATGTCCTCCTTGTCCAGCTCCTCCTGTGGCC
GGACCTTCCGTGTTTCTGTTCCCTCCAAAGCCTAA
GGACACCCTGATGATCAGCAGAACCCCTGAAGTGA
CCTGCGTGGTGGTGGACGTTTCCCAAGAGGATCCC
GAGGTGCAGTTCAATTGGTACGTGGACGGCGTGGA
AGTGCACAACGCCAAGACCAAGCCTAGAGAGGAAC
AGTTCAACAGCACCTACAGAGTGGTGTCCGTGCTG
ACCGTTCTGCACCAGGACTGGCTGAATGGCAAAGA
GTACAAGTGCAAGGTGTCCAACAAGGGCCTGCCTA
GCAGCATCGAGAAAACCATCAGCAAGGCCAAGGGC
CAGCCAAGAGAACCCCAGGTTTACACCCTGCCTCC
AAGCCAAGAGGAAATGACCAAGAACCAGGTGTCCC
TGACCTGCCTGGTCAAGGGCTTCTACCCTAGCGAC
ATTGCCGTGGAATGGGAGAGCAATGGCCAGCCTGA
GAACAACTACAAGACCACACCTCCTGTGCTGGACA
GCGACGGCAGCTTTTTTCTGTACTCCCGGCTGACC
GTGGACAAGAGCAGATGGCAAGAGGGCAACGTGTT
CAGCTGCAGCGTGATGCACGAAGCCCTGCACAACC
ACTACACCCAGAAGTCTCTGAGCCTGTCTCTCGGA
AAAGGTGGTGGCGGAGCTGGCGGAGGTGGTGCAGG
CGGTGGTGGATCTGAAGATTGCAACGAGCTGCCTC
CTCGGCGGAATACCGAGATTCTGACCGGATCTTGG
AGCGACCAGACATACCCTGAAGGCACCCAGGCCAT
CTACAAGTGTAGACCCGGCTACAGATCCCTGGGCA
ATGTGATCATGGTCTGCCGGAAAGGCGAGTGGGTT
GCCCTGAATCCTCTGAGAAAGTGCCAGAAGAGGCC
TTGCGGACACCCCGGCGATACACCTTTTGGCACAT
TCACCCTGACCGGCGGCAATGTGTTTGAGTATGGC
GTGAAAGCCGTGTACACCTGTAATGAGGGCTACCA
GCTGCTGGGCGAGATCAACTACAGAGAGTGTGATA
CCGACGGCTGGACCAACGACATCCCTATCTGCGAG
GTGGTCAAGTGCCTGCCTGTGACAGCCCCTGAGAA
TGGCAAGATCGTGTCCAGCGCCATGGAACCCGACA
GAGAGTATCACTTTGGCCAGGCCGTCAGATTCGTG
TGCAACTCTGGATACAAGATCGAGGGCGACGAGGA
AATGCACTGCAGCGACGACGGCTTCTGGTCCAAAG
AAAAGCCCAAATGCGTGGAAATCAGCTGCAAGTCC
CCTGACGTGATCAACGGCAGCCCCATCAGCCAGAA
GATTATCTACAAAGAGAACGAGCGGTTCCAGTATA
AGTGCAACATGGGCTACGAGTACAGCGAGCGGGGA
GATGCCGTGTGTACAGAATCTGGATGGCGGCCTCT
GCCTAGCTGCGAGGAAAAGAGCTGCGACAACCCCT
ACATTCCCAACGGCGACTACAGCCCTCTGCGGATC
AAACACAGAACCGGCGACGAGATCACCTACCAGTG
CAGAAACGGCTTTTACCCTGCCACCAGAGGCAACA
CCGCCAAGTGTACAAGCACAGGCTGGATCCCCGCT
CCTCGGTGTACACTGAAA
Compound M:
Amino Acid (SEQ ID NO: 123):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEDAAVECPPCPAPPVAGPSVFLFPPKPK
DTLMISRTPEVTCVVVDVSQEDPEVQFNWYVDGVE
VHNAKTKPREEQFNSTYRVVSVLTVLHQDWLNGKE
YKCKVSNKGLPSSIEKTISKAKGQPREPQVYTLPP
SQEEMTKNQVSLTCLVKGFYPSDIAVEWESNGQPE
NNYKTTPPVLDSDGSFFLYSRLTVDKSRWQEGNVF
SCSVMHEALHNHYTQKSLSLSLGKEDCNELPPRRN
TEILTGSWSDQTYPEGTQAIYKCRPGYRSLGNVIM
VCRKGEWVALNPLRKCQKRPCGHPGDTPFGTFTLT
GGNVFEYGVKAVYTCNEGYQLLGEINYRECDTDGW
TNDIPICEVVKCLPVTAPENGKIVSSAMEPDREYH
FGQAVRFVCNSGYKIEGDEEMHCSDDGFWSKEKPK
CVEISCKSPDVINGSPISQKIIYKENERFQYKCNM
GYEYSERGDAVCTESGWRPLPSCEEKSCDNPYIPN
GDYSPLRIKHRTGDEITYQCRNGFYPATRGNTAKC
TSTGWIPAPRCTLK
Nucleic Acid: (SEQ ID NO: 177):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGCGAAGAGGACGCCGCCGT
GGAATGTCCTCCTTGTCCTGCTCCTCCAGTGGCCG
GACCTTCCGTGTTTCTGTTCCCTCCAAAGCCTAAG
GACACCCTGATGATCAGCAGAACCCCTGAAGTGAC
CTGCGTGGTGGTGGACGTTTCCCAAGAGGATCCCG
AGGTGCAGTTCAATTGGTACGTGGACGGCGTGGAA
GTGCACAACGCCAAGACCAAGCCTAGAGAGGAACA
GTTCAACAGCACCTACAGAGTGGTGTCCGTGCTGA
CCGTTCTGCACCAGGACTGGCTGAATGGCAAAGAG
TACAAGTGCAAGGTGTCCAACAAGGGCCTGCCTAG
CAGCATCGAGAAAACCATCAGCAAGGCCAAGGGCC
AGCCAAGAGAACCCCAGGTTTACACCCTGCCTCCA
AGCCAAGAGGAAATGACCAAGAACCAGGTGTCCCT
GACCTGCCTGGTCAAGGGCTTCTACCCTAGCGACA
TTGCTGTGGAATGGGAGAGCAACGGCCAGCCTGAG
AACAACTACAAGACCACACCTCCTGTGCTGGACAG
CGACGGCAGCTTTTTTCTGTACTCCCGGCTGACCG
TGGACAAGAGCAGATGGCAAGAGGGCAACGTGTTC
AGCTGCAGCGTGATGCACGAAGCCCTGCACAACCA
CTACACCCAGAAGTCTCTGAGCCTGTCTCTGGGCA
AAGAGGACTGCAACGAGCTGCCTCCTCGGAGAAAT
ACCGAGATCCTGACCGGCTCTTGGAGCGACCAGAC
ATATCCAGAAGGCACCCAGGCCATCTACAAGTGCC
GGCCTGGATACAGATCCCTGGGCAATGTGATCATG
GTCTGCCGGAAAGGCGAGTGGGTTGCCCTGAATCC
TCTGAGAAAGTGCCAGAAGAGGCCTTGCGGACACC
CCGGCGATACACCTTTTGGCACATTCACCCTGACA
GGCGGCAATGTGTTCGAGTATGGCGTGAAGGCCGT
GTACACCTGTAATGAGGGCTACCAGCTGCTGGGCG
AGATCAACTACAGAGAGTGTGATACCGACGGCTGG
ACCAACGACATCCCTATCTGCGAGGTGGTCAAGTG
CCTGCCAGTGACAGCCCCTGAGAATGGCAAGATCG
TGTCCAGCGCCATGGAACCCGACAGAGAGTATCAC
TTTGGCCAGGCCGTCAGATTCGTGTGCAACTCCGG
ATACAAGATCGAGGGCGACGAGGAAATGCACTGCA
GCGACGACGGCTTCTGGTCCAAAGAAAAGCCCAAA
TGCGTGGAAATCAGCTGCAAGTCCCCTGACGTGAT
CAACGGCAGCCCCATCAGCCAGAAGATTATCTACA
AAGAGAACGAGCGGTTCCAGTATAAGTGCAACATG
GGCTACGAGTACAGCGAGCGGGGAGATGCCGTGTG
TACAGAATCTGGATGGCGGCCTCTGCCTAGCTGCG
AGGAAAAGAGCTGCGACAACCCCTACATTCCCAAC
GGCGACTACAGCCCTCTGCGGATCAAACACAGAAC
CGGCGACGAGATCACCTACCAGTGCAGAAACGGCT
TTTACCCCGCCACCAGAGGCAATACCGCCAAGTGT
ACAAGCACCGGCTGGATCCCTGCTCCACGGTGCAC
ACTGAAA
Compound N:
Amino Acid (SEQ ID NO: 124):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEVECPPCPAPPVAGPSVFLFPPKPKDTL
MISRTPEVTCVVVDVSQEDPEVQFNWYVDGVEVHN
AKTKPREEQFNSTYRVVSVLTVLHQDWLNGKEYKC
KVSNKGLPSSIEKTISKAKGQPREPQVYTLPPSQE
EMTKNQVSLTCLVKGFYPSDIAVEWESNGQPENNY
KTTPPVLDSDGSFFLYSRLTVDKSRWQEGNVFSCS
VMHEALHNHYTQKSLSLSLGKGGGGAGGGGAGGGG
SEDCNELPPRRNTEILTGSWSDQTYPEGTQAIYKC
RPGYRSLGNVIMVCRKGEWVALNPLRKCQKRPCGH
PGDTPFGTFTLTGGNVFEYGVKAVYTCNEGYQLLG
EINYRECDTDGWTNDIPICEVVKCLPVTAPENGKI
VSSAMEPDREYHFGQAVRFVCNSGYKIEGDEEMHC
SDDGFWSKEKPKCVEISCKSPDVINGSPISQKIIY
KENERFQYKCNMGYEYSERGDAVCTESGWRPLPSC
EEKSCDNPYIPNGDYSPLRIKHRTGDEITYQCRNG
FYPATRGNTAKCTSTGWIPAPRCTLK
Nucleic Acid: (SEQ ID NO: 178):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTGTGCGAAGAGGTGGAATGTCC
TCCTTGTCCAGCTCCTCCTGTGGCCGGACCTTCCG
TGTTTCTGTTCCCTCCAAAGCCTAAGGACACCCTG
ATGATCAGCAGAACCCCTGAAGTGACCTGCGTGGT
GGTGGACGTTTCCCAAGAGGATCCCGAGGTGCAGT
TCAATTGGTACGTGGACGGCGTGGAAGTGCACAAC
GCCAAGACCAAGCCTAGAGAGGAACAGTTCAACAG
CACCTACAGAGTGGTGTCCGTGCTGACCGTTCTGC
ACCAGGACTGGCTGAATGGCAAAGAGTACAAGTGC
AAGGTGTCCAACAAGGGCCTGCCTAGCAGCATCGA
GAAAACCATCAGCAAGGCCAAGGGCCAGCCAAGAG
AACCCCAGGTTTACACCCTGCCTCCAAGCCAAGAG
GAAATGACCAAGAACCAGGTGTCCCTGACCTGCCT
GGTCAAGGGCTTCTACCCTAGCGACATTGCCGTGG
AATGGGAGAGCAATGGCCAGCCTGAGAACAACTAC
AAGACCACACCTCCTGTGCTGGACAGCGACGGCAG
CTTTTTTCTGTACTCCCGGCTGACCGTGGACAAGA
GCAGATGGCAAGAGGGCAACGTGTTCAGCTGCAGC
GTGATGCACGAAGCCCTGCACAACCACTACACCCA
GAAGTCTCTGAGCCTGTCTCTCGGAAAAGGCGGAG
GCGGAGCTGGTGGTGGCGGAGCAGGCGGCGGAGGA
TCTGAAGATTGCAATGAGCTGCCTCCTCGGCGGAA
CACCGAGATTCTTACCGGATCTTGGAGCGACCAGA
CATACCCTGAGGGCACCCAGGCCATCTACAAGTGT
AGACCTGGCTACAGATCCCTGGGCAATGTGATCAT
GGTCTGCCGGAAAGGCGAGTGGGTTGCCCTGAATC
CTCTGAGAAAGTGCCAGAAGAGGCCTTGCGGACAC
CCCGGCGATACACCTTTTGGCACATTCACCCTGAC
CGGCGGCAATGTGTTTGAGTATGGCGTGAAGGCCG
TGTACACCTGTAATGAGGGCTACCAGCTGCTGGGC
GAGATCAACTACAGAGAGTGTGATACCGACGGCTG
GACCAACGACATCCCTATCTGCGAGGTGGTCAAGT
GCCTGCCTGTGACAGCCCCTGAGAATGGCAAGATC
GTGTCCAGCGCCATGGAACCCGACAGAGAGTATCA
CTTTGGCCAGGCCGTCAGATTCGTGTGCAACTCCG
GATACAAGATCGAGGGCGACGAGGAAATGCACTGC
AGCGACGACGGCTTCTGGTCCAAAGAAAAGCCCAA
ATGCGTGGAAATCAGCTGCAAGTCCCCTGACGTGA
TCAACGGCAGCCCCATCAGCCAGAAGATTATCTAC
AAAGAGAACGAGCGGTTCCAGTATAAGTGCAACAT
GGGCTACGAGTACAGCGAGCGGGGAGATGCCGTGT
GTACAGAATCTGGATGGCGGCCTCTGCCTAGCTGC
GAGGAAAAGAGCTGCGACAACCCCTACATTCCCAA
CGGCGACTACAGCCCTCTGCGGATCAAACACAGAA
CCGGCGACGAGATCACCTACCAGTGCAGAAACGGC
TTTTACCCCGCCACCAGAGGCAATACCGCCAAGTG
TACAAGCACCGGCTGGATCCCAGCTCCTAGATGCA
CACTGAAGTGATGA
Compound O:
Amino Acid (SEQ ID NO: 125):
EVQLVESGGGLVKPGGSLRLSCAASGRPVSNYAAA
WFRQAPGKEREFVSAINWQKTATYADSVKGRFTIS
RDNAKNSLYLQMNSLRAEDTAVYYCAAVFRVVAPK
TQYDYDYWGQGTLVTVSSEDCNELPPRRNTEILTG
SWSDQTYPEGTQAIYKCRPGYRSLGNVIMVCRKGE
WVALNPLRKCQKRPCGHPGDTPFGTFTLTGGNVFE
YGVKAVYTCNEGYQLLGEINYRECDTDGWTNDIPI
CEVVKCLPVTAPENGKIVSSAMEPDREYHFGQAVR
FVCNSGYKIEGDEEMHCSDDGFWSKEKPKCVEISC
KSPDVINGSPISQKIIYKENERFQYKCNMGYEYSE
RGDAVCTESGWRPLPSCEEKSCDNPYIPNGDYSPL
RIKHRTGDEITYQCRNGFYPATRGNTAKCTSTGWI
PAPRCTLK
Nucleic Acid: (SEQ ID NO: 179):
GAGGTGCAGCTGGTTGAATCTGGCGGAGGACTTGT
GAAGCCTGGCGGCTCTCTGAGACTGTCTTGTGCTG
CTTCTGGCAGACCCGTGTCTAATTACGCCGCTGCC
TGGTTTAGACAGGCCCCTGGCAAAGAGAGAGAGTT
CGTCAGCGCCATCAACTGGCAGAAAACCGCCACAT
ACGCCGACAGCGTGAAGGGCAGATTCACCATCAGC
CGGGACAACGCCAAGAACAGCCTGTACCTGCAGAT
GAACTCCCTGAGAGCCGAGGACACCGCCGTGTATT
ATTGTGCCGCCGTGTTTAGAGTGGTGGCCCCTAAG
ACACAGTACGACTACGATTACTGGGGCCAGGGCAC
CCTGGTTACCGTGTCTAGCGAGGATTGCAACGAGC
TGCCTCCTCGGAGAAACACCGAGATCCTGACAGGC
TCTTGGAGCGACCAGACATACCCTGAGGGCACCCA
GGCCATCTACAAGTGCAGACCTGGCTACAGATCCC
TGGGCAACGTGATCATGGTCTGCAGAAAAGGCGAG
TGGGTCGCCCTGAATCCTCTGAGAAAGTGCCAGAA
GAGGCCTTGCGGACACCCTGGCGATACCCCTTTTG
GCACATTCACACTGACCGGCGGCAACGTGTTCGAG
TATGGCGTGAAGGCCGTGTACACCTGTAACGAGGG
ATATCAGCTGCTGGGCGAGATCAACTACAGAGAGT
GTGATACCGACGGCTGGACCAACGACATCCCTATC
TGCGAGGTGGTCAAGTGCCTGCCTGTGACAGCCCC
TGAGAATGGCAAGATCGTGTCCAGCGCCATGGAAC
CCGACAGAGAGTATCACTTTGGCCAGGCCGTCAGA
TTCGTGTGCAACAGCGGCTATAAGATCGAGGGCGA
CGAGGAAATGCACTGCAGCGACGACGGCTTCTGGT
CCAAAGAAAAGCCTAAGTGCGTGGAAATCAGCTGC
AAGAGCCCCGACGTGATCAACGGCAGCCCTATCAG
CCAGAAGATCATCTACAAAGAGAACGAGCGGTTCC
AGTACAAGTGTAACATGGGCTACGAGTACAGCGAG
AGGGGCGACGCCGTGTGTACAGAATCTGGATGGCG
ACCTCTGCCTAGCTGCGAGGAAAAGAGCTGCGACA
ACCCTTACATCCCCAACGGCGACTACAGCCCTCTG
CGGATTAAGCACAGAACCGGCGACGAGATCACCTA
CCAGTGCAGAAATGGCTTCTACCCCGCCACCAGAG
GCAATACCGCCAAGTGTACAAGCACCGGCTGGATC
CCTGCTCCTCGGTGCACACTGAAA
Compound P:
Amino Acid (SEQ ID NO: 126):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEEVQLVESGGGLVKPGGSLRLSCAASGR
PVSNYAAAWFRQAPGKEREFVSAINWQKTATYADS
VKGRFTISRDNAKNSLYLQMNSLRAEDTAVYYCAA
VFRVVAPKTQYDYDYVVGQGTLVTVSSEDCNELPP
RRNTEILTGSWSDQTYPEGTQAIYKCRPGYRSLGN
VIMVCRKGEWVALNPLRKCQKRPCGHPGDTPFGTF
TLTGGNVFEYGVKAVYTCNEGYQLLGEINYRECDT
DGWTNDIPICEVVKCLPVTAPENGKIVSSAMEPDR
EYHFGQAVRFVCNSGYKIEGDEEMHCSDDGFWSKE
KPKCVEISCKSPDVINGSPISQKIIYKENERFQYK
CNMGYEYSERGDAVCTESGWRPLPSCEEKSCDNPY
IPNGDYSPLRIKHRTGDEITYQCRNGFYPATRGNT
AKCTSTGWIPAPRCTLK
Nucleic Acid: (SEQ ID NO: 180):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTGTGTGAAGAAGAGGTGCAGCT
GGTTGAGTCTGGCGGCGGACTTGTGAAACCTGGCG
GAAGCCTGAGACTGTCTTGTGCTGCTTCTGGCAGA
CCCGTGTCTAATTACGCCGCTGCCTGGTTTAGACA
GGCCCCTGGCAAAGAGAGAGAGTTCGTCAGCGCCA
TCAACTGGCAGAAAACCGCCACATACGCCGACAGC
GTGAAAGGCAGATTCACCATCAGCCGGGACAACGC
CAAGAACAGCCTGTACCTGCAGATGAACTCCCTGA
GAGCCGAGGACACCGCCGTGTATTATTGTGCCGCC
GTGTTTAGAGTGGTGGCCCCTAAGACACAGTACGA
CTACGATTACTGGGGCCAGGGCACCCTGGTTACCG
TGTCTAGCGAGGATTGCAACGAGCTGCCTCCTCGG
AGAAACACCGAGATCCTGACCGGATCTTGGAGCGA
CCAGACATACCCTGAAGGCACCCAGGCCATCTACA
AGTGCAGACCTGGCTACAGATCCCTGGGCAATGTG
ATCATGGTCTGCCGGAAAGGCGAGTGGGTTGCCCT
GAATCCTCTGAGAAAGTGCCAGAAGAGGCCTTGCG
GACACCCTGGCGATACCCCTTTTGGCACATTCACC
CTGACCGGCGGCAATGTGTTTGAGTATGGCGTGAA
GGCCGTGTACACCTGTAATGAGGGCTACCAGCTGC
TGGGCGAGATCAACTACAGAGAGTGTGATACCGAC
GGCTGGACCAACGACATCCCTATCTGCGAGGTGGT
CAAGTGCCTGCCTGTGACAGCCCCTGAGAATGGCA
AGATCGTGTCCAGCGCCATGGAACCCGACAGAGAG
TATCACTTTGGCCAGGCCGTCAGATTCGTGTGCAA
CTCCGGATACAAGATCGAGGGCGACGAGGAAATGC
ACTGCAGCGACGACGGCTTCTGGTCCAAAGAAAAG
CCCAAATGCGTGGAAATCAGCTGCAAGTCCCCTGA
CGTGATCAACGGCAGCCCCATCAGCCAGAAGATTA
TCTACAAAGAGAACGAGCGGTTCCAGTACAAGTGT
AACATGGGCTACGAGTACAGCGAGAGGGGCGACGC
CGTGTGTACAGAATCTGGATGGCGACCTCTGCCTA
GCTGCGAGGAAAAGAGCTGCGACAACCCCTACATT
CCCAACGGCGACTACAGCCCTCTGCGGATCAAACA
CAGAACCGGCGACGAGATCACCTACCAGTGCAGAA
ATGGCTTCTACCCCGCCACCAGAGGCAATACCGCC
AAGTGTACAAGCACCGGCTGGATCCCAGCTCCTCG
GTGCACACTGAAA
Compound Q:
Amino Acid (SEQ ID NO: 127):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGSEVQLVESGGGLVKPGGSLRLSC
AASGRPVSNYAAAWFRQAPGKEREFVSAINWQKTA
TYADSVKGRFTISRDNAKNSLYLQMNSLRAEDTAV
YYCAAVFRVVAPKTQYDYDYVVGQGTLVTVSSGGG
GSEDCNELPPRRNTEILTGSWSDQTYPEGTQAIYK
CRPGYRSLGNVIMVCRKGEWVALNPLRKCQKRPCG
HPGDTPFGTFTLTGGNVFEYGVKAVYTCNEGYQLL
GEINYRECDTDGWTNDIPICEVVKCLPVTAPENGK
IVSSAMEPDREYHFGQAVRFVCNSGYKIEGDEEMH
CSDDGFWSKEKPKCVEISCKSPDVINGSPISQKII
YKENERFQYKCNMGYEYSERGDAVCTESGWRPLPS
CEEKSCDNPYIPNGDYSPLRIKHRTGDEITYQCRN
GFYPATRGNTAKCTSTGWIPAPRCTLK
Nucleic Acid: (SEQ ID NO: 181):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCGGAGG
CTCTGAAGTGCAGCTTGTTGAGTCTGGCGGCGGAC
TTGTGAAACCTGGCGGAAGCCTGAGACTGTCTTGT
GCTGCTTCTGGCAGACCCGTGTCTAATTACGCCGC
TGCCTGGTTTAGACAGGCCCCTGGCAAAGAGAGAG
AGTTCGTCAGCGCCATCAACTGGCAGAAAACCGCC
ACATACGCCGACAGCGTGAAAGGCAGATTCACCAT
CAGCCGGGACAACGCCAAGAACAGCCTGTACCTGC
AGATGAACTCCCTGAGAGCCGAGGACACCGCCGTG
TATTATTGTGCCGCCGTGTTTAGAGTGGTGGCCCC
TAAGACACAGTACGACTACGATTACTGGGGCCAGG
GCACCCTGGTTACAGTTTCTTCTGGCGGAGGCGGC
AGCGAGGATTGCAATGAACTGCCTCCTCGGCGGAA
CACCGAGATCTTGACAGGATCTTGGAGCGACCAGA
CATACCCTGAGGGCACCCAGGCCATCTACAAGTGC
AGACCTGGCTACAGATCCCTGGGCAATGTGATCAT
GGTCTGCCGGAAAGGCGAGTGGGTTGCCCTGAATC
CTCTGAGAAAGTGCCAGAAGAGGCCTTGCGGACAC
CCTGGCGATACCCCTTTTGGCACATTCACCCTGAC
CGGCGGCAATGTGTTTGAGTATGGCGTGAAGGCCG
TGTACACCTGTAATGAGGGCTACCAGCTGCTGGGC
GAGATCAACTACAGAGAGTGTGATACCGACGGCTG
GACCAACGACATCCCTATCTGCGAGGTGGTCAAGT
GCCTGCCTGTGACAGCCCCTGAGAATGGCAAGATC
GTGTCCAGCGCCATGGAACCCGACAGAGAGTATCA
CTTTGGCCAGGCCGTCAGATTCGTGTGCAACTCCG
GATACAAGATCGAGGGCGACGAGGAAATGCACTGC
AGCGACGACGGCTTCTGGTCCAAAGAAAAGCCCAA
ATGCGTGGAAATCAGCTGCAAGTCCCCTGACGTGA
TCAACGGCAGCCCCATCAGCCAGAAGATTATCTAC
AAAGAGAACGAGCGGTTCCAGTACAAGTGTAACAT
GGGCTACGAGTACAGCGAGAGGGGCGACGCCGTGT
GTACAGAATCTGGATGGCGACCTCTGCCTAGCTGC
GAGGAAAAGAGCTGCGACAACCCCTACATTCCCAA
CGGCGACTACAGCCCTCTGCGGATCAAACACAGAA
CCGGCGACGAGATCACCTACCAGTGCAGAAATGGC
TTCTACCCCGCCACCAGAGGCAATACCGCCAAGTG
TACAAGCACCGGCTGGATCCCAGCTCCTCGGTGCA
CACTGAAA
Compound R:
Amino Acid (SEQ ID NO: 128):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGSGGGGSEVQLVESGGGLVKPGGS
LRLSCAASGRPVSNYAAAWFRQAPGKEREFVSAIN
WQKTATYADSVKGRFTISRDNAKNSLYLQMNSLRA
EDTAVYYCAAVFRVVAPKTQYDYDYVVGQGTLVTV
SSGGGGSGGGGSEDCNELPPRRNTEILTGSWSDQT
YPEGTQAIYKCRPGYRSLGNVIMVCRKGEWVALNP
LRKCQKRPCGHPGDTPFGTFTLTGGNVFEYGVKAV
YTCNEGYQLLGEINYRECDTDGWTNDIPICEVVKC
LPVTAPENGKIVSSAMEPDREYHFGQAVRFVCNSG
YKIEGDEEMHCSDDGFWSKEKPKCVEISCKSPDVI
NGSPISQKIIYKENERFQYKCNMGYEYSERGDAVC
TESGWRPLPSCEEKSCDNPYIPNGDYSPLRIKHRT
GDEITYQCRNGFYPATRGNTAKCTSTGWIPAPRCT
LK
Nucleic Acid: (SEQ ID NO: 182):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCGGAGG
CTCTGGCGGCGGAGGCTCTGAAGTGCAGCTTGTTG
AGTCTGGCGGCGGACTTGTGAAACCTGGCGGAAGC
CTGAGACTGTCTTGTGCTGCTTCTGGCAGACCCGT
GTCTAATTACGCCGCTGCCTGGTTTAGACAGGCCC
CTGGCAAAGAGAGAGAGTTCGTCAGCGCCATCAAC
TGGCAGAAAACCGCCACATACGCCGACAGCGTGAA
AGGCAGATTCACCATCAGCCGGGACAACGCCAAGA
ACAGCCTGTACCTGCAGATGAACTCCCTGAGAGCC
GAGGACACCGCCGTGTATTATTGTGCCGCCGTGTT
TAGAGTGGTGGCCCCTAAGACACAGTACGACTACG
ATTACTGGGGCCAGGGCACCCTGGTTACAGTTTCT
TCTGGTGGCGGAGGATCTGGCGGAGGCGGATCTGA
AGATTGCAACGAGCTGCCTCCTCGGCGGAATACCG
AGATTCTGACCGGATCTTGGAGCGACCAGACATAC
CCTGAAGGCACCCAGGCCATCTACAAGTGCAGACC
TGGCTACAGATCCCTGGGCAATGTGATCATGGTCT
GCCGGAAAGGCGAGTGGGTTGCCCTGAATCCTCTG
AGAAAGTGCCAGAAGAGGCCTTGCGGACACCCTGG
CGATACCCCTTTTGGCACATTCACCCTGACCGGCG
GCAATGTGTTTGAGTATGGCGTGAAGGCCGTGTAC
ACCTGTAATGAGGGCTACCAGCTGCTGGGCGAGAT
CAACTACAGAGAGTGTGATACCGACGGCTGGACCA
ACGACATCCCTATCTGCGAGGTGGTCAAGTGCCTG
CCTGTGACAGCCCCTGAGAATGGCAAGATCGTGTC
CAGCGCCATGGAACCCGACAGAGAGTATCACTTTG
GCCAGGCCGTCAGATTCGTGTGCAACTCCGGATAC
AAGATCGAGGGCGACGAGGAAATGCACTGCAGCGA
CGACGGCTTCTGGTCCAAAGAAAAGCCCAAATGCG
TGGAAATCAGCTGCAAGTCCCCTGACGTGATCAAC
GGCAGCCCCATCAGCCAGAAGATTATCTACAAAGA
GAACGAGCGGTTCCAGTACAAGTGTAACATGGGCT
ACGAGTACAGCGAGAGGGGCGACGCCGTGTGTACA
GAATCTGGATGGCGACCTCTGCCTAGCTGCGAGGA
AAAGAGCTGCGACAACCCCTACATTCCCAACGGCG
ACTACAGCCCTCTGCGGATCAAACACAGAACCGGC
GACGAGATCACCTACCAGTGCAGAAATGGCTTCTA
CCCTGCCACCAGAGGCAACACCGCCAAGTGTACAA
GCACAGGCTGGATCCCCGCTCCTCGGTGCACACTG
AAA
Compound S:
Amino Acid (SEQ ID NO: 129):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGSGGGGSGGGGSEVQLVESGGGLV
KPGGSLRLSCAASGRPVSNYAAAWFRQAPGKEREF
VSAINWQKTATYADSVKGRFTISRDNAKNSLYLQM
NSLRAEDTAVYYCAAVFRVVAPKTQYDYDYVVGQG
TLVTVSSGGGGSGGGGSGGGGSEDCNELPPRRNTE
ILTGSWSDQTYPEGTQAIYKCRPGYRSLGNVIMVC
RKGEWVALNPLRKCQKRPCGHPGDTPFGTFTLTGG
NVFEYGVKAVYTCNEGYQLLGEINYRECDTDGWTN
DIPICEVVKCLPVTAPENGKIVSSAMEPDREYHFG
QAVRFVCNSGYKIEGDEEMHCSDDGFWSKEKPKCV
EISCKSPDVINGSPISQKIIYKENERFQYKCNMGY
EYSERGDAVCTESGWRPLPSCEEKSCDNPYIPNGD
YSPLRIKHRTGDEITYQCRNGFYPATRGNTAKCTS
TGWIPAPRCTLK
Nucleic Acid: (SEQ ID NO: 183):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCGGAGG
CTCTGGCGGCGGAGGCTCTGGCGGCGGAGGCTCTG
AAGTGCAGCTTGTTGAGTCTGGCGGCGGACTTGTG
AAACCTGGCGGAAGCCTGAGACTGTCTTGTGCTGC
TTCTGGCAGACCCGTGTCTAATTACGCCGCTGCCT
GGTTTAGACAGGCCCCTGGCAAAGAGAGAGAGTTC
GTCAGCGCCATCAACTGGCAGAAAACCGCCACATA
CGCCGACAGCGTGAAAGGCAGATTCACCATCAGCC
GGGACAACGCCAAGAACAGCCTGTACCTGCAGATG
AACTCCCTGAGAGCCGAGGACACCGCCGTGTATTA
TTGTGCCGCCGTGTTTAGAGTGGTGGCCCCTAAGA
CACAGTACGACTACGATTACTGGGGCCAGGGCACC
CTGGTTACAGTTTCTTCTGGTGGCGGAGGATCTGG
CGGAGGTGGAAGCGGAGGCGGTGGATCTGAAGATT
GCAACGAGCTGCCTCCTCGGCGGAATACCGAGATT
CTGACCGGATCTTGGAGCGACCAGACATACCCTGA
AGGCACCCAGGCCATCTACAAGTGCAGACCTGGCT
ACAGATCCCTGGGCAATGTGATCATGGTCTGCCGG
AAAGGCGAGTGGGTTGCCCTGAATCCTCTGAGAAA
GTGCCAGAAGAGGCCTTGCGGACACCCTGGCGATA
CCCCTTTTGGCACATTCACCCTGACCGGCGGCAAT
GTGTTTGAGTATGGCGTGAAGGCCGTGTACACCTG
TAATGAGGGCTACCAGCTGCTGGGCGAGATCAACT
ACAGAGAGTGTGATACCGACGGCTGGACCAACGAC
ATCCCTATCTGCGAGGTGGTCAAGTGCCTGCCTGT
GACAGCCCCTGAGAATGGCAAGATCGTGTCCAGCG
CCATGGAACCCGACAGAGAGTATCACTTTGGCCAG
GCCGTCAGATTCGTGTGCAACTCCGGATACAAGAT
CGAGGGCGACGAGGAAATGCACTGCAGCGACGACG
GCTTCTGGTCCAAAGAAAAGCCCAAATGCGTGGAA
ATCAGCTGCAAGTCCCCTGACGTGATCAACGGCAG
CCCCATCAGCCAGAAGATTATCTACAAAGAGAACG
AGCGGTTCCAGTACAAGTGTAACATGGGCTACGAG
TACAGCGAGAGGGGCGACGCCGTGTGTACAGAATC
TGGATGGCGACCTCTGCCTAGCTGCGAGGAAAAGA
GCTGCGACAACCCCTACATTCCCAACGGCGACTAC
AGCCCTCTGCGGATCAAACACAGAACCGGCGACGA
GATCACCTACCAGTGCAGAAATGGCTTCTACCCTG
CCACCAGAGGCAACACCGCCAAGTGTACAAGCACA
GGCTGGATCCCCGCTCCTCGGTGCACACTGAAA
Compound T:
Amino Acid (SEQ ID NO: 130):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGSGGGGSGGGGSGGGGSEVQLVES
GGGLVKPGGSLRLSCAASGRPVSNYAAAWFRQAPG
KEREFVSAINWQKTATYADSVKGRFTISRDNAKNS
LYLQMNSLRAEDTAVYYCAAVFRVVAPKTQYDYDY
WGQGTLVTVSSGGGGSGGGGSGGGGSGGGGSEDCN
ELPPRRNTEILTGSWSDQTYPEGTQAIYKCRPGYR
SLGNVIMVCRKGEWVALNPLRKCQKRPCGHPGDTP
FGTFTLTGGNVFEYGVKAVYTCNEGYQLLGEINYR
ECDTDGWTNDIPICEVVKCLPVTAPENGKIVSSAM
EPDREYHFGQAVRFVCNSGYKIEGDEEMHCSDDGF
WSKEKPKCVEISCKSPDVINGSPISQKIIYKENER
FQYKCNMGYEYSERGDAVCTESGWRPLPSCEEKSC
DNPYIPNGDYSPLRIKHRTGDEITYQCRNGFYPAT
RGNTAKCTSTGWIPAPRCTLK
Nucleic Acid: (SEQ ID NO: 184):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTTTGTGAAGAAGGCGGCGGAGG
CTCTGGCGGCGGAGGCTCTGGCGGCGGAGGCTCTG
GCGGCGGAGGCTCTGAAGTGCAGCTTGTTGAGTCT
GGCGGCGGACTTGTGAAACCTGGCGGAAGCCTGAG
ACTGTCTTGTGCTGCTTCTGGCAGACCCGTGTCTA
ATTACGCCGCTGCCTGGTTTAGACAGGCCCCTGGC
AAAGAGAGAGAGTTCGTCAGCGCCATCAACTGGCA
GAAAACCGCCACATACGCCGACAGCGTGAAAGGCA
GATTCACCATCAGCCGGGACAACGCCAAGAACAGC
CTGTACCTGCAGATGAACTCCCTGAGAGCCGAGGA
CACCGCCGTGTATTATTGTGCCGCCGTGTTTAGAG
TGGTGGCCCCTAAGACACAGTACGACTACGATTAC
TGGGGCCAGGGCACCCTGGTTACAGTTTCTTCTGG
TGGCGGAGGATCTGGCGGAGGTGGAAGCGGAGGCG
GTGGTAGTGGCGGTGGTGGATCTGAGGATTGCAAC
GAGCTGCCTCCTCGGAGAAACACCGAGATCCTGAC
CGGATCTTGGAGCGACCAGACATACCCTGAAGGCA
CCCAGGCCATCTACAAGTGCAGACCTGGCTACAGA
TCCCTGGGCAATGTGATCATGGTCTGCCGGAAAGG
CGAGTGGGTTGCCCTGAATCCTCTGAGAAAGTGCC
AGAAGAGGCCTTGCGGACACCCTGGCGATACCCCT
TTTGGCACATTCACCCTGACCGGCGGCAATGTGTT
TGAGTATGGCGTGAAGGCCGTGTACACCTGTAATG
AGGGCTACCAGCTGCTGGGCGAGATCAACTACAGA
GAGTGTGATACCGACGGCTGGACCAACGACATCCC
TATCTGCGAGGTGGTCAAGTGCCTGCCTGTGACAG
CCCCTGAGAATGGCAAGATCGTGTCCAGCGCCATG
GAACCCGACAGAGAGTATCACTTTGGCCAGGCCGT
CAGATTCGTGTGCAACTCCGGATACAAGATCGAGG
GCGACGAGGAAATGCACTGCAGCGACGACGGCTTC
TGGTCCAAAGAAAAGCCCAAATGCGTGGAAATCAG
CTGCAAGTCCCCTGACGTGATCAACGGCAGCCCCA
TCAGCCAGAAGATTATCTACAAAGAGAACGAGCGG
TTCCAGTACAAGTGTAACATGGGCTACGAGTACAG
CGAGAGGGGCGACGCCGTGTGTACAGAATCTGGAT
GGCGACCTCTGCCTAGCTGCGAGGAAAAGAGCTGC
GACAACCCCTACATTCCCAACGGCGACTACAGCCC
TCTGCGGATCAAACACAGAACCGGCGACGAGATCA
CCTACCAGTGCAGAAATGGCTTCTACCCTGCCACC
AGAGGCAACACCGCCAAGTGTACAAGCACAGGCTG
GATCCCCGCTCCTCGGTGCACACTGAAA
Compound U:
Amino Acid (SEQ ID NO: 131):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEEVQLVESGGGLVKPGGSLRLSCAASGR
PVSNYAAAWFRQAPGKEREFVSAINWQKTATYADS
VKGRFTISRDNAKNSLYLQMNSLRAEDTAVYYCAA
VFRVVAPKTQYDYDYVVGQGTLVTVSSEDCNELPP
RRNTEILTGSWSDQTYPEGTQAIYKCRPGYRSLGN
VIMVCRKGEWVALNPLRKCQKRPCGHPGDTPFGTF
TLTGGNVFEYGVKAVYTCNEGYQLLGEINYRECDT
DGWTNDIPICEVVKCLPVTAPENGKIVSSAMEPDR
EYHFGQAVRFVCNSGYKIEGDEEMHCSDDGFWSKE
KPKCVEISCKSPDVINGSPISQKIIYKENERFQYK
CNMGYEYSERGDAVCTESGWRPLPSCEEKSCDNPY
IPNGDYSPLRIKHRTGDEITYQCRNGFYPATRGNT
AKCTSTGWIPAPRCTLKHHHHHH
Nucleic Acid: (SEQ ID NO: 185):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCAACAAGAGCGTGTGGTGCCAGGCCAACAATAT
GTGGGGCCCTACCAGACTGCCCACCTGTGTGTCTG
TGTTCCCTCTGGAATGCCCCGCTCTGCCCATGATC
CACAATGGCCACCACACAAGCGAGAACGTGGGATC
TATTGCCCCTGGCCTGAGCGTGACCTACAGCTGTG
AATCTGGCTATCTGCTCGTGGGCGAGAAGATCATC
AATTGCCTGAGCAGCGGCAAGTGGTCCGCTGTGCC
TCCTACATGTGAAGAGGCCAGATGCAAGAGCCTGG
GCAGATTCCCCAACGGCAAAGTGAAAGAGCCTCCA
ATCCTGAGAGTGGGCGTGACCGCCAACTTCTTCTG
TGACGAGGGCTATAGACTGCAGGGCCCTCCTAGCT
CTAGATGCGTTATCGCTGGACAGGGCGTCGCCTGG
ACAAAGATGCCTGTGTGTGAAGAAGAGGTGCAGCT
GGTTGAGTCTGGCGGCGGACTTGTGAAACCTGGCG
GAAGCCTGAGACTGTCTTGTGCTGCTTCTGGCAGA
CCCGTGTCTAATTACGCCGCTGCCTGGTTTAGACA
GGCCCCTGGCAAAGAGAGAGAGTTCGTCAGCGCCA
TCAACTGGCAGAAAACCGCCACATACGCCGACAGC
GTGAAAGGCAGATTCACCATCAGCCGGGACAACGC
CAAGAACAGCCTGTACCTGCAGATGAACTCCCTGA
GAGCCGAGGACACCGCCGTGTATTATTGTGCCGCC
GTGTTTAGAGTGGTGGCCCCTAAGACACAGTACGA
CTACGATTACTGGGGCCAGGGCACCCTGGTTACCG
TGTCTAGCGAGGATTGCAACGAGCTGCCTCCTCGG
AGAAACACCGAGATCCTGACCGGATCTTGGAGCGA
CCAGACATACCCTGAAGGCACCCAGGCCATCTACA
AGTGCAGACCTGGCTACAGATCCCTGGGCAATGTG
ATCATGGTCTGCCGGAAAGGCGAGTGGGTTGCCCT
GAATCCTCTGAGAAAGTGCCAGAAGAGGCCTTGCG
GACACCCTGGCGATACCCCTTTTGGCACATTCACC
CTGACCGGCGGCAATGTGTTTGAGTATGGCGTGAA
GGCCGTGTACACCTGTAATGAGGGCTACCAGCTGC
TGGGCGAGATCAACTACAGAGAGTGTGATACCGAC
GGCTGGACCAACGACATCCCTATCTGCGAGGTGGT
CAAGTGCCTGCCTGTGACAGCCCCTGAGAATGGCA
AGATCGTGTCCAGCGCCATGGAACCCGACAGAGAG
TATCACTTTGGCCAGGCCGTCAGATTCGTGTGCAA
CTCCGGATACAAGATCGAGGGCGACGAGGAAATGC
ACTGCAGCGACGACGGCTTCTGGTCCAAAGAAAAG
CCCAAATGCGTGGAAATCAGCTGCAAGTCCCCTGA
CGTGATCAACGGCAGCCCCATCAGCCAGAAGATTA
TCTACAAAGAGAACGAGCGGTTCCAGTACAAGTGT
AACATGGGCTACGAGTACAGCGAGAGGGGCGACGC
CGTGTGTACAGAATCTGGATGGCGACCTCTGCCTA
GCTGCGAGGAAAAGAGCTGCGACAACCCCTACATT
CCCAACGGCGACTACAGCCCTCTGCGGATCAAACA
CAGAACCGGCGACGAGATCACCTACCAGTGCAGAA
ATGGCTTCTACCCCGCCACCAGAGGCAATACCGCC
AAGTGTACAAGCACCGGCTGGATCCCAGCTCCTAG
ATGCACACTGAAGCACCACCACCATCACCAC
Compound X:
Amino Acid (SEQ ID NO: 132):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GNKSVWCQANNMWGPTRLPTCVSVFPLECPALPMI
HNGHHTSENVGSIAPGLSVTYSCESGYLLVGEKII
NCLSSGKWSAVPPTCEEARCKSLGRFPNGKVKEPP
ILRVGVTANFFCDEGYRLQGPPSSRCVIAGQGVAW
TKMPVCEEGGGGAGGGGAGGGGSVECPPCPAPPVA
GPSVFLFPPKPKDTLMISRTPEVTCVVVDVSQEDP
EVQFNWYVDGVEVHNAKTKPREEQFNSTYRVVSVL
TVLHQDWLNGKEYKCKVSNKGLPSSIEKTISKAKG
QPREPQVYTLPPSQEEMTKNQVSLTCLVKGFYPSD
IAVEWESNGQPENNYKTTPPVLDSDGSFFLYSRLT
VDKSRWQEGNVFSCSVMHEALHNHYTQKSLSLSLG
KGGGGAGGGGAGGGGSEDCNELPPRRNTEILTGSW
SDQTYPEGTQAIYKCRPGYRSLGNVIMVCRKGEWV
ALNPLRKCQKRPCGHPGDTPFGTFTLTGGNVFEYG
VKAVYTCNEGYQLLGEINYRECDTDGWTNDIPICE
VVKCLPVTAPENGKIVSSAMEPDREYHFGQAVRFV
CNSGYKIEGDEEMHCSDDGFWSKEKPKCVEISCKS
PDVINGSPISQKIIYKENERFQYKCNMGYEYSERG
DAVCTESGWRPLPSCEEKSCDNPYIPNGDYSPLRI
KHRTGDEITYQCRNGFYPATRGNTAKCTSTGWIPA
PRCTLK
Nucleic Acid: (SEQ ID NO: 188):
ATCAGCTGCGGCAGCCCCCCCCCCATCCTGAACGG
CCGGATCAGCTACTACAGCACCCCCATCGCCGTGG
GCACCGTGATCCGGTACAGCTGCAGCGGCACCTTC
CGGCTGATCGGCGAGAAGAGCCTGCTGTGCATCAC
CAAGGACAAGGTGGACGGCACCTGGGACAAGCCCG
CCCCCAAGTGCGAGTACTTCAACAAGTACAGCAGC
TGCCCCGAGCCCATCGTGCCCGGCGGCTACAAGAT
CCGGGGCAGCACCCCCTACCGGCACGGCGACAGCG
TGACCTTCGCCTGCAAGACCAACTTCAGCATGAAC
GGCAACAAGAGCGTGTGGTGCCAGGCCAACAACAT
GTGGGGCCCCACCCGGCTGCCCACCTGCGTGAGCG
TGTTCCCCCTGGAGTGCCCCGCCCTGCCCATGATC
CACAACGGCCACCACACCAGCGAGAACGTGGGCAG
CATCGCCCCCGGCCTGAGCGTGACCTACAGCTGCG
AGAGCGGCTACCTGCTGGTGGGCGAGAAGATCATC
AACTGCCTGAGCAGCGGCAAGTGGAGCGCCGTGCC
CCCCACCTGCGAGGAGGCCCGGTGCAAGAGCCTGG
GCCGGTTCCCCAACGGCAAGGTGAAGGAGCCCCCC
ATCCTGCGGGTGGGCGTGACCGCCAACTTCTTCTG
CGACGAGGGCTACCGGCTGCAGGGCCCCCCCAGCA
GCCGGTGCGTGATCGCCGGCCAGGGCGTGGCCTGG
ACCAAGATGCCCGTGTGCGAGGAGGGCGGCGGCGG
CGCCGGCGGCGGCGGCGCCGGCGGCGGCGGCAGCG
TGGAGTGCCCCCCCTGCCCCGCCCCCCCCGTGGCC
GGCCCCAGCGTGTTCCTGTTCCCCCCCAAGCCCAA
GGACACCCTGATGATCAGCCGGACCCCCGAGGTGA
CCTGCGTGGTGGTGGACGTGAGCCAGGAGGACCCC
GAGGTGCAGTTCAACTGGTACGTGGACGGCGTGGA
GGTGCACAACGCCAAGACCAAGCCCCGGGAGGAGC
AGTTCAACAGCACCTACCGGGTGGTGAGCGTGCTG
ACCGTGCTGCACCAGGACTGGCTGAACGGCAAGGA
GTACAAGTGCAAGGTGAGCAACAAGGGCCTGCCCA
GCAGCATCGAGAAGACCATCAGCAAGGCCAAGGGC
CAGCCCCGGGAGCCCCAGGTGTACACCCTGCCCCC
CAGCCAGGAGGAGATGACCAAGAACCAGGTGAGCC
TGACCTGCCTGGTGAAGGGCTTCTACCCCAGCGAC
ATCGCCGTGGAGTGGGAGAGCAACGGCCAGCCCGA
GAACAACTACAAGACCACCCCCCCCGTGCTGGACA
GCGACGGCAGCTTCTTCCTGTACAGCCGGCTGACC
GTGGACAAGAGCCGGTGGCAGGAGGGCAACGTGTT
CAGCTGCAGCGTGATGCACGAGGCCCTGCACAACC
ACTACACCCAGAAGAGCCTGAGCCTGAGCCTGGGC
AAGGGCGGCGGCGGCGCCGGCGGCGGCGGCGCCGG
CGGCGGCGGCAGCGAGGACTGCAACGAGCTGCCCC
CCCGGCGGAACACCGAGATCCTGACCGGCAGCTGG
AGCGACCAGACCTACCCCGAGGGCACCCAGGCCAT
CTACAAGTGCCGGCCCGGCTACCGGAGCCTGGGCA
ACGTGATCATGGTGTGCCGGAAGGGCGAGTGGGTG
GCCCTGAACCCCCTGCGGAAGTGCCAGAAGCGGCC
CTGCGGCCACCCCGGCGACACCCCCTTCGGCACCT
TCACCCTGACCGGCGGCAACGTGTTCGAGTACGGC
GTGAAGGCCGTGTACACCTGCAACGAGGGCTACCA
GCTGCTGGGCGAGATCAACTACCGGGAGTGCGACA
CCGACGGCTGGACCAACGACATCCCCATCTGCGAG
GTGGTGAAGTGCCTGCCCGTGACCGCCCCCGAGAA
CGGCAAGATCGTGAGCAGCGCCATGGAGCCCGACC
GGGAGTACCACTTCGGCCAGGCCGTGCGGTTCGTG
TGCAACAGCGGCTACAAGATCGAGGGCGACGAGGA
GATGCACTGCAGCGACGACGGCTTCTGGAGCAAGG
AGAAGCCCAAGTGCGTGGAGATCAGCTGCAAGAGC
CCCGACGTGATCAACGGCAGCCCCATCAGCCAGAA
GATCATCTACAAGGAGAACGAGCGGTTCCAGTACA
AGTGCAACATGGGCTACGAGTACAGCGAGCGGGGC
GACGCCGTGTGCACCGAGAGCGGCTGGCGGCCCCT
GCCCAGCTGCGAGGAGAAGAGCTGCGACAACCCCT
ACATCCCCAACGGCGACTACAGCCCCCTGCGGATC
AAGCACCGGACCGGCGACGAGATCACCTACCAGTG
CCGGAACGGCTTCTACCCCGCCACCCGGGGCAACA
CCGCCAAGTGCACCAGCACCGGCTGGATCCCCGCC
CCCCGGTGCACCCTGAAGTGATGA
Compound Y:
Amino Acid (SEQ ID NO: 144):
GKCGPPPPIDNGDITSFPLSVYAPASSVEYQCQNL
YQLEGNKRITCRNGQWSEPPKCLHSREIMENYNIA
LRWTAKQKLYSRTGESVEFVCKRGYRLSSRSHTLR
TTCWDGKLEYPTCAKRVECPPCPAPPVAGPSVFLF
PPKPKDTLMISRTPEVTCVVVDVSQEDPEVQFNWY
VDGVEVHNAKTKPREEQFNSTYRVVSVLTVLHQDW
LNGKEYKCKVSNKGLPSSIEKTISKAKGQPREPQV
YTLPPSQEEMTKNQVSLTCLVKGFYPSDIAVEWES
NGQPENNYKTTPPVLDSDGSFFLYSRLTVDKSRWQ
EGNVFSCSVMHEALHNHYTQKSLSLSLGKEDCNEL
PPRRNTEILTGSWSDQTYPEGTQAIYKCRPGYRSL
GNVIMVCRKGEWVALNPLRKCQKRPCGHPGDTPFG
TFTLTGGNVFEYGVKAVYTCNEGYQLLGEINYREC
DTDGWTNDIPICEVVKCLPVTAPENGKIVSSAMEP
DREYHFGQAVRFVCNSGYKIEGDEEMHCSDDGFWS
KEKPKCVEISCKSPDVINGSPISQKIIYKENERFQ
YKCNMGYEYSERGDAVCTESGWRPLPSCEEKSCDN
PYIPNGDYSPLRIKHRTGDEITYQCRNGFYPATRG
NTAKCTSTGWIPAPRCTLK
Nucleic Acid: (SEQ ID NO: 189):
GGAAAATGTGGCCCTCCTCCTCCTATCGACAACGG
CGACATTACCAGCTTTCCACTGTCTGTGTACGCCC
CTGCCAGCAGCGTGGAATACCAGTGCCAGAACCTG
TACCAGCTGGAAGGCAACAAGCGGATCACCTGTAG
AAACGGCCAGTGGTCCGAGCCTCCTAAGTGTCTGC
ACCCTTGCGTGATCAGCCGCGAGATCATGGAAAAC
TACAATATCGCCCTGCGGTGGACCGCCAAGCAGAA
GCTGTATAGCAGAACCGGCGAGTCCGTGGAATTCG
TGTGCAAGAGAGGCTACCGGCTGAGCAGCAGAAGC
CACACACTGAGAACCACCTGTTGGGACGGCAAGCT
GGAATACCCTACCTGTGCCAAGAGGGTCGAGTGCC
CTCCTTGTCCAGCTCCTCCTGTTGCCGGACCTAGC
GTGTTCCTGTTTCCTCCAAAGCCTAAGGACACCCT
GATGATCAGCAGAACCCCTGAAGTGACCTGCGTGG
TGGTGGACGTTTCCCAAGAGGATCCCGAGGTGCAG
TTCAATTGGTACGTGGACGGCGTGGAAGTGCACAA
CGCCAAGACCAAGCCTAGAGAGGAACAGTTCAACA
GCACCTACAGAGTGGTGTCCGTGCTGACCGTGCTG
CACCAGGATTGGCTGAACGGCAAAGAGTACAAGTG
CAAGGTGTCCAACAAGGGCCTGCCTAGCAGCATCG
AGAAAACCATCAGCAAGGCCAAGGGCCAGCCAAGA
GAACCCCAGGTTTACACCCTGCCTCCAAGCCAAGA
GGAAATGACCAAGAACCAGGTGTCCCTGACCTGCC
TGGTCAAGGGCTTCTACCCTTCCGATATCGCCGTG
GAATGGGAGAGCAATGGCCAGCCTGAGAACAACTA
CAAGACCACACCTCCTGTGCTGGACAGCGACGGCA
GCTTTTTTCTGTACTCCCGCCTGACCGTGGACAAG
AGCAGATGGCAAGAGGGCAACGTGTTCAGCTGCTC
TGTGATGCACGAGGCCCTGCACAACCACTACACCC
AGAAGTCTCTGAGCCTGAGCCTGGGCAAAGAGGAC
TGTAACGAGCTGCCTCCTCGGCGGAATACCGAGAT
TCTGACAGGCTCTTGGAGCGACCAGACATACCCTG
AGGGCACCCAGGCCATCTACAAGTGTAGACCTGGC
TACAGATCCCTGGGCAATGTGATCATGGTCTGCCG
GAAAGGCGAGTGGGTTGCCCTGAATCCTCTGCGGA
AGTGTCAGAAGAGGCCTTGCGGACATCCTGGCGAT
ACCCCTTTCGGCACATTCACCCTGACCGGCGGCAA
TGTGTTTGAGTATGGCGTGAAGGCCGTGTACACAT
GCAACGAGGGATATCAGCTGCTGGGCGAGATCAAC
TACAGAGAGTGTGATACCGACGGCTGGACCAACGA
CATCCCTATCTGCGAGGTTGTGAAGTGCCTGCCTG
TGACAGCCCCTGAGAATGGCAAGATCGTGTCCAGC
GCCATGGAACCCGACAGAGAGTATCACTTTGGCCA
GGCCGTCAGATTCGTGTGTAACTCCGGCTACAAGA
TCGAGGGCGACGAGGAAATGCACTGCAGCGACGAC
GGCTTCTGGTCCAAAGAAAAGCCCAAATGCGTGGA
AATCAGCTGCAAGAGCCCCGACGTGATCAACGGCA
GCCCTATCAGCCAGAAGATCATCTACAAAGAGAAC
GAGCGGTTCCAGTATAAGTGCAACATGGGCTACGA
GTACAGCGAGCGGGGAGATGCCGTGTGTACAGAAT
CTGGATGGCGGCCTCTGCCTAGCTGCGAGGAAAAG
AGCTGCGACAACCCTTACATCCCCAACGGCGATTA
CAGCCCACTGCGGATCAAACACAGAACAGGCGACG
AGATCACCTACCAGTGTCGGAACGGCTTTTACCCC
GCCACAAGAGGCAATACCGCCAAGTGTACAAGCAC
CGGCTGGATCCCTGCTCCTCGGTGCACACTGAAG
Compound Z:
Amino Acid (SEQ ID NO: 145):
EDCNELPPRRNTEILTGSWSDQTYPEGTQAIYKCR
PGYRSLGNVIMVCRKGEWVALNPLRKCQKRPCGHP
GDTPFGTFTLTGGNVFEYGVKAVYTCNEGYQLLGE
INYRECDTDGWTNDIPICEVVKCLPVTAPENGKIV
SSAMEPDREYHFGQAVRFVCNSGYKIEGDEEMHCS
DDGFWSKEKPKCVEISCKSPDVINGSPISQKIIYK
ENERFQYKCNMGYEYSERGDAVCTESGWRPLPSCE
EKSCDNPYIPNGDYSPLRIKHRTGDEITYQCRNGF
YPATRGNTAKCTSTGWIPAPRCTLKVECPPCPAPP
VAGPSVFLFPPKPKDTLMISRTPEVTCVVVDVSQE
DPEVQFNWYVDGVEVHNAKTKPREEQFNSTYRVVS
VLTVLHQDWLNGKEYKCKVSNKGLPSSIEKTISKA
KGQPREPQVYTLPPSQEEMTKNQVSLTCLVKGFYP
SDIAVEWESNGQPENNYKTTPPVLDSDGSFFLYSR
LTVDKSRWQEGNVFSCSVMHEALHNHYTQKSLSLS
LGKGKCGPPPPIDNGDITSFPLSVYAPASSVEYQC
QNLYQLEGNKRITCRNGQWSEPPKCLHSREIMENY
NIALRWTAKQKLYSRTGESVEFVCKRGYRLSSRSH
TLRTTCWDGKLEYPTCAKR
Nucleic Acid: (SEQ ID NO: 190):
GAGGATTGCAATGAGCTGCCTCCTCGGAGAAACAC
CGAGATCCTGACAGGCTCTTGGAGCGACCAGACAT
ACCCTGAGGGCACCCAGGCCATCTACAAGTGCAGA
CCTGGCTACAGATCCCTGGGCAACGTGATCATGGT
CTGCAGAAAAGGCGAGTGGGTCGCCCTGAATCCTC
TGAGAAAGTGCCAGAAGAGGCCTTGCGGACACCCT
GGCGATACCCCTTTTGGCACATTCACACTGACCGG
CGGCAACGTGTTCGAGTATGGCGTGAAGGCCGTGT
ACACCTGTAACGAGGGATATCAGCTGCTGGGCGAG
ATCAACTACAGAGAGTGTGATACCGACGGCTGGAC
CAACGACATCCCTATCTGCGAGGTGGTCAAGTGCC
TGCCTGTGACAGCCCCTGAGAATGGCAAGATCGTG
TCCAGCGCCATGGAACCCGACAGAGAGTATCACTT
TGGCCAGGCCGTCAGATTCGTGTGCAACAGCGGCT
ATAAGATCGAGGGCGACGAGGAAATGCACTGCAGC
GACGACGGCTTCTGGTCCAAAGAAAAGCCTAAGTG
CGTGGAAATCAGCTGCAAGAGCCCCGACGTGATCA
ACGGCAGCCCTATCAGCCAGAAGATCATCTACAAA
GAGAACGAGCGGTTCCAGTACAAGTGTAACATGGG
CTACGAGTACAGCGAGAGGGGCGACGCCGTGTGTA
CAGAATCTGGATGGCGACCTCTGCCTAGCTGCGAG
GAAAAGAGCTGCGACAACCCTTACATCCCCAACGG
CGACTACAGCCCTCTGCGGATTAAGCACAGAACCG
GCGACGAGATCACCTACCAGTGCAGAAATGGCTTC
TACCCCGCCACCAGAGGCAATACCGCCAAGTGTAC
AAGCACCGGCTGGATCCCTGCTCCTAGATGCACCC
TGAAGGTGGAATGCCCTCCTTGTCCTGCTCCTCCA
GTGGCCGGACCTTCCGTGTTTCTGTTCCCACCTAA
GCCTAAGGACACACTGATGATCAGCAGAACCCCTG
AAGTGACCTGCGTGGTGGTGGACGTTTCCCAAGAG
GATCCCGAGGTGCAGTTCAATTGGTACGTGGACGG
CGTGGAAGTGCACAACGCCAAGACCAAGCCTAGAG
AGGAACAGTTCAACAGCACCTACAGAGTGGTGTCC
GTGCTGACCGTGCTGCACCAGGATTGGCTGAACGG
CAAAGAGTATAAGTGCAAGGTGTCCAACAAGGGCC
TGCCTAGCAGCATCGAGAAAACCATCAGCAAGGCC
AAGGGCCAGCCAAGAGAGCCTCAGGTTTACACCCT
GCCTCCAAGCCAAGAGGAAATGACCAAGAACCAGG
TGTCCCTGACCTGCCTGGTCAAGGGCTTTTACCCT
TCCGATATCGCCGTGGAATGGGAGAGCAATGGCCA
GCCTGAGAACAACTACAAGACCACACCTCCTGTGC
TGGACAGCGACGGCAGCTTTTTTCTGTACTCCCGC
CTGACCGTGGACAAGAGCAGATGGCAAGAGGGCAA
TGTGTTCAGCTGCAGCGTGATGCACGAGGCCCTGC
ACAACCACTACACCCAGAAGTCTCTGAGCCTGAGC
CTCGGCAAGGGAAAGTGTGGACCTCCTCCTCCTAT
CGACAATGGCGACATCACCAGCTTTCCACTGTCTG
TGTACGCCCCTGCCAGCAGCGTTGAGTATCAGTGT
CAGAACCTGTACCAGCTGGAAGGCAACAAGCGGAT
CACCTGTAGAAACGGCCAGTGGTCCGAGCCTCCTA
AGTGTCTGCACCCTTGCGTGATCAGCCGCGAGATC
ATGGAAAACTACAATATCGCCCTGCGGTGGACCGC
CAAGCAGAAGCTGTATTCTAGAACAGGCGAGAGCG
TCGAGTTTGTGTGCAAGAGAGGCTACCGGCTGAGC
AGCAGAAGCCACACACTGAGAACCACCTGTTGGGA
CGGCAAGCTGGAATACCCTACCTGCGCCAAGAGA
Compound AA:
Amino Acid (SEQ ID NO: 146):
VECPPCPAPPVAGPSVFLFPPKPKDTLMISRTPEV
TCVVVDVSQEDPEVQFNWYVDGVEVHNAKTKPREE
QFNSTYRVVSVLTVLHQDWLNGKEYKCKVSNKGLP
SSIEKTISKAKGQPREPQVYTLPPSQEEMTKNQVS
LTCLVKGFYPSDIAVEWESNGQPENNYKTTPPVLD
SDGSFFLYSRLTVDKSRWQEGNVFSCSVMHEALHN
HYTQKSLSLSLGKGGGGAGGGGAGGGGSEDCNELP
PRRNTEILTGSWSDQTYPEGTQAIYKCRPGYRSLG
NVIMVCRKGEWVALNPLRKCQKRPCGHPGDTPFGT
FTLTGGNVFEYGVKAVYTCNEGYQLLGEINYRECD
TDGWTNDIPICEVVKCLPVTAPENGKIVSSAMEPD
REYHFGQAVRFVCNSGYKIEGDEEMHCSDDGFWSK
EKPKCVEISCKSPDVINGSPISQKIIYKENERFQY
KCNMGYEYSERGDAVCTESGWRPLPSCEEKSCDNP
YIPNGDYSPLRIKHRTGDEITYQCRNGFYPATRGN
TAKCTSTGWIPAPRCTLK
Nucleic Acid: (SEQ ID NO: 191):
GTGGAATGCCCTCCATGTCCTGCTCCTCCAGTGGC
CGGACCTTCCGTGTTTCTGTTCCCTCCAAAGCCTA
AGGACACCCTGATGATCAGCAGAACCCCTGAAGTG
ACCTGCGTGGTGGTGGACGTTTCCCAAGAGGATCC
CGAGGTGCAGTTCAATTGGTACGTGGACGGCGTGG
AAGTGCACAACGCCAAGACCAAGCCTAGAGAGGAA
CAGTTCAACAGCACCTACAGAGTGGTGTCCGTGCT
GACCGTGCTGCACCAGGATTGGCTGAACGGCAAAG
AGTACAAGTGCAAGGTGTCCAACAAGGGCCTGCCT
AGCAGCATCGAGAAAACCATCAGCAAGGCCAAGGG
CCAGCCAAGAGAACCCCAGGTTTACACCCTGCCTC
CAAGCCAAGAGGAAATGACCAAGAACCAGGTGTCC
CTGACCTGCCTGGTCAAGGGCTTCTACCCTTCCGA
TATCGCTGTGGAATGGGAGAGCAACGGCCAGCCTG
AGAACAACTACAAGACCACACCTCCTGTGCTGGAC
AGCGACGGCAGCTTTTTTCTGTACTCCCGCCTGAC
CGTGGACAAGAGCAGATGGCAAGAGGGCAACGTGT
TCAGCTGCTCTGTGATGCACGAGGCCCTGCACAAC
CACTACACCCAGAAGTCTCTGAGCCTGTCTCTCGG
AAAAGGCGGAGGCGGAGCTGGTGGTGGCGGAGCAG
GCGGCGGAGGATCTGAAGATTGCAATGAGCTGCCT
CCTCGGCGGAACACAGAGATCTTGACAGGCTCTTG
GAGCGACCAGACATACCCTGAGGGCACCCAGGCCA
TCTACAAGTGTAGACCTGGCTACCGCAGCCTGGGC
AATGTGATCATGGTCTGCAGAAAAGGCGAGTGGGT
CGCCCTGAATCCTCTGAGAAAGTGCCAGAAGAGGC
CTTGCGGACACCCCGGCGATACACCTTTTGGCACA
TTCACCCTGACCGGCGGCAATGTGTTTGAGTATGG
CGTGAAGGCCGTGTACACCTGTAACGAGGGATATC
AGCTGCTGGGCGAGATCAACTACAGAGAGTGTGAT
ACCGACGGCTGGACCAACGACATCCCTATCTGCGA
GGTGGTCAAGTGCCTGCCTGTGACAGCCCCTGAGA
ATGGCAAGATCGTGTCCAGCGCCATGGAACCCGAC
AGAGAGTATCACTTTGGCCAGGCCGTCAGATTCGT
GTGCAACAGCGGCTATAAGATCGAGGGCGACGAGG
AAATGCACTGCAGCGACGACGGCTTCTGGTCCAAA
GAAAAGCCCAAATGCGTGGAAATCAGCTGCAAGAG
CCCCGACGTGATCAACGGCAGCCCTATCAGCCAGA
AGATCATCTACAAAGAGAACGAGCGGTTCCAGTAT
AAGTGCAACATGGGCTACGAGTACAGCGAGCGGGG
AGATGCCGTGTGTACAGAATCTGGATGGCGGCCTC
TGCCTAGCTGCGAGGAAAAGAGCTGCGACAACCCT
TACATCCCCAACGGCGACTACAGCCCTCTGCGGAT
TAAGCACAGAACCGGCGACGAGATCACCTACCAGT
GCAGAAACGGCTTTTACCCCGCCACCAGAGGCAAT
ACCGCCAAGTGTACAAGCACCGGCTGGATCCCTGC
TCCTAGATGCACACTGAAG
Compound AB:
Amino Acid (SEQ ID NO: 147):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GQKSVWCQANNMWGPTRLPTCVSVFPGGGGSDAAV
ECPPCPAPPVAGPSVFLFPPKPKDTLMISRTPEVT
CVVVDVSQEDPEVQFNWYVDGVEVHNAKTKPREEQ
FNSTYRVVSVLTVLHQDWLNGKEYKCKVSNKGLPS
SIEKTISKAKGQPREPQVYTLPPSQEEMTKNQVSL
TCLVKGFYPSDIAVEWESNGQPENNYKTTPPVLDS
DGSFFLYSRLTVDKSRWQEGNVFSCSVMHEALHNH
YTQKSLSLSLGKGGGGAGGGGAGGGAGGGGSEDCN
ELPPRRNTEILTGSWSDQTYPEGTQAIYKCRPGYR
SLGNVIMVCRKGEWVALNPLRKCQKRPCGHPGDTP
FGTFTLTGGNVFEYGVKAVYTCNEGYQLLGEINYR
ECDTDGWTNDIPICEVVKCLPVTAPENGKIVSSAM
EPDREYHFGQAVRFVCNSGYKIEGDEEMHCSDDGF
WSKEKPKCVEISCKSPDVINGSPISQKIIYKENER
FQYKCNMGYEYSERGDAVCTESGWRPLPSCEEKSC
DNPYIPNGDYSPLRIKHRTGDEITYQCRNGFYPAT
RGNTAKCTSTGWIPAPRCTLK
Nucleic Acid: (SEQ ID NO: 192):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCCAGAAAAGCGTGTGGTGCCAGGCCAACAATAT
GTGGGGCCCTACCAGACTGCCCACCTGTGTTTCAG
TTTTTCCAGGCGGCGGAGGCTCTGATGCCGCTGTT
GAATGTCCTCCTTGTCCAGCTCCTCCTGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACTCCACCTACAGAGTGGTGTCCGTGCTGAC
CGTGCTGCACCAGGATTGGCTGAATGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTAGCGACAT
TGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCAGCGTGATGCACGAAGCCCTGCACAACCAC
TACACCCAGAAGTCTCTGAGCCTGTCTCTCGGAAA
AGGCGGAGGCGGAGCTGGTGGTGGCGGTGCTGGTG
GCGGAGCTGGCGGAGGTGGAAGTGAAGATTGCAAC
GAGCTGCCTCCTCGGCGGAATACCGAGATTCTGAC
AGGCTCTTGGAGCGACCAGACATACCCTGAGGGCA
CCCAGGCCATCTACAAGTGTAGACCTGGCTACCGC
AGCCTGGGCAATGTGATCATGGTCTGCAGAAAAGG
CGAGTGGGTCGCCCTGAATCCTCTGAGAAAGTGCC
AGAAGAGGCCTTGCGGACACCCCGGCGATACACCT
TTTGGCACATTCACCCTGACCGGCGGCAATGTGTT
TGAGTATGGCGTGAAGGCCGTGTACACCTGTAACG
AGGGATATCAGCTGCTGGGCGAGATCAACTACAGA
GAGTGTGATACCGACGGCTGGACCAACGACATCCC
TATCTGCGAGGTGGTCAAGTGCCTGCCTGTGACAG
CCCCTGAGAATGGCAAGATCGTGTCCAGCGCCATG
GAACCCGACAGAGAGTATCACTTTGGCCAGGCCGT
CAGATTCGTGTGCAACTCCGGATACAAGATCGAGG
GCGACGAGGAAATGCACTGCAGCGACGACGGCTTC
TGGTCCAAAGAAAAGCCCAAATGCGTGGAAATCAG
CTGCAAGAGCCCCGACGTGATCAACGGCAGCCCTA
TCAGCCAGAAGATCATCTACAAAGAGAACGAGCGG
TTCCAGTATAAGTGCAACATGGGCTACGAGTACAG
CGAGCGGGGAGATGCCGTGTGTACAGAATCTGGAT
GGCGGCCTCTGCCTAGCTGCGAGGAAAAGAGCTGC
GACAACCCTTACATCCCCAACGGCGACTACAGCCC
TCTGCGGATTAAGCACAGAACCGGCGACGAGATCA
CCTACCAGTGCAGAAACGGCTTTTACCCTGCCACC
AGAGGCAACACCGCCAAGTGTACAAGCACAGGCTG
GATCCCCGCTCCTCGGTGCACACTGAAA
Compound AC:
Amino Acid (SEQ ID NO: 148):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GQKSVWCQANNMWGPTRLPTCVSVFPGGGGSDAAV
ECPPCPAPPVAGPSVFLFPPKPKDTLMISRTPEVT
CVVVDVSQEDPEVQFNWYVDGVEVHNAKTKPREEQ
FNSTYRVVSVLTVLHQDWLNGKEYKCKVSNKGLPS
SIEKTISKAKGQPREPQVYTLPPSQEEMTKNQVSL
TCLVKGFYPSDIAVEWESNGQPENNYKTTPPVLDS
DGSFFLYSRLTVDKSRWQEGNVFSCSVMHEALHNH
YTQKSLSLSLGKGGGGAGGGGAGGGAGGGGSEDCN
ELPPRRNTEILTGSWSDQTYPEGTQAIYKCRPGYR
SLGNVIMVCRKGEWVALNPLRKCQKRPCGHPGDTP
FGTFTLTGGNVFEYGVKAVYTCNEGYQLLGEINYR
ECDTDGWTNDIPICEVVKCLPVTAPENGKIVSSAM
EPDREYHFGQAVRFVCNSGYKIEGDEEMHCSDDGF
WSKEKPKCVEISCKSPDVINGSPISQKIIYKENER
FQYKCNMGYEYSERGDAVCTESGWRPLPSCEEKS
Nucleic Acid: (SEQ ID NO: 193):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCCAGAAAAGCGTGTGGTGCCAGGCCAACAATAT
GTGGGGCCCTACCAGACTGCCCACCTGTGTTTCAG
TTTTTCCAGGCGGCGGAGGCTCTGATGCCGCTGTT
GAATGTCCTCCTTGTCCAGCTCCTCCTGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACTCCACCTACAGAGTGGTGTCCGTGCTGAC
CGTGCTGCACCAGGATTGGCTGAATGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTAGCGACAT
TGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCAGCGTGATGCACGAAGCCCTGCACAACCAC
TACACCCAGAAGTCTCTGAGCCTGTCTCTCGGAAA
AGGCGGAGGCGGAGCTGGTGGTGGCGGTGCTGGTG
GCGGAGCTGGCGGAGGTGGAAGTGAAGATTGCAAC
GAGCTGCCTCCTCGGCGGAATACCGAGATTCTGAC
AGGCTCTTGGAGCGACCAGACATACCCTGAGGGCA
CCCAGGCCATCTACAAGTGTAGACCTGGCTACCGC
AGCCTGGGCAATGTGATCATGGTCTGCAGAAAAGG
CGAGTGGGTCGCCCTGAATCCTCTGAGAAAGTGCC
AGAAGAGGCCTTGCGGACACCCCGGCGATACACCT
TTTGGCACATTCACCCTGACCGGCGGCAATGTGTT
TGAGTATGGCGTGAAGGCCGTGTACACCTGTAACG
AGGGATATCAGCTGCTGGGCGAGATCAACTACAGA
GAGTGTGATACCGACGGCTGGACCAACGACATCCC
TATCTGCGAGGTGGTCAAGTGCCTGCCTGTGACAG
CCCCTGAGAATGGCAAGATCGTGTCCAGCGCCATG
GAACCCGACAGAGAGTATCACTTTGGCCAGGCCGT
CAGATTCGTGTGCAACTCCGGATACAAGATCGAGG
GCGACGAGGAAATGCACTGCAGCGACGACGGCTTC
TGGTCCAAAGAAAAGCCCAAATGCGTGGAAATCAG
CTGCAAGAGCCCCGACGTGATCAACGGCAGCCCTA
TCAGCCAGAAGATCATCTACAAAGAGAACGAGCGG
TTCCAGTATAAGTGCAACATGGGCTACGAGTACAG
CGAGCGGGGAGATGCCGTGTGTACAGAATCTGGAT
GGCGGCCTCTGCCTAGCTGCGAAGAGAAGTCT
Compound AC:
Amino Acid (SEQ ID NO: 148):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GQKSVWCQANNMWGPTRLPTCVSVFPGGGGSDAAV
ECPPCPAPPVAGPSVFLFPPKPKDTLMISRTPEVT
CVVVDVSQEDPEVQFNWYVDGVEVHNAKTKPREEQ
FNSTYRVVSVLTVLHQDWLNGKEYKCKVSNKGLPS
SIEKTISKAKGQPREPQVYTLPPSQEEMTKNQVSL
TCLVKGFYPSDIAVEWESNGQPENNYKTTPPVLDS
DGSFFLYSRLTVDKSRWQEGNVFSCSVMHEALHNH
YTQKSLSLSLGKGGGGAGGGGAGGGAGGGGSEDCN
ELPPRRNTEILTGSWSDQTYPEGTQAIYKCRPGYR
SLGNVIMVCRKGEWVALNPLRKCQKRPCGHPGDTP
FGTFTLTGGNVFEYGVKAVYTCNEGYQLLGEINYR
ECDTDGWTNDIPICEVVKCLPVTAPENGKIVSSAM
EPDREYHFGQAVRFVCNSGYKIEGDEEMHCSDDGF
WSKEKPKCVEISCKSPDVINGSPISQKIIYKENER
FQYKCNMGYEYSERGDAVCTESGWRPLPSCEEKS
Nucleic Acid: (SEQ ID NO: 193):
ATCAGCTGTGGCAGCCCTCCACCTATCCTGAACGG
CAGAATCAGCTACTACAGCACCCCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCCAGAAAAGCGTGTGGTGCCAGGCCAACAATAT
GTGGGGCCCTACCAGACTGCCCACCTGTGTTTCAG
TTTTTCCAGGCGGCGGAGGCTCTGATGCCGCTGTT
GAATGTCCTCCTTGTCCAGCTCCTCCTGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACTCCACCTACAGAGTGGTGTCCGTGCTGAC
CGTGCTGCACCAGGATTGGCTGAATGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTAGCGACAT
TGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCAGCGTGATGCACGAAGCCCTGCACAACCAC
TACACCCAGAAGTCTCTGAGCCTGTCTCTCGGAAA
AGGCGGAGGCGGAGCTGGTGGTGGCGGTGCTGGTG
GCGGAGCTGGCGGAGGTGGAAGTGAAGATTGCAAC
GAGCTGCCTCCTCGGCGGAATACCGAGATTCTGAC
AGGCTCTTGGAGCGACCAGACATACCCTGAGGGCA
CCCAGGCCATCTACAAGTGTAGACCTGGCTACCGC
AGCCTGGGCAATGTGATCATGGTCTGCAGAAAAGG
CGAGTGGGTCGCCCTGAATCCTCTGAGAAAGTGCC
AGAAGAGGCCTTGCGGACACCCCGGCGATACACCT
TTTGGCACATTCACCCTGACCGGCGGCAATGTGTT
TGAGTATGGCGTGAAGGCCGTGTACACCTGTAACG
AGGGATATCAGCTGCTGGGCGAGATCAACTACAGA
GAGTGTGATACCGACGGCTGGACCAACGACATCCC
TATCTGCGAGGTGGTCAAGTGCCTGCCTGTGACAG
CCCCTGAGAATGGCAAGATCGTGTCCAGCGCCATG
GAACCCGACAGAGAGTATCACTTTGGCCAGGCCGT
CAGATTCGTGTGCAACTCCGGATACAAGATCGAGG
GCGACGAGGAAATGCACTGCAGCGACGACGGCTTC
TGGTCCAAAGAAAAGCCCAAATGCGTGGAAATCAG
CTGCAAGAGCCCCGACGTGATCAACGGCAGCCCTA
TCAGCCAGAAGATCATCTACAAAGAGAACGAGCGG
TTCCAGTATAAGTGCAACATGGGCTACGAGTACAG
CGAGCGGGGAGATGCCGTGTGTACAGAATCTGGAT
GGCGGCCTCTGCCTAGCTGCGAAGAGAAGTCT
Compound AD:
Amino Acid (SEQ ID NO: 149):
EPKSADKTHTCPPCPAPELLGGPSVFLFPPKPKDT
LMISRTPEVTCVVVDVSHEDPEVKFNWYVDGVEVH
NAKTKPREEQYNSTYRVVSVLTVLHQDWLNGKEYK
CKVSNKALPAPIEKTISKAKGQPREPQVYTLPPSR
DELTKNQVSLTCLVKGFYPSDIAVEWESNGQPENN
YKTTPPVLDSDGSFFLYSKLTVDKSRWQQGNVFSC
SVMHEALHNHYTQKSLSLSPGKGGGGAGGGGAGGG
GSEDCNELPPRRNTEILTGSWSDQTYPEGTQAIYK
CRPGYRSLGNVIMVCRKGEWVALNPLRKCQKRPCG
HPGDTPFGTFTLTGGNVFEYGVKAVYTCNEGYQLL
GEINYRECDTDGWTNDIPICEVVKCLPVTAPENGK
IVSSAMEPDREYHFGQAVRFVCNSGYKIEGDEEMH
CSDDGFWSKEKPKCVEISCKSPDVINGSPISQKII
YKENERFQYKCNMGYEYSERGDAVCTESGWRPLPS
CEEKSCDNPYIPNGDYSPLRIKHRTGDEITYQCRN
GFYPATRGNTAKCTSTGWIPAPRCTLK
Nucleic Acid: (SEQ ID NO: 194):
GAACCGAAGTCAGCTGACAAGACCCACACTTGCCC
TCCATGCCCTGCCCCTGAACTGCTTGGCGGGCCTT
CCGTGTTCCTGTTCCCCCCGAAACCTAAAGATACC
CTCATGATCTCGCGAACCCCGGAAGTGACTTGCGT
GGTCGTGGATGTGTCCCACGAGGATCCTGAAGTGA
AGTTCAATTGGTACGTGGATGGAGTGGAAGTCCAT
AACGCTAAGACGAAGCCGAGAGAGGAACAGTACAA
CTCGACCTACCGCGTGGTGTCCGTGCTCACCGTGC
TGCACCAAGACTGGCTGAACGGAAAGGAATACAAG
TGTAAAGTGTCCAACAAGGCCTTGCCAGCCCCTAT
CGAAAAGACCATATCAAAAGCAAAGGGACAGCCCA
GAGAGCCCCAGGTGTACACCCTGCCACCTTCCCGG
GATGAGCTGACCAAGAACCAAGTCTCCCTGACCTG
TCTGGTCAAGGGATTCTACCCCTCCGATATCGCGG
TCGAATGGGAGAGCAACGGACAACCCGAAAACAAC
TACAAGACTACCCCTCCCGTCCTCGACTCCGATGG
CTCGTTCTTCCTGTATTCGAAGTTGACTGTGGACA
AGTCCAGATGGCAGCAGGGCAACGTGTTCAGCTGC
AGCGTGATGCACGAGGCGCTGCACAATCATTACAC
CCAAAAGTCCCTGTCCTTGAGCCCTGGAAAGGGGG
GAGGAGGTGCAGGAGGAGGAGGCGCAGGAGGAGGA
GGTTCGGAGGACTGCAACGAGCTTCCACCGCGGAG
AAATACTGAAATTCTGACAGGCTCATGGTCTGATC
AGACTTACCCGGAAGGCACCCAGGCCATCTACAAA
TGTCGGCCCGGCTACAGGTCCCTCGGAAACGTGAT
CATGGTCTGCAGGAAGGGGGAATGGGTCGCCCTGA
ACCCGCTGAGAAAGTGCCAGAAGCGGCCATGTGGA
CACCCGGGAGACACTCCCTTCGGCACCTTTACCCT
GACCGGTGGAAACGTGTTCGAATACGGCGTGAAGG
CCGTGTACACTTGCAACGAAGGATATCAGCTTCTC
GGCGAGATCAACTATCGGGAATGCGACACCGATGG
CTGGACCAACGACATCCCTATCTGCGAAGTCGTCA
AGTGTCTCCCTGTGACTGCCCCGGAAAACGGAAAG
ATCGTGTCCTCCGCCATGGAACCTGACCGGGAATA
CCACTTTGGCCAAGCCGTGCGGTTCGTGTGCAACA
GCGGCTACAAAATTGAAGGAGATGAAGAAATGCAT
TGTAGCGATGACGGCTTCTGGTCCAAGGAGAAGCC
TAAGTGCGTGGAAATTAGCTGCAAGTCCCCCGACG
TGATCAACGGTTCCCCCATCTCCCAAAAGATTATC
TACAAGGAGAACGAGCGCTTCCAGTACAAGTGCAA
CATGGGATACGAGTACAGCGAGAGAGGGGACGCGG
TCTGCACCGAGTCCGGGTGGAGGCCTCTGCCGTCA
TGCGAAGAAAAGAGCTGCGACAACCCCTACATTCC
GAACGGAGACTACAGCCCGCTCAGGATCAAGCACC
GCACCGGGGATGAAATCACTTACCAATGCCGCAAC
GGATTCTATCCAGCGACTCGCGGGAATACCGCCAA
ATGCACCTCGACTGGTTGGATTCCGGCCCCAAGGT
GCACCCTGAAG
Compound AE:
Amino Acid (SEQ ID NO: 150):
EPKSADKTHTCPPCPAPELLGGPSVFLFPPKPKDT
LMISRTPEVTCVVVDVSHEDPEVKFNWYVDGVEVH
NAKTKPREEQYNSTYRVVSVLTVLHQDWLNGKEYK
CKVSNKALPAPIEKTISKAKGQPREPQVYTLPPSR
DELTKNQVSLTCLVKGFYPSDIAVEWESNGQPENN
YKTTPPVLDSDGSFFLYSKLTVDKSRWQQGNVFSC
SVMHEALHNHYTQKSLSLSPGKEDCNELPPRRNTE
ILTGSWSDQTYPEGTQAIYKCRPGYRSLGNVIMVC
RKGEWVALNPLRKCQKRPCGHPGDTPFGTFTLTGG
NVFEYGVKAVYTCNEGYQLLGEINYRECDTDGWTN
DIPICEVVKCLPVTAPENGKIVSSAMEPDREYHFG
QAVRFVCNSGYKIEGDEEMHCSDDGFWSKEKPKCV
EISCKSPDVINGSPISQKIIYKENERFQYKCNMGY
EYSERGDAVCTESGWRPLPSCEEKSCDNPYIPNGD
YSPLRIKHRTGDEITYQCRNGFYPATRGNTAKCTS
TGWIPAPRCTLK
Nucleic Acid: (SEQ ID NO: 195):
GAACCGAAGTCAGCTGACAAGACCCACACTTGCCC
TCCATGCCCTGCCCCTGAACTGCTTGGCGGGCCTT
CCGTGTTCCTGTTCCCCCCGAAACCTAAAGATACC
CTCATGATCTCGCGAACCCCGGAAGTGACTTGCGT
GGTCGTGGATGTGTCCCACGAGGATCCTGAAGTGA
AGTTCAATTGGTACGTGGATGGAGTGGAAGTCCAT
AACGCTAAGACGAAGCCGAGAGAGGAACAGTACAA
CTCGACCTACCGCGTGGTGTCCGTGCTCACCGTGC
TGCACCAAGACTGGCTGAACGGAAAGGAATACAAG
TGTAAAGTGTCCAACAAGGCCTTGCCAGCCCCTAT
CGAAAAGACCATATCAAAAGCAAAGGGACAGCCCA
GAGAGCCCCAGGTGTACACCCTGCCACCTTCCCGG
GATGAGCTGACCAAGAACCAAGTCTCCCTGACCTG
TCTGGTCAAGGGATTCTACCCCTCCGATATCGCGG
TCGAATGGGAGAGCAACGGACAACCCGAAAACAAC
TACAAGACTACCCCTCCCGTCCTCGACTCCGATGG
CTCGTTCTTCCTGTATTCGAAGTTGACTGTGGACA
AGTCCAGATGGCAGCAGGGCAACGTGTTCAGCTGC
AGCGTGATGCACGAGGCGCTGCACAATCATTACAC
CCAAAAGTCCCTGTCCTTGAGCCCTGGAAAGGAGG
ACTGCAACGAGCTTCCACCGCGGAGAAATACTGAA
ATTCTGACAGGCTCATGGTCTGATCAGACTTACCC
GGAAGGCACCCAGGCCATCTACAAATGTCGGCCCG
GCTACAGGTCCCTCGGAAACGTGATCATGGTCTGC
AGGAAGGGGGAATGGGTCGCCCTGAACCCGCTGAG
AAAGTGCCAGAAGCGGCCATGTGGACACCCGGGAG
ACACTCCCTTCGGCACCTTTACCCTGACCGGTGGA
AACGTGTTCGAATACGGCGTGAAGGCCGTGTACAC
TTGCAACGAAGGATATCAGCTTCTCGGCGAGATCA
ACTATCGGGAATGCGACACCGATGGCTGGACCAAC
GACATCCCTATCTGCGAAGTCGTCAAGTGTCTCCC
TGTGACTGCCCCGGAAAACGGAAAGATCGTGTCCT
CCGCCATGGAACCTGACCGGGAATACCACTTTGGC
CAAGCCGTGCGGTTCGTGTGCAACAGCGGCTACAA
AATTGAAGGAGATGAAGAAATGCATTGTAGCGATG
ACGGCTTCTGGTCCAAGGAGAAGCCTAAGTGCGTG
GAAATTAGCTGCAAGTCCCCCGACGTGATCAACGG
TTCCCCCATCTCCCAAAAGATTATCTACAAGGAGA
ACGAGCGCTTCCAGTACAAGTGCAACATGGGATAC
GAGTACAGCGAGAGAGGGGACGCGGTCTGCACCGA
GTCCGGGTGGAGGCCTCTGCCGTCATGCGAAGAAA
AGAGCTGCGACAACCCCTACATTCCGAACGGAGAC
TACAGCCCGCTCAGGATCAAGCACCGCACCGGGGA
TGAAATCACTTACCAATGCCGCAACGGATTCTATC
CAGCGACTCGCGGGAATACCGCCAAATGCACCTCG
ACTGGTTGGATTCCGGCCCCAAGGTGCACCCTGAA
G
Compound AF:
Amino Acid (SEQ ID NO: 151):
EDCNELPPRRNTEILTGSWSDQTYPEGTQAIYKCRP
GYRSLGNVIMVCRKGEWVALNPLRKCQKRPCGHPG
DTPFGTFTLTGGNVFEYGVKAVYTCNEGYQLLGEI
NYRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KSCDNPYIPNGDYSPLRIKHRTGDEITYQCRNGFY
PATRGNTAKCTSTGWIPAPRCTLKGGGGAGGGGAG
GGGSDKTHTCPPCPAPELLGGPSVFLFPPKPKDTL
MISRTPEVTCVVVDVSHEDPEVKFNWYVDGVEVHN
AKTKPREEQYNSTYRVVSVLTVLHQDWLNGKEYKC
KVSNKALPAPIEKTISKAKGQPREPQVYTLPPSRD
ELTKNQVSLTCLVKGFYPSDIAVEWESNGQPENNY
KTTPPVLDSDGSFFLYSKLTVDKSRWQQGNVFSCS
VMHEALHNHYTQKSLSLSPGK
Nucleic Acid: (SEQ ID NO: 196):
GAAGATTGCAACGAGCTTCCACCGCGGAGAAATAC
TGAAATTCTGACAGGCTCATGGTCTGATCAGACTT
ACCCGGAAGGCACCCAGGCCATCTACAAATGTCGG
CCCGGCTACAGGTCCCTCGGAAACGTGATCATGGT
CTGCAGGAAGGGGGAATGGGTCGCCCTGAACCCGC
TGAGAAAGTGCCAGAAGCGGCCATGTGGACACCCG
GGAGACACTCCCTTCGGCACCTTTACCCTGACCGG
TGGAAACGTGTTCGAATACGGCGTGAAGGCCGTGT
ACACTTGCAACGAAGGATATCAGCTTCTCGGCGAG
ATCAACTATCGGGAATGCGACACCGATGGCTGGAC
CAACGACATCCCTATCTGCGAAGTCGTCAAGTGTC
TCCCTGTGACTGCCCCGGAAAACGGAAAGATCGTG
TCCTCCGCCATGGAACCTGACCGGGAATACCACTT
TGGCCAAGCCGTGCGGTTCGTGTGCAACAGCGGCT
ACAAAATTGAAGGAGATGAAGAAATGCATTGTAGC
GATGACGGCTTCTGGTCCAAGGAGAAGCCTAAGTG
CGTGGAAATTAGCTGCAAGTCCCCCGACGTGATCA
ACGGTTCCCCCATCTCCCAAAAGATTATCTACAAG
GAGAACGAGCGCTTCCAGTACAAGTGCAACATGGG
ATACGAGTACAGCGAGAGAGGGGACGCGGTCTGCA
CCGAGTCCGGGTGGAGGCCTCTGCCGTCATGCGAA
GAAAAGAGCTGCGACAACCCCTACATTCCGAACGG
AGACTACAGCCCGCTCAGGATCAAGCACCGCACCG
GGGATGAAATCACTTACCAATGCCGCAACGGATTC
TATCCAGCGACTCGCGGGAATACCGCCAAATGCAC
CTCGACTGGTTGGATTCCGGCCCCAAGGTGCACCC
TGAAGGGCGGTGGCGGAGCGGGCGGAGGAGGAGCT
GGAGGGGGAGGCAGCGACAAGACCCACACTTGCCC
TCCATGCCCTGCCCCTGAACTGCTTGGCGGGCCTT
CCGTGTTCCTGTTCCCCCCGAAACCTAAAGATACC
CTCATGATCTCGCGAACCCCGGAAGTGACTTGCGT
GGTCGTGGATGTGTCCCACGAGGATCCTGAAGTGA
AGTTCAATTGGTACGTGGATGGAGTGGAAGTCCAT
AACGCTAAGACGAAGCCGAGAGAGGAACAGTACAA
CTCGACCTACCGCGTGGTGTCCGTGCTCACCGTGC
TGCACCAAGACTGGCTGAACGGAAAGGAATACAAG
TGTAAAGTGTCCAACAAGGCCTTGCCAGCCCCTAT
CGAAAAGACCATATCAAAAGCAAAGGGACAGCCCA
GAGAGCCCCAGGTGTACACCCTGCCACCTTCCCGG
GATGAGCTGACCAAGAACCAAGTCTCCCTGACCTG
TCTGGTCAAGGGATTCTACCCCTCCGATATCGCGG
TCGAATGGGAGAGCAACGGACAACCCGAAAACAAC
TACAAGACTACCCCTCCCGTCCTCGACTCCGATGG
CTCGTTCTTCCTGTATTCGAAGTTGACTGTGGACA
AGTCCAGATGGCAGCAGGGCAACGTGTTCAGCTGC
AGCGTGATGCACGAGGCGCTGCACAATCATTACAC
CCAAAAGTCCCTGTCCTTGAGCCCTGGAAAG
Compound AG:
Amino Acid (SEQ ID NO: 152):
EDCNELPPRRNTEILTGSWSDQTYPEGTQAIYKCRP
GYRSLGNVIMVCRKGEWVALNPLRKCQKRPCGHPG
DTPFGTFTLTGGNVFEYGVKAVYTCNEGYQLLGEI
NYRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KSCDNPYIPNGDYSPLRIKHRTGDEITYQCRNGFY
PATRGNTAKCTSTGWIPAPRCTLKGGGGAGGGGAG
GGGSVECPPCPAPPVAGPSVFLFPPKPKDTLMISR
TPEVTCVVVDVSQEDPEVQFNWYVDGVEVHNAKTK
PREEQFNSTYRVVSVLTVLHQDWLNGKEYKCKVSN
KGLPSSIEKTISKAKGQPREPQVYTLPPSQEEMTK
NQVSLTCLVKGFYPSDIAVEWESNGQPENNYKTTP
PVLDSDGSFFLYSRLTVDKSRWQEGNVFSCSVMHE
ALHNHYTQKSLSLSLGKGKCGPPPPIDNGDITSFP
LSVYAPASSVEYQCQNLYQLEGNKRITCRNGQWSE
PPKCLHPCVISREIMENYNIALRWTAKQKLYSRTG
ESVEFVCKRGYRLSSRSHTLRTTCWDGKLEYPTCA
KR
Nucleic Acid: (SEQ ID NO: 197):
GAGGATTGCAATGAGCTGCCTCCTCGGAGAAACAC
CGAGATCCTGACAGGCTCTTGGAGCGACCAGACAT
ACCCTGAGGGCACCCAGGCCATCTACAAGTGCAGA
CCTGGCTACAGATCCCTGGGCAACGTGATCATGGT
CTGCAGAAAAGGCGAGTGGGTCGCCCTGAATCCTC
TGAGAAAGTGCCAGAAGAGGCCTTGCGGACACCCT
GGCGATACCCCTTTTGGCACATTCACACTGACCGG
CGGCAACGTGTTCGAGTATGGCGTGAAGGCCGTGT
ACACCTGTAACGAGGGATATCAGCTGCTGGGCGAG
ATCAACTACAGAGAGTGTGATACCGACGGCTGGAC
CAACGACATCCCTATCTGCGAGGTGGTCAAGTGCC
TGCCTGTGACAGCCCCTGAGAATGGCAAGATCGTG
TCCAGCGCCATGGAACCCGACAGAGAGTATCACTT
TGGCCAGGCCGTCAGATTCGTGTGCAACAGCGGCT
ATAAGATCGAGGGCGACGAGGAAATGCACTGCAGC
GACGACGGCTTCTGGTCCAAAGAAAAGCCTAAGTG
CGTGGAAATCAGCTGCAAGAGCCCCGACGTGATCA
ACGGCAGCCCTATCAGCCAGAAGATCATCTACAAA
GAGAACGAGCGGTTCCAGTACAAGTGTAACATGGG
CTACGAGTACAGCGAGAGGGGCGACGCCGTGTGTA
CAGAATCTGGATGGCGACCTCTGCCTAGCTGCGAG
GAAAAGAGCTGCGACAACCCTTACATCCCCAACGG
CGACTACAGCCCTCTGCGGATTAAGCACAGAACCG
GCGACGAGATCACCTACCAGTGCAGAAATGGCTTC
TACCCCGCCACCAGAGGCAATACCGCCAAGTGTAC
AAGCACCGGCTGGATCCCTGCTCCTAGATGTACAC
TTAAAGGCGGAGGCGGAGCTGGTGGTGGCGGAGCA
GGCGGCGGAGGATCTGTTGAATGTCCTCCTTGTCC
TGCTCCTCCAGTGGCCGGACCTTCCGTGTTTCTGT
TCCCACCTAAGCCTAAGGACACACTGATGATCAGC
AGAACCCCTGAAGTGACCTGCGTGGTGGTGGACGT
TTCCCAAGAGGATCCCGAGGTGCAGTTCAATTGGT
ACGTGGACGGCGTGGAAGTGCACAACGCCAAGACC
AAGCCTAGAGAGGAACAGTTCAACAGCACCTACAG
AGTGGTGTCCGTGCTGACCGTGCTGCACCAGGATT
GGCTGAACGGCAAAGAGTATAAGTGCAAGGTGTCC
AACAAGGGCCTGCCTAGCAGCATCGAGAAAACCAT
CAGCAAGGCCAAGGGCCAGCCAAGAGAGCCTCAGG
TTTACACCCTGCCTCCAAGCCAAGAGGAAATGACC
AAGAACCAGGTGTCCCTGACCTGCCTGGTCAAGGG
CTTTTACCCTTCCGATATCGCCGTGGAATGGGAGA
GCAATGGCCAGCCTGAGAACAACTACAAGACCACA
CCTCCTGTGCTGGACAGCGACGGCAGCTTTTTTCT
GTACTCCCGCCTGACCGTGGACAAGAGCAGATGGC
AAGAGGGCAATGTGTTCAGCTGCAGCGTGATGCAC
GAGGCCCTGCACAACCACTACACCCAGAAGTCTCT
GAGCCTGAGCCTCGGCAAGGGAAAGTGTGGACCTC
CTCCTCCTATCGACAATGGCGACATCACCAGCTTT
CCACTGTCTGTGTACGCCCCTGCCAGCAGCGTTGA
GTATCAGTGTCAGAACCTGTACCAGCTGGAAGGCA
ACAAGCGGATCACCTGTAGAAACGGCCAGTGGTCC
GAGCCTCCTAAGTGTCTGCACCCTTGCGTGATCAG
CCGCGAGATCATGGAAAACTACAATATCGCCCTGC
GGTGGACCGCCAAGCAGAAGCTGTATTCTAGAACA
GGCGAGAGCGTCGAGTTTGTGTGCAAGAGAGGCTA
CCGGCTGAGCAGCAGAAGCCACACACTGAGAACCA
CCTGTTGGGACGGCAAGCTGGAATACCCTACCTGC
GCCAAGAGA
Compound AH:
Amino Acid (SEQ ID NO: 153):
EDCNELPPRRNTEILTGSWSDQTYPEGTQAIYKCRP
GYRSLGNVIMVCRKGEWVALNPLRKCQKRPCGHPG
DTPFGTFTLTGGNVFEYGVKAVYTCNEGYQLLGEI
NYRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KSCDNPYIPNGDYSPLRIKHRTGDEITYQCRNGFY
PATRGNTAKCTSTGWIPAPRCTLKVECPPCPAPPV
AGPSVFLFPPKPKDTLMISRTPEVTCVVVDVSQED
PEVQFNWYVDGVEVHNAKTKPREEQFNSTYRVVSV
LTVLHQDWLNGKEYKCKVSNKGLPSSIEKTISKAK
GQPREPQVYTLPPSQEEMTKNQVSLTCLVKGFYPS
DIAVEWESNGQPENNYKTTPPVLDSDGSFFLYSRL
TVDKSRWQEGNVFSCSVMHEALHNHYTQKSLSLSL
GKGGGGAGGGGAGGGGSGKCGPPPPIDNGDITSFP
LSVYAPASSVEYQCQNLYQLEGNKRITCRNGQWSE
PPKCLHPCVISREIMENYNIALRWTAKQKLYSRTG
ESVEFVCKRGYRLSSRSHTLRTTCWDGKLEYPTCA
KR
Nucleic Acid: (SEQ ID NO: 198):
GAGGATTGCAATGAGCTGCCTCCTCGGAGAAACAC
CGAGATCCTGACAGGCTCTTGGAGCGACCAGACAT
ACCCTGAGGGCACCCAGGCCATCTACAAGTGCAGA
CCTGGCTACAGATCCCTGGGCAACGTGATCATGGT
CTGCAGAAAAGGCGAGTGGGTCGCCCTGAATCCTC
TGAGAAAGTGCCAGAAGAGGCCTTGCGGACACCCT
GGCGATACCCCTTTTGGCACATTCACACTGACCGG
CGGCAACGTGTTCGAGTATGGCGTGAAGGCCGTGT
ACACCTGTAACGAGGGATATCAGCTGCTGGGCGAG
ATCAACTACAGAGAGTGTGATACCGACGGCTGGAC
CAACGACATCCCTATCTGCGAGGTGGTCAAGTGCC
TGCCTGTGACAGCCCCTGAGAATGGCAAGATCGTG
TCCAGCGCCATGGAACCCGACAGAGAGTATCACTT
TGGCCAGGCCGTCAGATTCGTGTGCAACAGCGGCT
ATAAGATCGAGGGCGACGAGGAAATGCACTGCAGC
GACGACGGCTTCTGGTCCAAAGAAAAGCCTAAGTG
CGTGGAAATCAGCTGCAAGAGCCCCGACGTGATCA
ACGGCAGCCCTATCAGCCAGAAGATCATCTACAAA
GAGAACGAGCGGTTCCAGTACAAGTGTAACATGGG
CTACGAGTACAGCGAGAGGGGCGACGCCGTGTGTA
CAGAATCTGGATGGCGACCTCTGCCTAGCTGCGAG
GAAAAGAGCTGCGACAACCCTTACATCCCCAACGG
CGACTACAGCCCTCTGCGGATTAAGCACAGAACCG
GCGACGAGATCACCTACCAGTGCAGAAATGGCTTC
TACCCCGCCACCAGAGGCAATACCGCCAAGTGTAC
AAGCACCGGCTGGATCCCTGCTCCTAGATGCACCC
TGAAGGTGGAATGCCCTCCTTGTCCTGCTCCTCCA
GTGGCCGGACCTTCCGTGTTTCTGTTCCCACCTAA
GCCTAAGGACACACTGATGATCAGCAGAACCCCTG
AAGTGACCTGCGTGGTGGTGGACGTTTCCCAAGAG
GATCCCGAGGTGCAGTTCAATTGGTACGTGGACGG
CGTGGAAGTGCACAACGCCAAGACCAAGCCTAGAG
AGGAACAGTTCAACAGCACCTACAGAGTGGTGTCC
GTGCTGACCGTGCTGCACCAGGATTGGCTGAACGG
CAAAGAGTATAAGTGCAAGGTGTCCAACAAGGGCC
TGCCTAGCAGCATCGAGAAAACCATCAGCAAGGCC
AAGGGCCAGCCAAGAGAGCCTCAGGTTTACACCCT
GCCTCCAAGCCAAGAGGAAATGACCAAGAACCAGG
TGTCCCTGACCTGCCTGGTCAAGGGCTTTTACCCT
TCCGATATCGCCGTGGAATGGGAGAGCAATGGCCA
GCCTGAGAACAACTACAAGACCACACCTCCTGTGC
TGGACAGCGACGGCAGCTTTTTTCTGTACTCCCGC
CTGACCGTGGACAAGAGCAGATGGCAAGAGGGCAA
TGTGTTCAGCTGCAGCGTGATGCACGAGGCCCTGC
ACAACCACTACACCCAGAAGTCTCTGAGCCTGTCT
CTCGGAAAAGGCGGAGGCGGAGCTGGTGGTGGCGG
AGCAGGCGGCGGAGGATCTGGAAAATGTGGACCTC
CTCCTCCTATCGACAATGGCGACATCACCAGCTTT
CCACTGTCTGTGTACGCCCCTGCCAGCAGCGTTGA
GTATCAGTGTCAGAACCTGTACCAGCTGGAAGGCA
ACAAGCGGATCACCTGTAGAAACGGCCAGTGGTCC
GAGCCTCCTAAGTGTCTGCACCCTTGCGTGATCAG
CCGCGAGATCATGGAAAACTACAATATCGCCCTGC
GGTGGACCGCCAAGCAGAAGCTGTATTCTAGAACA
GGCGAGAGCGTCGAGTTTGTGTGCAAGAGAGGCTA
CCGGCTGAGCAGCAGAAGCCACACACTGAGAACCA
CCTGTTGGGACGGCAAGCTGGAATACCCTACCTGC
GCCAAGAGA
Compound AI:
Amino Acid (SEQ ID NO: 154):
EDCNELPPRRNTEILTGSWSDQTYPEGTQAIYKCRP
GYRSLGNVIMVCRKGEWVALNPLRKCQKRPCGHPG
DTPFGTFTLTGGNVFEYGVKAVYTCNEGYQLLGEI
NYRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KSCDNPYIPNGDYSPLRIKHRTGDEITYQCRNGFY
PATRGNTAKCTSTGWIPAPRCTLKGGGGAGGGGAG
GGGSVECPPCPAPPVAGPSVFLFPPKPKDTLMISR
TPEVTCVVVDVSQEDPEVQFNWYVDGVEVHNAKTK
PREEQFNSTYRVVSVLTVLHQDWLNGKEYKCKVSN
KGLPSSIEKTISKAKGQPREPQVYTLPPSQEEMTK
NQVSLTCLVKGFYPSDIAVEWESNGQPENNYKTTP
PVLDSDGSFFLYSRLTVDKSRWQEGNVFSCSVMHE
ALHNHYTQKSLSLSLGKGGGGAGGGGAGGGGSGKC
GPPPPIDNGDITSFPLSVYAPASSVEYQCQNLYQL
EGNKRITCRNGQWSEPPKCLHPCVISREIMENYNI
ALRWTAKQKLYSRTGESVEFVCKRGYRLSSRSHTL
RTTCWDGKLEYPTCAKR
Nucleic Acid: (SEQ ID NO: 199):
GAGGATTGCAATGAGCTGCCTCCTCGGAGAAACAC
CGAGATCCTGACAGGCTCTTGGAGCGACCAGACAT
ACCCTGAGGGCACCCAGGCCATCTACAAGTGCAGA
CCTGGCTACAGATCCCTGGGCAACGTGATCATGGT
CTGCAGAAAAGGCGAGTGGGTCGCCCTGAATCCTC
TGAGAAAGTGCCAGAAGAGGCCTTGCGGACACCCT
GGCGATACCCCTTTTGGCACATTCACACTGACCGG
CGGCAACGTGTTCGAGTATGGCGTGAAGGCCGTGT
ACACCTGTAACGAGGGATATCAGCTGCTGGGCGAG
ATCAACTACAGAGAGTGTGATACCGACGGCTGGAC
CAACGACATCCCTATCTGCGAGGTGGTCAAGTGCC
TGCCTGTGACAGCCCCTGAGAATGGCAAGATCGTG
TCCAGCGCCATGGAACCCGACAGAGAGTATCACTT
TGGCCAGGCCGTCAGATTCGTGTGCAACAGCGGCT
ATAAGATCGAGGGCGACGAGGAAATGCACTGCAGC
GACGACGGCTTCTGGTCCAAAGAAAAGCCTAAGTG
CGTGGAAATCAGCTGCAAGAGCCCCGACGTGATCA
ACGGCAGCCCTATCAGCCAGAAGATCATCTACAAA
GAGAACGAGCGGTTCCAGTACAAGTGTAACATGGG
CTACGAGTACAGCGAGAGGGGCGACGCCGTGTGTA
CAGAATCTGGATGGCGACCTCTGCCTAGCTGCGAG
GAAAAGAGCTGCGACAACCCTTACATCCCCAACGG
CGACTACAGCCCTCTGCGGATTAAGCACAGAACCG
GCGACGAGATCACCTACCAGTGCAGAAATGGCTTC
TACCCCGCCACCAGAGGCAATACCGCCAAGTGTAC
AAGCACCGGCTGGATCCCTGCTCCTAGATGTACAC
TTAAAGGCGGAGGCGGAGCTGGTGGTGGCGGAGCA
GGCGGCGGAGGATCTGTTGAATGTCCTCCTTGTCC
TGCTCCTCCAGTGGCCGGACCTTCCGTGTTTCTGT
TCCCACCTAAGCCTAAGGACACACTGATGATCAGC
AGAACCCCTGAAGTGACCTGCGTGGTGGTGGACGT
TTCCCAAGAGGATCCCGAGGTGCAGTTCAATTGGT
ACGTGGACGGCGTGGAAGTGCACAACGCCAAGACC
AAGCCTAGAGAGGAACAGTTCAACAGCACCTACAG
AGTGGTGTCCGTGCTGACCGTGCTGCACCAGGATT
GGCTGAACGGCAAAGAGTATAAGTGCAAGGTGTCC
AACAAGGGCCTGCCTAGCAGCATCGAGAAAACCAT
CAGCAAGGCCAAGGGCCAGCCAAGAGAGCCTCAGG
TTTACACCCTGCCTCCAAGCCAAGAGGAAATGACC
AAGAACCAGGTGTCCCTGACCTGCCTGGTCAAGGG
CTTTTACCCTTCCGATATCGCCGTGGAATGGGAGA
GCAATGGCCAGCCTGAGAACAACTACAAGACCACA
CCTCCTGTGCTGGACAGCGACGGCAGCTTTTTTCT
GTACTCCCGCCTGACCGTGGACAAGAGCAGATGGC
AAGAGGGCAATGTGTTCAGCTGCAGCGTGATGCAC
GAGGCCCTGCACAACCACTACACCCAGAAGTCTCT
GAGCCTGTCTCTTGGAAAAGGTGGCGGTGGTGCTG
GCGGCGGTGGTGCAGGCGGTGGCGGATCTGGAAAA
TGTGGACCTCCTCCTCCTATCGACAATGGCGACAT
CACCAGCTTTCCACTGTCTGTGTACGCCCCTGCCA
GCAGCGTTGAGTATCAGTGTCAGAACCTGTACCAG
CTGGAAGGCAACAAGCGGATCACCTGTAGAAACGG
CCAGTGGTCCGAGCCTCCTAAGTGTCTGCACCCTT
GCGTGATCAGCCGCGAGATCATGGAAAACTACAAT
ATCGCCCTGCGGTGGACCGCCAAGCAGAAGCTGTA
TTCTAGAACAGGCGAGAGCGTCGAGTTTGTGTGCA
AGAGAGGCTACCGGCTGAGCAGCAGAAGCCACACA
CTGAGAACCACCTGTTGGGACGGCAAGCTGGAATA
CCCTACCTGCGCCAAGAGA
Compound AJ:
Amino Acid (SEQ ID NO: 155):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GQKSVWCQANNMWGPTRLPTCVSVFPGGGGSDAAE
RKCCVECPPCPAPPVAGPSVFLFPPKPKDTLMISR
TPEVTCVVVDVSQEDPEVQFNWYVDGVEVHNAKTK
PREEQFNSTYRVVSVLTVLHQDWLNGKEYKCKVSN
KGLPSSIEKTISKAKGQPREPQVYTLPPSQEEMTK
NQVSLTCLVKGFYPSDIAVEWESNGQPENNYKTTP
PVLDSDGSFFLYSRLTVDKSRWQEGNVFSCSVMHE
ALHNHYTQKSLSLSLGKGGGGAGGGGAGGGAGGGG
SEDCNELPPRRNTEILTGSWSDQTYPEGTQAIYKC
RPGYRSLGNVIMVCRKGEWVALNPLRKCQKRPCGH
PGDTPFGTFTLTGGNVFEYGVKAVYTCNEGYQLLG
EINYRECDTDGWTNDIPICEVVKCLPVTAPENGKI
VSSAMEPDREYHFGQAVRFVCNSGYKIEGDEEMHC
SDDGFWSKEKPKCVEISCKSPDVINGSPISQKIIY
KENERFQYKCNMGYEYSERGDAVCTESGWRPLPSC
EEKS
Nucleic Acid: (SEQ ID NO: 200):
ATTTCTTGTGGCTCTCCACCTCCTATCCTGAACGG
CCGGATCAGCTACTACAGCACACCTATCGCCGTGG
GCACCGTGATCAGATACAGCTGCTCTGGCACCTTC
CGGCTGATCGGAGAGAAGTCCCTGCTGTGCATCAC
CAAGGATAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACACCCTACAGACACGGCGATTCTG
TGACCTTCGCCTGCAAGACCAACTTCAGCATGAAC
GGCCAGAAAAGCGTGTGGTGCCAGGCCAACAACAT
GTGGGGACCTACCAGACTGCCCACCTGTGTGTCAG
TTTTTCCAGGCGGCGGAGGATCTGATGCCGCCGAG
AGAAAGTGCTGCGTGGAATGTCCTCCTTGTCCAGC
TCCTCCTGTGGCCGGACCTTCCGTGTTTCTGTTCC
CTCCAAAGCCTAAGGACACCCTGATGATCAGCAGA
ACCCCTGAAGTGACCTGCGTGGTGGTGGACGTTTC
CCAAGAGGATCCCGAGGTGCAGTTCAATTGGTACG
TGGACGGCGTGGAAGTGCACAACGCCAAGACCAAG
CCTAGAGAGGAACAGTTCAACAGCACCTACAGAGT
GGTGTCCGTGCTGACCGTGCTGCACCAGGATTGGC
TGAACGGCAAAGAGTACAAGTGCAAGGTGTCCAAC
AAGGGCCTGCCTAGCAGCATCGAGAAAACCATCAG
CAAGGCCAAGGGCCAGCCAAGAGAACCCCAGGTTT
ACACCCTGCCTCCAAGCCAAGAGGAAATGACCAAG
AACCAGGTGTCCCTGACCTGCCTGGTCAAGGGCTT
CTACCCTAGCGACATTGCCGTGGAATGGGAGAGCA
ATGGCCAGCCTGAGAACAACTACAAGACCACACCT
CCTGTGCTGGACAGCGACGGCAGCTTTTTTCTGTA
CTCCCGCCTGACCGTGGACAAGAGCAGATGGCAAG
AGGGCAACGTGTTCAGCTGCAGCGTGATGCACGAA
GCCCTGCACAACCACTACACCCAGAAGTCTCTGAG
CCTGTCTCTCGGAAAAGGCGGAGGCGGAGCTGGTG
GTGGCGGTGCTGGTGGCGGAGCTGGCGGAGGTGGA
AGTGAAGATTGCAACGAGCTGCCTCCTCGGCGGAA
TACCGAGATTCTGACAGGCTCTTGGAGCGACCAGA
CATACCCTGAGGGCACCCAGGCCATCTACAAGTGT
AGACCTGGCTACCGCAGCCTGGGCAATGTGATCAT
GGTCTGCAGAAAAGGCGAGTGGGTCGCCCTGAATC
CTCTGAGGAAGTGTCAGAAGAGGCCTTGCGGACAC
CCCGGCGATACACCTTTTGGCACATTCACCCTGAC
CGGCGGCAATGTGTTTGAGTATGGCGTGAAGGCCG
TGTACACCTGTAACGAGGGATATCAGCTGCTGGGC
GAGATCAACTACAGAGAGTGTGATACCGACGGCTG
GACCAACGACATCCCTATCTGCGAGGTGGTCAAGT
GCCTGCCTGTGACAGCCCCTGAGAATGGCAAGATC
GTGTCCAGCGCCATGGAACCCGACAGAGAGTATCA
CTTTGGCCAGGCCGTCAGATTCGTGTGCAACTCCG
GATACAAGATCGAGGGCGACGAGGAAATGCACTGC
AGCGACGACGGCTTCTGGTCCAAAGAAAAGCCCAA
ATGCGTGGAAATCAGCTGCAAGAGCCCCGACGTGA
TCAACGGCAGCCCTATCAGCCAGAAGATCATCTAC
AAAGAGAACGAGCGGTTCCAGTATAAGTGCAACAT
GGGCTACGAGTACAGCGAGCGGGGAGATGCCGTGT
GTACAGAATCTGGATGGCGGCCTCTGCCTAGCTGC
GAGGAAAAGTCT
Compound AK:
Amino Acid (SEQ ID NO: 156):
CVECPPCPAPPVAGPSVFLFPPKPKDTLMISRTPE
VTCVVVDVSQEDPEVQFNWYVDGVEVHNAKTKPRE
EQFNSTYRVVSVLTVLHQDWLNGKEYKCKVSNKGL
PSSIEKTISKAKGQPREPQVYTLPPSQEEMTKNQV
SLTCLVKGFYPSDIAVEWESNGQPENNYKTTPPVL
DSDGSFFLYSRLTVDKSRWQEGNVFSCSVMHEALH
NHYTQKSLSLSLGKGGGGAGGGGAGGGAGGGGSED
CNELPPRRNTEILTGSWSDQTYPEGTQAIYKCRPG
YRSLGNVIMVCRKGEWVALNPLRKCQKRPCGHPGD
TPFGTFTLTGGNVFEYGVKAVYTCNEGYQLLGEIN
YRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KS
Nucleic Acid: (SEQ ID NO: 201):
GAATGTCCTCCTTGTCCTGCTCCTCCAGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACAGCACCTACAGAGTGGTGTCCGTGCTGAC
CGTGCTGCACCAGGATTGGCTGAACGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTTCCGATAT
CGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCTCTGTGATGCACGAGGCCCTGCACAACCAC
TACACCCAGAAGTCTCTGAGCCTGTCTCTCGGAAA
AGGCGGAGGCGGAGCTGGTGGTGGCGGAGCAGGCG
GCGGTGCTGGCGGCGGAGGATCTGAAGATTGCAAT
GAGCTGCCTCCTCGGCGGAACACAGAGATCTTGAC
AGGCTCTTGGAGCGACCAGACATACCCTGAGGGCA
CCCAGGCCATCTACAAGTGTAGACCTGGCTACCGC
AGCCTGGGCAATGTGATCATGGTCTGCAGAAAAGG
CGAGTGGGTCGCCCTGAATCCTCTGAGAAAGTGCC
AGAAGAGGCCTTGCGGACACCCCGGCGATACACCT
TTTGGCACATTCACCCTGACCGGCGGCAATGTGTT
TGAGTATGGCGTGAAGGCCGTGTACACCTGTAACG
AGGGATATCAGCTGCTGGGCGAGATCAACTACAGA
GAGTGTGATACCGACGGCTGGACCAACGACATCCC
TATCTGCGAGGTGGTCAAGTGCCTGCCTGTGACAG
CCCCTGAGAATGGCAAGATCGTGTCCAGCGCCATG
GAACCCGACAGAGAGTATCACTTTGGCCAGGCCGT
CAGATTCGTGTGCAACAGCGGCTATAAGATCGAGG
GCGACGAGGAAATGCACTGCAGCGACGACGGCTTC
TGGTCCAAAGAAAAGCCCAAATGCGTGGAAATCAG
CTGCAAGAGCCCCGACGTGATCAACGGCAGCCCTA
TCAGCCAGAAGATCATCTACAAAGAGAACGAGCGG
TTCCAGTATAAGTGCAACATGGGCTACGAGTACAG
CGAGCGGGGAGATGCCGTGTGTACAGAATCTGGAT
GGCGGCCTCTGCCTAGCTGCGAGGAAAAGTCT
Compound AL:
Amino Acid (SEQ ID NO: 157):
CVECPPCPAPPVAGPSVFLFPPKPKDTLMISRTPE
VTCVVVDVSQEDPEVQFNWYVDGVEVHNAKTKPRE
EQFNSTYRVVSVLTVLHQDWLNGKEYKCKVSNKGL
PSSIEKTISKAKGQPREPQVYTLPPSQEEMTKNQV
SLTCLVKGFYPSDIAVEWESNGQPENNYKTTPPVL
DSDGSFFLYSRLTVDKSRWQEGNVFSCSVMHEALH
NHYTQKSLSLSLGKGGGGAGGGGAGGGAGGGGSKE
DCNELPPRRNTEILTGSWSDQTYPEGTQAIYKCRP
GYRSLGNVIMVCRKGEWVALNPLRKCQKRPCGHPG
DTPFGTFTLTGGNVFEYGVKAVYTCNEGYQLLGEI
NYRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KS
Nucleic Acid: (SEQ ID NO: 202):
GAATGTCCTCCTTGTCCTGCTCCTCCAGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACAGCACCTACAGAGTGGTGTCCGTGCTGAC
CGTGCTGCACCAGGATTGGCTGAACGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTTCCGATAT
CGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCTCTGTGATGCACGAGGCCCTGCACAACCAC
TACACCCAGAAGTCTCTGAGCCTGTCTCTCGGAAA
AGGCGGAGGCGGAGCTGGTGGTGGCGGAGCAGGCG
GCGGTGCTGGCGGCGGAGGATCTAAAGAAGATTGC
AACGAGCTGCCTCCTCGGCGGAATACCGAGATTCT
GACAGGCTCTTGGAGCGACCAGACATACCCTGAGG
GCACCCAGGCCATCTACAAGTGTAGACCTGGCTAC
CGCAGCCTGGGCAATGTGATCATGGTCTGCAGAAA
AGGCGAGTGGGTCGCCCTGAATCCTCTGAGAAAGT
GCCAGAAGAGGCCTTGCGGACACCCCGGCGATACA
CCTTTTGGCACATTCACCCTGACCGGCGGCAATGT
GTTTGAGTATGGCGTGAAGGCCGTGTACACCTGTA
ACGAGGGATATCAGCTGCTGGGCGAGATCAACTAC
AGAGAGTGTGATACCGACGGCTGGACCAACGACAT
CCCTATCTGCGAGGTGGTCAAGTGCCTGCCTGTGA
CAGCCCCTGAGAATGGCAAGATCGTGTCCAGCGCC
ATGGAACCCGACAGAGAGTATCACTTTGGCCAGGC
CGTCAGATTCGTGTGCAACAGCGGCTATAAGATCG
AGGGCGACGAGGAAATGCACTGCAGCGACGACGGC
TTCTGGTCCAAAGAAAAGCCCAAATGCGTGGAAAT
CAGCTGCAAGAGCCCCGACGTGATCAACGGCAGCC
CTATCAGCCAGAAGATCATCTACAAAGAGAACGAG
CGGTTCCAGTATAAGTGCAACATGGGCTACGAGTA
CAGCGAGCGGGGAGATGCCGTGTGTACAGAATCTG
GATGGCGGCCTCTGCCTAGCTGCGAGGAAAAGTCT
Compound AM:
Amino Acid (SEQ ID NO: 158):
CVECPPCPAPPVAGPSVFLFPPKPKDTLMISRTPE
VTCVVVDVSQEDPEVQFNWYVDGVEVHNAKTKPRE
EQFNSTYRVVSVLTVLHQDWLNGKEYKCKVSNKGL
PSSIEKTISKAKGQPREPQVYTLPPSQEEMTKNQV
SLTCLVKGFYPSDIAVEWESNGQPENNYKTTPPVL
DSDGSFFLYSRLTVDKSRWQEGNVFSCSVMHEALH
NHYTQKSLSLSLGKGGGGAGGGGAGGGAGGGGSRE
DCNELPPRRNTEILTGSWSDQTYPEGTQAIYKCRP
GYRSLGNVIMVCRKGEWVALNPLRKCQKRPCGHPG
DTPFGTFTLTGGNVFEYGVKAVYTCNEGYQLLGEI
NYRECDTDGWTNDIPICEVVKCLPVTAPENGKIVS
SAMEPDREYHFGQAVRFVCNSGYKIEGDEEMHCSD
DGFWSKEKPKCVEISCKSPDVINGSPISQKIIYKE
NERFQYKCNMGYEYSERGDAVCTESGWRPLPSCEE
KS
Nucleic Acid: (SEQ ID NO: 203):
GAATGTCCTCCTTGTCCTGCTCCTCCAGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACAGCACCTACAGAGTGGTGTCCGTGCTGAC
CGTGCTGCACCAGGATTGGCTGAACGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTTCCGATAT
CGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCTCTGTGATGCACGAGGCCCTGCACAACCAC
TACACCCAGAAGTCTCTGAGCCTGTCTCTCGGAAA
AGGCGGAGGCGGAGCTGGTGGTGGCGGAGCAGGCG
GCGGTGCTGGCGGCGGAGGATCTCGGGAAGATTGC
AACGAGCTGCCTCCTCGGCGGAATACCGAGATTCT
GACAGGCTCTTGGAGCGACCAGACATACCCTGAGG
GCACCCAGGCCATCTACAAGTGTAGACCTGGCTAC
CGCAGCCTGGGCAATGTGATCATGGTCTGCAGAAA
AGGCGAGTGGGTCGCCCTGAATCCTCTGAGAAAGT
GCCAGAAGAGGCCTTGCGGACACCCCGGCGATACA
CCTTTTGGCACATTCACCCTGACCGGCGGCAATGT
GTTTGAGTATGGCGTGAAGGCCGTGTACACCTGTA
ACGAGGGATATCAGCTGCTGGGCGAGATCAACTAC
AGAGAGTGTGATACCGACGGCTGGACCAACGACAT
CCCTATCTGCGAGGTGGTCAAGTGCCTGCCTGTGA
CAGCCCCTGAGAATGGCAAGATCGTGTCCAGCGCC
ATGGAACCCGACAGAGAGTATCACTTTGGCCAGGC
CGTCAGATTCGTGTGCAACAGCGGCTATAAGATCG
AGGGCGACGAGGAAATGCACTGCAGCGACGACGGC
TTCTGGTCCAAAGAAAAGCCCAAATGCGTGGAAAT
CAGCTGCAAGAGCCCCGACGTGATCAACGGCAGCC
CTATCAGCCAGAAGATCATCTACAAAGAGAACGAG
CGGTTCCAGTATAAGTGCAACATGGGCTACGAGTA
CAGCGAGCGGGGAGATGCCGTGTGTACAGAATCTG
GATGGCGGCCTCTGCCTAGCTGCGAGGAAAAGTCT
Compound AN:
Amino Acid (SEQ ID NO: 159):
CVECPPCPAPPVAGPSVFLFPPKPKDTLMISRTPE
VTCVVVDVSQEDPEVQFNWYVDGVEVHNAKTKPRE
EQFNSTYRVVSVLTVLHQDWLNGKEYKCKVSNKGL
PSSIEKTISKAKGQPREPQVYTLPPSQEEMTKNQV
SLTCLVKGFYPSDIAVEWESNGQPENNYKTTPPVL
DSDGSFFLYSRLTVDKSRWQEGNVFSCSVMHEALH
NHYTQKSLSLSLGKGGGGAGGGAGGGGSKEDCNEL
PPRRNTEILTGSWSDQTYPEGTQAIYKCRPGYRSL
GNVIMVCRKGEWVALNPLRKCQKRPCGHPGDTPFG
TFTLTGGNVFEYGVKAVYTCNEGYQLLGEINYREC
DTDGWTNDIPICEVVKCLPVTAPENGKIVSSAMEP
DREYHFGQAVRFVCNSGYKIEGDEEMHCSDDGFWS
KEKPKCVEISCKSPDVINGSPISQKIIYKENERFQ
YKCNMGYEYSERGDAVCTESGWRPLPSCEEKS
Nucleic Acid: (SEQ ID NO: 204):
GAATGTCCTCCTTGTCCTGCTCCTCCAGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACAGCACCTACAGAGTGGTGTCCGTGCTGAC
CGTGCTGCACCAGGATTGGCTGAACGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTTCCGATAT
CGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCTCTGTGATGCACGAGGCCCTGCACAACCAC
TACACCCAGAAGTCTCTGAGCCTGTCTCTCGGAAA
AGGCGGAGGCGGAGCTGGTGGTGGTGCTGGCGGCG
GAGGATCTAAAGAAGATTGCAACGAGCTGCCTCCT
CGGCGGAATACCGAGATTCTGACAGGCTCTTGGAG
CGACCAGACATACCCTGAGGGCACCCAGGCCATCT
ACAAGTGTAGACCTGGCTACCGCAGCCTGGGCAAT
GTGATCATGGTCTGCAGAAAAGGCGAGTGGGTCGC
CCTGAATCCTCTGAGAAAGTGCCAGAAGAGGCCTT
GCGGACACCCCGGCGATACACCTTTTGGCACATTC
ACCCTGACCGGCGGCAATGTGTTTGAGTATGGCGT
GAAGGCCGTGTACACCTGTAACGAGGGATATCAGC
TGCTGGGCGAGATCAACTACAGAGAGTGTGATACC
GACGGCTGGACCAACGACATCCCTATCTGCGAGGT
GGTCAAGTGCCTGCCTGTGACAGCCCCTGAGAATG
GCAAGATCGTGTCCAGCGCCATGGAACCCGACAGA
GAGTATCACTTTGGCCAGGCCGTCAGATTCGTGTG
CAACAGCGGCTATAAGATCGAGGGCGACGAGGAAA
TGCACTGCAGCGACGACGGCTTCTGGTCCAAAGAA
AAGCCCAAATGCGTGGAAATCAGCTGCAAGAGCCC
CGACGTGATCAACGGCAGCCCTATCAGCCAGAAGA
TCATCTACAAAGAGAACGAGCGGTTCCAGTATAAG
TGCAACATGGGCTACGAGTACAGCGAGCGGGGAGA
TGCCGTGTGTACAGAATCTGGATGGCGGCCTCTGC
CTAGCTGCGAGGAAAAGTCT
Compound AO:
Amino Acid (SEQ ID NO: 160):
CVECPPCPAPPVAGPSVFLFPPKPKDTLMISRTPE
VTCVVVDVSQEDPEVQFNWYVDGVEVHNAKTKPRE
EQFNSTYRVVSVLTVLHQDWLNGKEYKCKVSNKGL
PSSIEKTISKAKGQPREPQVYTLPPSQEEMTKNQV
SLTCLVKGFYPSDIAVEWESNGQPENNYKTTPPVL
DSDGSFFLYSRLTVDKSRWQEGNVFSCSVMHEALH
NHYTQKSLSLSLGKGGGGAGGGAGGGGSREDCNEL
PPRRNTEILTGSWSDQTYPEGTQAIYKCRPGYRSL
GNVIMVCRKGEWVALNPLRKCQKRPCGHPGDTPFG
TFTLTGGNVFEYGVKAVYTCNEGYQLLGEINYREC
DTDGWTNDIPICEVVKCLPVTAPENGKIVSSAMEP
DREYHFGQAVRFVCNSGYKIEGDEEMHCSDDGFWS
KEKPKCVEISCKSPDVINGSPISQKIIYKENERFQ
YKCNMGYEYSERGDAVCTESGWRPLPSCEEKS
Nucleic Acid: (SEQ ID NO: 205):
GAATGTCCTCCTTGTCCTGCTCCTCCAGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTTTCCCAAGAGGATCCCGA
GGTGCAGTTCAATTGGTACGTGGACGGCGTGGAAG
TGCACAACGCCAAGACCAAGCCTAGAGAGGAACAG
TTCAACAGCACCTACAGAGTGGTGTCCGTGCTGAC
CGTGCTGCACCAGGATTGGCTGAACGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTTTACACCCTGCCTCCAA
GCCAAGAGGAAATGACCAAGAACCAGGTGTCCCTG
ACCTGCCTGGTCAAGGGCTTCTACCCTTCCGATAT
CGCCGTGGAATGGGAGAGCAATGGCCAGCCTGAGA
ACAACTACAAGACCACACCTCCTGTGCTGGACAGC
GACGGCAGCTTTTTTCTGTACTCCCGCCTGACCGT
GGACAAGAGCAGATGGCAAGAGGGCAACGTGTTCA
GCTGCTCTGTGATGCACGAGGCCCTGCACAACCAC
TACACCCAGAAGTCTCTGAGCCTGTCTCTCGGAAA
AGGCGGAGGCGGAGCTGGTGGTGGTGCTGGCGGCG
GAGGATCTCGGGAAGATTGCAACGAGCTGCCTCCT
CGGCGGAATACCGAGATTCTGACAGGCTCTTGGAG
CGACCAGACATACCCTGAGGGCACCCAGGCCATCT
ACAAGTGTAGACCTGGCTACCGCAGCCTGGGCAAT
GTGATCATGGTCTGCAGAAAAGGCGAGTGGGTCGC
CCTGAATCCTCTGAGAAAGTGCCAGAAGAGGCCTT
GCGGACACCCCGGCGATACACCTTTTGGCACATTC
ACCCTGACCGGCGGCAATGTGTTTGAGTATGGCGT
GAAGGCCGTGTACACCTGTAACGAGGGATATCAGC
TGCTGGGCGAGATCAACTACAGAGAGTGTGATACC
GACGGCTGGACCAACGACATCCCTATCTGCGAGGT
GGTCAAGTGCCTGCCTGTGACAGCCCCTGAGAATG
GCAAGATCGTGTCCAGCGCCATGGAACCCGACAGA
GAGTATCACTTTGGCCAGGCCGTCAGATTCGTGTG
CAACAGCGGCTATAAGATCGAGGGCGACGAGGAAA
TGCACTGCAGCGACGACGGCTTCTGGTCCAAAGAA
AAGCCCAAATGCGTGGAAATCAGCTGCAAGAGCCC
CGACGTGATCAACGGCAGCCCTATCAGCCAGAAGA
TCATCTACAAAGAGAACGAGCGGTTCCAGTATAAG
TGCAACATGGGCTACGAGTACAGCGAGCGGGGAGA
TGCCGTGTGTACAGAATCTGGATGGCGGCCTCTGC
CTAGCTGCGAGGAAAAGTCT
Compound AP:
Amino Acid (SEQ ID NO: 161):
VECPPCPAPPVAGPSVFLFPPKPKDTLMISRTPEV
TCVVVDVSQEDPEVQFNWYVDGVEVHNAKTKPREE
QFNSTYRVVSVLTVLHQDWLNGKEYKCKVSNKGLP
SSIEKTISKAKGQPREPQVYTLPPSQEEMTKNQVS
LTCLVKGFYPSDIAVEWESNGQPENNYKTTPPVLD
SDGSFFLYSRLTVDKSRWQEGNVFSCSVMHEALHN
HYTQKSLSLSLGKGGGGAGGGGAGGGAGGGGSEDC
NELPPRRNTEILTGSWSDQTYPEGTQAIYKCRPGY
RSLGNVIMVCRKGEWVALNPLRKCQKRPCGHPGDT
PFGTFTLTGGNVFEYGVKAVYTCNEGYQLLGEINY
RECDTDGWTNDIPICEVVKCLPVTAPENGKIVSSA
MEPDREYHFGQAVRFVCNSGYKIEGDEEMHCSDDG
FWSKEKPKCVEISCKSPDVINGSPISQKIIYKENE
RFQYKCNMGYEYSERGDAVCTESGWRPLPSCEEKS
Nucleic Acid: (SEQ ID NO: 206):
GTTGAATGTCCTCCATGTCCTGCTCCTCCAGTGGC
CGGACCTTCCGTGTTTCTGTTCCCTCCAAAGCCTA
AGGACACCCTGATGATCAGCAGAACCCCTGAAGTG
ACCTGCGTGGTGGTGGACGTGTCCCAAGAGGACCC
TGAGGTGCAGTTCAATTGGTACGTGGACGGCGTGG
AAGTGCACAACGCCAAGACCAAGCCTAGAGAGGAA
CAGTTCAACAGCACCTACAGAGTGGTGTCCGTGCT
GACCGTGCTGCACCAGGATTGGCTGAACGGCAAAG
AGTACAAGTGCAAGGTGTCCAACAAGGGCCTGCCT
AGCAGCATCGAGAAAACCATCTCTAAGGCCAAGGG
CCAGCCTCGCGAACCTCAGGTTTACACCCTGCCTC
CAAGCCAAGAGGAAATGACCAAGAACCAGGTGTCC
CTGACCTGCCTGGTCAAGGGCTTTTACCCCTCCGA
TATCGCCGTGGAATGGGAGAGCAACGGCCAGCCTG
AGAACAACTACAAGACCACACCTCCTGTGCTGGAC
AGCGACGGCAGCTTTTTTCTGTACTCCCGCCTGAC
CGTGGACAAGAGCAGATGGCAAGAGGGCAACGTGT
TCAGCTGTAGCGTGATGCACGAGGCCCTGCACAAC
CACTACACCCAGAAGTCTCTGAGCCTGTCTCTCGG
AAAAGGCGGAGGTGGTGCTGGCGGAGGCGGAGCAG
GAGGTGGTGCAGGCGGCGGAGGATCTGAAGATTGC
AACGAGCTGCCTCCTCGGCGGAATACCGAGATTCT
GACAGGCTCTTGGAGCGACCAGACATACCCTGAGG
GCACCCAGGCCATCTACAAGTGTAGACCTGGCTAC
CGCAGCCTGGGCAATGTGATCATGGTCTGCAGAAA
AGGCGAGTGGGTCGCCCTGAATCCTCTGAGAAAGT
GCCAGAAGAGGCCTTGCGGACACCCAGGCGATACC
CCTTTTGGCACATTCACCCTGACCGGCGGCAATGT
GTTTGAGTACGGCGTGAAGGCCGTGTACACCTGTA
ATGAGGGCTACCAGCTGCTGGGCGAGATCAACTAC
AGAGAGTGTGACACCGACGGCTGGACCAACGACAT
CCCTATCTGCGAGGTGGTCAAGTGCCTGCCTGTGA
CAGCCCCTGAGAATGGCAAGATCGTGTCCAGCGCC
ATGGAACCCGATAGAGAGTACCACTTCGGCCAGGC
CGTCAGATTCGTGTGCAACAGCGGCTACAAGATCG
AGGGCGACGAGGAAATGCACTGCAGCGACGACGGC
TTCTGGTCCAAAGAAAAGCCCAAATGCGTGGAAAT
CAGCTGCAAGAGCCCCGACGTGATCAACGGCAGCC
CCATCAGCCAGAAGATCATCTACAAAGAGAACGAG
CGGTTCCAGTATAAGTGCAACATGGGCTACGAGTA
CAGCGAGAGGGGCGACGCCGTGTGTACAGAATCTG
GATGGCGGCCTCTGCCTAGCTGCGAAGAGAAGTCC
Compound AQ:
Amino Acid (SEQ ID NO: 162):
ISCGSPPPILNGRISYYSTPIAVGTVIRYSCSGTF
RLIGEKSLLCITKDKVDGTWDKPAPKCEYFNKYSS
CPEPIVPGGYKIRGSTPYRHGDSVTFACKTNFSMN
GQKSVWCQANNMWGPTRLPTCVSVFPGGGGSDAAV
ECPPCPAPPVAGPSVFLFPPKPKDTLMISRTPEVT
CVVVDVSQEDPEVQFNWYVDGVEVHNAKTKPREEQ
FNSTYRVVSVLTVLHQDWLNGKEYKCKVSNKGLPS
SIEKTISKAKGQPREPQVYTLPPSQEEMTKNQVSL
TCLVKGFYPSDIAVEWESNGQPENNYKTTPPVLDS
DGSFFLYSRLTVDKSRWQEGNVFSCSVMHEALHNH
YTQKSLSLSLGK
Nucleic Acid: (SEQ ID NO: 207):
ATCTCTTGTGGCTCTCCACCTCCTATCCTGAACGG
CCGGATCAGCTACTACAGCACCCCTATCGCTGTGG
GCACCGTGATCAGATACAGCTGCAGCGGCACCTTC
CGGCTGATCGGAGAGAAGTCCCTGCTGTGCATCAC
CAAGGACAAGGTGGACGGCACCTGGGACAAGCCTG
CTCCTAAGTGCGAGTACTTCAACAAGTACAGCAGC
TGCCCCGAGCCTATCGTGCCTGGCGGCTATAAGAT
CAGAGGCAGCACCCCATACAGACACGGCGACAGCG
TGACCTTTGCCTGCAAGACCAACTTCAGCATGAAC
GGCCAGAAAAGCGTGTGGTGCCAGGCCAACAACAT
GTGGGGACCTACCAGACTGCCCACCTGTGTGTCAG
TGTTTCCAGGCGGCGGAGGATCTGATGCCGCTGTG
GAATGTCCTCCTTGTCCAGCTCCTCCAGTGGCCGG
ACCTTCCGTGTTTCTGTTCCCTCCAAAGCCTAAGG
ACACCCTGATGATCAGCAGAACCCCTGAAGTGACC
TGCGTGGTGGTGGACGTGTCCCAAGAGGATCCTGA
GGTGCAGTTCAATTGGTACGTGGACGGCGTGGAAG
TGCACAACGCCAAGACCAAGCCTAGAGAGGAACAG
TTCAACAGCACCTACAGAGTGGTGTCCGTGCTGAC
CGTGCTGCACCAGGATTGGCTGAACGGCAAAGAGT
ACAAGTGCAAGGTGTCCAACAAGGGCCTGCCTAGC
AGCATCGAGAAAACCATCAGCAAGGCCAAGGGCCA
GCCAAGAGAACCCCAGGTGTACACACTGCCTCCAA
GCCAAGAGGAAATGACCAAGAACCAGGTGTCCCTG
ACCTGCCTGGTCAAGGGCTTCTACCCTTCCGATAT
CGCCGTGGAATGGGAGAGCAATGGCCAGCCTGAGA
ACAACTACAAGACCACACCTCCTGTGCTGGACAGC
GACGGCTCATTCTTCCTGTACAGCAGACTGACCGT
GGACAAGAGCAGATGGCAAGAGGGCAACGTGTTCA
GCTGCTCCGTGATGCACGAGGCCCTGCACAACCAC
TACACCCAGAAGTCTCTGAGCCTGAGCCTGGGCAA
G

1. A fusion protein having the structure, from N-terminus to C-terminus:
D1-L1-Fc-L2-D2,
wherein D1 comprises a fragment of complement factor H (FH) and/or a fragment of CR2;
L1 is absent or is an amino acid sequence of at least one amino acid;
Fc is an Fc domain, such as an Fc receptor binding domain;
L2 is absent or is an amino acid sequence of at least one amino acid; and
D2 comprises a fragment of FH and/or a fragment of CR2,
wherein D1 and D2 cannot both comprise a fragment of CR2.

2. The fusion protein of claim 1, wherein
(a) the fragment of FH of D1 comprises one or more FH short consensus repeat (SCR) domains, wherein the one or more SCR domains are selected from the group consisting of SCR 1, 2, 3, 4, 5, 19, and 20,
(b) the fragment of FH of D2 comprises one or more FH SCR domains,
wherein the one or more SCR domains are selected from the group consisting of SCR 1, 2, 3, 4, 5, 19, and 20,
(c) the fraament of CR2 of D1 comprises one or more CR2 SCR domains, wherein the one or more SCR domains are selected from the aroup consistina of SCR 1, 2, 3, and 4, and/or
(d) the fraament of CR2 of D2 comorises one or more CR2 SCR domains, wherein the one or more SCR domains are selected from the group consisting of SCR 1, 2, 3, and 4.

3. The fusion protein of claim 2, wherein the FH SCR domains are selected from the group consisting of SCR 1-4; 1-5; 1-4, 19, and 20; 1-5, 19, and 20; or 19 and 20 and/or the CR2 SCR domains are selected from the aroup consistina of: SCR 1-2, 1-3, or 1-4.

4-5. (canceled)

6. The fusion protein of claim 1, wherein D1 or D2 comprises a fragment of FH fused by L3 to a fragment of FH or CR2, wherein L3 is an amino acid sequence of at least one amino acid.

7-8. (canceled)

9. The fusion protein of claim 6, wherein the fragment of FH comprises SCR domains 19 and 20, the fragment of CR2 comprises SCR domains 1-2, and/or L3 is selected from the group consisting of: (G4A)2G4S, G4SDAA, GGGGAGGGGAGGGGS, GGGGSGGGGSGGGGS, G4S, (G4S)2, (G4S)3, (G4S)4, (G4S)5, (G4S)6, (EAAAK)3, PAPAP, G4SPAPAP, PAPAPG4S, GSTSGKSSEGKG, (GGGDS)2, (GGGES)2, GGGDSGGGGS, GGGASGGGGS, GGGESGGGGS, ASTKGP, ASTKGPSVFPLAP, G3P, G7P, PAPNLLGGP, G6, G12, APELPGGP, SEPQPQPG, (G3S2)3, GGGGGGGGGSGGGS, GGGGSGGGGGGGGGS, (GGSSS)3, (GS4)3, G4A(G4S)2, G4SG4AG4S, G3AS(G4S)2, G4SG3ASG4S, G4SAG3SG4S, (G4S)2AG3S, G4SAG3SAG3S, G4D(G4S)2, G4SG4DG4S, (G4D)2G4S, G4E(G4S)2, G4SG4EG4S, (G4E)2G4S, and G4SDA.

10-20. (canceled)

21. The fusion protein of claim 1, wherein:
(a) D1 comprises CR2 domains 1-2, wherein CR2 SCR 2 includes an N107Q substitution; L1 comprises G4SDAA; Fc comprises IgG2-G4 Fc; L2 comprises (G4A)2G3AG4S; and D2 comprises FH SCRs 1-4;
(b) D1 comprises CR2 domains 1-2, wherein CR2 SCR 2 includes an N107Q substitution; L1 comprises G4SDAA; Fc comprises IgG2-G4 Fc; L2 comprises (G4A)2G3AG4S; and D2 FH SCRs 1-5;
(c) D1 comprises CR2 SCR domains 1 and 2, wherein CR2 SCR 2 includes an N107Q substitution; L1 comprises G4SDAA; Fc comprises FLlgG2-G4 Fc; L2 comprises (G4A)2G3AG4S; and D2 comprises FH SCRs 1-4;
(d) D1 comprises FH SCR domains 1-5; L1 is absent; Fc comprises IgG2-G4 Fc; L2 is absent; and D2 comprises FH SCRs 19 and 20;
(e) D1 comprises FH SCR domains 1-5; L1 comprises (G4A)2G4S; Fc comprises IgG2-G4 Fc; L2 is absent; and D2 comprises FH SCRs 19 and 20;
(f) D1 comprises FH SCR domains 1-5; L1 is absent; comprises IgG2-G4 Fc; L2 comprises (G4A)2G4S; and D2 comprises FH SCRs 19 and 20;
(g) D1 comprises FH SCR domains 1-5; comprises (G4A)2G4S; Fc comprises IgG2-G4 Fc; L2 comprises (G4A)2G4S; and D2 comprises FH SCRs 19 and 20;
(h) D1 comprises FH SCR domains 19 and 20; L1 is absent; Fc comprises IgG2-G4 Fc; L2 is absent;
and D2 comprises FH SCRs 1-5;
(i) D1 comprises CR2 SCR domains 1-4; L1 comprises (G4A)2G4S; Fc comprises IgG2-G4 Fc; L2 comprises (G4A)2G4S; and D2 comprises FH SCRs 1-5;
(j) D1 comprises CR2 SCR domains 1-4, wherein CR2 SCR 2 comprises an N107Q substitution; L1 comprises (G4A)2G4S; Fc comprises IgG2-G4 Fc; L2 comprises (G4A)2G4S; and D2 comprises FH SCRs 1-5;
(k) D1 comprises CR2 SCR domains 1-4, wherein CR2 SCR 2 comprises a S109A substitution; L1 comprises (G4A)2G4S; Fc comprises IgG2-G4 Fc; L2 comprises (G4A)2G4S; and D2 comprises FH SCRs 1-5;
(l) D1 comprises CR2 SCR domains 1-4; L1 comprises G4SDAA; Fc comprises IgG2-G4 Fc; L2 comprises (G4S)4; and D2 comprises FH SCRs 1-5;
(m) D1 comprises CR2 SCR domains 1-4; L1 comprises G4SDAA; Fc comprises IgG2-G4 Fc; L2 comprises (G4S)2; and D2 comprises FH SCRs 1-5;
(n) D1 comprises CR2 SCR domains 1-4; L1 comprises G4SDAA; Fc comprises IgG2-G4 Fc; L2 comprises G4S; and D2 comprises FH SCRs 1-5;
(o) D1 comprises CR2 SCR domains 1-4; L1 is absent; Fc comprises IgG2-G4 Fc; L2 is absent; and D2 comprises FH SCRs 1-5;
(p) D1 comprises CR2 SCR domains 1-4; L1 is absent; Fc comprises IgG2-G4 Fc; L2 comprises (G4A)2G4S; and D2 comprises FH SCRs 1-5;
(q) D1 comprises CR2 SCR domains 1-4, wherein CR2 SCR 2 includes an N107Q substitution; L1 comprises G4SDAA; Fc comprises IgG2-G4 Fc; L2 comprises (G4A)2G3AG4S; and D2 comprises FH SCRs 1-5;
(r) D1 comprises CR2 SCR domains 1-4, wherein CR2 SCR 2 includes an N107Q substitution; L1 is absent; Fc comprises IgG2-G4 Fc; L2 comprises (G4A)2G3AG4S; and D2 comprises FH SCRs 1-5;
(s) D1 comprises CR2 SCR domains 1-2, wherein CR2 SCR 2 includes an N107Q substitution, wherein CR2 SCR 2 includes an N107Q substitution; L1 is absent; Fc comprises IgG2-G4 Fc; L2 comprises (G4A)2G3AG4S; and D2 comprises FH SCRs 1-5;
(t) D1 comprises CR2 SCRs 1-4, wherein CR2 SCR 2 includes an N107Q substitution; L1 comprises G4SDA; Fc comprises IgG2-G4 Fc; L2 comprises (G4A)2G3AG4S; and D2 comprises FH SCRs 1-4;
(u) D1 comprises CR2 SCRs 1-4, wherein CR2 SCR 2 includes an N107Q substitution; L1 is absent; Fc comprises IgG2-G4 Fc; L2 comprises (G4A)2G3AG4S; and D2 comprises FH SCRs 1-4;
(v) D1 comprises CR2 SCRs 1-2, wherein CR2 SCR 2 includes an N107Q substitution; L1 is absent; Fc comprises IgG2-G4 Fc; L2 comprises (G4A)2G3AG4S; and D2 comprises FH SCRs 1-4; or
(w) D1 comprises FH SCRs 19-20; L1 (G4A)2G4S; Fc comprises IgG2-G4 Fc; L2 (G4A)2G4S; and D2 comprises FH SCRs 1-4.

22. The fusion protein of claim 1, wherein the fusion protein comprises the amino acid sequence of any one of SEQ ID NOs: 114-124, 132, 144, 145, 147, 148, 152-155, 209, 210-215 or a variant thereof with up to 85% sequence identity thereto or with up to 10 amino acid substitutions, additions, or deletions.

23. (canceled)

24. A fusion protein comprising (a) a moiety comprising a fragment of complement receptor 2 (CR2); (b) a moiety comprising a fragment of complement factor H (FH); and (c) an anti-albumin VHH domain, wherein optionally (a), (b), and/or (c) may be fused by a linker.

25-49. (canceled)

50. The fusion protein of claim 1, wherein SCR2 of the fragment of CR2 comprises an N101Q substitution, an N107Q substitution, and/or a S109A substitution.

51. (canceled)

52. The fusion protein of claim 1, wherein the Fc domain comprises an Fc domain from a human immunoglobulin, is a chimeric Fc domain, or is a human immunoglobulin is selected from the group consisting of IgG1, IgG2, IgG3, and IgG4.

53-56. (canceled)

57. The fusion protein of claim 1, wherein L1 and/or L2 are selected from the group consisting of: (G₄A)₂G₃AG₄S, G₄SDAA, (G₄A)₂G₄S, G₄AG₃AG₄S, GGGGAGGGGAGGGGS, GGGGSGGGGSGGGGS, G₄S, (G₄S)₂, (G₄S)₃, (G₄S)₄, (G₄S)₅, (G₄S)₆, (EAAAK)₃, PAPAP, G₄SPAPAP, PAPAPG₄S, GSTSGKSSEGKG, (GGGDS)₂, (GGGES)₂, GGGDSGGGGS, GGGASGGGGS, GGGESGGGGS, ASTKGP, ASTKGPSVFPLAP, G₃P, G₇P, PAPNLLGGP, G₆, G₁₂, APELPGGP, SEPQPQPG, (G₃S₂)₃, GGGGGGGGGSGGGS, GGGGSGGGGGGGGGS, (GGSSS)₃, (GS₄)₃, G₄A(G₄S)₂, G₄SG₄AG4S, G3AS(G4S)2, G4SG3ASG4S, G4SAG3SG4S, (G4S)2AG3S, G4SAG3SAG3S, G4D(G4S)2, G4SG4DG4S, (G4D)2G4S, G4E(G4S)2, G4SG4EG4S, (G4E)2G4S, G4SDA, G4A, and (G4A)3.

58-75. (canceled)

76. A pharmaceutical composition comprising the fusion protein of claim 1 and a pharmaceutically acceptable carrier.

77. A nucleic acid or polynucleotide encoding the fusion protein of claim 1.

78. A vector comprising the nucleic acid of claim 77.

79. A host cell comprising the polynucleotide of claim 77 or a vector encoding the polynucleotide.

80. (canceled)

81. A method of producing the fusion protein of claim 1, comprising the steps of culturing one or more host cells comprising one or more nucleic acid molecules capable of expressing the fusion protein under conditions suitable for expression of the fusion protein, optionally wherein the method further comprises the step of obtaining the fusion protein from the cell culture or culture medium.

82. (canceled)

83. A method inhibiting the alternative complement pathway comprising administering the pharmaceutical composition of claim 76 to a subject in need thereof.

84-86. (canceled)

87. The method of claim 83, wherein the fusion protein is formulated for:
(a) daily, weekly, or monthly administration,
(b) intravenous, subcutaneous, intramuscular, oral, nasal, sublingual, intrathecal, and intradermal administration,
(c) administration at a dosage of between about 0.1 mg/kg to about 150 mg/kg, or
(d) administration in combination with an additional therapeutic agent.

88-89. (canceled)

90. The method of claim 83, wherein the subject has a disease mediated by alternate complement pathway dysregulation, wherein the disease is selected from the group consisting of paroxysmal nocturnal hemoglobinuria (PNH), atypical hemolytic uremic syndrome (aHUS), IgA nephrology, lupus nephritis, C3 glomerulopathy (C3G), dermatomyositis, systemic sclerosis, demyelinating polyneuropathy, pemphigus, membranous nephropathy, focal segmental glomerular sclerosis (FSGS), bullous pemphigoid, epidermolysis bullosa acquisita (EBA), ANCA vasculitis, hypocomplementemic urticarial vasculitis, immune complex small vessel vasculitis, an autoimmune necrotizing myopathy, rejection of a transplanted organ, antiphospholipid (aPL) Ab syndrome, glomerulonephritis, asthma, dense deposit disease (DDD), age related macular degeneration (AMD), systemic lupus erythematosus (SLE), rheumatoid arthritis (RA), multiple sclerosis (MS), traumatic brain injury (TBI), ischemia reperfusion injury, preeclampsia, and thrombic thrombocytopenic purpura (TTP).

91. The method of claim 83, wherein the subject is a mammal.

92. The method of claim 91, wherein the mammal is a human.

93. A kit comprising the fusion protein of claim 1 and, optionally, instructions for administering an effective amount of the fusion protein to a subject in need thereof.

94-104. (canceled)
